SEQUENCE LISTING (181 sequences)

SEQ ID NO: 1 - protein, 7096 aa, unidentified [sequence omitted]

SEQ ID NO: 2 - protein, 9 aa, Homo sapiens
VMVELVAEL

SEQ ID NO: 3 - protein, 9 aa, Homo sapiens
YLATALLTL

SEQ ID NO: 4 - protein, 9 aa, Homo sapiens
CLEASFNYL

SEQ ID NO: 5 - protein, 9 aa, Homo sapiens
WLMWLIINL

SEQ ID NO: 6 - protein, 9 aa, Homo sapiens
SLPGVFCGV

SEQ ID NO: 7 - protein, 9 aa, Homo sapiens
FLLNKEMYL

SEQ ID NO: 8 - protein, 9 aa, Homo sapiens
SMWALIISV

SEQ ID NO: 9 - protein, 9 aa, Homo sapiens
YLASGGQPI

SEQ ID NO: 10 - protein, 9 aa, Homo sapiens
KLSYGIATV

SEQ ID NO: 11 - protein, 9 aa, Homo sapiens
YLDAYNMMI

SEQ ID NO: 12 - protein, 9 aa, Homo sapiens
LLLDDFVEI

SEQ ID NO: 13 - protein, 9 aa, Homo sapiens
FVFLVLLPL

SEQ ID NO: 14 - protein, 9 aa, Homo sapiens
SIIAYTMSL

SEQ ID NO: 15 - protein, 9 aa, Homo sapiens
ALNTLVKQL

SEQ ID NO: 16 - protein, 9 aa, Homo sapiens
VLNDILSRL

SEQ ID NO: 17 - protein, 9 aa, Homo sapiens
RLQSLQTYV

SEQ ID NO: 18 - protein, 9 aa, Homo sapiens
FIAGLIAIV

SEQ ID NO: 19 - protein, 9 aa, Homo sapiens
FLAFVVFLL

SEQ ID NO: 20 - protein, 9 aa, Homo sapiens
FLLVTLAIL

SEQ ID NO: 21 - protein, 9 aa, Homo sapiens
FLLNKEMYL

SEQ ID NO: 22 - protein, 9 aa, Homo sapiens
IFLWLLWPV

SEQ ID NO: 23 - protein, 9 aa, Homo sapiens
GLMWLSYFI

SEQ ID NO: 24 - protein, 9 aa, Homo sapiens
HLVDFQVTI

SEQ ID NO: 25 - protein, 9 aa, Homo sapiens
IIFWFSLEL

SEQ ID NO: 26 - protein, 9 aa, Homo sapiens
YVVDDPCPI

SEQ ID NO: 27 - protein, 9 aa, Homo sapiens
YIDIGNYTV

SEQ ID NO: 28 - protein, 9 aa, Homo sapiens
YINVFAFPF

SEQ ID NO: 29 - protein, 9 aa, Homo sapiens
NVFAFPFTI

SEQ ID NO: 30 - protein, 8 aa, Homo sapiens
VMVELVAE

SEQ ID NO: 31 - protein, 8 aa, Homo sapiens
LATALLTL

SEQ ID NO: 32 - protein, 8 aa, Homo sapiens
CLEASFNY

SEQ ID NO: 33 - protein, 8 aa, Homo sapiens
LMWLIINL

SEQ ID NO: 34 - protein, 8 aa, Homo sapiens
SLPGVFCG

SEQ ID NO: 35 - protein, 8 aa, Homo sapiens
LLNKEMYL

SEQ ID NO: 36 - protein, 8 aa, Homo sapiens
SMWALIIS

SEQ ID NO: 37 - protein, 8 aa, Homo sapiens
LASGGQPI

SEQ ID NO: 38 - protein, 8 aa, Homo sapiens
KLSYGIAT

SEQ ID NO: 39 - protein, 8 aa, Homo sapiens
LDAYNMMI

SEQ ID NO: 40 - protein, 8 aa, Homo sapiens
LLLDDFVE

SEQ ID NO: 41 - protein, 8 aa, Homo sapiens
VFLVLLPL

SEQ ID NO: 42 - protein, 8 aa, Homo sapiens
SIIAYTMS

SEQ ID NO: 43 - protein, 8 aa, Homo sapiens
LNTLVKQL

SEQ ID NO: 44 - protein, 8 aa, Homo sapiens
VLNDILSR

SEQ ID NO: 45 - protein, 8 aa, Homo sapiens
LQSLQTYV

SEQ ID NO: 46 - protein, 8 aa, Homo sapiens
FIAGLIAI

SEQ ID NO: 47 - protein, 8 aa, Homo sapiens
LAFVVFLL

SEQ ID NO: 48 - protein, 8 aa, Homo sapiens
FLLVTLAI

SEQ ID NO: 49 - protein, 8 aa, Homo sapiens
LLNKEMYL

SEQ ID NO: 50 - protein, 8 aa, Homo sapiens
IFLWLLWP

SEQ ID NO: 51 - protein, 8 aa, Homo sapiens
LMWLSYFI

SEQ ID NO: 52 - protein, 8 aa, Homo sapiens
HLVDFQVT

SEQ ID NO: 53 - protein, 8 aa, Homo sapiens
IFWFSLEL

SEQ ID NO: 54 - protein, 8 aa, Homo sapiens
YVVDDPCP

SEQ ID NO: 55 - protein, 8 aa, Homo sapiens
IDIGNYTV

SEQ ID NO: 56 - protein, 8 aa, Homo sapiens
YINVFAFP

SEQ ID NO: 57 - protein, 8 aa, Homo sapiens
VFAFPFTI

SEQ ID NO: 58 - protein, 15 aa, Homo sapiens
KSAFYILPSIISNEK

SEQ ID NO: 59 - protein, 15 aa, Homo sapiens
ESPFVMMSAPPAQYE

SEQ ID NO: 60 - protein, 15 aa, Homo sapiens
PNMLRIMASLVLARK

SEQ ID NO: 61 - protein, 14 aa, Homo sapiens
RIKVQMLSDTLKNL

SEQ ID NO: 62 - protein, 15 aa, Homo sapiens
LDAYNMMISAGFSLW

SEQ ID NO: 63 - protein, 13 aa, Homo sapiens
MFVFLVLLPLVSS

SEQ ID NO: 64 - protein, 15 aa, Homo sapiens
FLAFVVFLLVTLAIL

SEQ ID NO: 65 - protein, 15 aa, Homo sapiens
FLLVTLAILTALRLC

SEQ ID NO: 66 - protein, 15 aa, Homo sapiens
LSYYKLGASQRVAGD

SEQ ID NO: 67 - protein, 15 aa, Homo sapiens
AEILLIIMRTFKVSI

SEQ ID NO: 68 - protein, 15 aa, Homo sapiens
MKIILFLALITLATC

SEQ ID NO: 69 - protein, 14 aa, Homo sapiens
IIFLALITLATCEL

SEQ ID NO: 70 - protein, 15 aa, Homo sapiens
SPIFLIVAAIVFITL

SEQ ID NO: 71 - protein, 15 aa, Homo sapiens
DFYLCFLAFLLFLVL

SEQ ID NO: 72 - protein, 15 aa, Homo sapiens
MKFLVFLGIITTVAA

SEQ ID NO: 73 - protein, 16 aa, Homo sapiens
KQQTVTLLPAADLDDF

SEQ ID NO: 74 - protein, 14 aa, Homo sapiens
KSAFYILPSIISNE

SEQ ID NO: 75 - protein, 14 aa, Homo sapiens
ESPFVMMSAPPAQY

SEQ ID NO: 76 - protein, 14 aa, Homo sapiens
PNMLRIMASLVLAR

SEQ ID NO: 77 - protein, 13 aa, Homo sapiens
RIKVQMLSDTLKN

SEQ ID NO: 78 - protein, 14 aa, Homo sapiens
LDAYNMMISAGFSL

SEQ ID NO: 79 - protein, 12 aa, Homo sapiens
MFVFLVLLPLVS

SEQ ID NO: 80 - protein, 14 aa, Homo sapiens
FLAFVVFLLVTLAI

SEQ ID NO: 81 - protein, 14 aa, Homo sapiens
FLLVTLAILTALRL

SEQ ID NO: 82 - protein, 14 aa, Homo sapiens
LSYYKLGASQRVAG

SEQ ID NO: 83 - protein, 14 aa, Homo sapiens
AEILLIIMRTFKVS

SEQ ID NO: 84 - protein, 14 aa, Homo sapiens
MKIILFLALITLAT

SEQ ID NO: 85 - protein, 13 aa, Homo sapiens
IIFLALITLATCE

SEQ ID NO: 86 - protein, 14 aa, Homo sapiens
SPIFLIVAAIVFIT

SEQ ID NO: 87 - protein, 14 aa, Homo sapiens
DFYLCFLAFLLFLV

SEQ ID NO: 88 - protein, 14 aa, Homo sapiens
MKFLVFLGIITTVA

SEQ ID NO: 89 - protein, 15 aa, Homo sapiens
KQQTVTLLPAADLDD

SEQ ID NO: 90 - protein, 14 aa, Homo sapiens
SAFYILPSIISNEK

SEQ ID NO: 91 - protein, 14 aa, Homo sapiens
SPFVMMSAPPAQYE

SEQ ID NO: 92 - protein, 14 aa, Homo sapiens
NMLRIMASLVLARK

SEQ ID NO: 93 - protein, 13 aa, Homo sapiens
IKVQMLSDTLKNL

SEQ ID NO: 94 - protein, 14 aa, Homo sapiens
DAYNMMISAGFSLW

SEQ ID NO: 95 - protein, 12 aa, Homo sapiens
FVFLVLLPLVSS

SEQ ID NO: 96 - protein, 14 aa, Homo sapiens
LAFVVFLLVTLAIL

SEQ ID NO: 97 - protein, 14 aa, Homo sapiens
LLVTLAILTALRLC

SEQ ID NO: 98 - protein, 14 aa, Homo sapiens
SYYKLGASQRVAGD

SEQ ID NO: 99 - protein, 14 aa, Homo sapiens
EILLIIMRTFKVSI

SEQ ID NO: 100 - protein, 14 aa, Homo sapiens
KIILFLALITLATC

SEQ ID NO: 101 - protein, 13 aa, Homo sapiens
IFLALITLATCEL

SEQ ID NO: 102 - protein, 14 aa, Homo sapiens
PIFLIVAAIVFITL

SEQ ID NO: 103 - protein, 14 aa, Homo sapiens
FYLCFLAFLLFLVL

SEQ ID NO: 104 - protein, 14 aa, Homo sapiens
KFLVFLGIITTVAA

SEQ ID NO: 105 - protein, 15 aa, Homo sapiens
QQTVTLLPAADLDDF

SEQ ID NO: 106 - protein, 25 aa, Homo sapiens
SQCVNLTTRTQLPPAYTNSFTRGVY

SEQ ID NO: 107 - protein, 23 aa, Homo sapiens
FSNVTWFHAIHVSGTNGTKRFDN

SEQ ID NO: 108 - protein, 31 aa, Homo sapiens
DAVDCALDPLSETKCTLKSFTVEKGIYQTSN

SEQ ID NO: 109 - protein, 40 aa, Homo sapiens
GTNTSNQVAVLYQDVNCTEVPVAIHADQLTPTWRVYSTGS

SEQ ID NO: 110 - protein, 75 aa, Homo sapiens
VCGPKKSTNLVKNKCVNFNFNGLTGTGVLTESNKKFLPFQQFGRDIADTTDAVRDPQTLEILDITPCSFGGVSVI

SEQ ID NO: 111 - protein, 62 aa, Homo sapiens
NLDSKVGGNYNYLYRLFRKSNLKPFERDISTEIYQAGSTPCNGVEGFNCYFPLQSYGFQPTE

SEQ ID NO: 112 - protein, 25 aa, Homo sapiens
YNSASFSTFKCYGVSPTKLNDLCFT

SEQ ID NO: 113 - protein, 35 aa, Homo sapiens
FPNITNLCPFGEVFNATRFASVYAWNRKRISNCVA

SEQ ID NO: 114 - protein, 40 aa, Homo sapiens
VNNTVYDPLQPELDSFKEELDKYFKNHTSPDVDLGDISGI

SEQ ID NO: 115 - protein, 18 aa, Homo sapiens
FSQILPDPSKPSKRSFIE

SEQ ID NO: 116 - protein, 22 aa, Homo sapiens
FGAGAALQIPFAMQMAYRFNGI

SEQ ID NO: 117 - protein, 23 aa, Homo sapiens
SQCVNLTTRTQLPPAYTNSFTRG

SEQ ID NO: 118 - protein, 21 aa, Homo sapiens
FSNVTWFHAIHVSGTNGTKRF

SEQ ID NO: 119 - protein, 29 aa, Homo sapiens
DAVDCALDPLSETKCTLKSFTVEKGIYQT

SEQ ID NO: 120 - protein, 38 aa, Homo sapiens
GTNTSNQVAVLYQDVNCTEVPVAIHADQLTPTWRVYST

SEQ ID NO: 121 - protein, 73 aa, Homo sapiens
VCGPKKSTNLVKNKCVNFNFNGLTGTGVLTESNKKFLPFQQFGRDIADTTDAVRDPQTLEILDITPCSFGGVS

SEQ ID NO: 122 - protein, 60 aa, Homo sapiens
NLDSKVGGNYNYLYRLFRKSNLKPFERDISTEIYQAGSTPCNGVEGFNCYFPLQSYGFQP

SEQ ID NO: 123 - protein, 23 aa, Homo sapiens
YNSASFSTFKCYGVSPTKLNDLC

SEQ ID NO: 124 - protein, 33 aa, Homo sapiens
FPNITNLCPFGEVFNATRFASVYAWNRKRISNC

SEQ ID NO: 125 - protein, 38 aa, Homo sapiens
VNNTVYDPLQPELDSFKEELDKYFKNHTSPDVDLGDIS

SEQ ID NO: 126 - protein, 16 aa, Homo sapiens
FSQILPDPSKPSKRSF

SEQ ID NO: 127 - protein, 20 aa, Homo sapiens
FGAGAALQIPFAMQMAYRFN

SEQ ID NO: 128 - protein, 23 aa, Homo sapiens
CVNLTTRTQLPPAYTNSFTRGVY

SEQ ID NO: 129 - protein, 21 aa, Homo sapiens
NVTWFHAIHVSGTNGTKRFDN

SEQ ID NO: 130 - protein, 29 aa, Homo sapiens
VDCALDPLSETKCTLKSFTVEKGIYQTSN

SEQ ID NO: 131 - protein, 38 aa, Homo sapiens
NTSNQVAVLYQDVNCTEVPVAIHADQLTPTWRVYSTGS

SEQ ID NO: 132 - protein, 73 aa, Homo sapiens
GPKKSTNLVKNKCVNFNFNGLTGTGVLTESNKKFLPFQQFGRDIADTTDAVRDPQTLEILDITPCSFGGVSVI

SEQ ID NO: 133 - protein, 60 aa, Homo sapiens
DSKVGGNYNYLYRLFRKSNLKPFERDISTEIYQAGSTPCNGVEGFNCYFPLQSYGFQPTE

SEQ ID NO: 134 - protein, 23 aa, Homo sapiens
SASFSTFKCYGVSPTKLNDLCFT

SEQ ID NO: 135 - protein, 33 aa, Homo sapiens
NITNLCPFGEVFNATRFASVYAWNRKRISNCVA

SEQ ID NO: 136 - protein, 38 aa, Homo sapiens
NTVYDPLQPELDSFKEELDKYFKNHTSPDVDLGDISGI

SEQ ID NO: 137 - protein, 16 aa, Homo sapiens
QILPDPSKPSKRSFIE

SEQ ID NO: 138 - protein, 20 aa, Homo sapiens
AGAALQIPFAMQMAYRFNGI

SEQ ID NO: 139 - DNA, 5912 bp, synthetic construct
  misc_feature 1..5912: note = Synthesized Vaccine Candidate 1
  source 1..5912: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacttcaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcagcgagatcttcaacgccaccaagttcagcagcgtgtacgcctgggacaggaggaagatcaacaactgcgtggccgactacagcttcctgtacaacagcgccagcttcagcaccttcaagtgctacggcgtgagcctgaacaagctgaacgacctgtgcttcaccaacgtgtacgccgacagcttcgtgatcaggggcgaccaggtgaagcagatcgcccccggccagaccggcaacatcgccgactacaactacaagctgcccgacgacttcaccggctgcgtgatcgcctggaacagcaagaagctggacagcaaggtggtgggcaaccacaagtacaggttcaggttcttcaggaagagcaacctgaagcccttcgagagggacatcagcaccgagatctaccaggtgggcaacaagccctgcaagggcgccaagggcctgaactgctacctgcccctgaagagctacggcttccagcccacctacggcgtgggctaccagccccacagggtggtggtgctgagcttcgagctgctgcacgccagcgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccaggagggccaggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacaaggtggaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 140 - DNA, 5912 bp, synthetic construct
  misc_feature 1..5912: note = Synthesized Vaccine Candidate 2
  source 1..5912: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtgaacttcaccaccaggacccagctgccccccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagagcaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtacagcagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccccatcaacctggtgagggacctgccccagggcttcagcgccctggagcccctggtggacctgcccatcggcatcaacatcaccaggttccagaccctgctggccctgcacaggagctacctgacccccggcgacagcagcagcggctggaccgccggcgccgccgcctactacgtgggctacctgcagcccaggaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagggtgcagcccaccgagagcatcgtgaggttccccaacatcaccaacctgtgccccttcagcgagatcttcaacgccaccaagttcagcagcgtgtacgcctgggacaggaggaagatcaacaactgcgtggccgactacagcttcctgtacaacagcgccagcttcagcaccttcaagtgctacggcgtgagcctgaacaagctgaacgacctgtgcttcaccaacgtgtacgccgacagcttcgtgatcaggggcgaccaggtgaagcagatcgcccccggccagaccggcaacatcgccgactacaactacaagctgcccgacgacttcaccggctgcgtgatcgcctggaacagcaagaagctggacagcaaggtggtgggcaaccacaagtacaggttcaggttcttcaggaagagcaacctgaagcccttcgagagggacatcagcaccgagatctaccaggtgggcaacaagccctgcaagggcgccaagggcctgaactgctacctgcccctgaagagctacggcttccagcccacctacggcgtgggctaccagccccacagggtggtggtgctgagcttcgagctgctgcacgccagcgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagggtgtacagcaccggcagcaacgtgttccagaccagggccggctgcctgatcggcgccgagcacgtgaacaacagctacgagtgcgacatccccatcggcgccggcatctgcgccagctaccagacccagaccaacagccccggcagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagggccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagaggagccccatcgaggacctgctgttcaacaaggtgaccctggccgacgccggcttcatcaagcagtacggcgactgcctgggcgacatcgccgccagggacctgatctgcgcccagaagttcaacggcctgaccgtgctgccccccctgctgaccgacgagatgatcgcccagtacaccagcgccctgctggccggcaccatcaccagcggctggaccttcggcgccggccccgccctgcagatccccttccccatgcagatggcctacaggttcaacggcatcggcgtgacccagaacgtgctgtacgagaaccagaagctgatcgccaaccagttcaacagcgccatcggcaagatccaggacagcctgagcagcacccccagcgccctgggcaagctgcaggacgtggtgaaccagaacgcccaggccctgaacaccctggtgaagcagctgagcagcaacttcggcgccatcagcagcgtgctgaacgacatcctgagcaggctggacccccccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 141 - DNA, 4927 bp, synthetic construct
  misc_feature 1..4927: note = Synthesized Vaccine Candidate 3
  source 1..4927: mol_type = other DNA
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggagaataaactagtattcttctggtccccacagactcagagagaacccgccaccatgttcgtgttcctggtgctgctgcccctggtgagcagccagtgcgtggaggccgccgccaagaagagctacggcttccagcccacctacgccgcctacgtggtgggcaaccacaagtacaggttcgccgcctactaccaggtgggcaacaagccctgcaaggccgcctactgcgtgatcgcctggaacagcaagaaggccgcctacaagggcgccaagggcctgaactgctacgccgcctacagccagtgcgtgaacttcaccaccagggccgcctacaacatcgccgactacaactacaagctggccgcctactacctgcccctgaagagctacggcttcgccgcctacaagtgctacggcgtgagcctgaacaaggccgcctactgcgtgatcgcctggaacagcaagaaggccgcctacatctacaagaccccccccatcaagtacgccgcctactgcgtggccgactacagcttcctgtacgccgcctacagcgtgtacgcctgggacaggaggaaggccgcctacaggttcttcaggaagagcaacctgaaggccgcctacgacatcagcaccgagatctaccaggtggccgcctactaccagccccacagggtggtggtgctggccgcctacgtggtgggcaaccacaagtacaggttcgccgcctacttcgtgatcaggggcgaccaggtgaaggccgcctacaacgccaccaagttcagcagcgtgtacgccgcctactaccaggtgggcaacaagccctgcaaggccgcctacaacgccaccaagttcagcagcgtgtacgccgcctacttcgtgatcaggggcgaccaggtgaaggccgcctacaagggcgccaagggcctgaactgctacgccgcctacaacctgtgccccttcagcgagatcttcgccgcctacgccagcgccaccgtgggaagcggagccacgaacttctctctgttaaagcaagcaggagatgttgaagaaaaccccgggcctcaactgctacctgcccctgaagagctacggcttccagcccacctacggccccggccccggcggcaaccacaagtacaggttcaggttcttcaggaagagcaacctgggccccggccccggccccttcgagagggacatcagcaccgagatctaccaggtgggcaacggccccggccccggcaagaagctggacagcaaggtggtgggcaaccacaagtacaggttcggccccggccccggcaagggcctgaactgctacctgcccctgaagagctacggcttccagggccccggccccggcctggtgctgctgcccctggtgagcagccagtgcgtgaacttcaccggccccggccccggcaggggcgaccaggtgaagcagatcgcccccggccagaccggcaacggccccggccccggcagcgccagcttcagcaccttcaagtgctacggcgtgagcctgaacggccccggccccggcaagctggacagcaaggtggtgggcaaccacaagtacaggttcaggggccccggccccggcttcgcccaggtgaagcagatctacaagaccccccccatcaagtacggccccggccccggcgccgactacagcttcctgtacaacagcgccagcttcagcaccttcggccccggccccggcgccaccaagttcagcagcgtgtacgcctgggacaggaggaagatcggccccggccccggcccccacagggtggtggtgctgagcttcgagctgctgcacgccagcggccccggccccggcttcgagagggacatcagcaccgagatctaccaggtgggcaacaagggccccggccccggcgccaagggcctgaactgctacctgcccctgaagagctacggcttcggccccggccccggcagcatcgtgaggttccccaacatcaccaacctgtgccccttcagcggccccggccccggcaacaactgcgtggccgactacagcttcctgtacaacagcgccagcggccccggccccggcaagggcgccaagggcctgaactgctacctgcccctgaagagctacggccccggccccggcctgtgccccttcagcgagatcttcaacgccaccaagttcagcagcggaagcggagccacgaacttctctctgttaaagcaagcaggagatgttgaagaaaaccccgggcctctgcgtgaacttcaccaccaggacccagctgccccccgcctacaccaacagcttcaccaggggcgtgtactacaagaagaacatcaccaacctgtgccccttcagcgagatcttcaacgccaccaagttcagcagcgtgtacgcctgggacaggaggaagaagatcaacaactgcgtggccgactacagcttcctgtacaacagcgccagcttcagcaccttcaagtgctacggcgtgagcctgaacaagctgaacgacctgaagaagaggggcgaccaggtgaagcagatcgcccccggccagaccggcaacatcgccgacaagaagaagaagctggacagcaaggtggtgggcaaccacaagtacaggttcaggttcttcaggaagagcaacctgaagcccttcgagagggacatcagcaccgagatctaccaggtgggcaacaagccctgcaagggcaagaagacctacggcgtgggctacaagaagctgcacgccagcgccaccgtgtgcggccccaagaagagcaccaacctgaagaaggtgaagcagatctacaagaccccccccatcaagtacttcggcggcttcaacttcagccagatcctgcccgaccccagcaagcccagcaagtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaa

SEQ ID NO: 142 - DNA, 63 bp, synthetic construct
  misc_feature 1..63: note = T2A Linker
  source 1..63: mol_type = other DNA
ggaagcggagagggcaggggaagtcttctaacatgcggggacgtggaggaaaatcccggcccc

SEQ ID NO: 143 - protein, 292 aa, synthetic construct
  REGION 1..292: note = SARS-CoV-like Spike-S1-NTD
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLK

SEQ ID NO: 144 - protein, 223 aa, synthetic construct
  REGION 1..223: note = SARS-CoV-2 Spike-S1-RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 145 - protein, 666 aa, synthetic construct
  REGION 1..666: note = CoV Spike S1-S2_S2
FNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 146 - protein, 1273 aa, synthetic construct
  REGION 1..1273: note = spike glycoprotein with a mutation 682-QQAQ-685 mutation
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 147 - protein, 1273 aa, synthetic construct
  REGION 1..1273: note = spike glycoprotein with two proline substitutions (K986P, V987P)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 148 - protein, 1273 aa, synthetic construct
  REGION 1..1273: note = spike glycoprotein with four proline substitutions(F817P,A892P, A899P, A942P)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 149 - protein, 1273 aa, synthetic construct
  REGION 1..1273: note = spike glycoprotein with six proline substitutions(F817P, A892P,A899P, A942P, K986P, V987P)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 150 - protein, 1273 aa, synthetic construct
  REGION 1..1273: note = spike glycoprotein with six proline substitutions(F817P, A892P,A899P, A942P, K986P, V987P) and a 682-QQAQ-685 mutation
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVTRAGCLIGAEHVNNSYECDIPIGAGICASYQTQTNRDPQTLEILDITPCSFGGVSVITPGTNTSNQVAVLYQDVNCTEVPVAIHADQLTPTWRVYSTGSNVFQSPQQAQSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 151 - protein, 1266 aa, synthetic construct
  REGION 1..1266: note = Spike Glycoprotein sequence with 36 mutations and 6 deletions (-)
MFVFLVLLPLVSSQCVNFTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFSEIFNATKFSSVYAWDRRKINNCVADYSFLYNSASFSTFKCYGVSLNKLNDLCFTNVYADSFVIRGDQVKQIAPGQTGNIADYNYKLPDDFTGCVIAWNSKKLDSKVVGNHKYRFRFRKSNLKPFERDISTEIYQVGNKPCKGAKGLNCYLPLKSYGFQPTYGVGYQPHRVVVLSFELLHASATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 152 - DNA, 69 bp, synthetic construct
  misc_feature 1..69: note = E2A Linker
  source 1..69: mol_type = other DNA
ggaagcggacagtgtactaattatgctctcttgaaattggctggagatgttgagagcaacccaggtccc

SEQ ID NO: 153 - protein, 9 aa, synthetic construct
  REGION 1..9: note = Mutated CD8+ epitope
VLNDILARL

SEQ ID NO: 154 - protein, 13 aa, synthetic construct
  REGION 1..13: note = Mutated CD4+ epitope
MFVFLVLLPLVSI

SEQ ID NO: 155 - protein, 25 aa, synthetic construct
  REGION 1..25: note = mutated B cell Epitope
IQCVNLTTRTQLPPAYTNSFTRGVY

SEQ ID NO: 156 - DNA, 303 bp, Homo sapiens
  source 1..303: mol_type = genomic DNA
atgaacaggaaggtgaccgccatcgccctggccgccatcatctgggccaccgccgcccagggcttcctgatgttcaagcagggcaggtgcctgtgcatcggccccggcatgaaggccgtgaagatggccgagatcgagaaggccagcgtgatctaccccagcaacggctgcgacaaggtggaggtgatcgtgaccatgaaggcccacaagaggcagaggtgcctggaccccaggagcaagcaggccaggctgatcatgcaggccatcgagaagaagaacttcctgaggaggcagaacatgtga

SEQ ID NO: 157 - DNA, 465 bp, Homo sapiens
  source 1..465: mol_type = genomic DNA
atgaaggtctccgcggcagccctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtccacaggtcaaggatgccaaagagagagggacagcaagtctggcaggatttcctgtatgactcccggctgaacaagggcaagctttgtcacccgaaagaaccgccaagtgtgtgccaacccagagaagaaatgggttcgggagtacatcaactctttggagatgagctaggatggagagtccttgaacctgaacttacacaaatttgcctgtttctgcttgctcttgtcctagcttgggaggcttcccctcactatcctaccccacccgctccttga

SEQ ID NO: 158 - DNA, 378 bp, Homo sapiens
  source 1..378: mol_type = genomic DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 159 - DNA, 297 bp, Homo sapiens
  source 1..297: mol_type = genomic DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 160 - DNA, 300 bp, Homo sapiens
  source 1..300: mol_type = genomic DNA
atgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatag

SEQ ID NO: 161 - DNA, 360 bp, Homo sapiens
  source 1..360: mol_type = genomic DNA
atgaaagttctaatctcttccctcctcctgttgctgccactaatgctgatgtccatggtctctagcagcctgaatccaggggtcgccagaggccacagggaccgaggccaggcttctaggagatggctccaggaaggcggccaagaatgtgagtgcaaagattggttcctgagagccccgagaagaaaattcatgacagtgtctgggctgccaaagaagcagtgcccctgtgatcatttcaagggcaatgtgaagaaaacaagacaccaaaggcaccacagaaagccaaacaagcattccagagcctgccagcaatttctcaaacaatgtcagctaagaagctttgctctgcctttgtag

SEQ ID NO: 162 - DNA, 453 bp, Homo sapiens
  source 1..453: mol_type = genomic DNA
atgaacctgtggctcctggcctgcctggtggccggcttcctgggagcctgggcccccgctgtccacacccaaggtgtctttgaggactgctgcctggcctaccactaccccattgggtgggctgtgctccggcgcgcctggacttaccggatccaggaggtgagcgggagctgcaatctgcctgctgcgatattctacctccccaagagacacaggaaggtgtgtgggaaccccaaaagcagggaggtgcagagagccatgaagctcctggatgctcgaaataaggtttttgcaaagctccaccacaacacgcagaccttccaagcaggccctcatgctgtaaagaagttgagttctggaaactccaagttatcatcgtccaagtttagcaatcccatcagcagcagtaagaggaatgtctccctcctgatatcagctaattcaggactgtga

SEQ ID NO: 163 - DNA, 384 bp, Homo sapiens
  source 1..384: mol_type = genomic DNA
atgcagcagagaggactcgccatcgtggccttggctgtctgtgcggccctacatgcctcagaagccatacttcccattgcctccagctgttgcacggaggtttcacatcatatttccagaaggctcctggaaagagtgaatatgtgtcgcatccagagagctgatggggattgtgacttggctgctgtcatccttcatgtcaagcgcagaagaatctgtgtcagcccgcacaaccatactgttaagcagtggatgaaagtgcaagctgccaagaaaaatggtaaaggaaatgtttgccacaggaagaaacaccatggcaagaggaacagtaacagggcacatcaggggaaacacgaaacatacggccataaaactccttattag

SEQ ID NO: 164 - DNA, 465 bp, Homo sapiens
  source 1..465: mol_type = genomic DNA
atgttccacgtgagcttcaggtacatcttcggcatcccccccctgatcctggtgctgctgcccgtgaccagcagcgagtgccacatcaaggacaaggagggcaaggcctacgagagcgtgctgatgatcagcatcgacgagctggacaagatgaccggcaccgacagcaactgccccaacaacgagcccaacttcttcaggaagcacgtgtgcgacgacaccaaggaggccgccttcctgaacagggccgccaggaagctgaagcagttcctgaagatgaacatcagcgaggagttcaacgtgcacctgctgaccgtgagccagggcacccagaccctggtgaactgcaccagcaaggaggagaagaacgtgaaggagcagaagaagaacgacgcctgcttcctgaagaggctgctgagggagatcaagacctgctggaacaagatcctgaagggcagcatctga

SEQ ID NO: 165 - DNA, 489 bp, Homo sapiens
  source 1..489: mol_type = genomic DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 166 - DNA, 462 bp, Homo sapiens
  source 1..462: mol_type = genomic DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 167 - DNA, 1733 bp, synthetic construct
  source 1..1733: mol_type = other DNA; note = CAG promoter
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 168 - DNA, 589 bp, synthetic construct
  source 1..589: mol_type = other DNA; note = CMV Promoter
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 169 - DNA, 635 bp, synthetic construct
  source 1..635: mol_type = other DNA; note = SP-B Promoter
gtatagggctgtctgggagccactccagggccacagaaatcttgtctctgactcagggtattttgttttctgttttgtgtaaatgctcttctgactaatgcaaaccatgtgtccatagaaccagaagatttttccaggggaaaaggtaaggaggtggtgagagtgtcctgggtctgcccttccagggcttgccctgggttaagagccaggcaggaagctctcaagagcattgctcaagagtagagggggcctgggaggcccagggaggggatgggaggggaacacccaggctgcccccaaccagatgccctccaccctcctcaacctccctcccacggcctggagaggtgggaccaggtatggaggcttgagagcccctggttggaggaagccacaagtccaggaacatgggagtctgggcagggggcaaaggaggcaggaacaggccatcagccaggacaggtggtaaggcaggcaggagtgttcctgctgggaaaaggtgggatcaagcacctggagggctcttcagagcaaagacaaacactgaggtcgctgccactcctacagagcccccacgccccgcccagctataaggggccatgcaccaagcagggtacccaggctgcagaggtgcc

SEQ ID NO: 170 - DNA, 1559 bp, synthetic construct
  source 1..1559: mol_type = other DNA; note = CD144 Promoter
catccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacggtgagtgggggctcccacactcccctccaccccaaacccgccaccctgcgcccaagatgggagggtcctcagcttccccatctgtagaatgggcatcgtcccactcccatgacagagaggctcc

SEQ ID NO: 171 - DNA, 39 bp, synthetic construct
  misc_feature 1..39: note = wild type native leader sequence
  source 1..39: mol_type = other DNA
atgttcgtgttcctggtgctgctgcccctggtgagcagc

SEQ ID NO: 172 - protein, 23 aa, synthetic construct
  REGION 1..23: note = Mutated B cell Epitope
FSNVTWFHAIHVSGTNGTKRFAN

SEQ ID NO: 173 - protein, 31 aa, synthetic construct; identical to SEQ ID NO: 108
  REGION 1..31: note = Mutated B cell Epitope

SEQ ID NO: 174 - protein, 62 aa, synthetic construct
  REGION 1..62: note = Mutated B cell Epitope
NLDSKVGGNYNYLYRLFRKSNLKPFERDISTEIYQAGSTPCNGVKGFNCYFPLQSYGFQPTY

SEQ ID NO: 175 - protein, 62 aa, synthetic construct
  REGION 1..62: note = Mutated B cell Epitope
NLDSKVGGNYNYRYRLFRKSNLKPFERDISTEIYQAGSTPCNGVEGFNCYFPLQSYGFQPTE

SEQ ID NO: 176 - protein, 75 aa, synthetic construct
  REGION 1..75: note = Mutated B cell Epitope
VCGPKKSTNLVKNKCVNFNFNGLTGTGVLTESNKKFLPFQQFGRDIDDTTDAVRDPQTLEILDITPCSFGGVSVI

SEQ ID NO: 177 - protein, 40 aa, synthetic construct
  REGION 1..40: note = Mutated B cell Epitope
GTNTSNQVAVLYQGVNCTEVPVAIHADQLTPTWRVYSTGS

SEQ ID NO: 178 - protein, 40 aa, synthetic construct; identical to SEQ ID NO: 114
  REGION 1..40: note = Mutated B cell Epitope

SEQ ID NO: 179 - DNA, 12 bp, synthetic construct
  misc_feature 1..12: note = 682-QQAQ-685 mutation
  source 1..12: mol_type = other DNA
cagcaggcccag

SEQ ID NO: 180 - DNA, 66 bp, synthetic construct
  misc_feature 1..66: note = P2A Linker
  source 1..66: mol_type = other DNA
ggaagcggagccacgaacttctctctgttaaagcaagcaggagatgttgaagaaaaccccgggcct

SEQ ID NO: 181 - DNA, 18 bp, synthetic construct
  misc_feature 1..18: note = 6-His Tag
  source 1..18: mol_type = other DNA
catcaccatcaccatcac

(19) United States
(12) Patent Application Publication
(10) Pub. No.: US 2023/0226173 A1
(43) Pub. Date: Jul. 20, 2023

(54) PAN-CORONAVIRUS VACCINE COMPOSITIONS

(71) Applicant: THE REGENTS OF THE UNIVERSITY OF CALIFORNIA, Oakland, CA (US)

(72) Inventor: Lbachir BenMohamed, Irvine, CA (US)

(21) Appl. No.: 18/046,462

(22) Filed: Oct. 13, 2022

Related U.S. Application Data
(63) Continuation-in-part of application No. PCT/US2021/027340, filed on Apr. 14, 2021.
(60) Provisional application No. 63/084,421, filed on Sep. 28, 2020.
(60) Provisional application No. 63/009,907, filed on Apr. 14, 2020.

(51) Int. Cl. A61K 39/215

(52) U.S. Cl. CPC A61K 39/215; A61K 2039/55522

(57) ABSTRACT

Pan-coronavirus recombinant vaccine compositions featuring whole proteins or sequences of proteins encompassing all mutations in variants of human and animal Coronaviruses (e.g., 36 mutations in spike protein) or a combination of mutated B cell epitopes, mutated combination of B cell epitopes, mutated CD4+ T cell epitopes, and mutated CD8+ T cell epitopes, at least one of which is derived from a non-spike protein. The mutated epitopes may comprise one or more mutations. The present invention also describes using several immuno-informatics and sequence alignment approaches to identify several human B cell, CD4+ and CD8+ T cell epitopes that are highly mutated. The vaccine compositions herein have the potential to provide long-lasting B and T cell immunity regardless of human and animal Coronaviruses mutations.

### CROSS-REFERENCES TO RELATED APPLICATIONS

This application is a continuation in part and claims benefit of PCT Application No. PCT/2021/027340 filed Apr. 14, 2021, which claims benefit of U.S. Provisional Application No. 63/084,421 filed Sep. 28, 2020, and U.S. Provisional Application No. 63/009,907 filed Apr. 14, 2020, the specifications of which are incorporated herein in their entirety by reference.

### FIELD OF THE INVENTION

The present invention relates to pan-coronavirus vaccines, for example viral vaccines, such as those directed to coronaviruses, e.g., pan-coronavirus vaccines.

### REFERENCE TO AN ELECTRONIC SEQUENCE LISTING

The contents of the electronic sequence listing (UCI 20.06C PCT-CIP.xml; Size: 211,927 bytes; and Date of Creation: Oct. 13, 2022) is herein incorporated by reference in its entirety.

### BACKGROUND OF THE INVENTION

Over the last two decades, there have been three deadly human outbreaks of Coronaviruses (CoVs) caused by emerging zoonotic CoVs: SARS-CoV, MERS-CoV, and the latest highly transmissible and deadly SARS-CoV-2, which has caused the current COVID-19 global pandemic. All three deadly CoVs originated from bats, the natural hosts, and transmitted to humans via various intermediate animal reservoirs (e.g., pangolins, civet cats and camels). Because there is currently no universal pan-Coronavirus vaccine available, it remains highly possible that other global COVID-like pandemics will emerge in the coming years, caused by yet another spillover of an unknown zoonotic bat-derived SARS-like Coronavirus (SL-CoV) into an unvaccinated human population.

Neutralizing antibodies and antiviral effector CD4⁺ and CD8⁺ T cells appear to be crucial in reducing viral load in the majority of infected asymptomatic and convalescent patients. However, very little information exists on the antigenic landscape and the repertoire of B-cell and CD4⁺ and CD8⁺ T cell epitopes that are mutated among human and bat Coronavirus strains.

### SUMMARY OF THE INVENTION

841,246 genome sequences for SARS-CoV-2 submitted to the global repository GISAID (Global Initiative on Sharing Avian flu Data) from over 200 countries as of Mar. 10, 2021. Over 4000 synonymous and nonsynonymous mutations have been reported to the Nextstrain database. Since its emergence in late 2019, SARS-CoV-2 has diversified into several different co-circulating variants. Currently variants are grouped into 13 major clades. 19A and 19B emerged in Wuhan and have been dominating the early outbreak. 20A emerged from 19A out of dominated the European outbreak in March and has since spread globafly. 20B and 20C are large genetically distinct subclades 20A emerged in early 2020. 20D to 20I have emerged over the summer of 2020 and include two “variants of concern” (VOC) with signature mutations S:N501Y.

Since its emergence in late 2019, SARS-CoV-2 has diversified into up to 593 different variants and co-circulating variants, with over 4000 synonymous and nonsynonymous mutations have been reported. The mutated epitopes are selected from the Variants Of Concern and Variants Of Interest based on these classification criteria: (1) 593 variants of interest/variants under investigation (VUI) are known as reported to the Global Initiative on Sharing Avian Influenza Data (GISAID). (2) Variants that appear to meet one or more of the undermentioned criteria may be labeled “variants of interest” or “variants under investigation” pending verification and validation of these properties: Increased transmissibility (1) Increased morbidity; (2) Increased transmissibility; (3) Increased mortality; (4) Increased risk of “long COVID”; (5) Ability to evade detection by diagnostic tests; (6) Decreased susceptibility to antiviral drugs (if and when such drugs are available; (7) Decreased susceptibility to neutralizing antibodies, either therapeutic (e.g., convalescent plasma or monoclonal antibodies) or in laboratory experiments; (8) Ability to evade natural immunity; (e.g., causing reinfections); (9) Ability to infect vaccinated individuals; (10) Increased risk of particular conditions such as multisystem inflammatory syndrome or long-haul COVID; (11) Increased affinity for particular demographic or clinical groups, such as children or immunocompromised individuals. Once validated, variants of Interest (VUI) are renamed “variants of concern” by monitoring organizations, such as the CDC (https://www.cdc.gov/coronavirus/2019-ncov/cases-updates/variant-survellance/variant-info.htmWConsequence). As of today 15, variants of concern (VOC) have been reported (as shown in slide-2). We have considered 11 VOC in our sequence homology analysis for which genome sequence has been available in public databases. A related category is “variant of high consequence”, used by the CDC if there is clear evidence that the effectiveness of prevention or Intervention measures for a particular variant is substantially reduced

While most mutations within the SARS-CoV-2 virus have no to minimal effects of the virus, other mutations can cause drastic changes in the virus's properties. For example, mutations may affect the transmission or severity of the virus, and additionally may impact the efficacy of vaccines currently being used to treat COVID-19. The present invention describes using SARS-CoV-2 variant epitopes as well as mutated epitopes to develop a coronavirus vaccine with the ability to protect against new emerging variants of the coronavirus.

The present invention also features pan-coronavirus recombinant vaccine compositions featuring whole proteins or sequences of proteins encompassing all mutations in variants of human and animal Coronaviruses (e.g., 36 mutations in spike protein) or a combination of mutated B cell epitopes, mutated combination of B cell epitopes, mutated CD4+ T cell epitopes, and mutated CD8+ T cell epitopes, at least one of which is derived from a non-spike protein. The mutated epitopes may comprise one or more mutations. The present invention also describes using several immuno-informatics and sequence alignment approaches to identify several human B cell, CD4+ and CD8+ T cell epitopes that are highly mutated. The vaccine compositions herein have the potential to provide long-lasting B and T cell immunity regardless of human and animal Coronaviruses mutations.

The present invention is not limited to vaccine compositions for use in humans. The present invention includes vaccine compositions for use in other animals such as dogs, cats, etc.

The recombinant vaccine compositions herein have the potential to provide lasting B and T cell immunity regardless of Coronaviruses variant. This may be due at least partly because the vaccine compositions target highly mutated structural and non-structural Coronavirus antigens, such as Coronavirus Spike protein, in combination with other Coronavirus structural and non-structural antigens with a low mutation rate found in perhaps every human and animal Coronaviruses variants and strains.

The present invention is also related to selecting highly mutated structural (e.g., spike protein) and non-structural Coronavirus antigens inside the virus (e.g., non-spike protein such as nucleocapsid), which may be viral proteins that are normally not necessarily under mutation pressure by the immune system.

The present invention provides pan-Coronavirus recombinant vaccine compositions, e.g., multi-epitope, pan-coronavirus recombinant vaccine compositions.

In certain embodiments, the vaccine compositions are for use in humans. In certain embodiments, the vaccine compositions are for use in animals, such as but not limited to mice, cats, dogs, non-human primates, other animals susceptible to coronavirus infection, other animals that may function as preclinical animal models for coronavirus infections, etc.

As used herein, the term “multi-epitope” refers to a composition comprising more than one B and T cell epitope wherein at least: one CD4 and/or CD8 T cell epitope is MHC-restricted and recognized by a TCR, and at least one epitope is a B cell epitope.

As used herein, the term “recombinant vaccine composition” may refer to one or more proteins or peptides encoded by one or more recombinant genes, e.g., genes that have been cloned into one or more systems that support the expression of said gene(s). The term “recombinant vaccine composition” may refer to the recombinant genes or the system that supports the expression of said recombinant genes.

For example, the present invention provides a coronavirus recombinant vaccine composition, the composition comprising at least two of: one or more coronavirus B-cell target epitopes; one or more cornavirus CD4+ T cell target epitopes; one or more coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising at least two of: whole spike protein; one or more coronavirus CD4+ T cell target epitopes; one or more coronavirus CD8+ T cell target epitopes: wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising at least two of: at least a portion of spike protein, the portion of spike protein comprising a trimerized SARS-CoV-2 receptor-binding domain (RBD); one or more coronavirus CD4+ T cell target epitopes; one or more coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

The present invention also provides a coronavirus recombinant vaccine composition, the composition comprising: one or more coronavirus B-cell target epitopes; one or more coronavirus CD4+ T cell target epitopes; and one or more coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising: whole spike protein; one or more coronavirus CD4+ T cell target epitopes; and one or more coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising: at least a portion of spike protein, the portion of spike protein comprising a trimerized SARS-CoV-2 receptor-binding domain (RBD); one or more coronavirus CD4+ T cell target epitopes; and one or more coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

The present invention also provides a coronavirus recombinant vaccine composition, the composition comprising an antigen delivery system encoding at least two of: one or more mutated coronavirus B-cell target epitopes; one or more mutated coronavirus CD4+ T cell target epitopes: and/or one or more mutated coronavirus CD8+ T cell target epitopes: wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising an antigen delivery system encoding at least two of: whole spike protein; one or more mutated coronavirus CD4+ T cell target epitopes; and/or one or more mutated coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising an antigen delivery system encoding at least two of: at least a portion of spike protein, the portion of spike protein comprising a trimerized SARS-CoV-2 receptor-binding domain (RBD); one or more mutated coronavirus CD4+ T cell target epitopes; and/or one or more mutated coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

The present invention also provides a coronavirus recombinant vaccine composition, the composition comprising an antigen delivery system encoding: one or more mutated coronavirus B-cell target epitopes; one or more mutated coronavirus CD4+ T cell target epitopes; and one or more mutated coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising an antigen delivery system encoding: whole spike protein; one or more mutated coronavirus CD4+ T cell target epitopes; and one or more mutated coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising an antigen delivery system encoding: at least a portion of spike protein, the portion of spike protein comprising a trimerized SARS-CoV-2 receptor-binding domain (RBD); one or more mutated coronavirus CD4+ T cell target epitopes; and one or more mutated coronavirus CD8+ T cell target epitopes; wherein the epitopes are derived from a human coronavirus, an animal coronavirus, or a combination thereof; wherein at least one epitope is derived from a non-spike protein. In some embodiments, the composition induces immunity to only the epitopes.

The present invention also provides a coronavirus recombinant vaccine composition, the composition comprising an antigen delivery system, the antigen delivery system encodes: an antigen, the composition comprises at least two of: one or more coronavirus B-cell target epitopes; one or more coronavirus CD4+ T cell target epitopes; or one or more coronavirus CD8+ T cell target epitopes; wherein the epitopes are derived from a human coronavirus, an animal coronavirus, or a combination thereof; wherein at least one epitope is derived from a non-spike protein (in some embodiments the composition induces immunity to only the epitopes); a T cell attracting chemokine; and a composition that promotes T cell proliferation; wherein the epitopes are derived from a human coronavirus, an animal coronavirus, or a combination thereof; wherein at least one epitope is derived from a non-spike protein. In some embodiments, the composition induces Immunity to only the epitopes.

Likewise, the present invention provides a coronavirus recombinant vaccine composition, the composition comprising an antigen delivery system encoding: whole spike protein; one or more mutated coronavirus CD4+ T cell target epitopes; and/or one or more mutated coronavirus CD8+ T cell target epitopes; a T cell attracting chemokine; and a composition that promotes T cell proliferation; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising an antigen delivery system encoding: at least a portion of spike protein, the portion of spike protein comprising a trimerized SARS-CoV-2 receptor-binding domain (RBD); one or more mutated coronavirus CD4+ T cell target epitopes; and/or one or more mutated coronavirus CD8+ T cell target epitopes; a T cell attracting chemokine; and a composition that promotes T cell proliferation; wherein the epitopes are derived from a human coronavirus, an animal coronavirus, or a combination thereof; wherein at least one epitope is derived from a non-spike protein. In some embodiments, the composition induces immunity to only the epitopes.

The present invention also provides a coronavirus recombinant vaccine composition, the composition comprising an antigen delivery system encoding: one or more mutated coronavirus B-cell target epitopes; one or more mutated coronavirus CD4+ T cell target epitopes; and one or more mutated coronavirus CD8+ T cell target epitopes; a T cell attracting chemokine; and a composition that promotes T cell proliferation; wherein the epitopes are derived from a human coronavirus, an animal coronavirus, or a combination thereof; wherein at least one epitope is derived from a non-spike protein. In some embodiments, the composition induces immunity to only the epitopes.

Likewise, the present invention provides a coronavirus recombinant vaccine composition, the composition comprising an antigen delivery system encoding: whole spike protein; one or more mutated coronavirus CD4+ T cell target epitopes; and one or more mutated coronavirus CD8+ T cell target epitopes; a T cell attracting chemokine; and a composition that promotes T cell proliferation; wherein the epitopes are derived from a human coronavirus, an animal coronavirus, or a combination thereof; wherein at least one epitope is derived from a non-spike protein. In some embodiments, the composition induces Immunity to only the epitopes.

Likewise, the present invention provides a coronavirus recombinant vaccine composition, the composition comprising an antigen delivery system encoding: at least a portion of spike protein, the portion of spike protein comprising a trimerized SARS-CoV-2 receptor-binding domain (RBD); one or more mutated coronavirus CD4+ T cell target epitopes; and one or more mutated coronavirus CD8+ T cell target epitopes; a T cell attracting chemokine; and a composition that promotes T cell proliferation wherein the epitopes are derived from a human coronavirus, an animal coronavirus, or a combination thereof; wherein at least one epitope is derived from a non-spike protein. In some embodiments, the composition Induces immunity to only the epitopes.

Referring to any of the embodiments herein, in certain embodiments, at least one epitope has a mutation. In certain embodiments, at least one epitope has a mutation as compared to its corresponding epitope in SARS-CoV-2 isolate Wuhan-Hu-1.

In some embodiments, the mutation is one or a combination of: a D614G mutation, a T445C mutation, a C6286T mutation, a C26801G mutation, a C4543T mutation, a G5629T mutation, a C11497T mutation, a T26876C mutation, a C241T mutation, a C913T mutation, a C3037T mutation, a C5986T mutation, a C14676T mutation, a C15279T mutation, a T16176C mutation, a G174T mutation, a C241T mutation, a C3037T mutation, a C28253T mutation, a C241T mutation, a T733C mutation, a C2749T mutation, a C3037T mutation, a A6319G mutation, a A6613G mutation, a C12778T mutation, a C13880T mutation, a A28877T mutation, a G28878C mutation, a C2395T mutation, a T2597C mutation, a T24349C mutation, a G27890T mutation, a A28272T mutation, a C8047T mutation, a C28651T mutation, a G4960T mutation, a C6070T mutation, a C7303T mutation, a C7564T mutation, a C10279T mutation, a C10525T mutation, a C10582T mutation, a C27804T mutation, a C241T mutation, a C1498T mutation, a A1807G mutation, a G2659A mutation, a C3037T mutation, a T8593C mutation, a C9593T mutation, a C18171T mutation, a A20724G mutation, a C24748T mutation, a A28699G mutation, a G29543T mutation, a C241T mutation, a C3037T mutation, a A20262G mutation, a A28271- mutation, a C241T mutation, a G1942T mutation, a C3037T mutation, a A9085G mutation, a C14805T mutation, a C241T mutation, a C3037T mutation, a C21811A mutation, a T29194C mutation, a T29377 mutation, or combination thereof. In some embodiments, the mutation is one or more mutations in the spike (S) protein. In some embodiments, the mutation is one or a combination of A22V, S477N, H69-, V70-, Y144-, N501Y, A570D, P681H, D80A, D215G, L241-, L242-, A243-, K417N, E484K, N501Y, A701V. L18F, K417T, E484K, N501Y, H655Y, S13I, W152C, L452R, S439K, S98F, D80Y, A626S, Vi122L, A67V, H69-, V70-, Y144-, E484K, Q877H, F888L, LSF, T95I, D253G, E484K, A701V, Q677H, Q677P or a combination thereof. In some embodiments, the mutation is one or more mutations in the nucleocapsid (N) protein. In some embodiments, the mutation is one or a combination of A220V, M234I, A376T, R203K, G204R, T205I, P80R, R203K, G204R, P199L, S186Y, D377Y, S2-, D3Y, A12G, P199L, M234I, P67S, P199L, D377Y, P67S, P199L or a combination thereof. In some embodiments, the mutation is one or more mutations in the Envelope (E) protein. In some embodiments, the mutation is P71L. In some embodiments, the mutation is one or more mutations in the ORF3a protein. In some embodiments, the mutation is one or a combination of Q38R, G172R, V202L, P42L or a combination thereof. In some embodiments, the mutation is one or more mutations in the ORF7a protein. In some embodiments, the mutation is R80I. In some embodiments, the mutation is one or more mutations in the ORF8 protein. In some embodiments, the mutation is Q27*, T11I. or a combination thereof. In some embodiments, the mutation is one or more mutations in the ORF10 protein. In some embodiments, the mutation is V30L. In some embodiments, the mutation is one or more mutations in the ORF1b protein. In some embodiments, the mutation is one or a combination of A176S, V767L, K1141R, E1184D, D1183Y, P255T, Q1011H, N1653D, R2613C, N1653D, R2613C or a combination thereof. In some embodiments, the mutation is one or more mutations in the ORF1a protein. In some embodiments, the mutation is one or a combination of S3875-, G3676-, F3677-, S3675-, G3676-, F3677-, S3675-, G3676-, F3677-, 14205V, I2501T, T945I, T1567I, Q3346K, V3475F, M3862I, S3675-, G3676-, F3677-, S3875-, G3876-, F3877-, T265I, L3352F, T265I, L3352F or a combination thereof.

In some embodiments, the epitopes are each asymptomatic epitopes. In some embodiments, the composition lacks symptomatic epitopes.

In some embodiments, the non-spike protein is ORF1ab protein, ORF3a protein, Envelope protein, Membrane glycoprotein, ORF6 protein, ORF7a protein, ORF7b protein, ORF8 protein, Nucleocapsid protein and ORF10 protein.

In some embodiments, the human coronavirus is SARS-CoV-2 original strain. In some embodiments, the human coronavirus is a SARS-CoV-2 variant. In some embodiments, the animal coronavirus is a bat coronavirus, a pangolin coronavirus, a civet cat coronavirus, a mink coronavirus, a camel coronavirus, or a coronavirus from another animal susceptible to coronavirus infection.

In some embodiments, one or more of the at least two target epitopes is in the form of a large sequence.

In some embodiments, the large sequence is derived from one or more whole protein sequences expressed by SARS-CoV-2 or a SARS-CoV-2 variant. In some embodiments, the large sequence is derived from one or more partial protein sequences expressed by SARS-CoV-2 or a SARS-CoV-2 variant.

In some embodiments, the SARS-CoV-2 variant epitope is derived from one or more of: strain B.1.177; strain B.1.160, strain B.1.1.7; strain B.1.351; strain P.1; strain B.1.427/B.1.429; strain B.1.258; strain B.1.221; strain B.1.367; strain B.1.1.277; strain B.1.1.302; strain B.1.525; strain B.1.526, strain S:677H, or strain S:677P.

In some embodiments, the target epitopes are derived from structural proteins, non-structural proteins, or a combination thereof. In some embodiments, the target epitopes are derived from a SARS-CoV-2 protein selected from a group consisting of: ORF1ab protein, Spike glycoprotein, ORF3a protein, Envelope protein, Membrane glycoprotein, ORF6 protein, ORF7a protein, ORF7b protein, ORF8 protein, Nucleocapsid protein an ORF10 protein. In some embodiments, the ORF1ab protein comprises nonstructural protein (Nsp) 1, Nsp2, Nsp3, Nsp4, Nsp5, Nsp6, Nsp7, Nsp8, Nsp9, Nsp10, Nsp11, Nsp12, Nsp13, Nsp14, Nsp15 and Nsp16.

In some embodiments, the epitopes are derived from SARS-CoV-2 or a SARS-CoV-2 variant and restricted to human HLA class 1 and 2 haplotypes. In some embodiments, the epitopes are derived from SARS-CoV-2 or a SARS-CoV-2 variant and restricted to cat and dog MHC class 1 and 2 haplotypes.

In some embodiments, the one or more coronavirus CD8+ T cell target epitopes are selected from: spike glycoprotein, Envelope protein, ORF1ab protein, ORF7a protein, ORF8a protein, ORF10 protein, or a combination thereof. In some embodiments, the epitope comprises a D614G mutation. In some embodiments, the one or more mutated epitopes are highly mutated among human and animal coronaviruses. In some embodiments, the one or more mutated epitopes are derived from at least one of SARS-CoV-2 protein.

In some embodiments, the one or more mutated epitopes are derived from one or more of: one or more SARS-CoV-2 human strains or variants in current circulation: one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. In some embodiments, the one or more SARS-CoV-2 human strains or variants in current circulation are selected from: strain B.1.177; strain B.1.160, strain B.1.1.7; strain B.1.351; strain P.1; strain B.1.427/B.1.429; strain B.1.258; strain B.1.221; strain B.1.367; strain B.1.1.277; strain B.1.1.302; strain B.1.525; strain B.1.526, strain S:677H, and strain S:877P. In some embodiments, the one or more coronaviruses that cause the common cold are selected from: 229E alpha coronavirus, NL63 alpha coronavirus, OC43 beta coronavirus, and HKU1 beta coronavirus. In some embodiments, the mutated epitopes are selected from Variants Of Concern or Variants Of Interest.

In some embodiments, the one or more CD8+ T cell epitopes are among the 20 most highly mutated CD8+ T cell epitopes identified in a sequence alignment and analysis of a particular number of coronavirus sequences. In some embodiments, the one or more CD4+ T cell epitopes are among the 20 most highly mutated CD4+ T cell epitopes identified in a sequence alignment and analysis of a particular number of coronavirus sequences. In some embodiments, the one or more B cell epitopes are among the 30 most highly mutated B cell epitopes identified in a sequence alignment and analysis of a particular number of coronavirus sequences.

In some embodiments, the one or more coronavirus CD8+ T cell target epitopes are selected from: spike glycoprotein, Envelope protein, ORF1ab protein, ORF7a protein, ORF8a protein, ORF10 protein, or a combination thereof.

In some embodiments, the one or more coronavirus CD8+ T cell target epitopes are selected from: S2-10, S1220-1228, S1000-1008, S958-968, E20-28, ORF1ab1675-1683, ORF1ab2363-2371, ORF1ab3013-3021, ORF1ab3183-3191, ORF1ab5470-5478, ORF1ab6749-6757, ORF7b28-34, ORF8a73-81, ORF103-11, and ORF105-13. In some embodiments, the one or more coronavirus CD8+ T cell target epitopes are selected from SEQ ID NO: 2-29. In some embodiments, the one or more coronavirus CD8+ T cell target epitopes are selected from SEQ ID NO: 30-57. In some embodiments, the one or more coronavirus CD4+ T cell target epitopes are selected from: spike glycoprotein, Envelope protein, Membrane protein, Nucleocapsid protein, ORF1a protein, ORF1ab protein, ORF6 protein, ORF7a protein, ORF7b protein, ORF8 protein, or a combination thereof. In some embodiments, the one or more coronavirus CD4+ T cell target epitopes are selected from: ORF1a1350-1385, ORF1ab5019-5033, ORF612-26, ORF1ab6088-8102, ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15. In some embodiments, the one or more coronavirus CD4+ T cell target epitopes are selected from SEQ ID NO: 58-73. In some embodiments, the one or more coronavirus CD4+ T cell target epitopes are selected from SEQ ID NO: 74-105. In some embodiments, the one or more coronavirus B cell target epitopes are selected from Spike glycoprotein. In some embodiments, the one or more coronavirus B cell target epitopes are selected from: S287-317, S524-598, S601-640, S802-819, S888-909, S369-393, S440-501, S1133-1172, S329-363, and S13-37. In some embodiments, the one or more coronavirus B cell target epitopes are selected from SEQ ID NO: 106-116. In some embodiments, the one or more coronavirus B cell target epitopes are selected from SEQ ID NO: 117-138.

In some embodiments, the composition comprises 2-20 CD8+ T cell target epitopes. In some embodiments, the composition comprises 2-20 CD4+ T cell target epitopes. In some embodiments, the composition comprises 2-20 B cell target epitopes.

In some embodiments, the one or more coronavirus B cell target epitopes are in the form of a large sequence. In some embodiments, the large sequence is full length spike glycoprotein. In some embodiments, the large sequence is a partial spike glycoprotein.

In some embodiments, the spike glycoprotein has two consecutive proline substitutions at amino acid positions 986 and 987. In some embodiments, the spike glycoprotein has single amino acid substitutions at amino acid positions comprising Tyr-83 and Tyr-489, Gln-24 and Asn-487. In some embodiments, the spike protein comprises Tyr-489 and Asn-487. In some embodiments, the spike protein comprises Gln-493. In some embodiments, the spike protein comprises Tyr-505. In some embodiments, the composition comprises a trimerized SARS-CoV-2 receptor-binding domain (RBD). In some embodiments, the trimerized SARS-CoV-2 receptor-binding domain (RBD) sequence is modified by the addition of a T4 fibritin-derived foldon trimerization domain. In some embodiments, the composition comprises a mutation 682-RRAR-685→682-QQAQ-685 in the S1-S2 cleavage site. In some embodiments, the spike glycoprotein has 38 point mutations. The present invention includes the compositions herein in the form of a nucleoside-modified mRNA pan-CoV vaccine composition.

In some embodiments, the composition comprises a trimerized SARS-CoV-2 receptor-binding domain (RBD) and one or more highly mutated SARS-CoV-2 sequences selected from structural proteins and non-structural proteins.

In some embodiments, the composition is encapsulated in a lipid nanoparticle.

In some embodiments, the structural protein is nucleoprotein. In some embodiments, the non-structural protein is Nsp4. In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display.

In some embodiments, the composition incorporates a good manufacturing practice-grade mRNA drug substance that encodes the trimerized SARS-CoV-2 spike glycoprotein RBD antigen together with the one or more highly mutated structural and non-structural SARS-CoV-2 antigens.

In some embodiments, the sequence for the antigen is GenBank accession number, MN908947.3.

In some embodiments, the composition comprises at least one proline substitution. In some embodiments, the composition comprises at least two proline substitutions. In some embodiments, the proline substitution is at position K986 and V987. In some embodiments, the composition comprises K986P and V987P mutations.

As previously discussed, in certain embodiments, the one or more mutated coronavirus B cell target epitopes are in the form of a large sequence, e.g., whole spike protein or partial spike protein (e.g., a portion of whole spike protein). In some embodiments, the whole spike protein or portion thereof is in its stabilized conformation. In certain embodiments, the transmembrane anchor of the spike protein (or portion thereof) has an intact S1-S2 cleavage site. In certain embodiments, the spike glycoprotein has two consecutive proline substitutions at amino acid positions 988 and 987, e.g., for stabilization. In certain embodiments, the spike protein or portion thereof has an amino acid substitution at amino acid position Tyr-83. In certain embodiments, the spike protein or portion thereof has an amino acid substitution at amino acid position Tyr-489. In certain embodiments, the spike protein or portion thereof has an amino acid substitution at amino acid position Gln-24. In certain embodiments, the spike protein or portion thereof has an amino acid substitution at amino acid position Asn-487. In certain embodiments, the spike protein or portion thereof has an amino acid substitution at one or more of: Tyr-83, Tyr-489, Gln-24, Gln-493, and Asn-487, e.g., the spike protein or portion thereof may comprise Tyr-489 and Asn-487, the spike protein or portion thereof may comprise Gln-493, the spike protein or portion thereof may comprise Tyr-505, etc. Tyr-489 and Asn-487 may help with interaction with Tyr 83 and Gln-24 on ACE-2. Gln-493 may help with interaction with Glu-35 and Lys-31 on ACE-2. Tyr-505 may help with interaction with Glu-37 and Arg-393 on ACE-2.

In certain embodiments, the composition comprises a mutation 682-RRAR-685-682-QQAQ-685 in the S1-S2 cleavage site. In certain embodiments, the composition comprises at least one proline substitution. In certain embodiments, the composition comprises at least two proline substitutions, e.g., at position K988 and V987.

In certain embodiments, a target epitope derived from the spike glycoprotein is RBD. In certain embodiments, a target epitope derived from the spike glycoprotein is NTD. In certain embodiments, a target epitope derived from the spike glycoprotein is one or more epitopes, e.g., comprising both the RBD and NTD regions. In certain embodiments, a target epitope derived from the spike glycoprotein is recognized by neutralizing and blocking antibodies. In certain embodiments, a target epitope derived from the spike glycoprotein induces neutralizing and blocking antibodies. In certain embodiments, a target epitope derived from the spike glycoprotein induces neutralizing and blocking antibodies that recognize and neutralize the virus. In certain embodiments, a target epitope derived from the spike glycoprotein induces neutralizing and blocking antibodies that recognize the spike protein.

In certain embodiments, each of the target epitopes are separated by a linker. In certain embodiments, a portion of the target epitopes are separated by a linker. In certain embodiments, the linker is from 2-10 amino acids in length. In certain embodiments, the linker is from 3-12 amino acids in length. In certain embodiments, the linker is from 5-15 amino acids in length. In certain embodiments, the linker is 10 or more amino acids in length. Non-limiting examples of linkers include AAY, KK, and GPGPG.

In some embodiments, the composition comprises the addition of a T4 fibritin-derived foldon trimerization domain. In some embodiments, the addition of a T4 fibritin-derived foldon trimerization domain increases immunogenicity by multivalent display.

In certain embodiments, the composition further comprises a T cell attracting chemokine. For example, the composition may further comprise one or a combination of CCL5, CXCL9, CXCL10, CXCL11, or a combination thereof.

In certain embodiments, the composition further comprises a composition that promotes T cell proliferation. For example, the composition may further comprise IL-7, IL-15, IL-2, or a combination thereof.

In certain embodiments, the composition further comprises a molecular adjuvant. For example, the composition may further comprise one or a combination of CpG (e.g., CpG polymer) or flagellin.

In certain embodiments, the composition comprises a tag. For example, the epitopes may be in the form of a single antigen, wherein the composition comprises a tag. In certain embodiments, the epitopes are in the form of two or more antigens, wherein one or more of the antigens comprise a tag. Non-limiting examples of tags include a His tag.

In certain embodiments, the transmembrane anchor of the spike protein has an intact S1-S2 cleavage site. In certain embodiments, the spike protein is in Its stabilized conformation. In certain embodiments, the spike protein is stabilized with proline substitutions at amino acid positions 986 and 987 at the top of the central helix in the S2 subunit.

In some embodiments, the composition comprises full-length spike protein. In some embodiments, the composition comprises full-length spike protein or partial spike protein.

In certain embodiments, the vaccine composition is for humans. In certain embodiments, the vaccine composition is for animals. In certain embodiments, the animals are cats and dogs.

In certain embodiments, the target epitope derived from the Spike glycoprotein is RBD. In certain embodiments, the target epitope derived from the Spike glycoprotein is NTD. In certain embodiments, the target epitope derived from the Spike glycoprotein Includes both the RBD and NTD regions. In certain embodiments, the target epitopes derived from the spike glycoprotein are recognized by neutralizing and blocking antibodies. In certain embodiments, the target epitope derived from the spike glycoprotein induces neutralizing and blocking antibodies. In certain embodiments, the target epitope derived from the spike glycoprotein Induces neutralizing and blocking antibodies that recognize and neutralize the virus. In certain embodiments, the target epitope derived from the spike glycoprotein induces neutralizing and blocking antibodies that recognize the spike protein. In certain embodiments, the ORF1ab protein comprises nonstructural protein (Nsp) 1, Nsp2, Nsp3, Nsp4, Nsp5, Nsp8, Nsp7, Nsp8. Nsp9, Nsp10, Nsp11, Nsp12, Nsp13, Nsp14, Nsp15 and Nsp16.

In certain embodiments, the linker comprises T2A. In certain embodiments, the linker is selected from T2A, E2A, and P2A. In certain embodiments, a different linker is disposed between each open reading frame.

In certain embodiments, the composition is for delivery with lipid nanoparticles.

In certain embodiments, the composition comprises a trimerized SARS-CoV-2 receptor-binding domain (RBD). In certain embodiments, the trimerized SARS-CoV-2 receptor-binding domain (RBD) sequence is modified by the addition of a T4 fibritin-derived foldon trimerization domain.

In certain embodiments, the “antigen delivery system” may refer to two delivery systems, e.g., a portion of the epitopes (or other components such as chemokines, etc.) may be encoded by one delivery system and a portion of the epitopes (or other components) may be encoded by a second delivery system (or a third delivery system, etc.).

Referring to the antigen delivery system, in certain embodiments the antigen delivery system Is an adeno-associated viral vector-based antigen delivery system. Non-limiting examples include an adeno-associated virus vector type 8 (AAV8 serotype) or an adeno-associated virus vector type 9 (AAV9 serotype). In certain embodiments, the antigen delivery system is a vesicular stomatitis virus (VSV) vector. In certain embodiments, the antigen delivery system is an adenovirus (e.g., Ad26, Ad5, Ad35, etc.)

The target epitopes are operatively linked to a promoter. In certain embodiments, the promoter Is a generic promoter (e.g., CMV, CAG, etc.). In certain embodiments, the promoter is a lung-specific promoter (e.g., SpB, CD144). In certain embodiments, all of the target epitopes are operatively linked to the same promoter. In certain embodiments, a portion of the target epitopes are operatively linked to a first promoter and a portion of the target epitopes are operatively linked to a second promoter. In certain embodiments, the target epitopes are operatively linked to two or more promoters, e.g., a portion are operatively linked to a first promoter, a portion is operatively linked to a second promoter, etc. In certain embodiments, the target epitopes are operatively linked to three or more promoters, e.g., a portion is operatively linked to a first promoter, a portion is operatively linked to a second promoter, a portion is operatively linked to a third promoter, etc. In certain embodiments, the first promoter is the same as the second promoter. In certain embodiments the second promoter is different from the first promoter. In certain embodiments, the promoter is a generic promoter (e.g., CMV, CAG, etc.). In certain embodiments, the promoter is a lung-specific promoter (e.g., SpB, CD144) promoter.

In certain embodiments, the antigen delivery system encodes a T cell attracting chemokine. In certain embodiments, the antigen delivery system encodes a composition that promotes T cell proliferation. In certain embodiments, the antigen delivery system encodes both a T cell attracting chemokine and a composition that promotes T cell proliferation. In certain embodiments, the antigen delivery system encodes a molecular adjuvant. In certain embodiments, the antigen delivery system encodes a T cell attracting chemokine, a composition that promotes T cell proliferation and a molecular adjuvant. In certain embodiments, the antigen delivery system encodes a T cell attracting chemokine and a molecular adjuvant. In some embodiments, the antigen delivery system encodes a composition that promotes T cell proliferation and a molecular adjuvant.

In certain embodiments, the T cell attracting chemokine is CCL5, CXCL9, CXCL10, CXCL11, or a combination thereof. In certain embodiments, the composition that promotes T cell proliferation is IL-7 or IL-15 or IL-2. In some embodiments, the molecular adjuvant is CpG (e.g., CpG polymer), flagellin, etc.).

In certain embodiments, the T cell attracting chemokine is operatively linked to a lung-specific promoter (e.g., SpB, CD144). In certain embodiments, the T cell attracting chemokine is operatively linked to a generic promoter (e.g., CMV, CAG, etc.). In certain embodiments, the composition that promotes T cell proliferation is operatively linked to a lung-specific promoter (e.g., SpB, CD144). In certain embodiments, the composition that promotes T cell proliferation is operatively linked to a generic promoter (e.g., CMV, CAG, etc.). In certain embodiments, the molecular adjuvant is operatively linked to a lung-specific promoter (e.g., SpB, CD144). In certain embodiments, the molecular adjuvant is operatively linked to a generic promoter (e.g., CMV, CAG, etc.). In certain embodiments, the T cell attracting chemokine and the composition that promotes T cell proliferation are driven by the same promoter. In certain embodiments, the T cell attracting chemokine and the composition that promotes T cell proliferation are driven by different promoters. In certain embodiments, the molecular adjuvant, the T cell attracting chemokine, and the composition that promotes T cell proliferation are driven by the same promoter. In certain embodiments, the molecular adjuvant, the T cell attracting chemokine, and the composition that promotes T cell proliferation are driven by different promoters. In certain embodiments, the molecular adjuvant and the composition that promotes T cell proliferation are driven by different promoters. In certain embodiments, the molecular adjuvant and the T cell attracting chemokine are driven by different promoters.

In certain embodiments, the T cell attracting chemokine and the composition promoting T cell proliferation are separated by a inker. In certain embodiments, the linker comprises T2A. In certain embodiments, the linker comprises E2A. In certain embodiments, the linker comprises P2A. In certain embodiments, the linker is selected from T2A, E2A, and P2A.

Referring to the antigen delivery system, in certain embodiments, a linker is disposed between each open reading frame. In certain embodiments, a different linker is disposed between each open reading from. In certain embodiments, the same linker may be used between particular open reading frames and a different linker may be used between other open reading frames.

In some embodiments, the vaccine composition is administered using modified RNA, adeno-associated virus, or an adenovirus.

The composition herein may be used to prevent a coronavirus disease in a subject. The composition herein may be used to prevent a coronavirus infection prophylactically in a subject. The composition herein may be used to elicit an immune response in a subject. The term “subject” herein may refer to a human, a non-human primate, an animal such as a mouse, rat, cat, dog, other animal that is susceptible to coronavirus infection, or other animal used for preclinical modeling. The composition herein may prolong an immune response induced by the multi-epitope pan-coronavirus recombinant vaccine composition and increases T-cell migration to the lungs. In certain embodiments, the composition induces resident memory T cells (Trm). In some embodiments, the vaccine composition induces efficient and powerful protection against the coronavirus disease or infection. In some embodiments, the vaccine composition induces production of antibodies (Abs), CD4+ T helper (Th1) cells, and CD8+ cytotoxic T-cells (CTL). In some embodiments, the composition that promotes T cell proliferation helps to promote long term immunity. In some embodiments, the T-cell attracting chemokine helps pull T-cells from circulation into the lungs.

In certain embodiments, the composition further comprises a pharmaceutical carrier.

The present invention includes any of the vaccine compositions described herein, e.g., the aforementioned vaccine compositions for delivery with nanoparticles, e.g., lipid nanoparticles. For example, the present invention includes the vaccine compositions herein encapsulated in a lipid nanoparticle.

In some embodiments, the vaccine composition comprises a nucleoside-modified mRNA vaccine composition comprising a vaccine composition as described herein.

The present invention includes the compositions described herein comprising and/or encoding a trimerized SARS-CoV-2 receptor-binding domain (RBD) and one or more highly mutated SARS-CoV-2 sequences selected from structural proteins (e.g., nucleoprotein, etc.) and non-structural protein (e.g., Nsp4, etc.). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display.

In certain embodiments, the composition incorporates a good manufacturing practice-grade mRNA drug substance that encodes the trimerized SARS-CoV-2 spike glycoprotein RBD antigen together with the one or more highly mutated structural and non-structural SARS-CoV-2 antigens. In certain embodiments, the sequence for an antigen is GenBank accession number, MN908947.3.

The present invention also features a coronavirus recombinant vaccine composition comprising one of SEQ ID NO: 139-141.

In some embodiments, a mutated target epitope is one that is one of the 5 most mutated epitopes (for its epitope type, e.g., B cell, CD4 T cell, CD8 T cell) Identified in a sequence alignment and analysis. In some embodiments, a mutated target epitope is one that is one of the 10 most mutated epitopes (for its epitope type, e.g., B cell, CD4 T cell, CD8 T cell) Identified in a sequence alignment and analysis. In some embodiments, a mutated target epitope is one that is one of the 15 most mutated epitopes (for its epitope type, e.g., B cell, CD4 T cell, CD8 T cell) identified in a sequence alignment and analysis. In some embodiments, a mutated target epitope is one that is one of the 20 most mutated epitopes (for its epitope type, e.g., B cell, CD4 T cell, CD8 T cell) identified in a sequence alignment and analysis. In some embodiments, a mutated target epitope is one that is one of the 25 most mutated epitopes (for its epitope type, e.g., B cell, CD4 T cell, CD8 T cell) identified in a sequence alignment and analysis. In some embodiments, a mutated target epitope is one that is one of the 30 most mutated epitopes (for Its epitope type, e.g., B cell, CD4 T cell, CD8 T cell) identified in a sequence alignment and analysis. In some embodiments, a mutated target epitope is one that is one of the 35 most mutated epitopes (for Its epitope type, e.g., B cell, CD4 T cell, CD8 T cell) identified in a sequence alignment and analysis. In some embodiments, a mutated target epitope is one that is one of the 40 most mutated epitopes (for its epitope type, e.g., B cell, CD4 T cell, CD8 T cell) identified in a sequence alignment and analysis. In some embodiments, a mutated target epitope is one that is one of the 50 most mutated epitopes (for its epitope type, e.g., B cell, CD4 T cell, CD8 T cell) identified in a sequence alignment and analysis. Examples of sequence alignments and analyses. Are described herein. For example, steps or methods for selecting or identifying mutated epitopes may first include performing a sequence alignment and analysis of a particular number of coronavirus sequences to determine sequence similarity or identity amongst the group of analyzed sequences. In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses Isolated from animals selected from a group consisting of bats, pangolins, civet cats, minks, camels, and other animal receptive to coronaviruses; and/or one or more coronaviruses that cause the common cold.

The present invention also features methods of producing pan-coronavirus recombinant vaccine compositions of the present invention.

For example, in some embodiments, the method comprises selecting at least two of: one or more coronavirus B-cell epitopes; one or more coronavirus CD4+ T cell epitopes; one or more coronavirus CD8+ T cell epitopes. The epitopes are derived from a human coronavirus, an animal coronavirus, or a combination thereof. At least one epitope is derived from a non-spike protein. In some embodiments, the composition induces immunity to only the epitopes. The method further comprises synthesizing an antigen or antigens comprising the selected epitopes (or a combination of antigens that collectively comprise the selected epitopes). In some embodiments, the method comprises selecting: one or more mutated coronavirus B-cell epitopes; one or more mutated coronavirus CD4+ T cell epitopes; and one or more mutated coronavirus CD8+ T cell epitopes. At least one epitope is derived from a non-spike protein. In some embodiments, the composition induces immunity to only the epitopes. The method further comprises synthesizing an antigen comprising the selected epitopes (or a combination of antigens that collectively comprise the selected epitopes). In some embodiments, the method further comprises introducing the vaccine composition to a pharmaceutical carrier. The steps for selecting the one or more mutated epitopes are disclosed herein. Methods for synthesizing recombinant proteins are well known to one of ordinary skill in the art. The vaccine compositions are disclosed herein. In some embodiments, the vaccine composition is in the form of DNA, RNA, modified RNA, protein (or peptide), or a combination thereof.

In some embodiments, the method comprises selecting: one or more coronavirus B-cell epitopes; one or more coronavirus CD4+ T cell epitopes; and one or more coronavirus CD8+ T cell epitopes. The epitopes are derived from a human coronavirus, an animal coronavirus, or a combination thereof. At least one epitope is derived from a non-spike protein. In some embodiments, the composition induces immunity to only the epitopes. The method further comprises synthesizing an antigen delivery system encoding the selected epitopes. In some embodiments, the method further comprises introducing the vaccine composition to a pharmaceutical carrier. The steps for selecting the one or more mutated epitopes are disclosed herein. Methods for synthesizing antigen delivery systems are well known to one of ordinary skill in the art. The vaccine compositions are disclosed herein. In some embodiments, the vaccine composition is in the form of DNA, RNA, modified RNA, protein (or peptide), or a combination thereof.

The present invention also features methods for preventing coronavirus disease. The method comprises administering to a subject a therapeutically effective amount of a pan-coronavirus recombinant vaccine composition according to the present invention, wherein the composition elicits an immune response in the subject and helps prevent coronavirus disease.

The present invention also features methods for preventing a coronavirus infection prophylactically in a subject. In some embodiments, the method comprises administering to the subject a prophylactically effective amount of a pan-coronavirus recombinant vaccine composition according to the present invention, wherein the vaccine composition prevents coronavirus infection.

The present invention also features methods for eliciting an immune response in a subject, comprising administering to the subject a composition according to the present invention, wherein the vaccine composition elicits an immune response in the subject. The present invention also features methods comprising: administering to a subject a pan-coronavirus recombinant vaccine composition according to the present invention, wherein the composition prevents virus replication in the lungs, the brain, and other compartments where the virus replicates. The present invention also features methods comprising: administering to the subject a pan-coronavirus recombinant vaccine composition according to the present invention, wherein the composition prevents cytokine storm in the lungs, the brain, and other compartments where the virus replicates. The present invention also features methods comprising: administering to the subject a pan-coronavirus recombinant vaccine composition according to the present invention, wherein the composition prevents inflammation or inflammatory response in the lungs, the brain, and other compartments where the virus replicates. The present invention also features methods comprising: administering to the subject a pan-coronavirus recombinant vaccine composition according to the present invention, wherein the composition improves homing and retention of T cells in the lungs, the brain, and other compartments where the virus replicates. The present invention also features methods for preventing coronavirus disease in a subject; the method comprising: administering to the subject a pan-coronavirus recombinant vaccine composition according to the present invention, wherein the composition induces memory B and T cells. The present invention also features methods for prolonging an immune response induced by a pan-coronavirus recombinant vaccine and increasing T-cell migration to the lungs, the method comprising: co-expressing a T-cell attracting chemokine, a composition that promotes T cell proliferation, and a pan-coronavirus recombinant vaccine according to the present invention. The present invention also features methods for prolonging the retention of memory T-cell into the lungs Induced by a pan coronavirus vaccine and increasing virus-specific tissue resident memory T-cells (TRM cells), the method comprising: co-expressing a T-cell attracting chemokine, a composition that promotes T cell proliferation, and a pan-coronavirus recombinant vaccine according to the present invention. The present invention also features methods comprising: administering to the subject a pan-coronavirus recombinant vaccine composition according to the present invention, wherein the composition prevents the development of mutation and variants of a coronavirus.

For the sake of brevity, it is noted that the vaccine compositions referred to in the aforementioned methods include the vaccine compositions previously discussed, the embodiments described below, and the embodiments in the figures.

In some embodiments, the vaccine composition is administered through an intravenous route (i.v.), an intranasal route (i.n.), or a sublingual route (s.l.) route.

In some embodiments, the vaccine composition is administered using modified RNA, adeno-associated virus, or an adenovirus.

As previously discussed, the composition herein may be used to prevent a coronavirus disease in a subject. The composition herein may be used to prevent a coronavirus infection prophylactically in a subject. The composition herein may be used to elicit an immune response in a subject. The term “subject” herein may refer to a human, a non-human primate, an animal such as a mouse, rat, cat, dog, other animal that is susceptible to coronavirus infection, or other animal used for preclinical modeling. The composition herein may prolong an immune response induced by the multi-epitope pan-coronavirus recombinant vaccine composition and increases T-cell migration to the lungs. In certain embodiments, the composition induces resident memory T cells (Trm). In some embodiments, the vaccine composition induces efficient and powerful protection against the coronavirus disease or infection. In some embodiments, the vaccine composition induces production of antibodies (Abs), CD4+ T helper (Th1) cells, and CD8+ cytotoxic T-cells (CTL). In some embodiments, the composition that promotes T cell proliferation helps to promote long term immunity. In some embodiments, the T-cell attracting chemokine helps pull T-cells from circulation into the lungs.

The present invention also features oligonucleotide compositions. For example, the present invention includes oligonucleotides disclosed in the sequence listings. The present invention also includes oligonucleotides in the form of antigen delivery systems. The present invention also includes oligonucleotides encoding the mutated epitopes disclosed herein. The present invention also includes oligonucleotide compositions comprising one or more oligonucleotides encoding any of the vaccine compositions according to the present invention. In some embodiments, the oligonucleotide comprises DNA. In some embodiments, the oligonucleotide comprises modified DNA. In some embodiments, the oligonucleotide comprises RNA. In some embodiments, the oligonucleotide comprises modified RNA. In some embodiments, the oligonucleotide comprises mRNA. In some embodiments, the oligonucleotide comprises modified mRNA.

The present invention also features peptide compositions. For example, the present invention includes peptides disclosed in the sequence listings. The present invention also includes peptide compositions comprising any of the vaccine compositions according to the present invention. The present invention also includes peptide compositions comprising any of the mutated epitopes according to the present invention.

For the sake of brevity, it is noted that the vaccine compositions referred to in the aforementioned oligonucleotide and peptide compositions include the vaccine compositions previously discussed, the embodiments described below, and the embodiments in the figures.

The present invention also features a method comprising: administering a first pan-coronavirus recombinant vaccine dose using a first delivery system, and administering a second vaccine dose using a second delivery system, wherein the first and second delivery system are different. In some embodiments, the first delivery system may comprise a RNA, a modified mRNA, or a peptide delivery system. In some embodiments, the second delivery system may comprise a RNA, a modified mRNA, or a peptide delivery system. In some embodiments, the peptide delivery system is an adenovirus or an adeno-associated virus. In some embodiments, the adenovirus delivery system is Ad26, Ad5, Ad35, or a combination thereof. In some embodiments, the adeno-associated delivery system is AAV8 or AAV9. In some embodiments, the peptide delivery system is a vesicular stomatitis virus (VSV) vector. In some embodiments, the second vaccine dose is administered 14 days after the first vaccine dose.

The present invention also features a method comprising: administering a pan-coronavirus recombinant vaccine composition according to the present invention: and administering at least one T-cell attracting chemokine after administering the pan-coronavirus recombinant vaccine composition. In some embodiments, the vaccine composition is administered via a RNA, a modified mRNA, or a peptide delivery system. In some embodiments, the T-cell attracting chemokine is administered via a RNA, a modified mRNA, or a peptide delivery system. In some embodiments, the peptide delivery system is an adenovirus or an adeno-associated virus. In some embodiments, the adenovirus delivery system is Ad26, Ad5, Ad35, or a combination thereof.

In some embodiments, the adeno-associated delivery system is AAV8 or AAV9. In some embodiments, the peptide delivery system is a vesicular stomatitis virus (VSV) vector. In some embodiments, the T-cell attracting chemokine is administered 8 days after administering days after the vaccine composition. In some embodiments, the T-cell attracting chemokine is administered 14 days after administering days after the vaccine composition. In some embodiments, the T-cell attracting chemokine is administered 30 days after administering days after the vaccine composition. In some embodiments, the T-cell attracting chemokine is CCL5, CXCL9, CXCL10, CXCL11, or a combination thereof. The present invention also features a method comprising: administering a pan-coronavirus recombinant vaccine composition according to the present invention; administering at least one T-cell attracting chemokine after administering the pan-coronavirus recombinant vaccine composition; and administering at least one cytokine after administering the T-cell attracting chemokine. In some embodiments, the vaccine composition is administered via a RNA, a modified mRNA, or a peptide delivery system. In some embodiments, the T-cell attracting chemokine is administered via a RNA, a modified mRNA, or a peptide delivery system. In some embodiments, the cytokine is administered via a RNA, a modified mRNA, or a peptide delivery system. In some embodiments, the peptide delivery system is an adenovirus or an adeno-associated virus. In some embodiments, the adenovirus delivery system is Ad26, Ad5, Ad35, or a combination thereof. In some embodiments, the adeno-associated delivery system is AAV8 or AAV9. In some embodiments, the peptide delivery system is a vesicular stomatitis virus (VSV) vector. In some embodiments, the T-cell attracting chemokine is administered 14 days after administering the vaccine composition. In some embodiments, the T-cell attracting chemokine is CCL5, CXCL9, CXCL10, CXCL11, or a combination thereof. In some embodiments, the cytokine is administered 10 days after administering the T-cell attracting chemokine. In some embodiments, the cytokine is IL-7, IL-15, IL2 or a combination thereof.

The present invention also features a method comprising: administering a pan-coronavirus recombinant vaccine composition according to the present invention; administering one or more T-cell attracting chemokine after administering the pan-coronavirus recombinant vaccine composition; and administering one or more mucosal chemokine(s). In some embodiments, the vaccine composition is administered using modified RNA, adeno-associated virus, or an adenovirus. In some embodiments, the T-cell attracting chemokine is administered via a RNA, a modified mRNA, or a peptide delivery system. In some embodiments, the mucosal chemokine is administered via a RNA, a modified mRNA, or a peptide delivery system. In some embodiments, the adeno-associated virus is AAV8 or AAV9. In some embodiments, the adenovirus is Ad26, Ad5, Ad35, or a combination thereof. In some embodiments, the T-cell attracting chemokine is administered 14 days after administering the vaccine composition. In some embodiments, the T-cell attracting chemokine is CCL5, CXCL9, CXCL10, CXCL11, or a combination thereof. In some embodiments, the mucosal chemokine is administered 10 days after administering the T-cell attracting chemokine. In some embodiments, the mucosal chemokine is CCL25, CCL28, CXCL14, or CXCL17, or a combination thereof.

For the sake of brevity, it is noted that the vaccine compositions referred to in the aforementioned methods include the vaccine compositions previously discussed, the embodiments described below, and the embodiments in the figures.

As previously discussed, in some embodiments, the vaccine compositions are for use in humans. In some embodiments, the vaccine compositions are for use in animals, e.g., cats, dogs, etc. In some embodiments, the vaccine comprises human CXCL-11 and/or human IL-7 (or IL-15, IL-2). In some embodiments, the vaccine composition comprises animal CLCL-11 and/or animal IL-7 (or IL-15, IL-2).

The present invention includes vaccine compositions in the form of a rVSV-panCoV vaccine composition. The present invention Includes vaccine compositions in the form of a rAdV-panCoV vaccine composition.

The present invention also includes nucleic acids for use in the vaccine compositions herein. The present invention also includes vectors for use in the vaccine compositions herein. The present invention also includes fusion proteins for use in the vaccine compositions herein. The present invention also includes immunogenic compositions for use in the vaccine compositions herein.

The vaccine compositions herein may be designed to elicit both high levels of virus-blocking and virus-neutralizing antibodies as well as CD4+ T cells and CD8+ T cells in adults 18 to 55 years. The vaccine compositions herein may be designed to elicit both high levels of virus-blocking and virus-neutralizing antibodies as well as CD4+ T cells and CD8+ T cells in adults 55 to 65 years of age. The vaccine compositions herein may be designed to elicit both high levels of virus-blocking and virus-neutralizing antibodies as well as CD4+ T cells and CD8+ T cells in adults 65 to 85 years of age. The vaccine compositions herein may be designed to elicit both high levels of virus-blocking and virus-neutralizing antibodies as well as CD4+ T cells and CD8+ T cells in adults 85 to 100 years of age. The vaccine compositions herein may be designed to elicit both high levels of virus-blocking and virus-neutralizing antibodies as well as CD4+ T cells and CD8+ T cells in children 12 to 18 years of age. The vaccine compositions herein may be designed to elicit both high levels of virus-blocking and virus-neutralizing antibodies as well as CD4+ T cells and CD8+ T cells in children under 12 years of age.

The present invention is not limited to vaccine compositions. For example, in certain embodiments, one or more of the epitopes are used for detecting coronavirus and/or diagnosing coronavirus Infection.

The present invention also provides a coronavirus recombinant vaccine composition comprising one or more coronavirus B-cell target epitopes and one or more coronavirus CD4+ T cell target epitopes, or one or more coronavirus CD8+ T cell target epitopes and one or more coronavirus CD4+ T cell target epitopes, wherein the one or more coronavirus B-cell target epitopes are derived from a human coronavirus, an animal coronavirus, or a combination thereof; the one or more coronavirus CD4+ T cell target epitopes are derived from a human coronavirus, an animal coronavirus, or a combination thereof; and/or the one or more coronavirus CD8+ T cell target epitopes are derived from a human coronavirus, an animal coronavirus, or a combination thereof; wherein at least one epitope has a mutation as compared to its corresponding epitope in SARS-CoV-2 isolate Wuhan-Hu-1; wherein at least one epitope is derived from a non-spike protein. In some embodiments, the composition induces immunity to only the epitopes.

In some embodiments, the human coronavirus is SARS-CoV-2 original strain. In some embodiments, the human coronavirus is a SARS-CoV-2 variant. In some embodiments, one or more of the epitopes is in the form of a large sequence. In some embodiments, the large sequence is derived from one or more whole or partial protein sequences expressed by SARS-CoV-2 or a SARS-CoV-2 variant. In some embodiments, the SARS-CoV-2 variant epitope is derived from one or more of: strain B.1.177; strain B.1.160, strain B.1.1.7; strain B.1.351; strain P.1; strain B.1.427/B.1.429; strain B.1.258; strain B.1.221; strain B.1.367; strain B.1.1.277; strain B.1.1.302; strain B.1.525; strain B.1.526, strain S:677H, or strain S:677P.

In some embodiments, the mutation is selected from: a D614G mutation, a T445C mutation, a C6286T mutation, a C26801G mutation, a C4543T mutation, a G5629T mutation, a C11497T mutation, a T26876C mutation, a C241T mutation, a C913T mutation, a C3037T mutation, a C5986T mutation, a C14876T mutation, a C15279T mutation, a T16176C mutation, a G174T mutation, a C241T mutation, a C3037T mutation, a C28253T mutation, a C241T mutation, a T733C mutation, a C2749T mutation, a C3037T mutation, a A6319G mutation, a A6613G mutation, a C12778T mutation, a C13860T mutation, a A28877T mutation, a G28878C mutation, a C2395T mutation, a T2597C mutation, a T24349C mutation, a G27890T mutation, a A28272T mutation, a C8047T mutation, a C28651T mutation, a G4960T mutation, a C6070T mutation, a C7303T mutation, a C7564T mutation, a C10279T mutation, a C10525T mutation, a C10582T mutation, a C27804T mutation, a C241T mutation, a C1498T mutation, a A1807G mutation, a G2659A mutation, a C3037T mutation, a T8593C mutation, a C9593T mutation, a C18171T mutation, a A20724G mutation, a C24748T mutation, a A28699G mutation, a G29543T mutation, a C241T mutation, a C3037T mutation, a A20262G mutation, a A28271- mutation, a C241T mutation, a G1942T mutation, a C3037T mutation, a A9085G mutation, a C14805T mutation, a C241T mutation, a C3037T mutation, a C21811A mutation, a T29194C mutation, a T29377 mutation, or combination thereof.

In some embodiments, the one or more coronavirus CD8+ T cell target epitopes are selected from: S2-10, S1220-1228, S1000-1008, S958-966, E20-28, ORF1ab1675-1683, ORF1ab2383-2371, ORF1ab3013-3021, ORF1ab3183-3191, ORF1ab5470-5478, ORF1ab6749-6757, ORF7b26-34, ORF8a73-81, ORF103-11, and ORF105-13. In some embodiments, the one or more coronavirus CD4+ T cell target epitopes are selected from: ORF1a1350-1365, ORF1ab5019-5033, ORF612-26, ORF1ab6088-6102, ORF1ab6420-8434, ORF1a1801-1815, S1-13, E26-40, E20-34, M176-190. N388-403, ORF7a3-17, ORF7a1-15, ORF7b8-22, ORF7a98-112, and ORF81-15. In some embodiments, the one or more coronavirus B cell target epitopes are selected from: S287-317, S524-598, S601-640, S802-819, S888-909, S369-393, S440-501, S1133-1172, S329-363, and S13-37.

In some embodiments, the one or more coronavirus B cell target epitopes is in the form of whole spike protein or partial spike protein. In some embodiments, the whole spike protein or partial spike protein has an intact S1-S2 cleavage site. In some embodiments, the spike protein is stabilized with proline substitutions at amino acid positions 986 and 987. In some embodiments, the composition comprises 2-20 CD8+ T cell target epitopes.

In some embodiments, the composition comprises 2-20 CD4+ T cell target epitopes. In some embodiments, the composition comprises 2-20 B cell target epitopes.

The present invention also features a coronavirus recombinant vaccine composition, the composition comprising an antigen delivery system encoding at least two of: one or more coronavirus B-cell target epitopes derived from a human coronavirus, an animal coronavirus, or a combination thereof; one or more coronavirus CD4+ T cell target epitopes derived from a human coronavirus, an animal coronavirus, or a combination thereof; and/or one or more coronavirus CD8+ T cell target epitopes derived from a human coronavirus, an animal coronavirus, or a combination thereof; wherein at least one epitope has a mutation as compared to its corresponding epitope in SARS-CoV-2 isolate Wuhan-Hu-1; wherein at least one epitope is derived from a non-spike protein. In some embodiments, the composition induces immunity to only the epitopes.

In some embodiments, the antigen delivery system is an adeno-associated viral vector-based antigen delivery system. In some embodiments, the adeno-associated viral vector is an adeno-associated virus vector type 8 (AAV8 serotype) or an adeno-associated virus vector type 9 (AAV9 serotype). In some embodiments, the antigen delivery system is an adenovirus delivery system or a vesicular stomatitis virus (VSV) delivery system. In some embodiments, the antigen delivery system is an mRNA delivery system. In some embodiments, the antigen delivery system further encodes a T cell attracting chemokine. In some embodiments, the antigen delivery system further encodes a composition that promotes T cell proliferation. In some embodiments, the antigen delivery system further encodes a molecular adjuvant. In some embodiments, the antigen (e.g., epitopes) is operatively linked to a lung-specific promoter. In some embodiments, the one or more coronavirus B cell target epitopes is in the form of whole spike protein or partial spike protein. In some embodiments, the whole spike protein or partial spike protein has an intact S1-S2 cleavage site. In some embodiments, the spike protein is stabilized with proline substitutions at amino acid positions 986 and 987.

The present invention also features a coronavirus recombinant vaccine composition comprising an antigen delivery system encoding one or more coronavirus B-cell target epitopes and one or more coronavirus CD4+ T cell target epitopes, or one or more coronavirus CD8+ T cell target epitopes and one or more coronavirus CD4+ T cell target epitopes, wherein the one or more coronavirus B-cell target epitopes are derived from a human coronavirus, an animal coronavirus, or a combination thereof; the one or more coronavirus CD4+ T cell target epitopes are derived from a human coronavirus, an animal coronavirus, or a combination thereof; and/or the one or more coronavirus CD8+ T cell target epitopes are derived from a human coronavirus, an animal coronavirus, or a combination thereof; wherein at least one epitope has a mutation as compared to its corresponding epitope in SARS-CoV-2 isolate Wuhan-Hu-1; wherein at least one epitope is derived from a non-spike protein. In some embodiments, the composition induces Immunity to only the epitopes.

The present invention also includes the corresponding nucleic acid sequences for any of the protein sequences herein. The present invention also Includes the corresponding protein sequences for any of the nucleic acid sequences herein.

Embodiments herein may comprise whole spike protein or a portion of spike protein. Whole spike protein and a portion thereof is not limited to a wild type or original sequence and may include spike protein or a portion thereof with one or more modifications and/or mutations, such as point mutations, deletions, etc., including the mutations described herein such as those for improving stability.

Embodiments of the present invention can be freely combined with each other if they are not mutually exclusive.

Any feature or combination of features described herein are included within the scope of the present invention provided that the features Included in any such combination are not mutually inconsistent as will be apparent from the context, this specification, and the knowledge of one of ordinary skill in the art. Additional advantages and aspects of the present invention are apparent in the following detailed description and claims.

### DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic view of an example of a multi-epitope pan-coronavirus recombinant vaccine composition. CD8+ T cell epitopes are shown with a square, CD4+ T cell epitopes are shown with a circle and B-cell epitopes are shown with a diamond. Each shape (square, circle, or diamond) may represent a variety of different epitopes and is not limited to a singular epitope. The multi-epitope pan-coronavirus vaccines are not limited to a specific combination of epitopes as shown. The multi-epitope pan-coronavirus vaccines may comprise a various number of individual CD8+, CD4+, or B cell epitopes.

FIG. 2A shows an evolutionary comparison of genome sequences among beta-Coronavirus strains isolated from humans and animals. A phylogenetic analysis performed between SARS-CoV-2 strain sp (obtained from humans (Homo Sapiens (black)), along with the animal's SARS-like Coronaviruses genome sequence (SL-CoVs) sequences obtained from bats (Rhinolophus affinis, Rhinolophus malayanus (red)), pangolins (Manis javanica (blue)), civet cats (Paguma larvata (green)), and camels (Camelus dromedaries (Brown)). The included SARS-CoV/MERS-CoV strains are from previous outbreaks (obtained from humans (Urbani, MERS-CoV, OC43, NL63, 229E, HKU1-genotype-B), bats (WIV16, WIV1, YNLF-31C, Rs672, recombinant strains), camel (Camelus dromedaries, (KT368891.1, MN514967.1, KF917527.1, NC_028752.1), and civet (Civet007, A022, B039)). The human SARS-CoV-2 genome sequences are represented from six continents.

FIG. 2B shows an evolutionary analysis performed among the human-SARS-CoV-2 genome sequences reported from six continents and SARS-CoV-2 genome sequences obtained from bats (Rhinolophus affinis, Rhinolophus malayanus), and pangolins (Manis javanica)).

FIG. 3A shows lungs, heart, kidneys, intestines, brain, and testicles express ACE2 receptors and are targeted by SARS-CoV-2 virus. SARS-CoV-2 virus docks on the Angiotensin converting enzyme 2 (ACE2) receptor via spike surface protein.

FIG. 3B shows a System Biology Analysis approach utilized in the present invention.

FIG. 4A shows examples of binding capacities of virus-derived CD4+ T cell epitope peptides to soluble HLA-DR molecules. CD4+ T cell peptides were submitted to ELISA binding assays specific for HLA-DR molecules. Reference non-viral peptides were used to validate each assay. Data are expressed as relative activity (ratio of the IC₅₀ of the peptides to the IC₅₀ of the reference peptide) and are the means of two experiments. Peptide epitopes with high affinity binding to HLA-DR molecules have IC₅₀ below 250 and are indicated in bold. IC₅₀ above 250 indicates peptide epitopes that failed to bind to tested HLA-DR molecules.

FIG. 4B shows an example of potential epitopes binding with high affinity to HLA-A*0201 and stabilizing expression on the surface of target cells: Predicted and measured binding affinity of genome-derived peptide epitopes to soluble HLA-A*0201 molecule (IC₅₀ nM). The binding capacities of a virus CD8 T cell epitope peptide to soluble HLA-A*0201 molecules. CD8 T cell peptides were submitted to ELISA binding assays specific for HLA-A*0201 molecules. Reference non-viral peptides were used to validate each assay. Data are expressed as relative activity (ratio of the IC₅₀ to the peptide to the IC₅₀ of the reference peptide) and are the means of two experiments. Peptide epitopes with high affinity binding to HLA-A*0201 molecules have IC₅₀ below 100 and are indicated in bold. IC₅₀ above 100 indicates peptide epitopes that failed to bind to tested HLA-A*0201 molecules.

FIG. 5 shows a sequence homology analysis to screen conservancy of potential SARS-CoV-2-derived human CD8+ T cell epitopes. Shown are the comparison of sequence homology for the potential CD8+ T cell epitopes among 81,963 SARS-CoV-2 strains (that currently circulate in 190 countries on 6 continents), the 4 major “common cold” Coronaviruses that cased previous outbreaks (i.e. hCoV-OC43, hCoV-229E, hCoV-HKU1-Genotype B, and hCoV-NL63), and the SL-CoVs that were Isolated from bats, civet cats, pangolins and camels. Epitope sequences highlighted in yellow present a high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels, as described herein. Homo Sapiens-black, bats (Rhinolophus affinis, Rhinolophus malayanus-red), pangolins (Manis javanica-blue), civet cats (Paguma larvata-green), and camels (Camelus dromedaries-brown).

FIG. 6A shows docking of highly mutated SARS-CoV-2-derived human CD8+ T cell epitopes to HLA-A*02:01 molecules, e.g., docking of the 27 high-affinity CD8+ T cell binder peptides to the groove of HLA-A*02:01 molecules.

FIG. 6B shows a summary of the interaction similarity scores of the 27 high-affinity CD8+ T cell epitope peptides to HLA-A*02:01 molecules determined by protein-peptide molecular docking analysis. Black columns depict CD8+ T cell epitope peptides with high interaction similarity scores.

FIG. 7A shows an experimental design show CD8+ T cells are specific to highly mutated SARS-CoV-2 epitopes detected in COVID-19 patients and unexposed healthy individuals: PBMCs from HLA-A*02:01 positive COVID-19 patients (n=30) and controls unexposed healthy individuals (n=10) were isolated and stimulated overnight with 10 μM of each of the 27 SARS-CoV-2-derived CD8+ T cell epitopes. The number of IFN-γ-producing cells were quantified using ELISpot assay.

FIG. 7B shows the results from FIG. 7A. Dotted lines represent threshold to evaluate the relative magnitude of the response: a mean SFCs between 25 and 50 correspond to a medium/intermediate response whereas a strong response is defined for a mean SFCs>50.

FIG. 7C shows the results from experiments where PBMCs from HLA-A*02:01 positive COVID-19 patients were further stimulated for an additional 5 hours in the presence of mAbs specific to CD107a and CD107b, and Golgi-plug and Golgi-stop. Tetramers specific to Spike epitopes, CD107a/b and CD69 and TNF-expression were then measured by FACS. Representative FACS plot showing the frequencies of Tetramer+CD8+ T cells, CD107a/b+CD8+ T cells. CD69+CD8+ T cells and TNF-+CD8+ T cells following priming with a group of 4 Spike CD8+ T cell epitope peptides. Average frequencies of tetramer+CD8+ T cells, CD107a/b+CD8+ T cells, CD69+CD8+ T cells and TNF-+CD8+ T cells.

FIG. 8A shows a timeline of immunization and immunological analyses for experiments testing the immunogenicity of genome-wide Identified human SARS-CoV-2 CD8+ T epitopes in HLA-A*02:01//HLA-DRB1 double transgenic mice. Eight groups of age-matched HLA-A*02:01 transgenic mice (n=3) were immunized subcutaneously, on days 0 and 14, with a mixture of four SARS-CoV-2-derived human CD8+ T cell peptide epitopes mixed with PADRE CD4+ T helper epitope, delivered in alum and CpG1826 adjuvants. As a negative control, mice received adjuvants alone (mock-immunized).

FIG. 8B shows the gating strategy used to characterize spleen-derived CD8+ T cells. Lymphocytes were identified by a low forward scatter (FSC) and low side scatter (SSC) gate. Singlets were selected by plotting forward scatter area (FSC-A) vs. forward scatter height (FSC-H). CD8 positive cells were then gated by the expression of CD8 and CD3 markers.

FIG. 8C shows a representative ELISpot images (left panel) and average frequencies (right panel) of IFN-γ-producing cell spots from splenocytes (106 cells/well) stimulated for 48 hours with 10 μM of 10 Immunodominant CD8+ T cell peptides and 1 subdominant CD8+ T cell peptide out of the total pool of 27 CD8+ T cell peptides derived from SARS-CoV-2 structural and non-structural proteins. The number on the top of each ELISpot image represents the number of IFN-γ-producing spot forming T cells (SFC) per one million splenocytes.

FIG. 8D shows a representative FACS plot (left panel) and average frequencies (right panel) of IFN-γ and TNF-production by, and CD107a/b and CD69 expression on 10 immunodominant CD8+ T cell peptides and 1 subdominant CD8+ T cell peptide out of the total pool of 27 CD8+ T cell peptides derived from SARS-CoV-2 structural and non-structural proteins determined by FACS. Numbers indicate frequencies of IFN-γ+CD8+ T cells, CD107+CD8+ T cells, CD69+CD8+ T cells and TNF-+CD8+ T cells, detected in 3 immunized mice.

FIG. 9 shows the SARS-CoV/SARS-CoV-2 genome encodes two large non-structural genes ORF1a (green) and ORF1b (gray), encoding 16 non-structural proteins (NSP1-NSP16). The genome encodes at least six accessory proteins (shades of light grey) that are unique to SARS-CoV/SARS-CoV-2 in terms of number, genomic organization, sequence, and function. The common SARS-CoV, SARS-CoV-2 and SL-CoVs-derived human B (blue). CD4+ (green) and CD8+ (black) T cell epitopes are shown. Structural and non-structural open reading frames utilized in this study were from SARS-CoV-2-Wuhan-Hu-1 strain (NCBI accession number MN908947.3, SEQ ID NO: 1). The amino acid sequence of the SARS-CoV-2-Wuhan-Hu-1 structural and non-structural proteins was screened for human B. CD4+ and CD8+ T cell epitopes using different computational algorithms as described herein. Shown are genome-wide identified SARS-CoV-2 human B cell epitopes (in blue), CD4+ T cell epitopes (in green), CD8+ T cell epitopes (in black) that are highly mutated between human and animal Coronaviruses.

FIG. 10 shows the identification of highly mutated potential SARS-CoV-2-derived human CD4+ T cell epitopes that bind with high affinity to HLA-DR molecules: Out of a total of 9,594 potential HLA-DR-restricted CD4+ T cell epitopes from the whole genome sequence of SARS-CoV-2-Wuhan-Hu-1 strain (MN908947.3), 16 epitopes that bind with high affinity to HLA-DRB1 molecules were selected. The conservancy of the 16 CD4+ T cell epitopes was analyzed among human and animal Coronaviruses. Shown are the comparison of sequence homology for the 16 CD4+ T cell epitopes among 81,963 SARS-CoV-2 strains (that currently circulate in 6 continents), the 4 major “common cold” Coronaviruses that cased previous outbreaks (i.e. hCoV-OC43, hCoV-229E, hCoV-HKU1, and hCoV-NL63), and the SL-CoVs that were Isolated from bats, civet cats, pangolins and camels. Epitope sequences highlighted in green present high degree of homology among the currently circulating 81,983 SARS-CoV-2 strains and at least a 50% conservancy among two or more humans SARS-CoV strains from previous outbreaks, and the SL-CoV strains isolated from bats, civet cats, pangolins and camels, as described in Materials and Methods. Homo Sapiens-black, bats (Rhinolophus affinis, Rhinolophus malayanus-red), pangolins (Manis javanica-blue), civet cats (Paguma larvata-green), and camels (Camelus dromedaries-brown).

FIG. 11A the molecular docking of highly mutated SARS-CoV-2 CD4+ T cell epitopes to HLA-DRB1 molecules. Molecular docking of 16 CD4+ T cell epitopes, mutated among human SARS-CoV-2 strains, previous humans SARS/MERS-CoV and bat SL-CoVs into the groove of the HLA-DRB1 protein crystal structure (PDB accession no: 4UQ3) was determined using the GalaxyPepDock server. The 16 CD4+ T cell epitopes are promiscuous restricted to HLA-DRB1*01:01, HLA-DRB1*11:01, HLA-DRB1*15:01, HLA-DRB1*03:01 and HLA-DRB1*04:01 alleles. The CD4+ T cell peptides are shown in ball and stick structures, and the HLA-DRB1 protein crystal structure is shown as a template. The prediction accuracy is estimated from a linear model as the relationship between the fraction of correctly predicted binding she residues and the template-target similarity measured by the protein structure similarity score (TM score) and interaction similarity score (Sinter) obtained by linear regression. Sinter shows the similarity of the amino acids of the CD8+ T cell peptides aligned to the contacting residues in the amino acids of the HLA-DRB1 template structure.

FIG. 11B shows histograms representing interaction similarity score of CD4+ T cells specific epitopes observed from the protein-peptide molecular docking analysis.

FIG. 12A shows an experimental design to show CD4+ T cells are specific to highly mutated SARS-CoV-2 epitopes detected in COVID-19 patients and unexposed healthy individuals: PBMCs from HLA-DRB1 positive COVID-19 patients (n=30) and controls unexposed healthy individuals (n=10) were isolated and stimulated for 48 hrs. with 10 μM of each of the 16 SARS-CoV-2-derived CD4+ T cell epitopes. The number of IFN-producing cells were quantified using ELISpot assay.

FIG. 12B shows the results from FIG. 12A. Dotted lines represent a threshold to evaluate the relative magnitude of the response: a mean SFCs between 25 and 50 correspond to a medium/intermediate response, whereas a strong response is defined for a mean SFCs>50. PBMCs from HLA-DRB1-positive COVID-19 patients

FIG. 12C shows the results from further stimulating for an additional 5 hours in the presence of mAbs specific to CD107a and CD107b, and Golgi-plug and Golgi-stop. Tetramers specific to two Spike epitopes, CD107a/b and CD69 and TNF-alpha expressions were then measured by FACS. Representative FACS plot showing the frequencies of Tetramer+CD4+ T cells, CD107a/b+CD4+ T cells, CD69+CD4+ T cells and TNF-+CD4+ T cells following priming with a group of 2 Spike CD4+ T cell epitope peptides. Average frequencies are shown for tetramer+CD4+ T cells, CD107a/b+CD4+ T cells, CD69+CD4+ T cells and TNF-+CD4+ T cells.

FIG. 13A shows a timeline of immunization and Immunological analyses for testing immunogenicity of genome-wide identified human SARS-CoV-2 CD4+ T epitopes in HLA-A*02:01/HLA-DRB1 double transgenic mice. Four groups of age-matched HLA-DRB1 transgenic mice (n=3) were immunized subcutaneously, on days 0 and 14, with a mixture of four SARS-CoV-2-derived human CD4+ T cell peptide epitopes delivered in alum and CpG1826 adjuvants. As a negative control, mice received adjuvants alone (mock-immunized).

FIG. 13B shows the gating strategy used to characterize spleen-derived CD4+ T cells. CD4 positive cells were gated by the CD4 and CD3 expression markers.

FIG. 13C shows the representative ELISpot images (left panel) and average frequencies (right panel) of IFN-γ-producing cell spots from splenocytes (106 cells/well) stimulated for 48 hours with 10 μM of 7 immunodominant CD4+ T cell peptides and 1 subdominant CD4+ T cell peptide out of the total pool of 16 CD4+ T cell peptides derived from SARS-CoV-2 structural and non-structural proteins. The number of IFN-γ-producing spot forming T cells (SFC) per one million of total cells is presented on the top of each ELISpot image.

FIG. 13D shows the representative FACS plot (left panel) and average frequencies (right panel) show IFN-γ and TNF-α-production by, and CD107a/b and CD69 expression on 7 Immunodominant CD4+ T cell peptides and 1 subdominant CD4+ T cell peptide out of the total pool of 16 CD4+ T cell peptides derived from SARS-CoV-2 determined by FACS. The numbers Indicate percentages of IFN-γ+CD4+ T cells, CD107+CD4+ T cells, CD69+CD4+ T cells and TNF-α+CD4+ T cells detected in 3 Immunized mice.

FIG. 14 shows the conservation of Spike-derived B cell epitopes among human, bat, civet cat, pangolin, and camel coronavirus strains: Multiple sequence alignment performed using ClustalW among 29 strains of SARS coronavirus (SARS-CoV) obtained from human, bat, civet, pangolin, and camel. This includes 7 human SARS/MERS-CoV strains (SARS-CoV-2-Wuhan (MN908947.3), SARS-HCoV-Urbani (AY278741.1), CoV-HKU1-Genotype-B (AY884001), CoV-OC43 (KF923903), CoV-NL63 (NC005831), CoV-229E (KY983587), MERS (NC019843)); 8 bat SARS-CoV strains (BAT-SL-CoV-WIV16 (KT444582), BAT-SL-CoV-WIV1 (KF367457.1), BAT-SL-CoV-YNLF31C (KP886808.1), BAT-SARS-CoV-RS672 (FJ588686.1), BAT-CoV-RATG13 (MN996532.1), BAT-CoV-YN01 (EPIISL412976), BAT-CoV-YNO2 (EPIISL412977), BAT-CoV-19-ZXC21 (MG772934.1); 3 Civet SARS-CoV strains (SARS-CoV-Civet007 (AY572034.1), SARS-CoV-A022 (AY686863.1), SARS-CoV-B039 (AY686864.1)); 9 pangolin SARS-CoV strains (PCoV-GX-P2V(MT072864.1), PCoV-GX-P5E(MT040336.1), PCoV-GX-P5L (MT040335.1), PCoV-GX-P1E (MT040334.1), PCoV-GX-P4L (MT040333.1), PCoV-MP789 (MT084071.1), PCoV-GX-P3B (MT072865.1), PCoV-Guangdong-P2S (EPIISL410544), PCoV-Guangdong (EPIISL410721)); 4 camel SARS-CoV strains (Camel-CoV-HKU23 (KT368891.1), DcCoV-HKU23 (MN514967.1), MERS-CoV-Jeddah (KF917527.1), Riyadh/RY141 (NC028752.1)) and 1 recombinant strain (FJ211859.1)). Regions highlighted with blue color represent the sequence homology. The B cell epitopes, which showed at least 50% conservancy among two or more strains of the SARS Coronavirus or possess receptor-binding domain (RBD) specific amino acids were selected as candidate epitopes.

FIG. 15A shows the docking of SARS-CoV-2 Spike glycoprotein-derived B cell epitopes to human ACE2 receptor, e.g., molecular docking of 22 B-cell epitopes, identified from the SARS-CoV-2 Spike glycoprotein, with ACE2 receptors. B cell epitope peptides are shown in ball and stick structures whereas the ACE2 receptor protein is shown as a template. S471-501 and S369-393 peptide epitopes possess receptor binding domain region specific amino acid residues. The prediction accuracy is estimated from a linear model as the relationship between the fraction of correctly predicted binding site residues and the template-target similarity measured by the protein structure similarity score and interaction similarity score (Sinter) obtained by linear regression. Sinter shows the similarity of amino acids of the B-cell peptides aligned to the contacting residues in the amino acids of the ACE2 template structure. Higher Sinter score represents a more significant binding affinity among the ACE2 molecule and B-cell peptides.

FIG. 15B shows the summary of the interaction similarity score of 22 B cells specific epitopes observed from the protein-peptide molecular docking analysis. B cell epitopes with high interaction similarity scores are indicated in black.

FIG. 16A shows the timeline of immunization and immunological analyses for testing to show IgG antibodies are specific to SARS-CoV-2 Spike protein-derived B-cell epitopes in immunized B6 mice and in convalescent COVID-19 patients. A total of 22 SARS-CoV-2 derived B-cell epitope peptides selected from SARS-CoV-2 Spike protein and tested in B6 mice were able to induce antibody responses. Four groups of age-matched B6 mice (n=3) were immunized subcutaneously, on days 0 and 14, with a mixture of 4 or 5 SARS-CoV-2 derived B-cell peptide epitopes emulsified in alum and CpG1826 adjuvants. Alum/CpG1826 adjuvants alone were used as negative controls (mock-Immunized).

FIG. 16B shows the frequencies of IgG-producing CD3(−)CD138(+)B220(+) plasma B cells were determined in the spleen of immunized mice by flow cytometry. For example, FIG. 16B shows the gating strategy was as follows: Lymphocytes were identified by a low forward scatter (FSC) and low side scatter (SSC) gate. Singlets were selected by plotting forward scatter area (FSC-A) versus forward scatter height (FSC-H). B cells were then gated by the expression of CD3(−) and B220(+) cells and CD138 expression on plasma B cells determined.

FIG. 16C shows the frequencies of IgG-producing CD3(−)CD138(+)B220(+) plasma B cells were determined in the spleen of immunized mice by flow cytometry. For example, FG 15C shows a representative FACS plot (left panels) and average frequencies (right panel) of plasma B cells detected in the spleen of immunized mice. The percentages of plasma CD138(−)B220(+)B cells are indicated on the top left of each dot plot.

FIG. 16D shows SARS-CoV-2 derived B-cell epitopes-specific IgG responses were quantified in immune serum, 14 days post-second immunization (i.e. day 28), by ELISpot (Number of IgG(+)Spots). Representative ELISpot images (left panels) and average frequencies (right panel) of anti-peptide specific IgG-producing B cell spots (1×106 splenocytes/well) following 4 days in vitro B cell polyclonal stimulation with mouse Poly-S(Immunospot). The top/left of each ELISpot image shows the number of IgG-producing B cells per half a million cells. ELISA plates were coated with each individual immunizing peptide.

FIG. 16E shows the B-cell epitopes-specific IgG concentrations (μg/mL) measured by ELISA in levels of IgG detected in peptide-immunized B6 mice, after subtraction of the background measured from mock-vaccinated mice. The dashed horizontal line indicates the limit of detection.

FIG. 16F and FIG. 16G show the B-cell epitopes-specific IgG concentrations (μg/mL) measured by ELISA in Level of IgG specific to each of the 22 Spike peptides detected SARS-CoV-2 infected patients (n=40), after subtraction of the background measured from healthy non-exposed individuals (n=10). Black bars and gray bars show high and medium immunogenic B cell peptides, respectively. The dashed horizontal line indicates the limit of detection.

FIG. 17 shows an example of a whole spike protein comprising mutations Including 6 proline mutations. The 6 proline mutations comprise single point mutations F817P, A892P, A899P, A942P, K986P and V987P. Additionally, the spike protein comprises a 682-QQAQ-685 mutation of the furin cleavage site for protease resistance. In some embodiments, the K986P and V987P Mutations allow for perfusion stabilization. Note MFVFLVLLPLVSS (SEQ ID NO: 63), ATGTTCGTGTTCCTGGTGCTGCTGCCCCTGGTGAGCAGC (SEQ ID NO: 171), and CAGCAGGCCCAG (SEQ ID NO: 179), are shown in FIG. 17.

FIG. 18 shows a schematic representation of a prototype Coronavirus vaccine of the present invention. The present invention is not limited to the prototype coronavirus vaccines as shown. non limiting examples of vaccine compositions described herein.

FIG. 19 shows schematic views of non-limiting examples of vaccine compositions showing an optional molecular adjuvant, T cell attracting chemokine, and/or composition for promoting T cell proliferation, as well as non-limiting examples of orientations of said optional molecular adjuvant. T cell attracting chemokine, and/or composition for promoting T cell proliferation.

FIG. 20 shows a non-limiting example of an adeno-associated virus vector comprising a multi-epitope pan-coronavirus vaccine composition operably linked to a lung specific promoter (e.g. SP-B promoter or a CD144 promoter). Additionally, the multi-epitope pan-coronavirus vaccine composition comprises a His tag. The adeno-associated virus vector also comprises an adjuvant (e.g. CpG) operable linked to a lung specific promoter (e.g. SP-B promoter or a CD144 promoter).

FIG. 21 shows a non-limiting example of an adeno-associated virus vector comprising a multi-epitope pan-coronavirus vaccine composition operably linked to a lung specific promoter (e.g. s SP-B promoter or a CD144 promoter). Additionally, the multi-epitope pan-coronavirus vaccine composition comprises a His tag. The adeno-associated virus vector also comprises an adjuvant (e.g. flagellin) operable linked to a second lung specific promoter (e.g. SP-B promoter or a CD144 promoter).

FIG. 22 shows a non-limiting example of an adeno-associated virus vector comprising a multi-epitope pan-coronavirus vaccine composition operably linked to a generic promoter (e.g. a CMV promoter or a CAG promoter). Additionally, the multi-epitope pan-coronavirus vaccine composition comprises a His tag. The adeno-associated virus vector also comprises at least one T cell enhancement composition (e.g. IL-7, or CXCL11) operably linked to a second generic promoter (e.g. a CMV promoter or a CAG promoter). The additional T-cell enhancement composition improves the immunogenicity and long-term memory of the multi-epitope pan-coronavirus vaccine composition by co-expressing IL-7 cytokine and T-cell attracting chemokine CXCL11, both driven with another CMV promoter and linked with a T2A spacer in AAV9 vector.

FIG. 23 shows a non-limiting example of an adeno-associated virus vector comprising a multi-epitope pan-coronavirus vaccine composition operably linked to a generic promoter (e.g. a CMV promoter or a CAG promoter). Additionally, the multi-epitope pan-coronavirus vaccine composition comprises a His tag and at least one T cell enhancement composition (e.g. IL-7, or CXCL11). to improve the immunogenicity and long-term memory the multi-epitope pan-coronavirus vaccine composition is driven with a single CMV promoter and co-expressed in AAV9 vector with IL-7 cytokine and T-cell attracting chemokine CXCL11 driven with same CMV promoter and linked with a T2A spacer.

FIG. 24 shows non-limiting examples of how the target epitopes of the compositions described herein may be arranged. In addition to a string of epitopes (i.e. “string-of-peals”), the composition of the present invention may also feature a spike protein or portion thereof in combination with target epitopes

FIG. 25A shows a non-limiting example of a method for delivering the vaccine composition described herein using a “prime/pull” regimen in humans. The method comprises administering a pan-coronavirus recombinant vaccine composition and further administering at least one T-cell attracting chemokine (e.g. CXCL11) after administering the pan-coronavirus recombinant vaccine composition.

FIG. 25B shows a non-limiting example of a method for delivering the vaccine composition described herein using a “prime/boost” regimen in humans. The method comprises administering a first composition, e.g., a first pan-coronavirus recombinant vaccine composition dose using a first delivery system and further administering a second composition, e.g., a second vaccine composition dose using a second delivery system. In some embodiments, the first delivery system and the second delivery system are different.

FIG. 25C shows a non-limiting example of a method for delivering the vaccine composition described herein using a “prime/pull/keep” regimen in humans to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition.

FIG. 25D shows a non-limiting example of a method for delivering the vaccine composition described herein using a “prime/pull/boost” regimen in humans to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition. The method further comprises administering at least one cytokine after administering the T-cell attracting chemokine (e.g. IL-7, IL-5, or IL-2).

FIG. 26A shows a non-limiting example of a method for delivering the vaccine composition described herein using a “prime/pull” regimen in domestic animals (e.g. cats or dogs). The method comprises administering a pan-coronavirus recombinant vaccine composition and further administering at least one T-cell attracting chemokine (e.g. CXCL11) after administering the pan-coronavirus recombinant vaccine composition.

FIG. 28B shows a non-limiting example of a method for delivering the vaccine composition described herein using a “prime/boost” regimen in domestic animals (e.g. cats or dogs). The method comprises administering a first composition, e.g., a first pan-coronavirus recombinant vaccine composition dose using a first delivery system and further administering a second composition, e.g., a second vaccine composition dose using a second delivery system. In some embodiments, the first delivery system and the second delivery system are different.

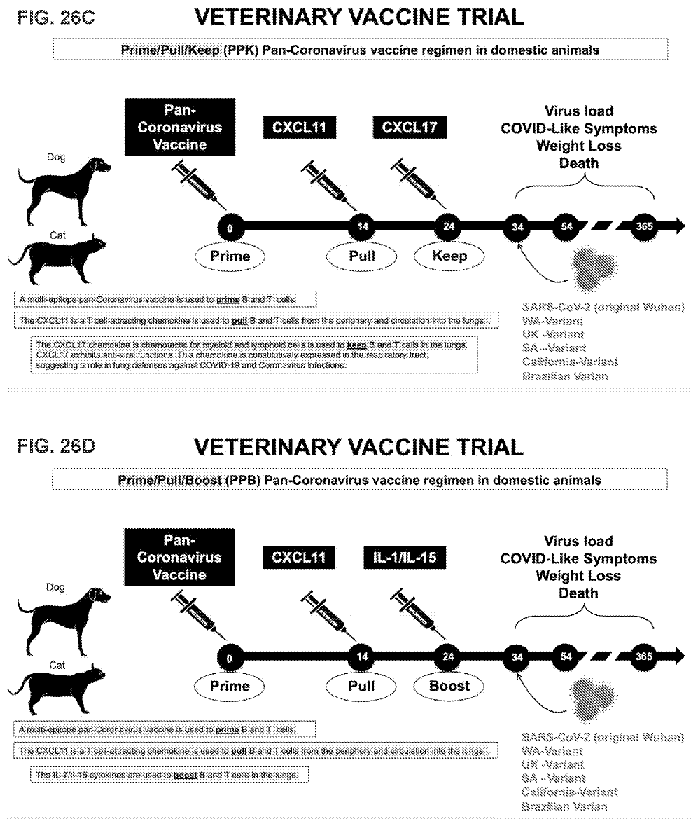

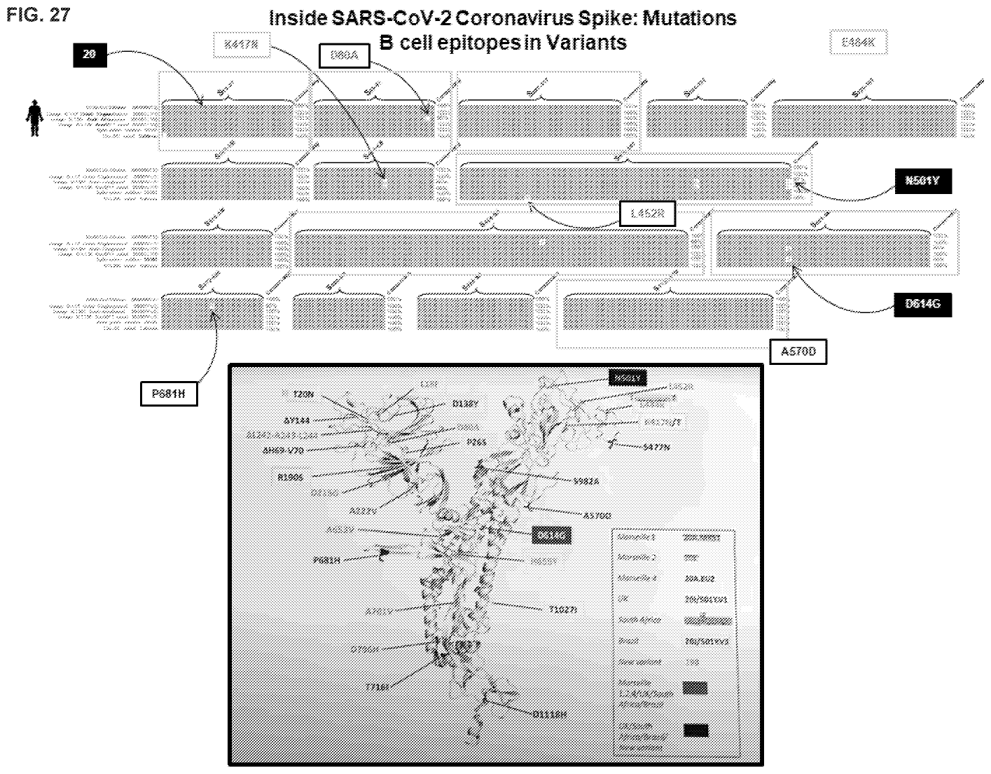

FIG. 26C shows a non-limiting example of a method for delivering the vaccine composition described herein using a “prime/pull/keep” regimen in domestic animals (e.g. cats or dogs) to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition.

FIG. 26D shows a non-limiting example of a method for delivering the vaccine composition described herein using a “prime/pull/boost” regimen in domestic animals (e.g. cats or dogs) to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition. The method further comprises administering at least one cytokine after administering the T-cell attracting chemokine (e.g. IL-7, IL-5, or IL-2).

FIG. 27 shows non-limiting examples of SARS-CoV-2 Coronavirus spike glycoprotein mutations within the B cell epitopes in various variants.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990: and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods In Enzymology (M. P. Deutshcer, ed., (1990) Academic Press, Inc.); PCR Protocols: A Guide to Methods and Applications (Innis, et al. 1990. Academic Press. San Diego, Calif.). Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ad. E. J. Murray, The Humana Press Inc., Clifton. N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and not intended to be limiting.

As used herein, the terms “immunogenic protein, polypeptide, or peptide” or “antigen” refer to polypeptides or other molecules (or combinations of polypeptides and other molecules) that are immunologically active in the sense that once administered to the host, it is able to evoke an immune response of the humoral and/or cellular type directed against the protein. In embodiments, the protein fragment has substantially the same Immunological activity as the total protein. Thus, a protein fragment according to the disclosure can comprises or consists essentially of or consists of at least one epitope or antigenic determinant. An “immunogenic” protein or polypeptide, as used herein, may include the full-length sequence of the protein, analogs thereof, or immunogenic fragments thereof. “Immunogenic fragment” refers to a fragment of a protein which includes one or more epitopes and thus elicits the immunological response described above.

Synthetic antigens are also included within the definition, for example, poly-epitopes, flanking epitopes, and other recombinant or synthetically derived antigens. Immunogenic fragments for purposes of the disclosure may feature at least about 1 amino acid, at least about 3 amino acids, at least about 5 amino acids, at least about 10-15 amino acids, or about 15-25 amino acids or more amino acids, of the molecule. There is no critical upper limit to the length of the fragment, which could comprise nearly the full-length of the protein sequence, or the full-length of the protein sequence, or even a fusion protein comprising at least one epitope of the protein.

As used herein, the term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

As used herein, the term “immunological response” to a composition or vaccine refers to the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. The host may display either a therapeutic or protective immunological response so resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the term “variant” refers to a substantially similar sequence. For polynucleotides, a variant comprises a deletion and/or addition and/or change of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or an amino acid sequence, respectively. Variants of a particular polynucleotide of the disclosure (e.g., the reference polynucleotide) can also be evaluated by comparison of the percent sequence identity between the polypeptide encoded by a variant polynucleotide and the polypeptide encoded by the reference polynucleotide. “Variant” protein is intended to mean a protein derived from the native protein by deletion or addition of one or more amino acids at one or more sites in the native protein and/or substitution of one or more amino acids at one or more sites in the native protein. Variant proteins encompassed by the present disclosure are biologically active, that is they have the ability to elicit an immune response.

The HLA-DR/HLA-A*0201/hACE2 triple transgenic mouse model referred to herein is a novel susceptible animal model for pre-clinical testing of human COVID-19 vaccine candidates derived from crossing ACE2 transgenic mice with the unique HLA-DR/HLA-A*0201 double transgenic mice. ACE2 transgenic mice are a hACE2 transgenic mouse model expressing human ACE2 receptors in the lung, heart, kidney and intestine (Jackson Laboratory, Bar Harbor, Me.). The HLA-DR/HLA-A*0201 double transgenic mice are “humanized” HLA double transgenic mice expressing Human Leukocyte Antigen HLA-A*0201 class I and HLA DR*0101 class II in place of the corresponding mouse MHC molecules (which are knocked out). The HLA-A*0201 haplotype was chosen because it is highly represented (>50%) in the human population, regardless of race or ethnicity. The HLA-DR/HLA-A*0201/hACE2 triple transgenic mouse model is a “humanized” transgenic mouse model and has three advantages: (1) it is susceptible to human SARS-CoV2 infection; (2) it develops symptoms similar to those seen in COVID-19 in humans; and (3) it develops CD4⁺ T cells and CD8⁺ T cells response to human epitopes. The novel HLA-DR/HLA-A*0201/hACE2 triple transgenic mouse model of the present invention may be used in the pre-clinical testing of safety, immunogenicity and protective efficacy of the human multi-epitope COVID-19 vaccine candidates of the present invention.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” If the progression of a disease is reduced or halted. That is, “treatment” Includes not just the improvement of symptoms or decrease of markers of the disease, but also a cessation or slowing of progress or worsening of a symptom that would be expected in absence of treatment. Beneficial or desired clinical results Include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” also includes ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

As used herein, the term “carrier” or “pharmaceutically acceptable carrier” or “pharmaceutically acceptable vehicle” refers to any appropriate or useful carrier or vehicle for Introducing a composition to a subject. Pharmaceutically acceptable carriers or vehicles may be conventional but are not limited to conventional vehicles. For example, E. W. Martin, Remington's Pharmaceutical Sciences, Mack Publishing Co., Easton, Pa., 15th Edition (1975) and D. B. Troy, ed. Remington: The Science and Practice of Pharmacy, Lippincott Williams & Wilkins, Baltimore Md. and Philadelphia, Pa., 21^{st} Edition (2006) describe compositions and formulations suitable for pharmaceutical delivery of one or more therapeutic compounds or molecules. Carriers (e.g., pharmaceutical carriers, pharmaceutical vehicles, pharmaceutical compositions, pharmaceutical molecules, etc.) are materials generally known to deliver molecules, proteins, cells and/or drugs and/or other appropriate material into the body. In general, the nature of the carrier will depend on the nature of the composition being delivered as well as the particular mode of administration being employed. In addition to biologically-neutral carriers, pharmaceutical compositions administered may contain minor amounts of non-toxic auxiliary substances, such as wetting or emulsifying agents, preservatives, and pH buffering agents and the like. Patents that describe pharmaceutical carriers include, but are not limited to: U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, the disclosures of which are incorporated in their entirety by reference herein. The carrier may, for example, be solid, liquid (e.g., a solution), foam, a gel, the like, or a combination thereof. In some embodiments, the carrier comprises a biological matrix (e.g., biological fibers, etc.). In some embodiments, the carrier comprises a synthetic matrix (e.g., synthetic fibers, etc.). In certain embodiments, a portion of the carrier may comprise a biological matrix and a portion may comprise synthetic matrix.

As used herein “coronavirus” may refer to a group of related viruses such as but not limited to severe acute respiratory syndrome (SARS), middle east respiratory syndrome (MERS), and severe acute respiratory syndrome coronavirus 2 (SARS-CoV-2). All the coronaviruses cause respiratory tract infection that range from mild to lethal in mammals. Several non-limiting examples of Coronavirus strains are described herein. In some embodiments, the compositions may protect against any Sarbecoviruses including but not limited to SARS-CoV1 or SARS-CoV2.

As used herein, “severe acute respiratory syndrome coronavirus 2 (SARS-CoV2)” is a betacoronavirus that causes Coronavirus Disease 19 (COVID-19).

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects

The terms “administering”, and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like.

A composition can also be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering a composition comprising the vaccine composition, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

A composition can also be administered by buccal delivery or by sublingual delivery. As used herein “buccal delivery” may refer to a method of administration in which the compound is delivered through the mucosal membranes lining the cheeks. In some embodiment, for a buccal delivery the vaccine composition is placed between the gum and the cheek of a patient. As used herein “sublingual delivery” may refer to a method of administration in which the compound is delivered through the mucosal membrane under the tongue. In some embodiments, for a sublingual delivery the vaccine composition is administered under the tongue of a patient.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration Involves use of a slow release or sustained release system such that a constant dosage is maintained. See, for example, U.S. Pat. No. 3,610,795, which is Incorporated by reference herein.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting. Embodiments of the present invention can be freely combined with each other if they are not mutually exclusive.

### Multi-Epitope Pan-Coronavirus Vaccines

The present invention features Coronavirus vaccine compositions, methods of use, and methods of producing said vaccines, methods of preventing coronavirus infections, etc. The present invention also provides methods of testing said vaccines, e.g., using particular animal models and clinical trials. The vaccine compositions herein can induce efficient and powerful protection against the coronavirus disease or infection, e.g., by inducing the production of antibodies (Abs), CD4⁺ T helper (Th1) cells, and CD⁺8 cytotoxic T-cells (CTL).

The vaccine compositions, e.g., the antigens, herein feature multiple epitopes, which helps provide multiple opportunities for the body to develop an immune response for preventing an Infection.

In certain embodiments, the epitopes comprise mutations from variant strains of human coronaviruses and/or animal coronaviruses (e.g., coronaviruses isolated from animals susceptible to coronavirus Infections). In other embodiments, the epitopes are highly mutated among human coronaviruses and/or animal coronaviruses (e.g., coronaviruses isolated from animals susceptible to coronavirus infections). The vaccines herein may be designed to be effective against past, current, and future coronavirus outbreaks.

The target epitopes may be derived from structural (e.g., spike glycoprotein, envelope protein, membrane protein, nucleoprotein) or non-structural proteins of the coronaviruses.

In some embodiments, the vaccine composition comprises one or more coronavirus B-cell target epitopes; one or more coronavirus CD4⁺ T cell target epitopes; and one or more coronavirus CD8⁺ T cell target epitopes. In some embodiments, the vaccine composition comprises one or more coronavirus B-cell target epitopes and one or more coronavirus CD4⁺ T cell target epitopes. In some embodiments, the vaccine composition comprises one or more coronavirus B-cell target epitopes and one or more coronavirus CD8⁺ T cell target epitopes. In some embodiments, the vaccine composition comprises one or more coronavirus CD8⁺ target epitopes and one or more coronavirus CD4⁺ T cell target epitopes. In some embodiments, the vaccine composition comprises one or more coronavirus CD8⁺ target epitopes. In some embodiments, the vaccine composition comprises one or more coronavirus CD4⁺ target epitopes. In some embodiments, the vaccine composition comprises one or more coronavirus B cell target epitopes.

In some embodiments, the vaccine composition comprises mutated target epitopes. In some embodiments, the vaccine composition comprises mutated target epitopes. In some embodiments, the vaccine composition comprises a combination of mutated and mutated target epitopes

As will be discussed herein, in certain embodiments, the vaccine composition comprises whole spike protein, one or more coronavirus CD4⁺ T cell target epitopes; and one or more coronavirus CD8⁺ T cell target epitopes. In certain embodiments, the vaccine composition comprises at least a portion of the spike protein (e.g., wherein the portion comprises a trimerized SARS-CoV-2 receptor-binding domain (RBD)), one or more coronavirus CD4⁺ T cell target epitopes; and one or more coronavirus CD8⁺ T cell target epitopes.

In certain embodiments, the vaccine composition comprises one or more coronavirus B cell target epitopes, one or more coronavirus CD4 T cell target epitopes: and one or more coronavirus CD8⁺ T cell target epitopes. For example, in certain embodiments, the vaccine composition comprises 4 B cell target epitopes, 15 CD8⁺ T cell target epitopes, and 6 CD4⁺ T cell target epitopes. The present invention is not limited to said combination of epitopes.

In certain embodiments, the vaccine composition comprises 1-10 B cell target epitopes. In certain embodiments, the vaccine composition comprises 2-10 B cell target epitopes. In certain embodiments, the vaccine composition comprises 2-15 B cell target epitopes. In certain embodiments, the vaccine composition comprises 2-20 B cell target epitopes. In certain embodiments, the vaccine composition comprises 2-30 B cell target epitopes. In certain embodiments, the vaccine composition comprises 2-15 B cell target epitopes. In certain embodiments, the vaccine composition comprises 2-5 B cell target epitopes. In certain embodiments, the vaccine composition comprises 5-10 B cell target epitopes. In certain embodiments, the vaccine composition comprises 5-15 B cell target epitopes. In certain embodiments, the vaccine composition comprises 5-20 B cell target epitopes. In certain embodiments, the vaccine composition comprises 5-25 B cell target epitopes. In certain embodiments, the vaccine composition comprises 5-30 B cell target epitopes. In certain embodiments, the vaccine composition comprises 10-20 B cell target epitopes. In certain embodiments, the vaccine composition comprises 10-30 B cell target epitopes.

In certain embodiments, the vaccine composition comprises 1-10 CD8⁺ T cell target epitopes. In certain embodiments, the vaccine composition comprises 2-10 CD8⁺ T cell target epitopes. In certain embodiments, the vaccine composition comprises 2-15 CD8⁺ T cell target epitopes. In certain embodiments, the vaccine composition comprises 2-20 CD8⁺ T cell target epitopes. In certain embodiments, the vaccine composition comprises 2-30 CD8⁺ T cell target epitopes. In certain embodiments, the vaccine composition comprises 2-15 CD8⁺ T cell target epitopes. In certain embodiments, the vaccine composition comprises 2-5 CD8⁺ T cell target epitopes. In certain embodiments, the vaccine composition comprises 5-10 CD8⁺ T cell target epitopes. In certain embodiments, the vaccine composition comprises 5-15 CD8⁺ T cell target epitopes. In certain embodiments, the vaccine composition comprises 5-20 CD8⁺ T cell target epitopes. In certain embodiments, the vaccine composition comprises 5-25 CD8⁺ T cell target epitopes. In certain embodiments, the vaccine composition comprises 5-30 CD8⁺ T cell target epitopes. In certain embodiments, the vaccine composition comprises 10-20 CD8⁺ T cell target epitopes. In certain embodiments, the vaccine composition comprises 10-30 CD8⁺ T cell target epitopes.

In certain embodiments, the vaccine composition comprises 1-10 CD4⁺ T cell target epitopes. In certain embodiments, the vaccine composition comprises 2-10 CD4⁺ T cell target epitopes. In certain embodiments, the vaccine composition comprises 2-15 CD4⁺ T cell target epitopes. In certain embodiments, the vaccine composition comprises 2-20 CD4⁺ T cell target epitopes. In certain embodiments, the vaccine composition comprises 2-30 CD4⁺ T cell target epitopes. In certain embodiments, the vaccine composition comprises 2-15 CD4⁺ T cell target epitopes. In certain embodiments, the vaccine composition comprises 2-5 CD4⁺ T cell target epitopes. In certain embodiments, the vaccine composition comprises 5-10 CD4⁺ T cell target epitopes. In certain embodiments, the vaccine composition comprises 5-15 CD4⁺ T cell target epitopes. In certain embodiments, the vaccine composition comprises 5-20 CD4⁺ T cell target epitopes. In certain embodiments, the vaccine composition comprises 5-25 CD4⁺ T cell target epitopes. In certain embodiments, the composition comprises 5-30 CD4⁺ T cell target epitopes. In certain embodiments, the vaccine composition comprises 10-20 CD4⁺ T cell target epitopes. In certain embodiments, the vaccine composition comprises 10-30 CD4⁺ T cell target epitopes.

Table 1 below further describes various non-limiting combinations of numbers of CD4 T cell target epitopes, CD8⁺ T cell target epitopes, and B cell target epitopes. The present invention is not limited to the examples described herein. In some embodiments, the target epitopes may be mutated, mutated, or a combination thereof.

TABLE 1

| | # B Cell | # CD8* T Cell | # CD4* T Cell |
|---|---|---|---|
| Example | Epitopes | Epitopes | Epitopes |
| 1 | 4 | 15 | 6 |
| 2 | 5 | 10 | 7 |
| 3 | 4 | 12 | 8 |
| 4 | 1 | 16 | 9 |
| 5 | 2 | 2 | 2 |
| 6 | 1 | 5 | 5 |
| 7 | 4 | 6 | 6 |
| 8 | 3 | 12 | 4 |
| 9 | 3 | 3 | 3 |
| 10 | 1 | 14 | 8 |
| 11 | 2 | 10 | 5 |
| 12 | 4 | 9 | 3 |
| 13 | 3 | 3 | 7 |
| 14 | 5 | 11 | 4 |
| 15 | 2 | 8 | 6 |
| 16 | 3 | 9 | 8 |
| 17 | 2 | 10 | 4 |
| 18 | 4 | 6 | 7 |
| 19 | 3 | 14 | 3 |
| 20 | 2 | 4 | 5 |

The epitopes may be each separated by a linker. In certain embodiments, the linker allows for an enzyme to cleave between the target epitopes. The present invention is not limited to particular linkers or particular lengths of linkers. As an example, in certain embodiments, one or more epitopes may be separated by a linker 2 amino acids in length. In certain embodiments, one or more epitopes may be separated by a linker 3 amino acids in length. In certain embodiments, one or more epitopes may be separated by a linker 4 amino acids in length. In certain embodiments, one or more epitopes may be separated by a linker 5 amino acids in length. In certain embodiments, one or more epitopes may be separated by a linker 6 amino acids in length. In certain embodiments, one or more epitopes may be separated by a linker 7 amino acids in length. In certain embodiments, one or more epitopes may be separated by a linker 8 amino acids in length. In certain embodiments, one or more epitopes may be separated by a linker 9 amino acids in length. In certain embodiments, one or more epitopes may be separated by a linker 10 amino acids in length. In certain embodiments, one or more epitopes may be separated by a linker from 2 to 10 amino acids in length.

Linkers are well known to one of ordinary skill in the art. Non-limiting examples of linkers include AAY, KK, and GPGPG. For example, in certain embodiments, one or more CD8⁺ T cell epitopes are separated by AAY. In some embodiments, one or more CD4⁺ T cell epitopes are separated by GPGPG. In certain embodiments, one or more B cell epitopes are separated by KK. In certain embodiments, KK is a linker between a CD4⁺ T cell epitope and a B cell epitope. In certain embodiments, KK is a linker between a CD8⁺ T cell epitope and a B cell epitope. In certain embodiments, KK is a linker between a CD8⁺ T cell epitope and a CD4⁺ T cell epitope. In certain embodiments, AAY is a linker between a CD4 T cell epitope and a B cell epitope. In certain embodiments, AAY is a linker between a CD8⁺ T cell epitope and a B cell epitope. In certain embodiments, AAY is a linker between a CD8⁺ T cell epitope and a CD4⁺ T cell epitope. In certain embodiments, GPGPG is a linker between a CD4⁺ T cell epitope and a B cell epitope. In certain embodiments, GPGPG is a linker between a CD8⁺ T cell epitope and a B cell epitope. In certain embodiments, GPGPG is a linker between a CD8⁺ T cell epitope and a CD4 T cell epitope.

The target epitopes may be derived from structural proteins, non-structural proteins, or a combination thereof. For example, structural proteins may include spike proteins (S), envelope proteins (E), membrane proteins (M), or nucleoproteins (N).

In some embodiments, the target epitopes are derived from at least one SARS-CoV-2 protein. The SARS-CoV-2 proteins may include ORF1ab protein, Spike glycoprotein, ORF3a protein, Envelope protein, Membrane glycoprotein, ORF6 protein, ORF7a protein, ORF7b protein, ORF8 protein, Nucleocapsid protein, and ORF10 protein. The ORF1ab protein provides nonstructural proteins (Nsp) such as Nsp1, Nsp2, Nsp3 (Papain-like protease), Nsp4, Nsp5 (3C-like protease), Nsp6, Nsp7, Nsp8, Nsp9, Nsp10, Nsp11, Nsp12 (RNA polymerase), Nsp13 (5′ RNA triphosphatase enzyme), Nsp14 (guanosineN7-methyltransferase), Nsp15 (endoribonuclease), and Nsp16 (2′-O-ribose-methyltransferase).

The SARS-CoV-2 has a genome length of 29,903 base pairs (bps) ssRNA (SEQ ID NO: 1). Generally, the region between 266-21555 bps codes for ORF1ab polypeptide: the region between 21583-25384 bps codes for one of the structural proteins (spike protein or surface glycoprotein); the region between 25393-26220 bps codes for the ORF3a gene; the region between 26245-26472 bps codes for the envelope protein; the region between 26523-27191 codes for the membrane glycoprotein (or membrane protein); the region between 27202-27387 bps codes for the ORF6 gene; the region between 27394-27759 bps codes for the ORF7a gene; the region between 27894-28259 bps codes for the ORF8 gene; the region between 28274-29533 bps codes for the nucleocapsid phosphoprotein (or the nucleocapsid protein); and the region between 29558-29674 bps codes for the ORF10 gene.

The one or more CD8⁺ T cell target epitopes may be derived from a protein selected from: spike glycoprotein. Envelope protein, ORF1ab protein, ORF7a protein, ORF8a protein, ORF10 protein, or a combination thereof. The one or more CD4⁺ T cell target epitopes may be derived from a protein selected from: spike glycoprotein, Envelope protein, Membrane protein, Nucleocapsid protein. ORF1a protein, ORF1ab protein, ORF6 protein, ORF7a protein, ORF7b protein, ORF8 protein, or a combination thereof. The one or more B cell target epitopes may be derived from the spike protein.

### Mutations

The present invention features a coronavirus vaccine composition. In some embodiments, the composition comprises at least two of: one or more coronavirus B cell target epitopes, one or more coronavirus CD4+ T cell target epitopes; or one or more coronavirus CD8+ T cell target epitopes. In some embodiments, the epitopes are derived from a human coronavirus, an animal coronavirus, or a combination thereof. In certain embodiments, at least one of the epitopes is derived from a non-spike protein. In certain embodiments the composition induced immunity only to the epitopes.

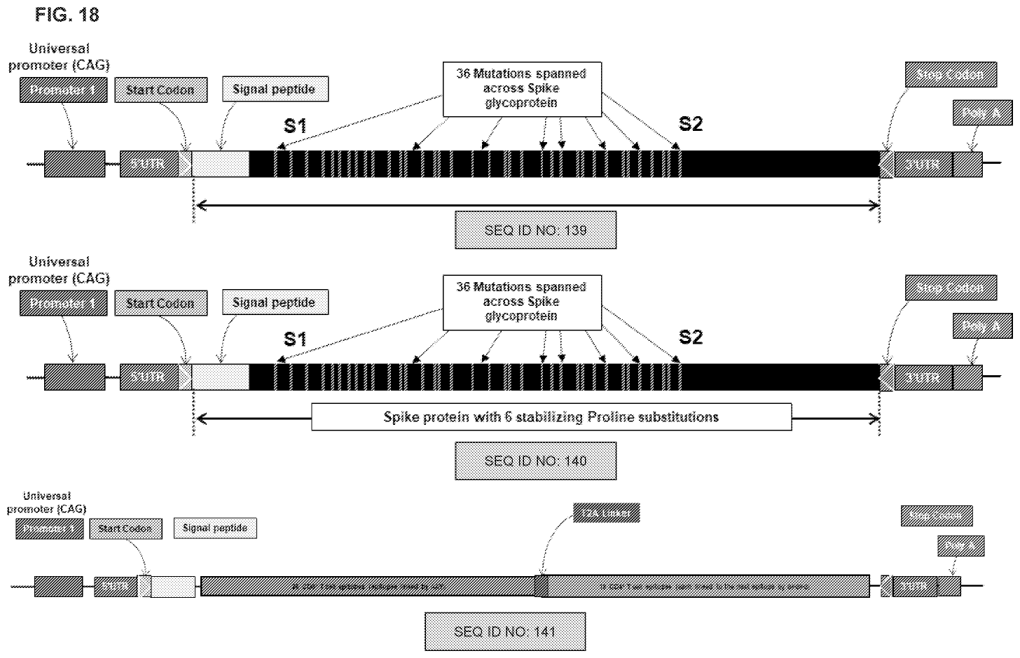

For example, the present invention features pan-coronavirus recombinant vaccine compositions featuring whole proteins or sequences of proteins encompassing all mutations in variants of human and animal Coronaviruses (e.g., 38 mutations in spike protein shown in FIG. 18) or a combination of mutated B cell epitopes, mutated combination of B cell epitopes, mutated CD4+ T cell epitopes, and mutated CD8+ T cell epitopes, at least one of which is derived from a non-spike protein. The mutated epitopes may comprise one or more mutations. The present invention also describes using several immuno-informatics and sequence alignment approaches to Identify several human B cell, CD4+ and CD8+ T cell epitopes that are highly mutated.

In some embodiments, the human coronavirus is the SARS-CoV-2 original strain. e.g., SARS-CoV-2 isolate Wuhan-Hu-1. In some embodiments, the human coronavirus is a SARS-CoV-2 variant, such as but not limited to a variant of SARS-CoV-2 isolate Wuhan-Hu-1.

As used herein, “variant” may refer to a strain having one or more nucleic acid or amino acid mutations as compared to the original strain (such as but not limited to SARS-CoV-2 isolate Wuhan-Hu-1). In some embodiments, the SARS-CoV-2 variant epitope is derived from one or more of: strain B.1.177; strain B.1.180, strain B.1.1.7; strain B.1.351; strain P.1; strain B.1.427/8.1.429; strain B.1.258; strain B.1.221; strain B.1.387; strain B.1.1.277; strain B.1.1.302; strain B.1.525; strain B.1.526, strain S:677H, or strain S:877P.

In some embodiments, the animal coronavirus is a coronaviruses Isolated from animals selected from a group consisting of bats, pangolins, civet cats, minks, camels, and other animal receptive to coronaviruses.

Additionally, other coronaviruses may be used for determining mutated epitopes (including human SARS-CoVs as well as animal CoVs (e.g., bats, pangolins, civet cats, minks, camels, etc.)) that meet the criteria to be classified as “variants of concern” or “variants of interest.” Coronavirus variants that appear to meet one or more of the undermentioned criteria may be labeled “variants of interest” or “variants under investigation” pending verification and validation of these properties. In some embodiments, the criteria may include increased transmissibility, increased morbidity, increased mortality, increased risk of “long COVID”, ability to evade detection by diagnostic tests, decreased susceptibility to antiviral drugs (if and when such drugs are available), decreased susceptibility to neutralizing antibodies, either therapeutic (e.g., convalescent plasma or monoclonal antibodies) or in laboratory experiments, ability to evade natural immunity (e.g., causing reinfections), ability to infect vaccinated individuals, increased risk of particular conditions such as multisystem inflammatory syndrome or long-haul COVID or increased affinity for particular demographic or clinical groups, such as children or immunocompromised individuals. Once validated, variants of interest are renamed “variant of concern” by monitoring organizations, such as the CDC.

The vaccine composition may comprise mutated epitopes or large sequences. As used herein, the term “mutated” or “mutation” may refer to a change in one or more nucleic acids (or amino acids) as compared to the original sequence. In some embodiments, a nucleic acid mutation may be synonymous or non-synonymous.

In some embodiments, the epitope may comprise a D614G mutation, a T445C mutation, a C6288T mutation, a C26801G mutation, a C4543T mutation, a G5629T mutation, a C11497T mutation, a T26878C mutation, a C241T mutation, a C913T mutation, a C3037T mutation, a C5986T mutation, a C14678T mutation, a C15279T mutation, a T16176C mutation, a G174T mutation, a C241T mutation, a C3037T mutation, a C28253T mutation, a C241T mutation, a T733C mutation, a C2749T mutation, a C3037T mutation, a A6319G mutation, a A6813G mutation, a C12778T mutation, a C13860T mutation, a A28877T mutation, a G28878C mutation, a C2395T mutation, a T2597C mutation, a T24349C mutation, a G27890T mutation, a A28272T mutation, a C8047T mutation, a C28651T mutation, a G4980T mutation, a C6070T mutation, a C7303T mutation, a C7564T mutation, a C10279T mutation, a C10525T mutation, a C10582T mutation, a C27804T mutation, a C241T mutation, a C1498T mutation, a A1807G mutation, a G2659A mutation, a C3037T mutation, a T8593C mutation, a C9593T mutation, a C18171T mutation, a A20724G mutation, a C24748T mutation, a A28899G mutation, a G29543T mutation, a C241T mutation, a C3037T mutation, a A20262G mutation, a A28271- mutation, a C241T mutation, a G1942T mutation, a C3037T mutation, a A9085G mutation, a C14805T mutation, a C241T mutation, a C3037T mutation, a C21811A mutation, a T29194C mutation, a T29377 mutation, or combination thereof.

In some embodiments, the mutation may be a point mutation. In other embodiments, the mutation may be a single point mutation (such as the above mentioned mutations). In other embodiments, a single point mutation may be substitutions, deletions, or inversions.

In some embodiments, the mutations may be in any of the SARS-CoV-2 proteins which may include ORF1ab protein, Spike glycoprotein, ORF3a protein, Envelope protein, Membrane glycoprotein, ORF6 protein, ORF7a protein, ORF7b protein, ORF8 protein, Nucleocapsid protein, or ORF10 protein.

In some embodiments, mutations in the spike (S) protein may include but are not limited to A22V, S477N, H69-, V70-, Y144-, N501Y, A570D, P681H, D80A, D215G, L241-, L242-, A243-, K417N, E484K, N501Y, A701V, L18F, K417T, E484K, N501Y, H855Y, S13I, W152C, L452R, S439K, S98F, D80Y, A626S, V1122L, A67V, H69-, V70-, Y144-, E484K, Q677H, F888L, L5F, T95I, D253G, E484K, A701V, Q677H, Q677P or a combination thereof (also see FIG. 27)

As previously discussed, in some embodiments, the composition comprises spike protein or portion thereof. Non-limiting examples of spike proteins with and without mutations are listed in Table 2.

TABLE 2

| | | SEQ ID |
|---|---|---|
| | Sequence: | NO: |
| SARS-CoV-like | SQCVNLTTRTQLPPAYTNSFTRGVYYPDKVFRSSVLHSTQDLFLPF | 143 |
| Spike-S1-NTD | FSNVTWFHAIHVSGTNGTKRFDNPVLPFNDGVYFASTEKSNIIRGWI | |
| 13 bp-304 bp | FGTTLDSKTQSLLIVNNATNVVIKVCEFQFCNDPFLGVYYHKNNKSW | |
| | MESEFRVYSSANNCTFEYVSQPFLMDLEGKQGNFKNLREFVFKNID | |
| | GYFKIYSKHTPINLVRDLPQGFSALEPLVDLPIGINITRFQTLLALHRS | |
| | YLTPGDSSSGWTAGAAAYYVGYLQPRTFLLKYNENGTITDAVDCAL | |
| | DPLSETKCTLK | |
| SARS-CoV-2 | RVQPTESIVRFPNITNLCPFGEVFNATRFASVYAWNRKRISNCVADY | 144 |
| Spike-S1-RBD | SVLYNSASFSTFKCYGVSPTKLNDLCFTNVYADSFVIRGDEVRQIAP | |
| 319 bp-541 bp | GQTGKIADYNYKLPDDFTGCVIAWNSNNLDSKVGGNYNYLYRLFRK | |
| | SNLKPFERDISTEIYQAGSTPCNGVEGFNCYFPLQSYGFQPTNGVG | |
| | YQPYRVVVLSFELLHAPATVCGPKKSTNLVKNKCVNF | |
| CoV Spike S1- | FNGLTGTGVLTESNKKFLPFQQFGRDIADTTDAVTRAGCLIGAEHVN | 145 |
| S2_S2 | NSYECDIPIGAGICASYQTQTNRDPQTLEILDITPCSFGGVSVITPGT | |
| 543 bp-1,208 bp | NTSNQVAVLYQDVNCTEVPVAIHADQLTPTWRVYSTGSNVFQSPR | |
| | RARSVASQSIIAYTMSLGAENSVAYSNNSIAIPTNFTISVTTEILPVSM | |
| | TKTSVDCTMYICGDSTECSNLLLQYGSFCTQLNRALTGIAVEQDKNT | |
| | QEVFAQVKQIYKTPPIKDFGGFNFSQILPDPSKPSKRSFIEDLLFNKV | |
| | TLADAGFIKQYGDCLGDIAARDLICAQKFNGLTVLPPLLTDEMIAQYT | |
| | SALLAGTITSGWTFGAGAALQIPFAMQMAYRFNGIGVTQNVLYENQ | |
| | KLIANQFNSAIGKIQDSLSSTASALGKLQDVNQNAQALNTLVKQLS | |
| | SNFGAISSVLNDILSRLDKVEAEVQIDRLITGRLQSLQTYVTQQLIRAA | |
| | EIRASANLAATKMSECVLGQSKRVDFCGKGYHLMSFPQSAPHGVV | |
| | FLHVTYVPAQEKNFTTAPAICHDGKAHFPREGVFVSNGTHWFVTQR | |
| | NFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQEL | |
| | GKYEQ | |
| spike glycoprotein | MFVFLVLLPLVSSQCVNLTTRTQLPPAYTNSFTRGVYYPDKVFRSS | 146 |
| with a mutation | VLHSTQDLFLPFFSNVTWFHAIHVSGTNGTKRFDNPVLPFNDGVYF | |
| 682-RRAR-685 → | ASTEKSNIIRGWIFGTTLDSKTQSLLIVNNATNVVIKVCEFQFCNDPF | |
| 682-QQAQ-685 in | LGVYYHKNNKSWMESEFRVYSSANNCTFEYVSQPFLMDLEGKQG | |
| the S1-S2 | NFKNLREFVFKNIDGYFKIYSKHTPINLVRDLPQGFSALEPLVDLPIGI | |
| cleavage site | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVVV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPQQAQSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSFIEDLLFNKVTLADAGFIKQYGDCL | |
| | GDIAARDLICAQKFNGLTVLPPLLTDEMIAQYTSALLAGTITSGWTFG | |
| | AGAALQIPFAMQMAYRFNGIGVTQNVLYENQKLIANQFNSAIGKIQD | |
| | SLSSTASALGKLQDVVNQNAQALNTLVKQLSSNFGAISSVLNDILSR | |
| | LDKVEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDVVIGIVNNTVYDPLQPELDSFKEELDKYFKNHTSPDVDLGDIS | |
| | GINASVVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 147 |
| with two proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNVVIKVCEFQFCNDPF | |
| (K986P, V987P) | LGVYYHKNNKSWMESEFRVYSSANNCTFEYVSQPFLMDLEGKQG | |
| | NFKNLREFVFKNIDGYFKIYSKHTPINLVRDLPQGFSALEPLVDLPIGI | |
| | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVVV | |
| | LSFELLHAPATVCGPKKSTNLVKNKCVNFNFNGLTGTGVLTESNKK | |
| | FLPFQQFGRDIADTTDAVRDPQTLEILDITPCSFGGVSVITPGTNTSN | |
| | QVAVLYQDVNCTEVPVAIHADQLTPTWRVYSTGSNVFQTRAGCLIG | |
| | AEHVNNSYECDIPIGAGICASYQTQTNSPRRARSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSFIEDLLFNKVTLADAGFIKQYGDCL | |
| | GDIAARDLICAQKFNGLTVLPPLLTDEMIAQYTSALLAGTITSGWTFG | |
| | AGAALQIPFAMQMAYRFNGIGVTQNVLYENQKLIANQFNSAIGKIQD | |
| | SLSSTASALGKLQDVVNQNAQALNTLVKQLSSNFGAISSVLNDILSR | |
| | LDPPEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDVVIGIVNNTVYDPLQPELDSFKEELDKYFKNHTSPDVDLGDIS | |
| | GINASVVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 148 |
| with four proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNVVIKVCEFQFCNDPF | |
| (F817P, A892P, | LGVYYHKNNKSWMESEFRVYSSANNCTFEYVSQPFLMDLEGKQG | |
| A899P, A942P) | NFKNLREFVFKNIDGYFKIYSKHTPINLVRDLPQGFSALEPLVDLPIGI | |
| | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVVV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPRRARSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPL | |
| | KDFGGFNFSQILPDPSKPSKRSPIEDLLFNKVTLADAGFIKQYGDCL | |
| | GDIAARDLICAQKFNGLTVLPPLLTDEMIAQYTSALLAGTITSGWTFG | |
| | AGPALQIPFPMQMAYRFNGIGVTQNVLYENQKLIANQFNSAIGKIQD | |
| | SLSSTPSALGKLQDVVNQNAQALNTLVKQLSSNFGAISSVLNDILSR | |
| | LDKVEAEVQIDRLITGRLQSLQTYVTQQLIRAAEIRASANLAATKMSE | |
| | CVLGQSKRVDFCGKGYHLMSFPQSAPHGVFLHVTYVPAQEKNFT | |
| | TAPAICHDGKAHFPREGVFVSNGTHWFVTQRNFYEPQIITTDNTFVS | |
| | GNCDVVIGIVNNTVYDPLQPELDSFKEELDKYFKNHTSPDVDLGDIS | |
| | GINASVVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 149 |
| with six proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNVVIKVCEFQFCNDPF | |
| (F817P, A892P, | LGVYYHKNNKSWMESEFRVYSSANNCTFEYVSQPFLMDLEGKQG | |
| A899P, A942P, | NFKNLREFVFKNIDGYFKIYSKHTPINLVRDLPQGFSALEPLVDLPIGI | |
| K986P, V987P) | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVVV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPRRARSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSPIEDLLFNKVTLADAGFIKQYGDCL | |
| | GDIAARDLICAQKFNGLTVLPPLLTDEMIAQYTSALLAGTITSGWTFG | |
| | AGPALQIPFPMQMAYRFNGIGVTQNVLYENQKLIANQFNSAIGKIQD | |
| | SLSSTPSALGKLQDVVNQNAQALNTLVKQLSSNFGAISSVLNDILSR | |
| | LDPPEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDVVIGIVNNTVYDPLQPELDSFKEELDKYFKNHTSPDVDLGDIS | |
| | GINASVVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 150 |
| with six proline | VLHSTQDLFLPFFSNVTWFHAIHVSGTNGTKRFDNPVLPFNDGVYF | |
| substitutions | ASTEKSNIIRGWFGTTLDSKTQSLLIVNNATNVVIKVCEFQFCNDPF | |
| (F817P, A892P, | LGVYYHKNNKSWMESEFRVYSSANNCTFEYVSQPFLMDLEGKQG | |
| A899P, A942P, | NFKNLREFVFKNIDGYFKIYSKHTPINLVRDLPQGFSALEPLVDLPIGI | |
| K986P, V987P) | NITRFQTLLALHRSYLTPGDSSSGWTAGAAAYYVGYLQPRTFLLKY | |
| and a 682-RRAR- | NENGTITDAVDCALDPLSETKCTLKSFTVEKGIYQTSNFRVQPTESIV | |
| 685 → 682-QQAQ- | RFPNITNLCPFGEVFNATRFASVYAWNRKRISNCVADYSVLYNSAS | |
| 685 mutation | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVVV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPQQAQSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSPIEDLLFNKVTLADAGFIKQYGDCL | |
| | GDIAARDLICAQKFNGLTVLPPLLTDEMIAQYTSALLAGTITSGWTFG | |
| | AGPALQIPFPMQMAYRFNGIGVTQNVLYENQKLIANQFNSAIGKIQD | |
| | SLSSTPSALGKLQDVVNQNAQALNTLVKQLSSNFGAISSVLNDILSR | |
| | LDPPEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDVVIGIVNNTVYDPLQPELDSFKEELDKYFKNHTSPDVDLGDIS | |
| | GINASVVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| Spike Glycoprotein | M F V F L V L L P L V S S Q C V N F T T R T Q L P P A Y T N S F T | 151 |
| sequence with 36 | R G V Y Y P D K V F R S S V L H S T Q D L F L P F F S N V T W F | |
| mutations and 6 | H A I - S G T N G T K R F D N P V L P F N D G V Y F A S T E K S | |
| deletions (-) | N I I R G W I F G T T L D S K T Q S L L I V N N A T N V V I K V C E | |
| | F Q F C N D P F L G V - Y H K N N K S W M E S E F R V Y S S A N | |
| | N C T F E Y V S Q P F L M D L E G K Q G N F K N L R E F V F K N I | |
| | D G Y F K I Y S K H T P I N L V R D L P Q G F S A L E P L V D L P I | |
| | G I N I T R F Q T L - - - H R S Y L T P G D S S S G W T A G A A A Y | |
| | Y V G Y L Q P R T F L L K Y N E N G T I T D A V D C A L D P L S E | |
| | T K C T L K S F T V E K G I Y Q T S N F R V Q P T E S I V R F P N I | |
| | T N L C P F S E I F N A T K F S S V Y A W D R R K I N N C V A D Y | |
| | S F L Y N S A S F S T F K C Y G V S L N K L N D L C F T N V Y A D | |
| | S F V I R G D Q V K Q I A P G Q T G N I A D Y N Y K L P D D F T G | |
| | C V I A W N S K K L D S K V V G N H K Y R F R F - R K S N L K P F | |
| | E R D I S T E I Y Q V G N K P C K G A K G L N C Y L P L K S Y G F | |
| | Q P T Y G V G Y Q P H R V V V L S F E L L H A S A T V C G P K K | |
| | S T N L V K N K C V N F N F N G L T G T G V L T E S N K K F L P F | |
| | Q Q F G R D I A D T T D A V R D P Q T L E I L D I T P C S F G G V | |
| | S V I T P G T N T S N Q V A V L Y Q D V N C T E V P V A I H A D Q | |
| | L T P T W R V Y S T G S N V F Q T R A G C L I G A E H V N N S Y | |
| | E C D I P I G A G I C A S Y Q T Q T N S P R R A R S V A S Q S I I A | |
| | Y T M S L G A E N S V A Y S N N S I A I P T N F T I S V T T E I L P | |
| | V S M T K T S V D C T M Y I C G D S T E C S N L L L Q Y G S F C T | |
| | Q L N R A L T G I A V E Q D K N T Q E V F A Q V K Q I Y K T P P I | |
| | K Y F G G F N F S Q I L P D P S K P S K R S F I E D L L F N K V T L | |
| | A D A G F I K Q Y G D C L G D I A A R D L I C A Q K F N G L T V L | |
| | P P L L T D E M I A Q Y T S A L L A G T I T S G W T F G A G A A L | |
| | Q I P F A M Q M A Y R F N G I G V T Q N V L Y E N Q K L I A N Q F | |
| | N S A I G K I Q D S L S S T A S A L G K L Q D V V N Q N A Q A L N | |
| | T L V K Q L S S N F G A I S S V L N D I L S R L D K V E A E V Q I D | |
| | R L I T G R L Q S L Q T Y V T Q Q L I R A A E I R A S A N L A A T K | |
| | M S E C V L G Q S K R V D F C G K G Y H L M S F P Q S A P H G V | |
| | V F L H V T Y V P A Q E K N F T T A P A I C H D G K A H F P R E G | |
| | V F V S N G T H W F V T Q R N F Y E P Q I T T D N T F V S G N C | |
| | D V V I G I V N N T V Y D P L Q P E L D S F K E E L D K Y F K N H | |
| | T S P D V D L G D I S G I N A S V V N I Q K E I D R L N E V A K N L | |
| | N E S L I D L Q E L G K Y E Q Y I K W P W Y I W L G F I A G L I A I | |
| | V M V T I M L C C M T S C C S C L K G C C S C G S C C K F D E D | |
| | D S E P V L K G V K L H Y T | |
| Wild type native | MFVFLVLLPLVSS | 63 |
| leader sequence | | |

In some embodiments, the mutations in the nucleocapsid (N) protein may include but are not limited to A220V, M234I, A376T, R203K, G204R, T205I, P80R, R203K, G204R, P199L, S186Y, D377Y, S2-, D3Y, A12G, P199L, M234I, P67S, P199L, D377Y, P67S, P199L or a combination thereof.

In some embodiments, the mutations in the Envelope (E) protein may include but are not limited to P71L. In some embodiments, the mutations in the ORF3a protein may Include but are not limited to Q38R, G172R, V202L, P42L or a combination thereof.

In some embodiments, the mutations in the ORF7a protein may include but are not limited to R80I. In some embodiments, the mutations in the ORF8 protein may Include but are not limited to Q27, T11I, or a combination thereof. In some embodiments, mutation in the ORF10 protein may Include but are not limited to V30L.

In some embodiments, the mutations in the ORF1b protein may include but are not limited to A176S, V767L, K1141R, E1184D, D1183Y, P255T, Q1011H, N1653D, R2613C, N1653D, or a combination thereof.

In some embodiments, the mutations in the ORF1a protein may Include but are not limited to S3675-, G3676-, F3677-, S3675-, G3676-, F3677-, S3675-, G3676-, F3677-, 14205V, I2501T, T945I, T15871, Q3346K, V3475F, M3862I, S3875-, G3678-, F3677-, S3675-, G3678-, F3677-, T2851, L3352F, T265I, L3352F or a combination thereof.

In some embodiments, the vaccine composition comprises one or more coronavirus B-cell target epitopes; one or more coronavirus CD4⁺ T cell target epitopes; and one or more coronavirus CD8⁺ T cell target epitopes. In some embodiments, the vaccine composition comprises one or more coronavirus B-cell target epitopes and one or more coronavirus CD4⁺ T cell target epitopes. In some embodiments, the vaccine composition comprises one or more coronavirus B-cell target epitopes and one or more coronavirus CD8⁺ T cell target epitopes. In some embodiments, the vaccine composition comprises one or more coronavirus CD8⁺ target epitopes and one or more coronavirus CD4⁺ T cell target epitopes. In some embodiments, the vaccine composition comprises one or more coronavirus CD8⁺ target epitopes. In some embodiments, the vaccine composition comprises one or more coronavirus CD4⁺ target epitopes. In some embodiments, the vaccine composition comprises one or more coronavirus B cell target epitopes.

In some embodiments, the one or more of the at least two target epitopes may be in the form of a large sequence. In some embodiments, the large sequence is derived from one or more whole protein sequences expressed by SARS-CoV-2 or a SARS-CoV-2 variant. In other embodiments, the large sequence is derived from one or more partial protein sequences expressed by SARS-CoV-2 or a SARS-CoV-2 variant.

The target epitopes may be derived from structural proteins, non-structural proteins, or a combination thereof. For example, structural proteins may include spike proteins (S), envelope proteins (E), membrane proteins (M), or nucleoproteins (N).

In some embodiments, the target epitopes are derived from at least one SARS-CoV-2 protein. The SARS-CoV-2 proteins may include ORF1ab protein. Spike glycoprotein, ORF3a protein, Envelope protein, Membrane glycoprotein, ORF6 protein, ORF7a protein, ORF7b protein, ORF8 protein, Nucleocapsid protein, and ORF10 protein. The ORF1ab protein provides nonstructural proteins (Nsp) such as Nsp1, Nsp2, Nsp3 (Papain-like protease), Nsp4, Nsp5 (3C-like protease), Nsp6, Nsp7, Nsp8, Nsp9, Nsp10, Nsp11, Nsp12 (RNA polymerase), Nsp13 (5′ RNA triphosphatase enzyme), Nsp14 (guanosineN7-methyltransferase), Nsp15 (endoribonuclease), and Nsp16 (2′-O-ribose-methyltransferase).

The target epitopes may be restricted to human HLA class 1 and 2 haplotypes. In some embodiments, the target epitopes are restricted to cat and dog MHC class 1 and 2 haplotypes.

### Conserved Epitopes

In certain embodiments, the vaccine composition comprises one or more mutated epitopes in combination with one or more mutated epitopes.

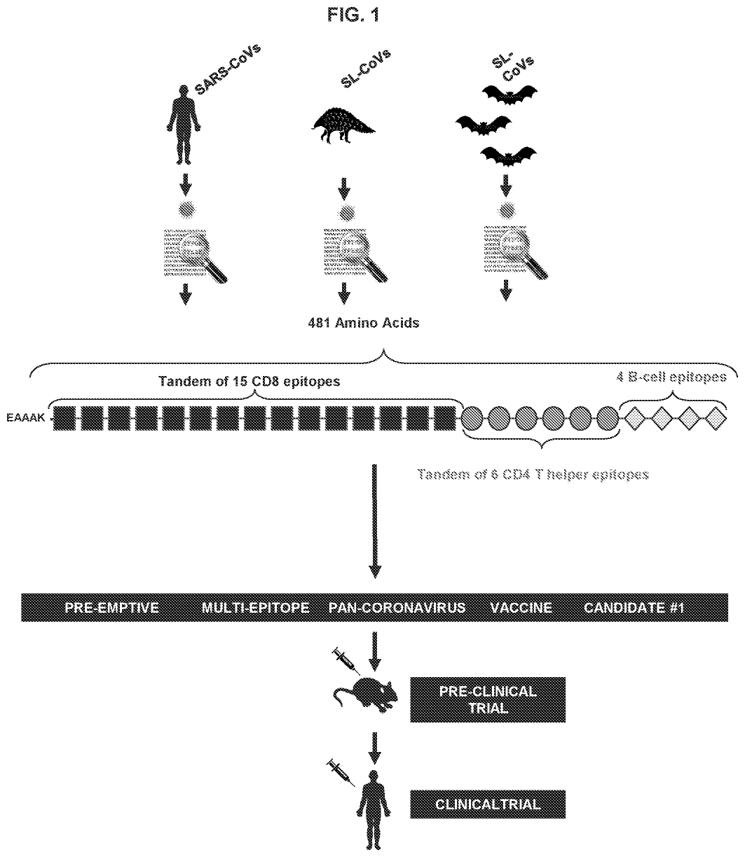

The present invention describes the identification of mutated B cell, CD4⁺ T cell, and CD8⁺ T cell epitopes. For example, FIG. 1 shows a schematic of the development of a pre-emptive multi-epitope pan coronavirus vaccine featuring multiple mutated B cell epitopes, multiple mutated CD8+ T cell epitopes, and multiple CD4⁺ T cell epitopes. The epitopes are derived from sequence analysis of many coronaviruses.

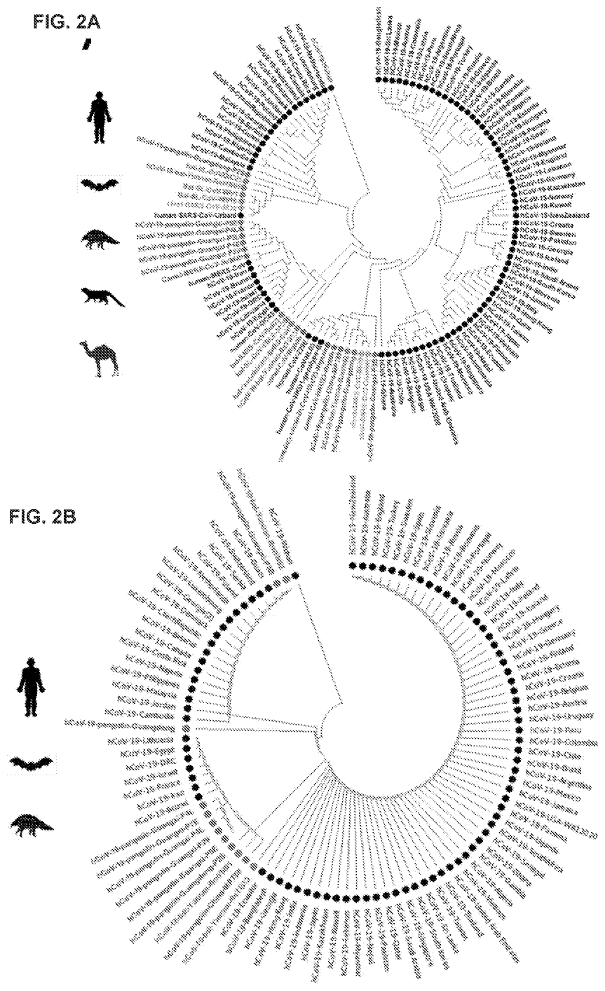

Coronaviruses used for determining mutated epitopes may include human SARS-CoVs as well as animal CoVs (e.g., bats, pangolins, civet cats, minks, camels, etc.) as described herein. As an example, FIG. 2A and FIG. 2B show an evolutionary comparison of genome sequences among beta-coronavirus strains isolated from humans and animals. FIG. 2A shows a phylogenetic analysis performed between SARS-CoV-2 strains (obtained from humans (Homo Sapiens (black)), along with the animal's SARS-like Coronaviruses genome sequence (SL-CoVs) sequences obtained from bats (Rhinolophus affinis, Rhinolophus malayanus (red)), pangolins (Manis javanica (blue)), civet cats (Paguma larvata (green)), and camels (Camelus dromedaries (Brown)). The included SARS-CoV/MERS-CoV strains are from previous outbreaks (obtained from humans (Urbani, MERS-CoV, OC43, NL63, 229E, HKU1-genotype-B), bats (WIV16, WIV1, YNLF-31C, Rs672, recombinant strains), camel (Camelus dromedaries, (KT388891.1, MN514967.1, KF917527.1, NC_028752.1), and civet (Civet007, A022, B039)). The human SARS-CoV-2 genome sequences are represented from six continents. FIG. 2B shows an evolutionary analysis performed among the human-SARS-CoV-2 genome sequences reported from six continents and SARS-CoV-2 genome sequences obtained from bats (Rhinolophus affinis, Rhinolophus malayanus), and pangolins (Manis javanica)).

Additionally, other coronaviruses may be used for determining mutated epitopes (including human SARS-CoVs as well as animal CoVs (e.g., bats, pangolins, civet cats, minks, camels, etc.)) that meet the criteria to be classified as “variants of concern” or “variants of interest.” Coronavirus variants that appear to meet one or more of the undermentioned criteria may be labeled “variants of interest” or “variants under investigation” pending verification and validation of these properties. In some embodiments, the criteria may include increased transmissibility, increased morbidity, increased mortality, increased risk of “long COVID”, ability to evade detection by diagnostic tests, decreased susceptibility to antiviral drugs (if and when such drugs are available), decreased susceptibility to neutralizing antibodies, either therapeutic (e.g., convalescent plasma or monoclonal antibodies) or in laboratory experiments, ability to evade natural immunity (e.g., causing reinfections), ability to infect vaccinated individuals, increased risk of particular conditions such as multisystem inflammatory syndrome or long-haul COVID or Increased affinity for particular demographic or clinical groups, such as children or immunocompromised individuals. Once validated variants of interest are renamed “variant of concern” by monitoring organizations, such as the CDC.

The mutated epitopes may be derived from structural (e.g., spike glycoprotein, envelope protein, membrane protein, nucleoprotein) or non-structural proteins of the coronaviruses (e.g., any of the 16 NSPs encoded by ORF1a/b).

In some embodiments, one or more epitopes are highly mutated among one or a combination of: SARS-CoV-2 human strains, SL-CoVs isolated from bats, SL-CoVs isolated from pangolin, SL-CoVs isolated from civet cats, and MERS strains Isolated from camels. For example, in certain embodiments, an epitopes is highly mutated among one or a combination of: at least 50,000 SARS-CoV-2 human strains, five SL-CoVs isolated from bats, five SL-CoVs isolated from pangolin, three SL-CoVs isolated from civet table cats, and four MERS strains isolated from camels. In certain embodiments, one or more epitopes are highly mutated among one or a combination of: at least 80,000 SARS-CoV-2 human strains, five SL-CoVs isolated from bats, five SL-CoVs isolated from pangolin, three SL-CoVs isolated from civet cats, and four MERS strains isolated from camels. In certain embodiments, one or more epitopes are highly mutated among one or a combination of: at least 50,000 SARS-CoV-2 human strains in circulation during the COVI-19 pandemic, at least one CoV that caused a previous human outbreak, five SL-CoVs Isolated from bats, five SL-CoVs isolated from pangolin, three SL-CoVs Isolated from civet cats, and four MERS strains isolated from camels. In certain embodiments, one or more epitopes are highly mutated among at least 1 SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. In certain embodiments, one or more epitopes are highly mutated among at least 1,000 SARS-CoV-2 human strains in current circulation, at least two CoVs that has caused a previous human outbreak, at least two SL-CoVs isolated from bats, at least two SL-CoVs isolated from pangolin, at least two SL-CoVs isolated from civet cats, and at least two MERS strains isolated from camels. In certain embodiments, one or more epitopes are highly mutated among one or a combination of: at least one SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. The present invention is not limited to the aforementioned coronavirus strains that may be used to identify mutated epitopes.

In certain embodiments, one or more of the mutated epitopes are derived from one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. SARS-CoV-2 human strains and variants in current circulation may include the original SARS-CoV-2 strain (SARS-CoV-2 isolate Wuhan-Hu-1), and several variants of SARS-CoV-2 including but not limited to Spain strain B.1.177; Australia strain B.1.160, England strain B.1.1.7; South Africa strain B.1.351; Brazil strain P.1; California strain B.1.427/B.1.429; Scotland strain B.1.258; Belgium/Netherlands strain B.1.221; Norway/France strain B.1.367; Norway/Denmark.UK strain B.1.1.277; Sweden strain B.1.1.302; North America, Europe, Asia, Africa, and Australia strain B.1.525; and New York strain B.1.526. The present invention is not limited to the aforementioned variants of SARS-CoV-2 and encompasses variants identified in the future. The one or more coronaviruses that cause the common cold may include but are not limited to strains 229E (alpha coronavirus), NL63 (alpha coronavirus), OC43 (beta coronavirus), HKU1 (beta coronavirus).

As used herein, the term “mutated” refers to an epitope that is among the most highly mutated epitopes identified in a sequence alignment and analysis for its particular epitopes type (e.g., B cell, CD4 T cell, CD8 T cell). For example, the mutated epitopes may be the 5 most highly mutated epitopes identified (for the particular type of epitope). In some embodiments, the mutated epitopes may be the 10 most highly mutated epitopes identified (for the particular type of epitope). In some embodiments, the mutated epitopes may be the 15 most highly mutated epitopes identified (for the particular type of epitope). In some embodiments, the mutated epitopes may be the 20 most highly mutated epitopes identified (for the particular type of epitope). In some embodiments, the mutated epitopes may be the 25 most highly mutated epitopes identified (for the particular type of epitope). In some embodiments, the mutated epitopes may be the 30 most highly mutated epitopes identified (for the particular type of epitope). In some embodiments, the mutated epitopes may be the 40 most highly mutated epitopes identified (for the particular type of epitope). In some embodiments, the mutated epitopes may be the 50 most highly mutated epitopes identified (for the particular type of epitope). In some embodiments, the mutated epitopes may be the 50% most highly mutated epitopes identified (for the particular type of epitope). In some embodiments, the mutated epitopes may be the 60% most highly mutated epitopes identified (for the particular type of epitope). In some embodiments, the mutated epitopes may be the 70% most highly mutated epitopes identified (for the particular type of epitope). In some embodiments, the mutated epitopes may be the 80% most highly mutated epitopes identified (for the particular type of epitope). In some embodiments, the mutated epitopes may be the 90% most highly mutated epitopes identified (for the particular type of epitope). In some embodiments, the mutated epitopes may be the 95% most highly mutated epitopes identified (for the particular type of epitope). In some embodiments, the mutated epitopes may be the 99% most highly mutated epitopes identified (for the particular type of epitope). The present invention is not limited to the aforementioned thresholds.

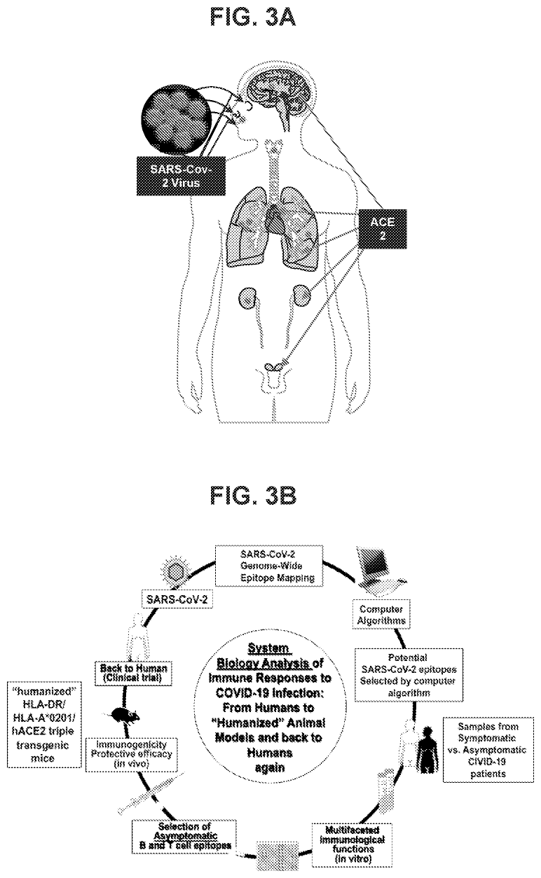

FIG. 3B shows an example of a systems biology approach utilized in the present invention.

For certain embodiments herein, the epitopes that are selected may be those that achieve a particular score in a binding assay (for binding to an HLA molecule, for example.) For example, in some embodiments, the epitopes selected have an IC₅₀ score of 250 or less in an ELISA binding assay (e.g., an ELISA binding assay specific for HLA-DR/peptide combination, HLA-A*0201/peptide combination, etc.), or the equivalent of the IC₅₀ score of 250 or less in a different binding assay. Binding assays are well known to one of ordinary skill in the art.

The mutated epitopes may be restricted to human HLA class 1 and 2 haplotypes. In some embodiments, the mutated epitopes are restricted to cat and dog MHC class 1 and 2 haplotypes.

For any of the embodiments herein, the epitopes that are selected may be those that achieve a particular score in a binding assay (for binding to an HLA molecule, for example.) For example, in some embodiments, the epitopes selected have an IC₅₀ score of 250 or less in an ELISA binding assay (e.g., an ELISA binding assay specific for HLA-DR/peptide combination, HLA-A*0201/peptide combination, etc.), or the equivalent of the IC₅₀ score of 250 or less in a different binding assay. Binding assays are well known to one of ordinary skill in the art.

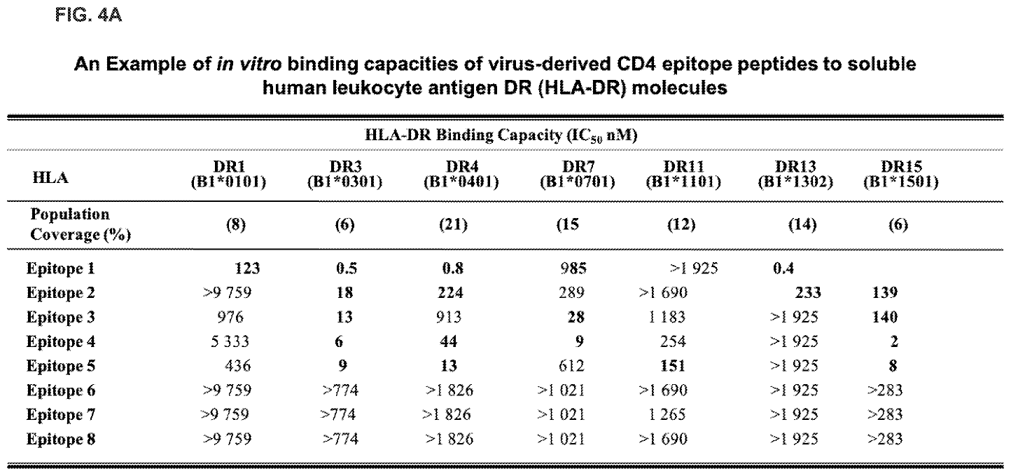

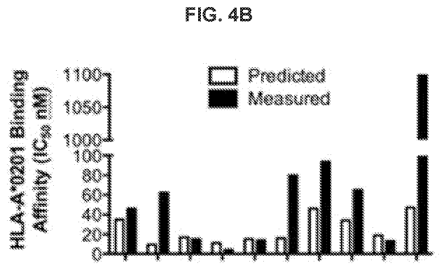

FIG. 4A shows examples of binding capacities of virus-derived CD4+ T cell epitope peptides to soluble HLA-DR molecules. CD4+ T cell peptides were submitted to ELISA binding assays specific for HLA-DR molecules. Reference non-viral peptides were used to validate each assay. Data are expressed as relative activity (ratio of the IC₅₀ of the peptides to the IC₅₀ of the reference peptide) and are the means of two experiments. Peptide epitopes with high affinity binding to HLA-DR molecules have IC₅₀ below 250 and are indicated in bold. IC₅₀ above 250 indicates peptide epitopes that failed to bind to tested HLA-DR molecules.

FIG. 4B shows an example of potential epitopes binding with high affinity to HLA-A*0201 and stabilizing expression on the surface of target cells: Predicted and measured binding affinity of genome-derived peptide epitopes to soluble HLA-A*0201 molecule (IC₅₀ nM). The binding capacities of a virus CD8 T cell epitope peptide to soluble HLA-A*0201 molecules. CD8 T cell peptides were submitted to ELISA binding assays specific for HLA-A*0201 molecules. Reference non-viral peptides were used to validate each assay. Data are expressed as relative activity (ratio of the IC₅₀ to the peptide to the IC₅₀ of the reference peptide) and are the means of two experiments. Peptide epitopes with high affinity binding to HLA-A*0201 molecules have IC₅₀ below 100 and are indicated in bold. IC₅₀ above 100 Indicates peptide epitopes that failed to bind to tested HLA-A*0201 molecules.

### CD8+ Epitopes

The present invention features a plurality of CD8+ T cell epitopes which may comprise one or more mutations. In some embodiments, a mutation may be synonymous or non-synonymous. In some embodiments, the mutation may be a point mutation. In other embodiments, the mutation may be a single point mutation (such as the above mentioned mutations). In other embodiments, a single point mutation may be substitutions, deletions, or inversions

Table 3: below describes the sequences for the mutated epitope regions. Bolded amino acids Indicate amino acids that have been mutated when compared to the SARS-CoV-2-Wuhan (MN908947.3) strain.

| | | SEQ ID |
|---|---|---|
| CD8⁺ Epitope | Sequence | NO: |
| S₉₇₆₋₈₉₄ | VLNDILARL | 153 |

Examples of methods for identifying potential CD8+ T cell epitopes and screening conservancy of potential CD8+ T cell epitopes are described herein. The present invention is not limited to the particular software systems disclosed, and other software systems are accessible to one of ordinary skill in the art for such methods. The present invention is not limited to the specific haplotypes used herein. For example, one of ordinary skill in the art may select alternative molecules (e.g., HLA molecules) for molecular docking studies.

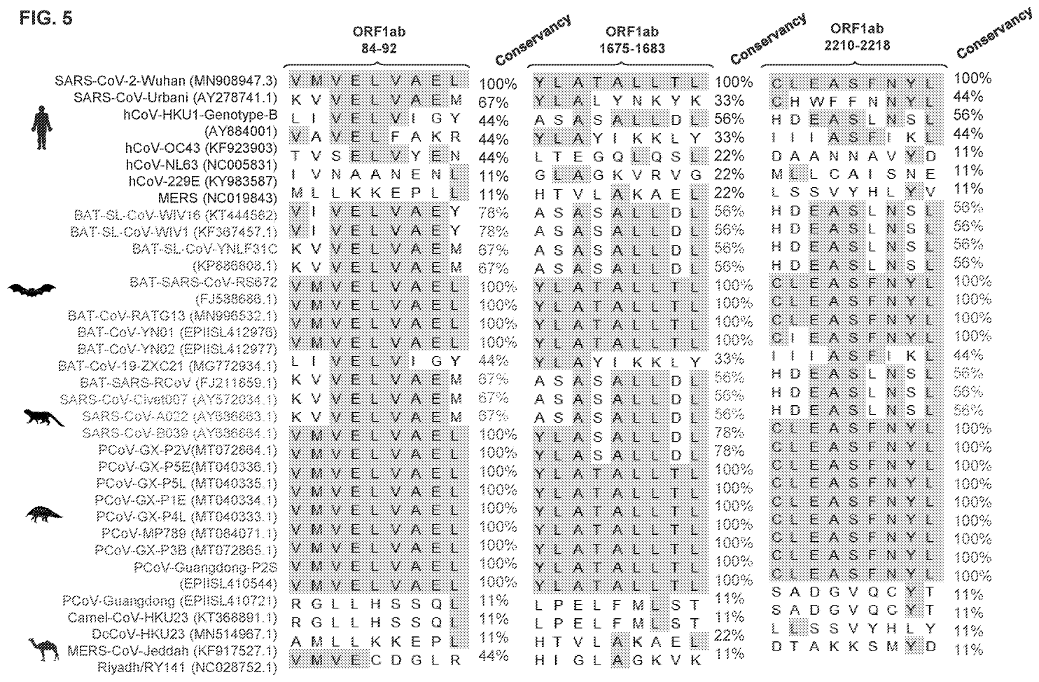

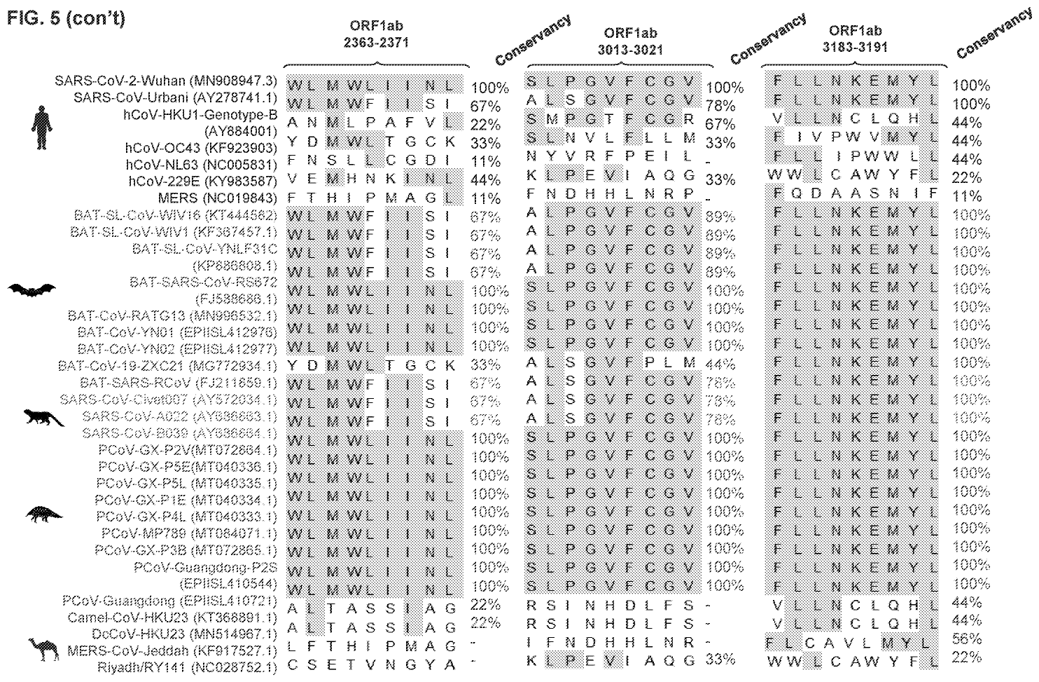

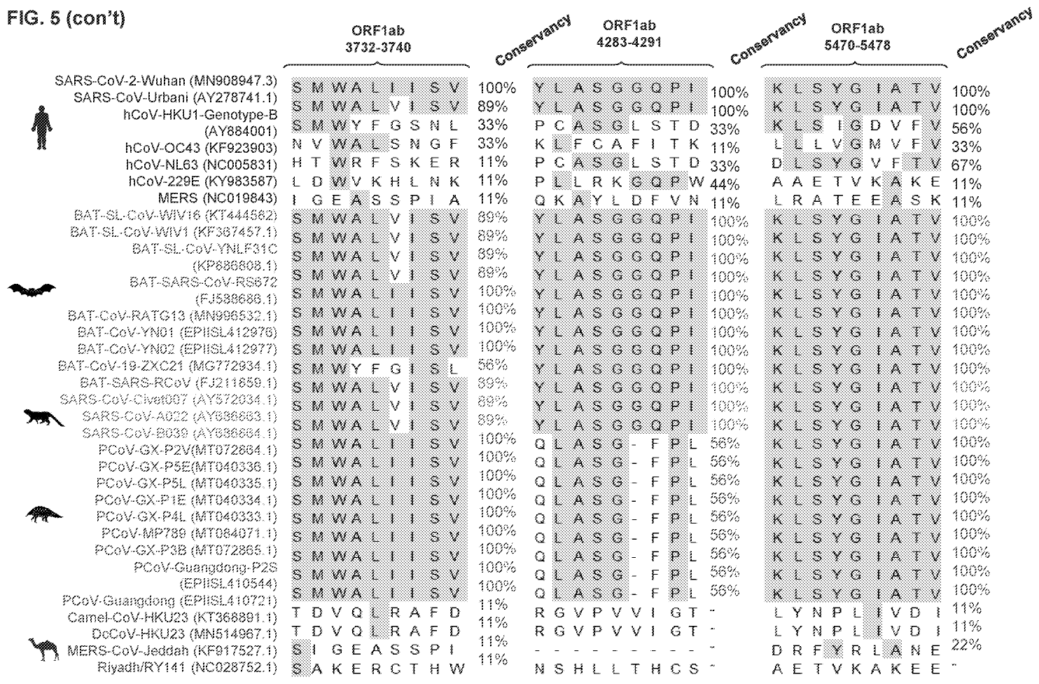

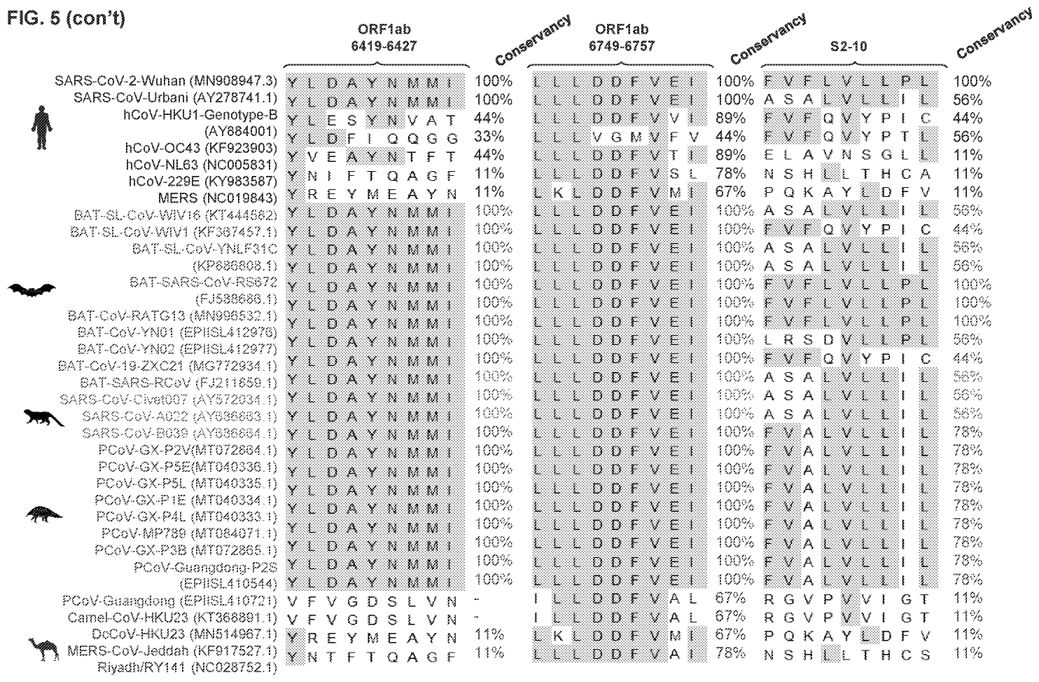

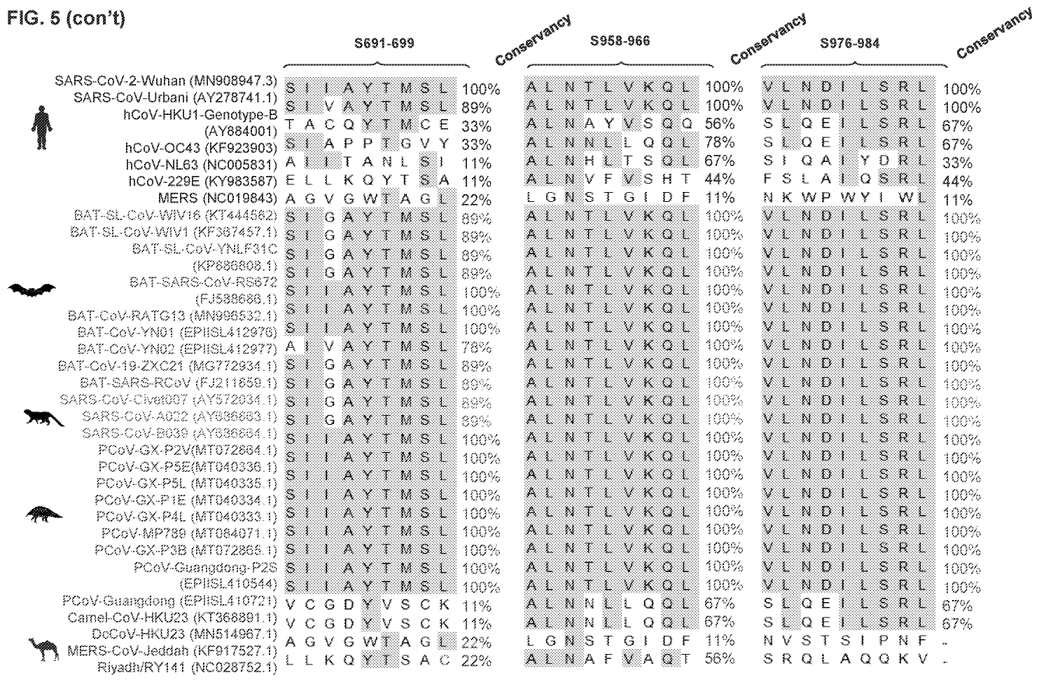

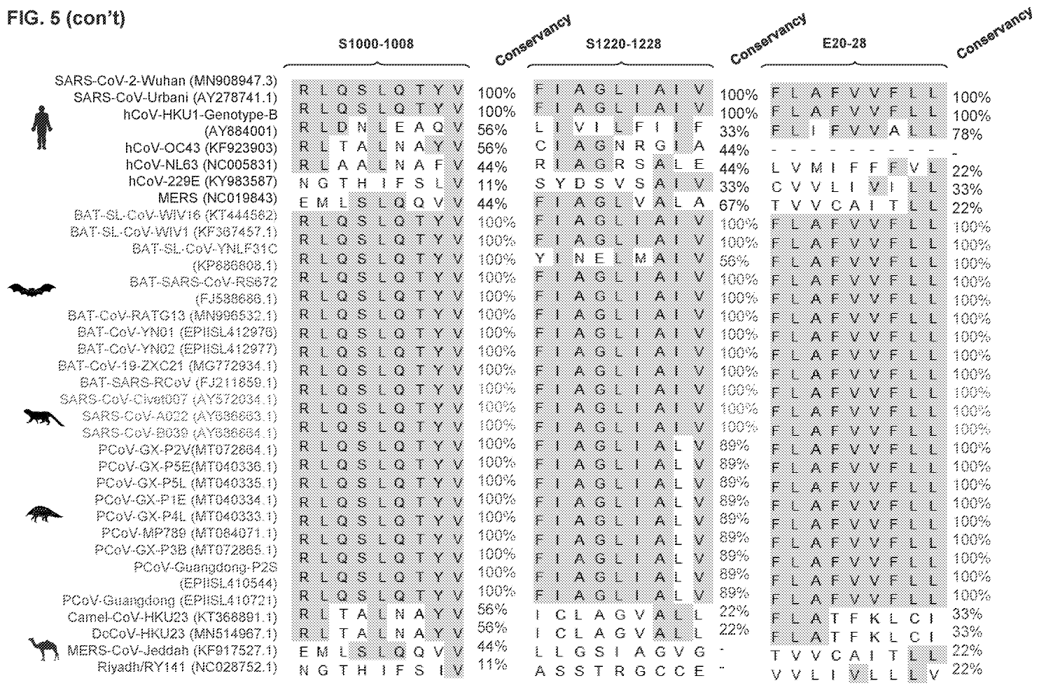

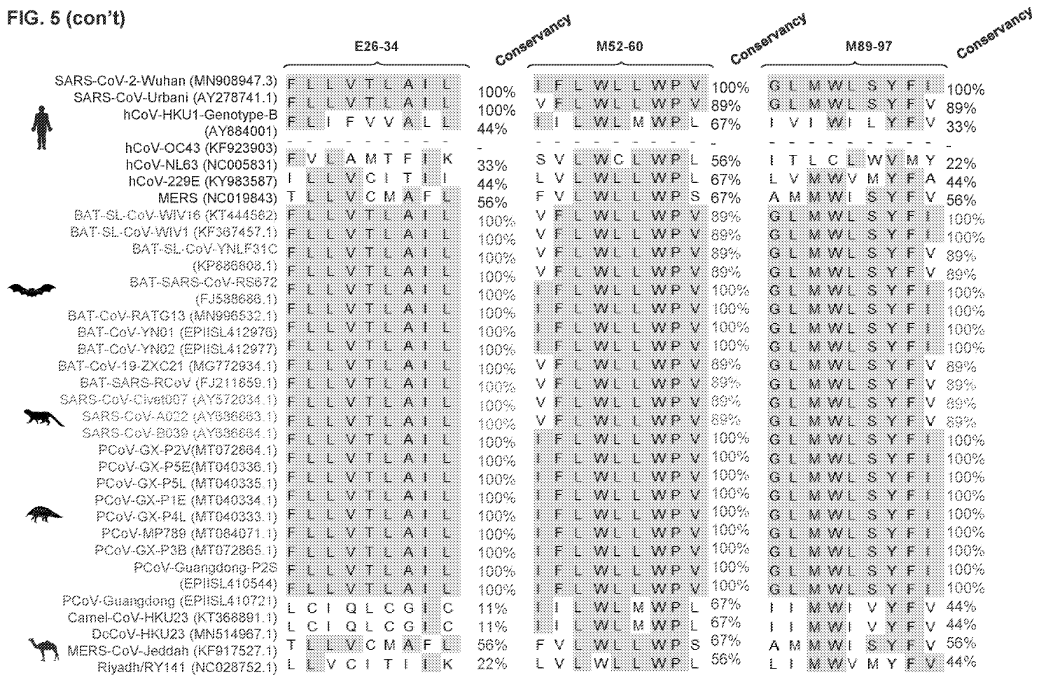

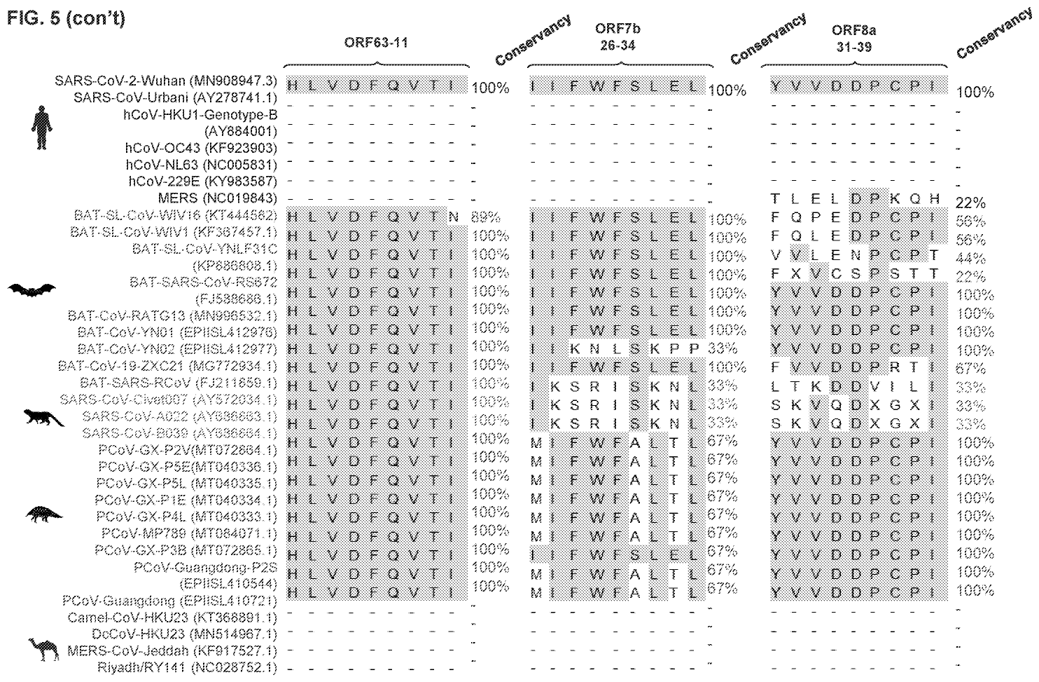

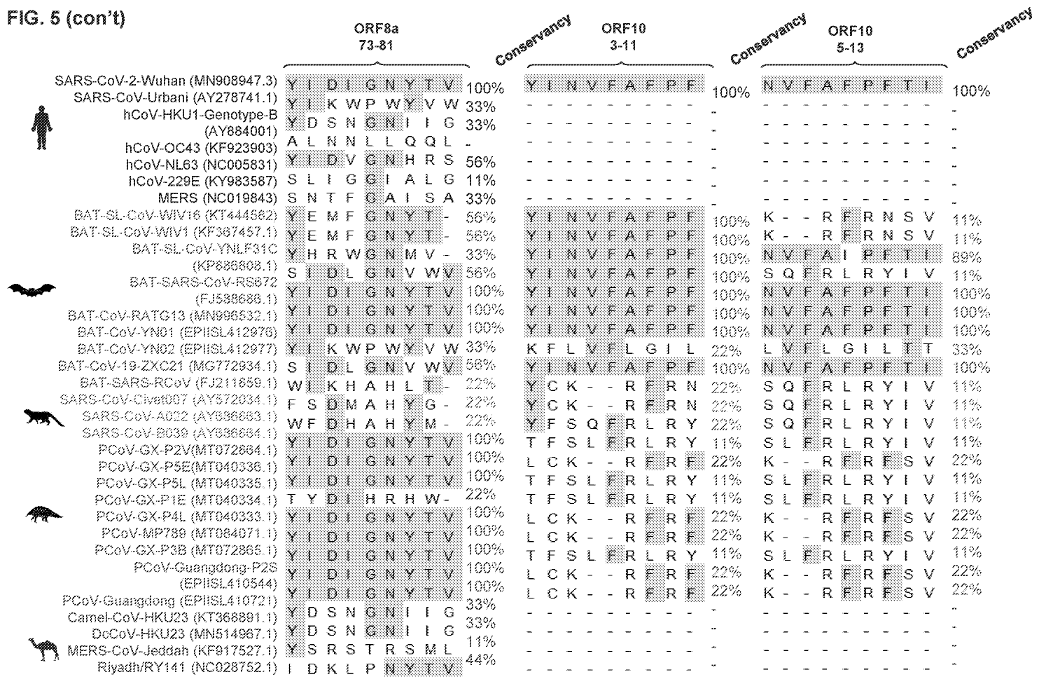

FIG. 5 shows sequence homology analysis for screening conservancy of potential CD8+ T cell epitopes, e.g., the comparison of sequence homology for the potential CD8+ T cell epitopes among 81,963 SARS-CoV-2 strains (that currently circulate in 190 countries on 6 continents), the 4 major “common cold” Coronaviruses that cased previous outbreaks (e.g., hCoV-OC43, hCoV-229E, hCoV-HKU1-Genotype B, and hCoV-NL63), and the SL-CoVs that were isolated from bats, civet cats, pangolins and camels. Epitope sequences highlighted in yellow present a high degree of homology among the currently circulating 81,963 SARS-CoV-2 strains and at least a 50% conservancy among two or more humans SARS-CoV strains from previous outbreaks, and the SL-CoV strains Isolated from bats, civet cats, pangolins and camels.

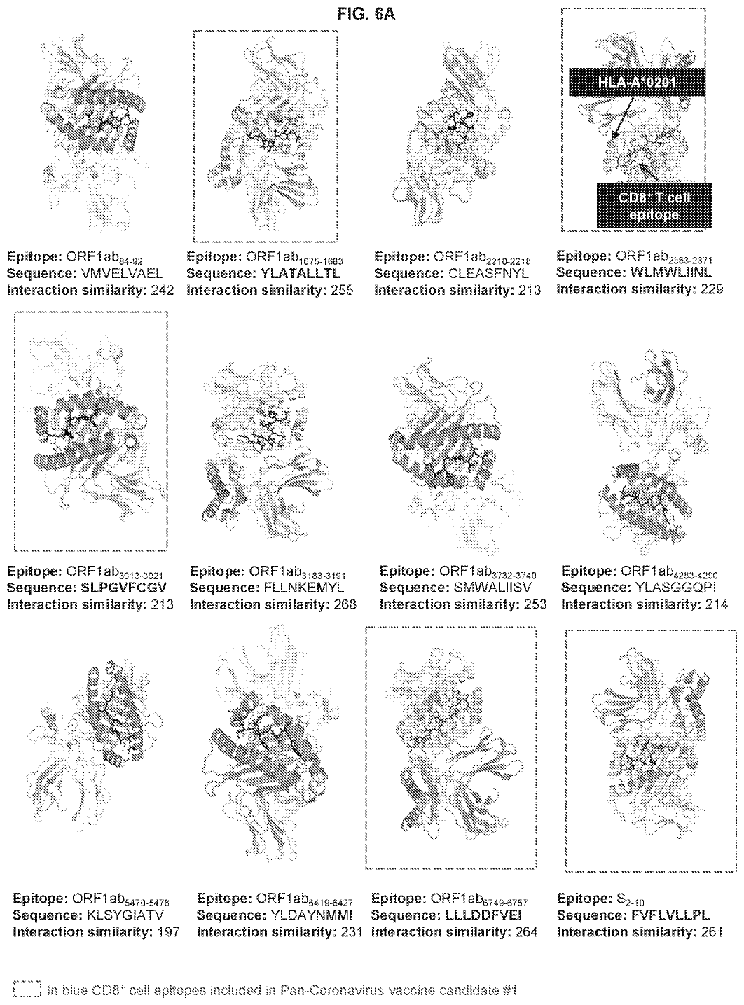

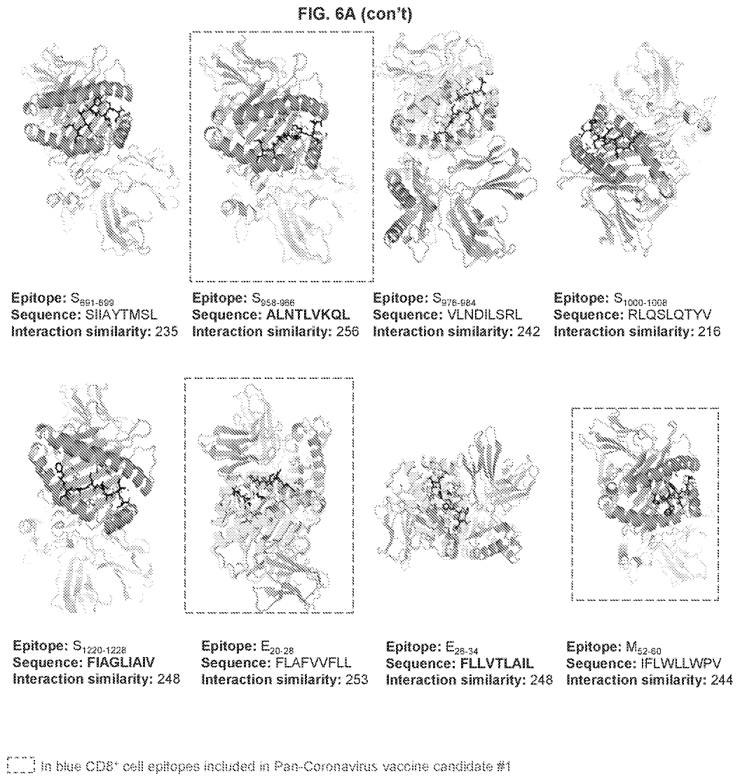

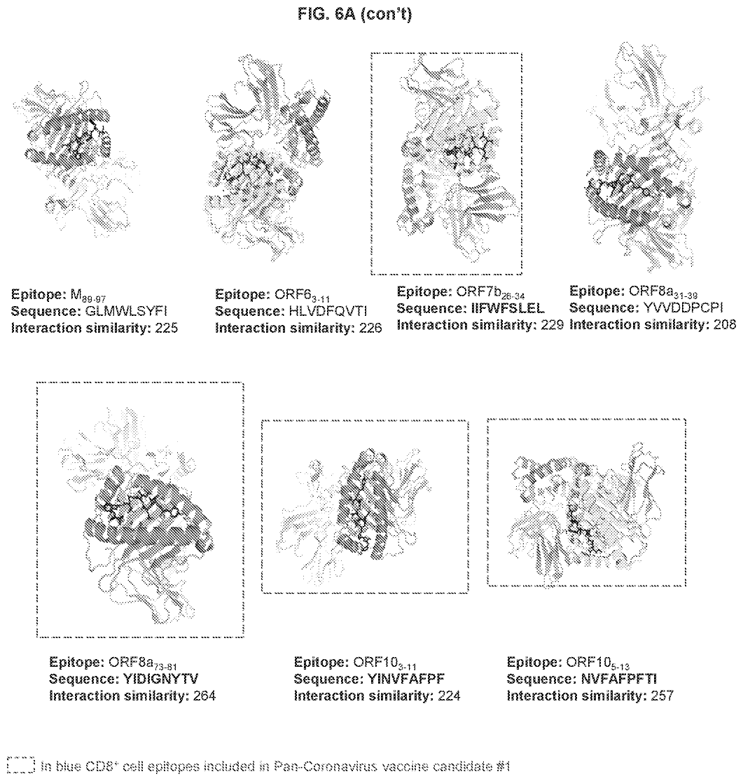

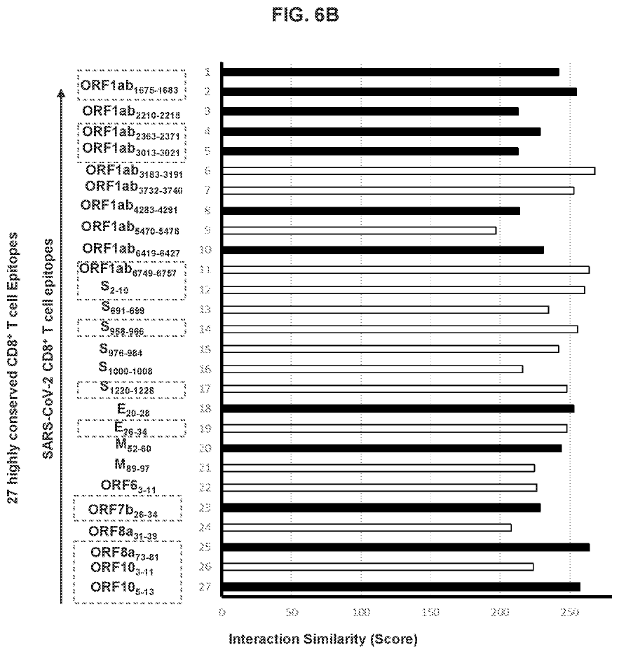

From the analysis, 27 CD8+ T cell epitopes were selected as being highly mutated. FIG. 6A and FIG. 6B show the docking of the mutated epitopes to the groove of HLA-A*02:01 molecules as well as the interaction scores determined by protein-peptide molecular docking analysis.

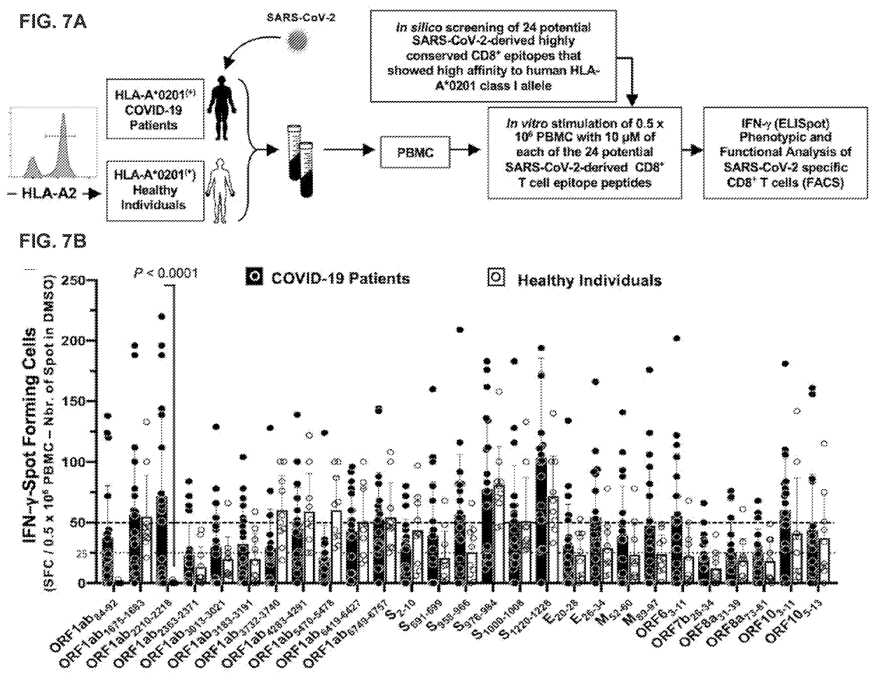

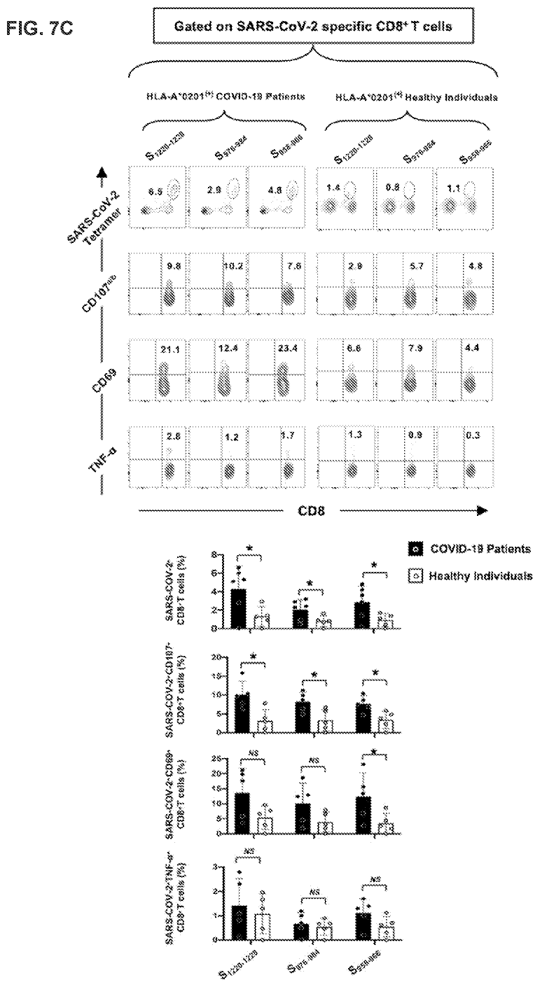

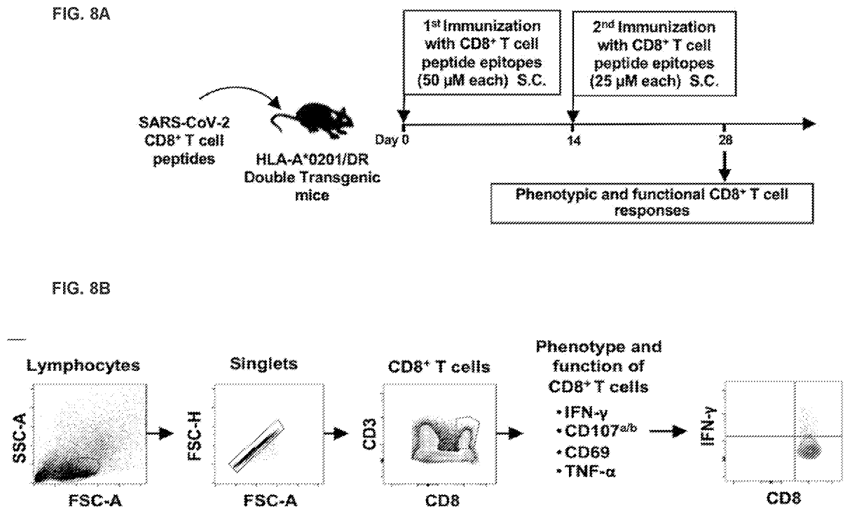

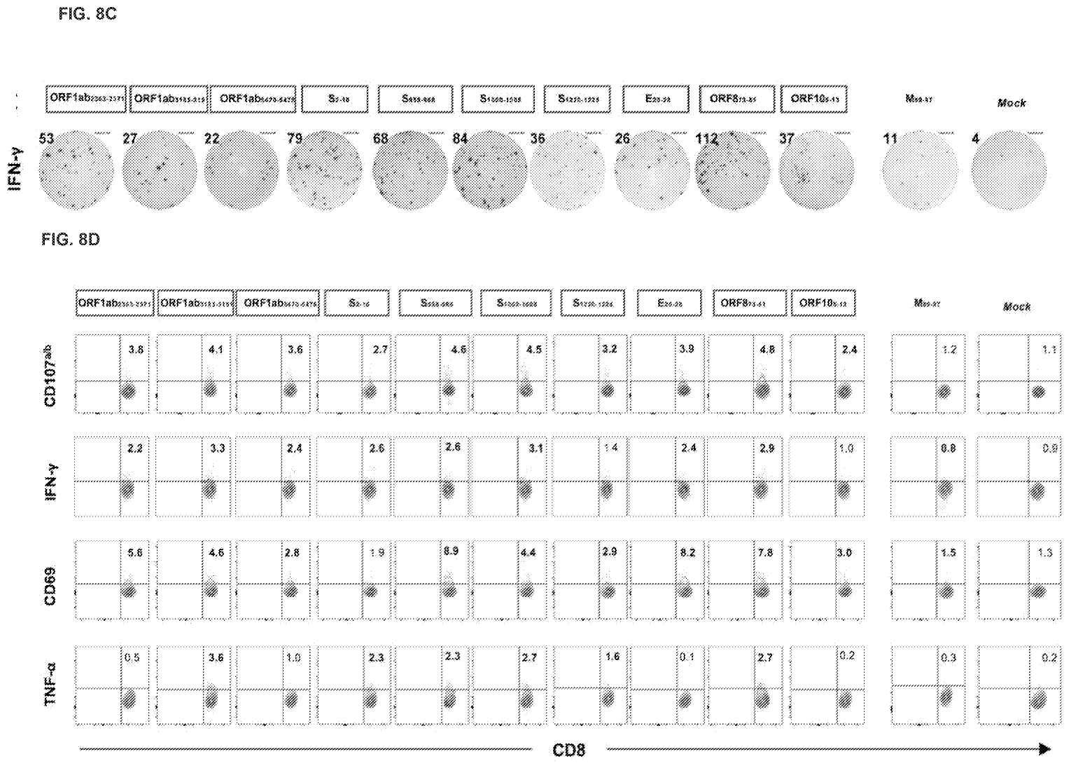

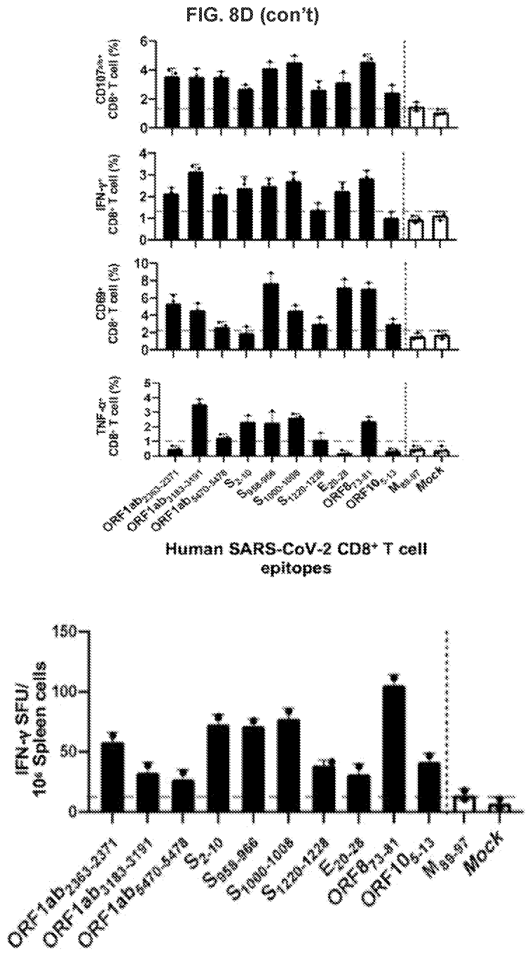

FIG. 7A, FIG. 7B, and FIG. 7C show that CD8+ T cells specific to several highly mutated SARS-CoV-2 epitopes disclosed herein were detected in COVID-19 patients and unexposed healthy individuals. FIG. 8A, FIG. 8B, FIG. 8C, and FIG. 8D show immunogenicity of the identified SARS-CoV-2 CD8+ T cell epitopes.

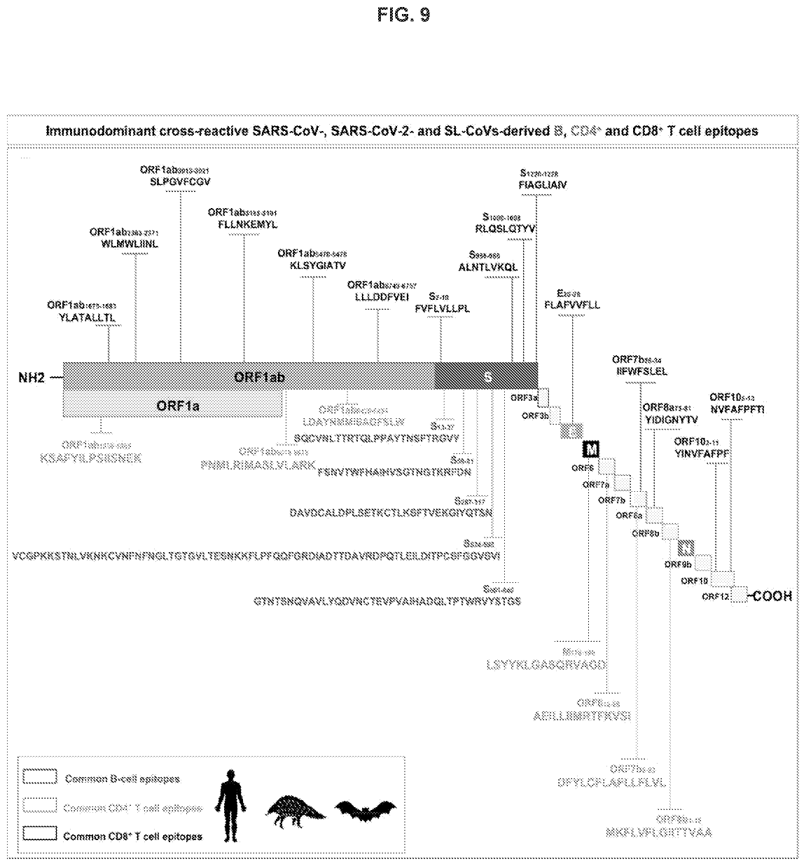

The CD8⁺ T cell target epitopes discussed above include S₂₋₁₀, S₁₂₂₀₋₁₂₂₈, S₁₀₀₀₋₁₀₀₈, S₉₅₈₋₈₆₆, E₂₀₋₂₈, ORF1ab₁₆₇₅₋₁₆₈₃, ORF1ab₂₃₆₃₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₆₋₃₄, ORF8a₇₃₋₈₁, ORF10₃₋₁₁, and ORF10₅₋₁₃. FIG. 9 shows the genome-wide location of the epitopes. Thus, in certain embodiments, the vaccine composition may comprise one or more CD8⁺ T cell epitopes selected from: S₂₋₁₀, S₁₂₂₀₋₁₁₂₈, S₁₀₀₀₋₁₀₀₈, S₉₅₈₋₉₆₆, E₂₀₋₂₈, ORF1ab₁₆₇₅₋₁₆₈₃, ORF1ab₂₃₆₃₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₆₋₃₄, ORF8a₃₋₁₁, ORF10₃₋₁₁, ORF10₅₋₁₃, or a combination thereof. Table 4 below describes the sequences for the aforementioned epitope regions.

TABLE 4

| CD8⁺ T Cell | Epitope | SEQ ID | CD8⁺ T Cell | Epitope | SEQ ID |
|---|---|---|---|---|---|
| Epitope | Sequence | NO: | Epitope | Sequence | NO: |
| ORF1ab₈₄₋₉₂ | VMVELVAEL | 2 | S₉₇₆₋₉₈₄ | VLNDILSRL | 16 |
| ORF1ab₁₆₇₅₋₁₆₈₃ | YLATALLTL | 3 | S₁₀₀₀₋₁₀₀₈ | RLQSLQTYV | 17 |
| ORF1ab₂₂₁₀₋₂₂₁₈ | CLEASFNYL | 4 | S₁₂₂₀₋₁₂₂₈ | FIAGLIAIV | 18 |
| ORF1ab₂₃₆₃₋₂₃₇₁ | WLMWLIINL | 5 | E₂₀₋₂₈ | FLAFWVFLL | 19 |
| ORF1ab₃₀₁₃₋₃₀₂₁ | SLPGVFCGV | 6 | E₂₆₋₃₄ | FLLVTLAIL | 20 |
| ORF1ab₃₁₈₃₋₃₁₉₁ | FLLNKEMYL | 7 | E₂₆₋₃₄ | FLLNKEMYL | 21 |
| ORF1ab₃₇₃₂₋₃₇₄₀ | SMWALIISV | 8 | M₅₂₋₆₀ | IFLWLLWPV | 22 |
| ORF1ab₄₂₈₃₋₄₂₉₁ | YLASGGQPI | 9 | M₈₉₋₉₇ | GLMWLSYFI | 23 |
| ORF1ab₅₄₇₀₋₅₄₇₈ | KLSYGIATV | 10 | ORF6₃₋₁₁ | HLVDFQVTi | 24 |
| ORF1ab₆₄₁₉₋₆₄₂₇ | YLDAYNMMI | 11 | ORF7b₂₆₋₃₄ | IIFWFSLEL | 25 |
| ORF1ab₆₇₄₉₋₆₇₅₇ | LLLDDFVEI | 12 | ORF8a₃₁₋₃₉ | YVVDDPCPI | 26 |
| S₂₋₁₀ | FVFLVLLPL | 13 | ORF8a₇₃₋₈₁ | YIDIGNYTV | 27 |
| S₆₉₁₋₆₉₉ | SIIAYTMSL | 14 | ORF10₃₋₁₁ | YINVFAFPF | 28 |
| S₉₅₈₋₉₈₆ | ALNTLVKQL | 15 | ORF10₅₋₁₃ | NVFAFPFTL | 29 |

The present invention is not limited to the aforementioned CD8⁺ T cell epitopes. For example, the present invention also Includes variants of the aforementioned CD8⁺ T cell epitopes, for example sequences wherein the aforementioned CD8⁺ T cell epitopes are truncated by one amino acid (examples shown below in Table 5).

TABLE 5

| CD8⁺ T Cell | Sequence with | | CD8⁺ T Cell | Sequence with | |
|---|---|---|---|---|---|
| Epitope | Single AA | SEQ ID | Epitope | Single AA | SEQ ID |
| Origin: | Truncation | NO: | Origin: | Truncation | NO: |
| ORF1ab₈₄₋₉₂ | VMVELVAE | 30 | S₉₇₆₋₉₈₄ | VLNDILSR | 44 |
| ORF1ab₁₆₇₅₋₁₆₈₃ | LATALLTL | 31 | S₁₀₀₀₋₁₀₀₈ | LQSLQTYV | 45 |
| ORF1ab₂₂₁₀₋₂₂₁₈ | CLEASFNY | 32 | S₁₂₂₀₋₁₂₂₈ | FIAGLIAI | 46 |
| ORF1ab₂₃₆₃₋₂₃₇₁ | LMWLIINL | 33 | E₂₀₋₂₈ | LAFVVFLL | 47 |
| ORF1ab₃₀₁₃₋₂₀₂₁ | SLPGVFCG | 34 | E₂₆₋₃₄ | FLLVTLAL | 48 |
| ORF1ab₃₁₈₃₋₃₁₉₁ | LLNKEMYL | 35 | E₂₆₋₃₄ | LLNKEMYL | 49 |
| ORF1ab₃₇₃₂₋₃₇₄₀ | SMWALIIS | 36 | M₅₂₋₆₀ | IFLWLLWP | 50 |
| ORF1ab₄₂₈₃₋₄₂₉₁ | LASGGQPI | 37 | M₈₉₋₉₇ | LMWLSYFI | 51 |
| ORF1ab₅₄₇₀₋₅₄₇₈ | KLSYGIAT | 38 | ORF6₃₋₁₁ | HLVDFQVT | 52 |
| ORF1ab₆₄₁₉₋₆₄₂₇ | LDAYNMMI | 39 | ORF7b₂₆₋₃₄ | IFWFSLEL | 53 |
| ORF1ab₆₇₄₉₋₆₇₅₇ | LLLDDFVE | 40 | ORF8a₃₁₋₃₉ | YWVDOPCP | 54 |
| S₂₋₁₀ | VFLVLLPL | 41 | ORF8a₇₃₋₈₁ | IDIGNYTV | 55 |
| S₆₉₁₋₆₉₉ | SIIAYTMS | 42 | ORF10₃₋₁₁ | YINVFAFP | 56 |
| S₉₅₈₋₉₆₆ | LNTLVKQL | 43 | ORF10₅₋₁₃ | VFAFPFTI | 57 |

The present invention is not limited to the aforementioned CD8⁺ T cell epitopes.

### CD4+ Epitopes

The present invention features a plurality of CD4+ T cell epitopes which may comprise one or more mutations. In some embodiments, a mutation may be synonymous or non-synonymous. In some embodiments, the mutation may be a point mutation. In other embodiments, the mutation may be a single point mutation (such as the above-mentioned mutations). In other embodiments, a single point mutation may be substitutions, deletions, or inversions

Table 6: below describes the sequences for the mutated epitope regions. Bolded amino acids indicate amino acids that have been mutated when compared to the SARS-CoV-2-Wuhan (MN908947.3) strain.

| | | SEQ ID |
|---|---|---|
| CD4⁺ Epitope | Sequence | NO: |
| S₁₋₁₃ | MFVFLVLLPLVSI | 154 |

Examples of methods for identifying potential CD4+ T cell epitopes and screening conservancy of potential CD4+ T cell epitopes are described herein. The present invention is not limited to the particular software systems disclosed, and other software systems are accessible to one of ordinary skill in the art for such methods. The present invention is not limited to the specific haplotypes used herein. For example, one of ordinary skill in the art may select alternative molecules (e.g., HLA molecules) for molecular docking studies.

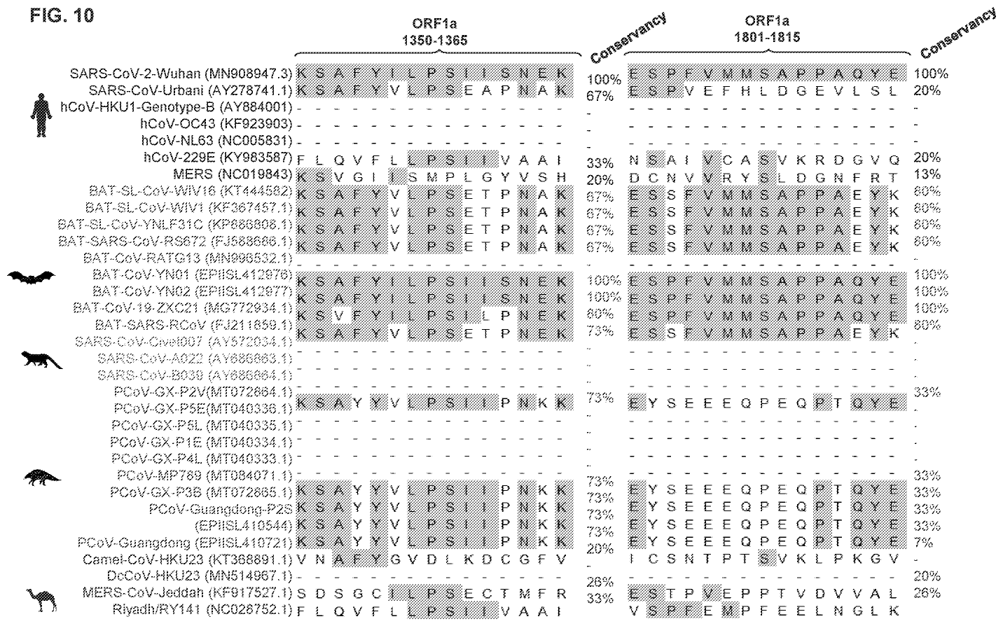

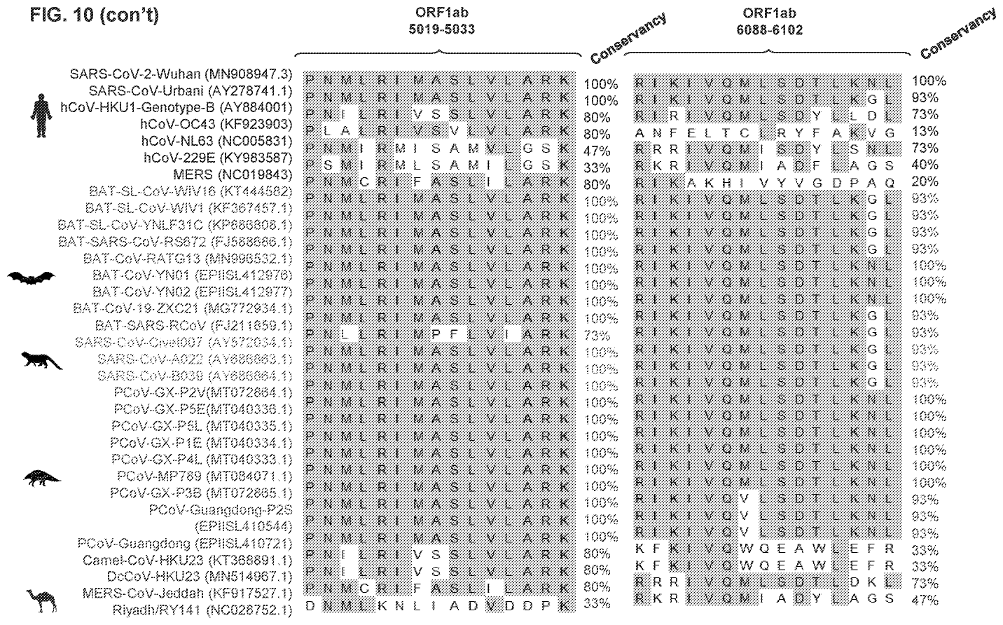

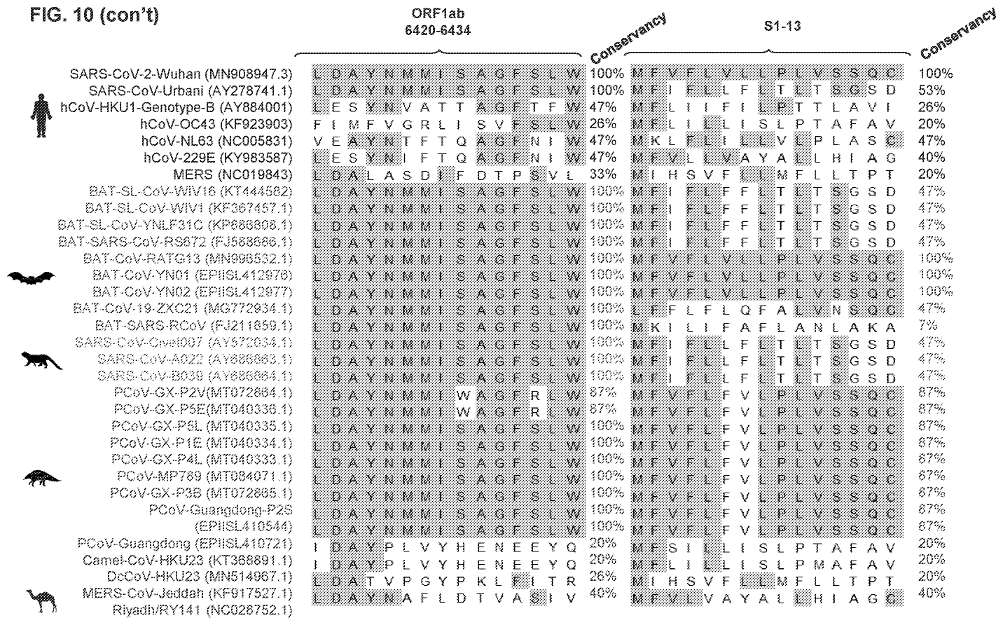

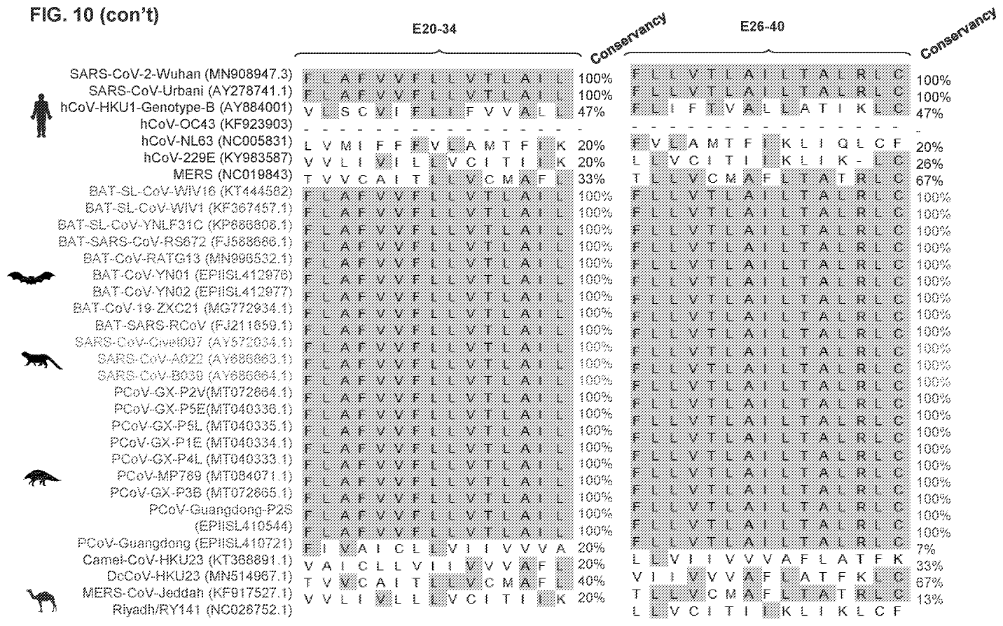

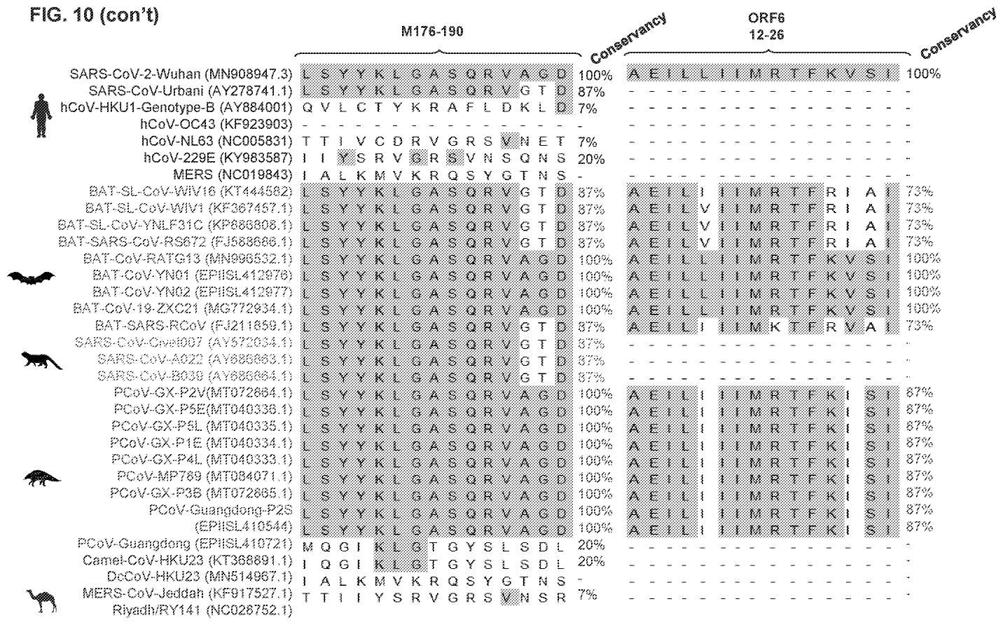

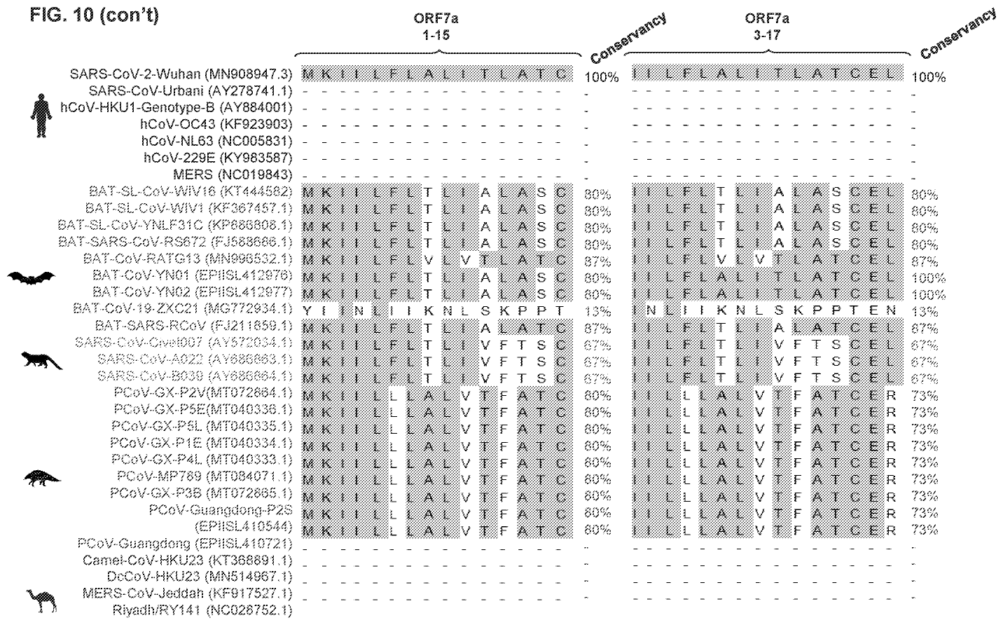

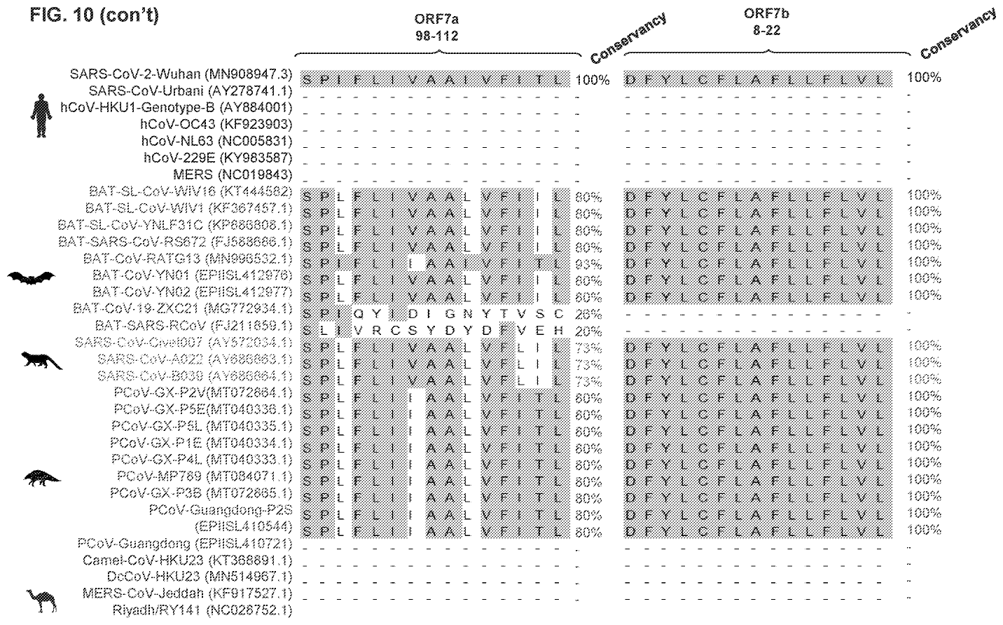

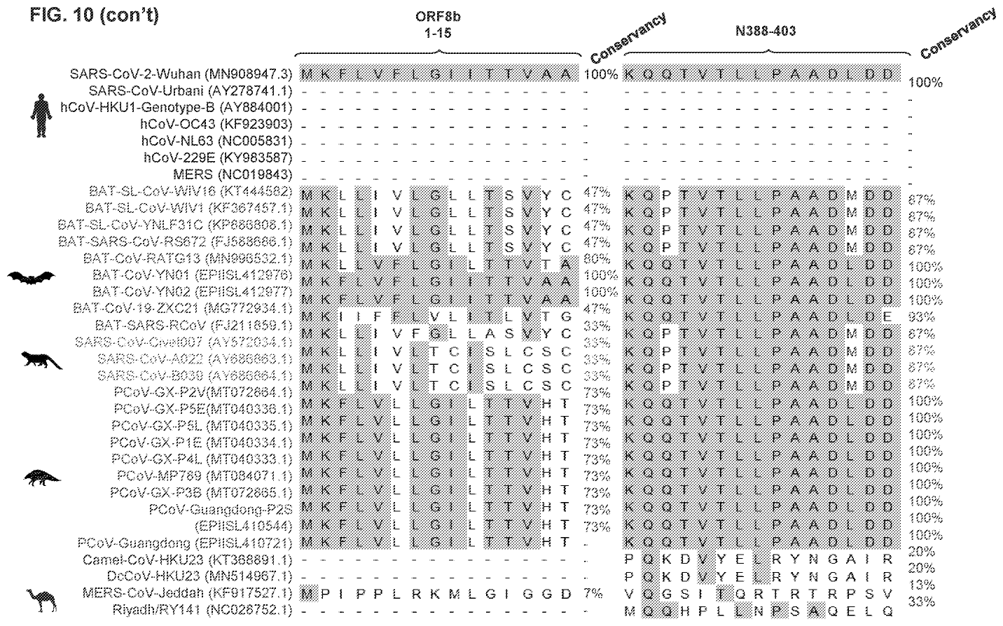

FIG. 10 shows the identification of highly mutated potential SARS-CoV-2-derived human CD4+ T cell epitopes that bind with high affinity to HLA-DR molecules. Out of a total of 9,594 potential HLA-DR-restricted CD4+ T cell epitopes from the whole genome sequence of SARS-CoV-2-Wuhan-Hu-1 strain (MN908947.3), 16 epitopes that bind with high affinity to HLA-DRB1 molecules were selected. The conservancy of the 16 CD4+ T cell epitopes was analyzed among human and animal Coronaviruses. Shown are the comparison of sequence homology for the 16 CD4+ T cell epitopes among 81,963 SARS-CoV-2 strains (that currently circulate in 6 continents), the 4 major “common cold” Coronaviruses that cased previous outbreaks (i.e. hCoV-OC43, hCoV-229E, hCoV-HKU1, and hCoV-NL63), and the SL-CoVs that were isolated from bats, civet cats, pangolins and camels. Epitope sequences highlighted in green present high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels.

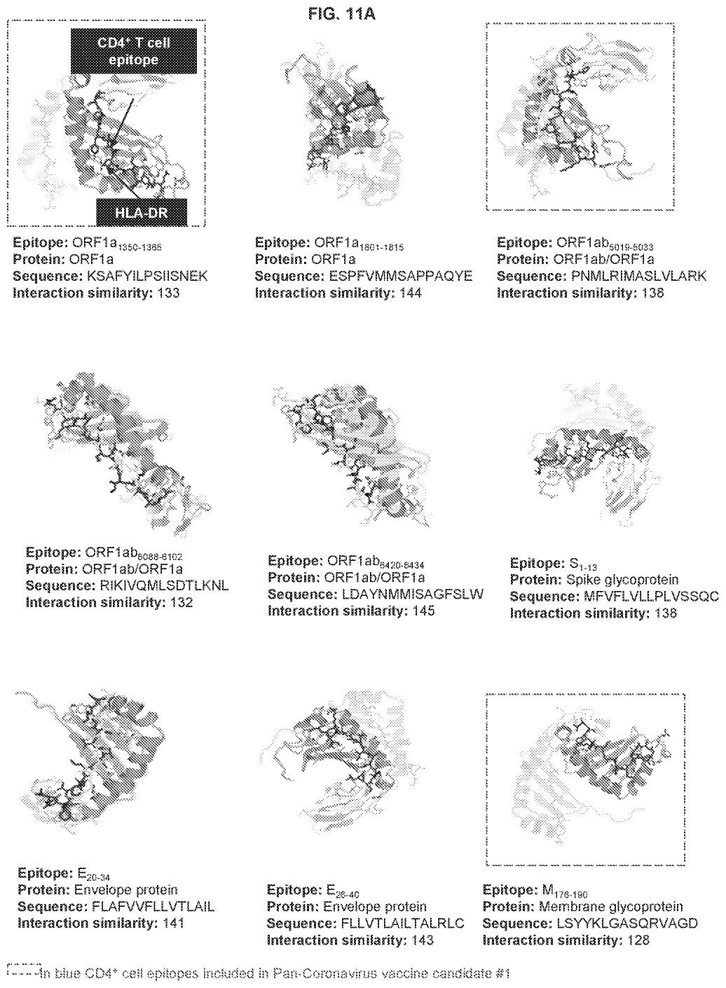

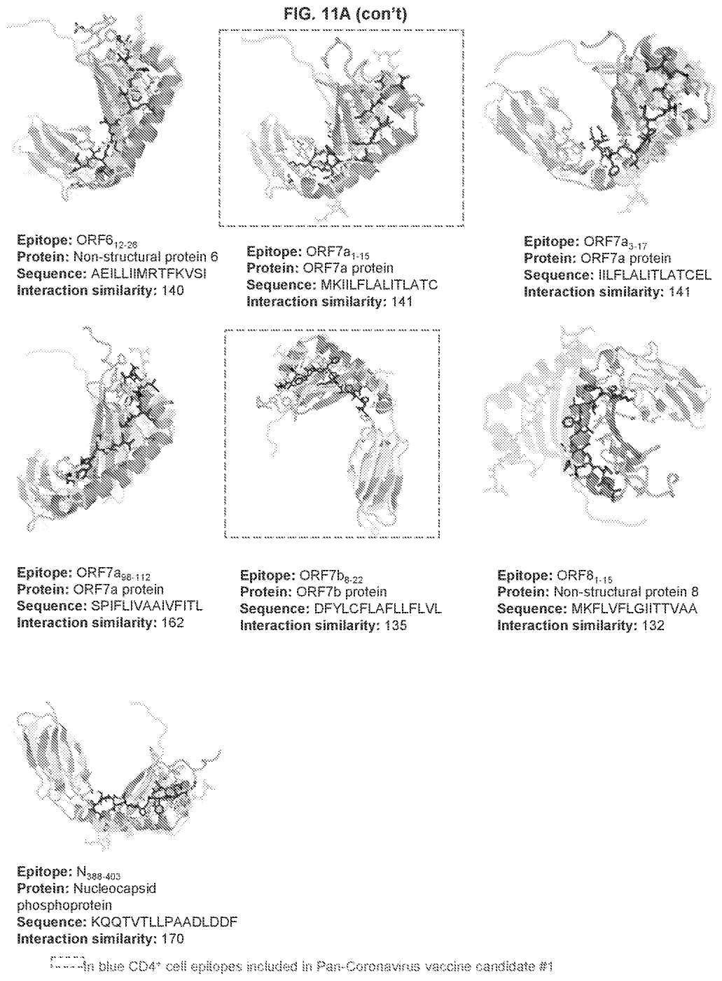

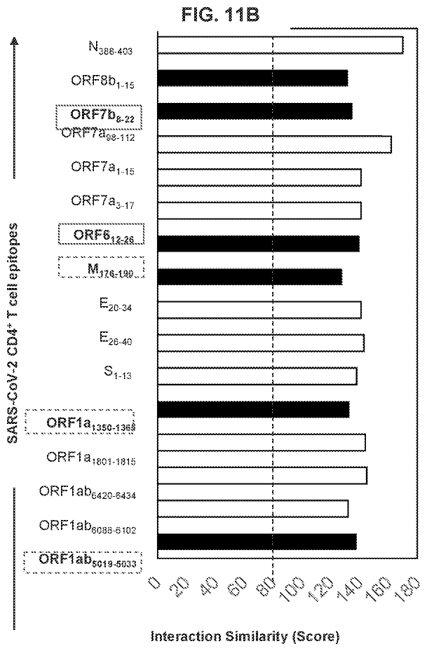

From the analysis, 16 CD4+ T cell epitopes were selected as being highly mutated. FIG. 11A and FIG. 11B show the docking of the mutated epitopes to the groove of HLA-A*02:01 molecules as well as the interaction scores determined by protein-peptide molecular docking analysis.

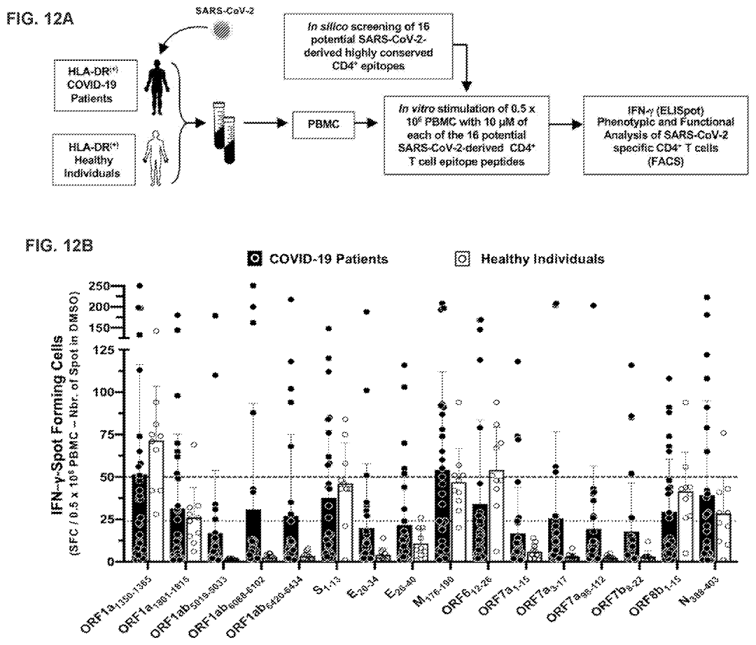

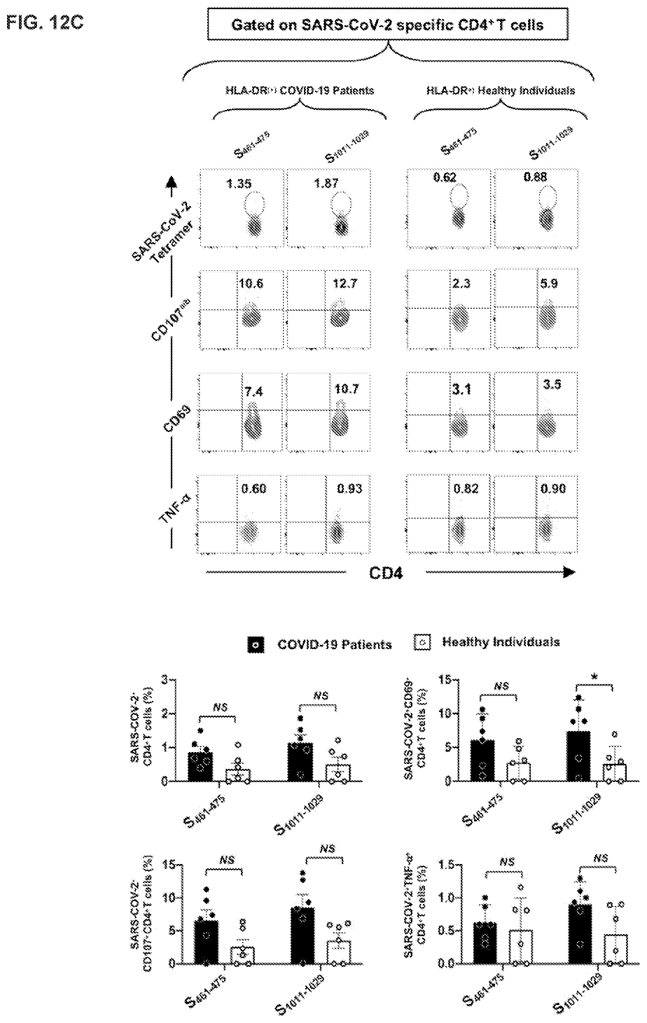

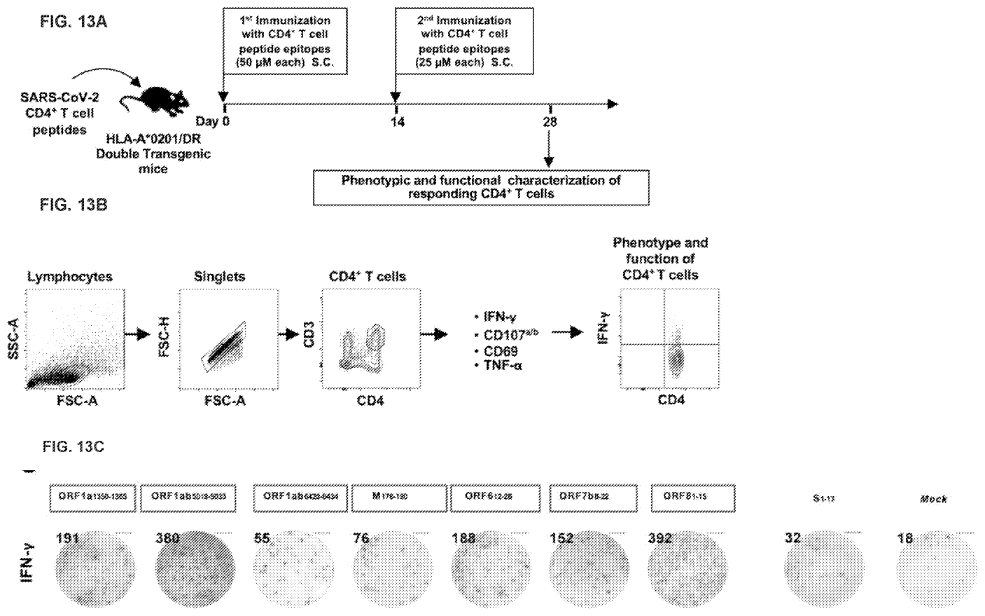

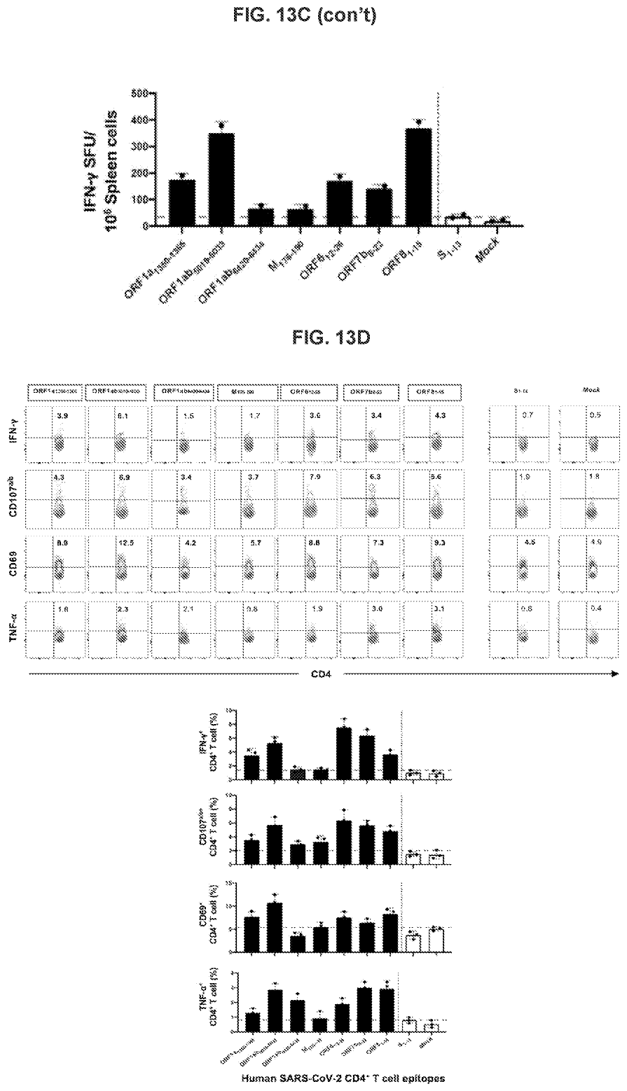

FIG. 12A, FIG. 12B, and FIG. 12C show that CD4+ T cells specific to several highly mutated SARS-CoV-2 epitopes disclosed herein were detected in COVID-19 patients and unexposed healthy individuals. FIG. 13A, FIG. 13B, FIG. 13C, and FIG. 13D show Immunogenicity of the identified SARS-CoV-2 CD4+ T cell epitopes.

The CD4⁺ T cell target epitopes discussed above include ORF1a₁₃₅₀₋₁₃₆₅, ORF1ab₅₀₁₉₋₅₀₃₃, ORF6₁₂₋₂₆, ORF1ab₆₀₈₈₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₆₋₁₉₀, N₃₆₈₋₄₀₃, ORF7a₃₋₁₇, ORF7a₁₋₁₅, ORF7b₈₋₂₂, ORF7a₉₈₋₁₁₂, and ORF8₁₋₁₅. FIG. 9 shows the genome-wide location of the epitopes. Thus, in certain embodiments, the vaccine composition may comprise one or more CD4⁺ T cell target epitopes selected from ORF1a₁₃₅₀₋₁₃₆₅, ORF1ab₅₀₁₉₋₅₀₃₃, ORF6₁₂₋₂₆, ORF1ab₆₀₈₈₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₆₋₁₉₀, N₃₈₈₋₄₀₃, ORF7a₃₋₁₇, ORF7a₁₋₁₅, ORF7b₈₋₂₂, ORF7a₉₈₋₁₁₂, ORF8₁₋₁₅, or a combination thereof. Table 7 below describes the sequences for the aforementioned epitope regions.

TABLE 7

| | | SEQ | | | SEQ |
|---|---|---|---|---|---|
| CD4⁺ T Cell | | ID | CD4⁺ T Cell | | ID |
| Epitope | Epitope Sequence | NO: | Epitope | Epitope Sequence | NO: |
| ORF1a₁₃₅₀₋₁₃₆₅ | KSAFYILPSIISNEK | 58 | M₁₇₆₋₁₉₀ | LSYYKLGASQRVAGD | 66 |
| ORF1a₁₈₀₁₋₁₈₁₅ | ESPFVMMSAPPAQYE | 59 | ORF6₁₂₋₂₆ | AEILLIIMRTFKVSI | 67 |
| ORF1ab₅₀₁₉₋₅₀₃₃ | PNMLRIMASLVLARK | 60 | ORF7a₁₋₅ | MKIILFLALITLATC | 68 |
| ORF1ab₆₀₈₈₋₆₁₀₂ | RIKVQMLSDTLKNL | 61 | ORF7a₃₋₁₇ | IIFLALITLATCEL | 69 |
| ORF1ab₆₄₂₀₋₆₄₃₄ | LDAYNMMISAGFSLW | 62 | ORF7a₉₈₋₁₁₂ | SPIFLIVAAIVFITL | 70 |
| S₁₋₁₃ | MFVFLVLLPLVSS | 63 | ORF7b₈₋₂₂ | DFYLCFLAFLLFLVL | 71 |
| E₂₀₋₃₄ | FLAFVVFLLVTLAIL | 64 | ORF8b₁₋₁₅ | MKFLVFLGIITTVAA | 72 |
| E₂₆₋₄₀ | FLLVTLAILTALRLC | 65 | N₃₈₈₋₄₀₃₁ | KQQTVTLLPAADLDDF | 73 |

The present invention is not limited to the aforementioned CD4⁺ T cell epitopes. For example, the present invention also includes variants of the aforementioned CD4⁺ T cell epitopes, for example sequences wherein the aforementioned CD4⁺ T cell epitopes are truncated by one or more amino acids or extended by one or more amino acids (examples shown below in Table 8).

TABLE 8

| | Sequence with | SEQ | CD4⁺ T Cell | Sequence with | SEQ |
|---|---|---|---|---|---|
| CD4⁺ T Cell | Single | ID | Epitope | Single | ID |
| Epitope Origin | AA Truncation | NO: | Origin | AA Truncation | NO: |
| ORF1a₁₃₅₀₋₁₃₆₅ | KSAFYILPSIISNE | 74 | ORF1a₁₃₅₀₋₁₃₆₅ | SAFYILPSIISNEK | 90 |
| ORF1a₁₈₀₁₋₁₈₁₅ | ESPFVMMSAPPAQY | 75 | ORF1a₁₈₀₁₋₁₈₁₅ | SPFVMMSAPPAQYE | 91 |
| ORF1ab₅₀₁₉₋₅₀₃₃ | PNMLRIMASLVLAR | 76 | ORF1ab₅₀₁₉₋₅₀₃₃ | NMLRIMASLVLARK | 92 |
| ORF1ab₆₀₈₈₋₆₁₀₂ | RIKVQMLSDTLKN | 77 | QRF1ab₆₀₈₆₋₆₁₀₂ | IKVOMLSDTLKNL | 93 |
| ORF1ab₆₄₂₀₋₆₄₃₄ | LDAYNMMISAGFSL | 78 | ORF1ab₆₄₂₀₋₆₄₃₄ | DAYNMMISAGFSLW | 94 |
| S₁₋₁₃ | MFVFLVLLPLVS | 79 | S₁₋₁₃ | FVFLVLLPLVSS | 95 |
| E₂₀₋₃₄ | FLAFVVFLLVTLAL | 86 | E₂₀₋₃₄ | LAFVVFLLVTLAIL | 96 |
| E₂₆₋₄₀ | FLLVTLAILTALRL | 81 | E₂₆₋₄₀ | LLVTLAILTALRLC | 97 |
| M₁₇₆₋₁₉₀ | LSYYKLGASQRVAG | 82 | M₁₇₆₋₁₉₀ | SYYKLGASQRVAGD | 98 |
| ORF6₁₂₋₂₆ | AEILLIIMRTFKVS | 83 | ORF6₁₂₋₂₆ | EILLIIMRTFKVS | 99 |
| ORF7a₁₋₁₅ | MKIILFLALITLAT | 84 | ORF7a₁₋₁₅ | KIILFLALITLATC | 160 |
| ORF7a₃₋₁₇ | IIFLALITLATCE | 85 | ORF7a₃₋₁₇ | IFLALITLATCEL | 101 |
| ORF7a₉₆₋₁₁₂ | SPIFLIVAAIVFIT | 86 | ORF7a₉₆₋₁₁₂ | PIFLIVAAIVFITL | 102 |
| ORF7b₈₋₂₂ | DFYLCFLAFLLFLV | 87 | ORF7b₈₋₂₂ | FYLCFLAFLLFLVL | 103 |
| ORF8b₁₋₁₅ | MIKFLVFLGIITTVA | 88 | ORF8b₁₋₁₅ | KFLVFLGIITTVAA | 104 |
| N₃₈₈₋₄₀₃₁ | KQQTVTLLPAADLDD | 89 | N₃₈₈₋₄₀₃₁ | QQTVTLLPAADLDDF | 105 |

The present invention is not limited to the aforementioned CD4⁺ T cell epitopes.

### B Cell Epitopes

The present invention features a plurality of B cell epitopes which may comprise one or more mutations. In some embodiments, a mutation may be synonymous or non-synonymous. In some embodiments, the mutation may be a point mutation. In other embodiments, the mutation may be a single point mutation (such as the above mentioned mutations). In other embodiments, a single point mutation may be substitutions, deletions, or Inversions.

Table 9: below describes the sequences for the mutated epitope regions. Bolded amino acids indicate amino acids that have been mutated when compared to the SARS-CoV-2-Wuhan (MN908947.3) strain.

| B Cell | | SEQ ID |
|---|---|---|
| Epitope | Sequence | NO: |
| S₁₃₋₃₇ | IQCVNLTTRTQLPPAYTNSFTRGVY | 155 |
| S₅₉₋₈₁ | FSNVTWFHAIHVSGTNGTKRFAN | 172 |
| S₂₈₇₋₃₁₇ | DAVDCALDPLSETKCTLKSFTVEKGIYQTSN | 173 |
| S₄₄₀₋₅₀₁ | NLDSKVGGNYNYLYRLFRKSNLKPFERDISTEIYQAGSTPCNGVKGFNC | 174 |
| | YFPLQSYGFQPTY | |
| S₄₄₀₋₅₀₁ | NLDSKVGGNYNYRYRLFRKSNLKPFERDISTEIYQAGSTPCNGVEGFNC | 175 |
| | YFPLQSYGFQPTE | |
| S₅₂₄₋₅₉₈ | VCGPKKSTNLVKNKCVNFNFNGLTGTGVLTESNKKFLPFQQFGRDIDDT | 176 |
| | TDAVRDPQTLEILDITPCSFGGVSVI | |
| S₆₀₁₋₆₄₀ | GTNTSNQVAVLYQGVNCTEVPVAIHADQLTPTWRVYSTGS | 177 |
| S₁₁₃₃₋₁₁₇₂ | VNNTVYDPLQPELDSFKEELDKYFKNHTSPDVDLGDISGI | 178 |

The present invention is not limited to the aforementioned B cell epitopes. For example, the present invention may also include other variants of the aforementioned B cell epitopes.

Examples of methods for identifying potential B cell epitopes and screening conservancy of potential B cell epitopes are described herein. The present invention is not limited to the particular software systems disclosed, and other software systems are accessible to one of ordinary skill in the art for such methods.

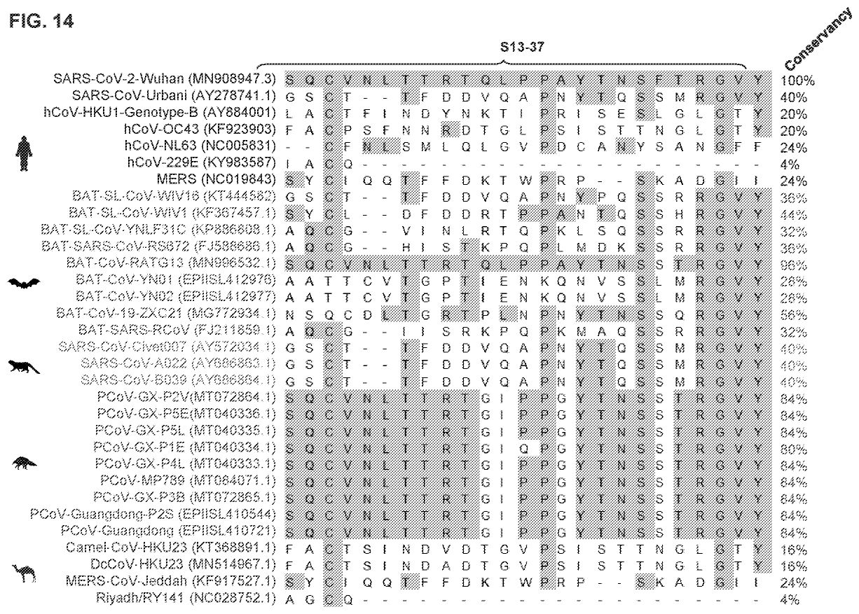

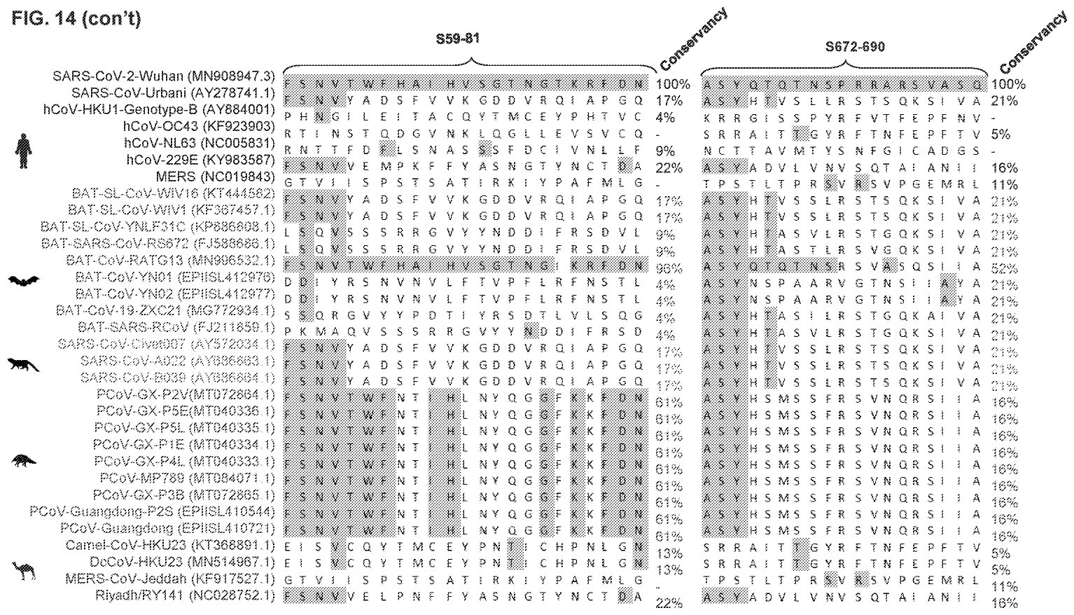

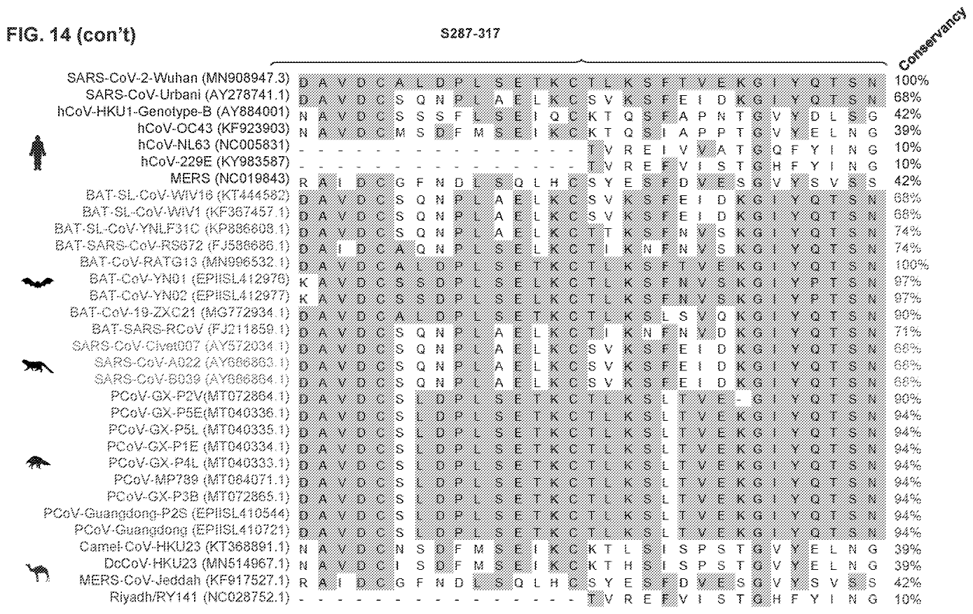

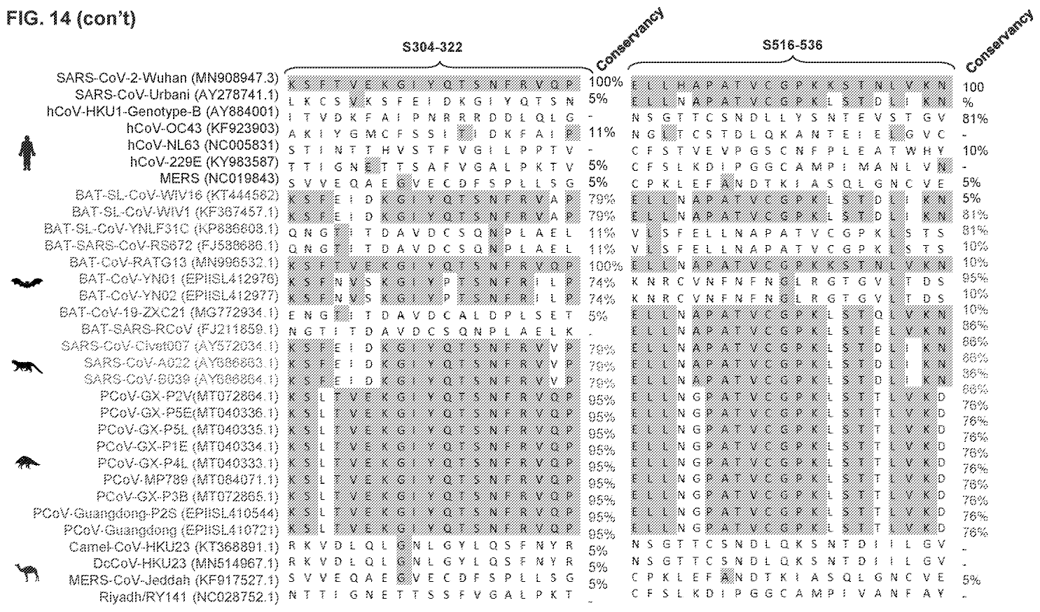

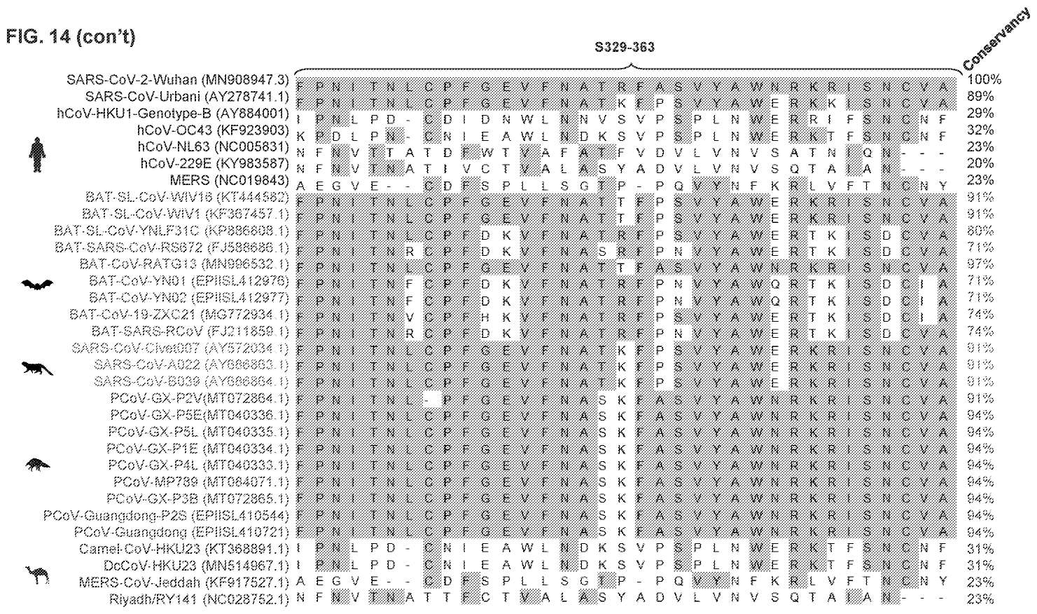

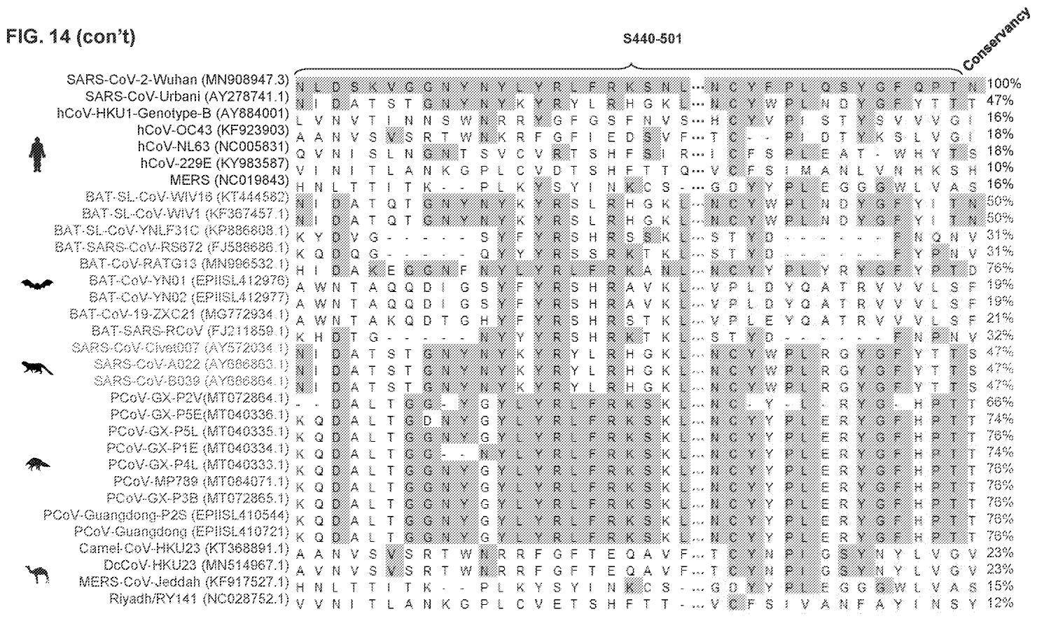

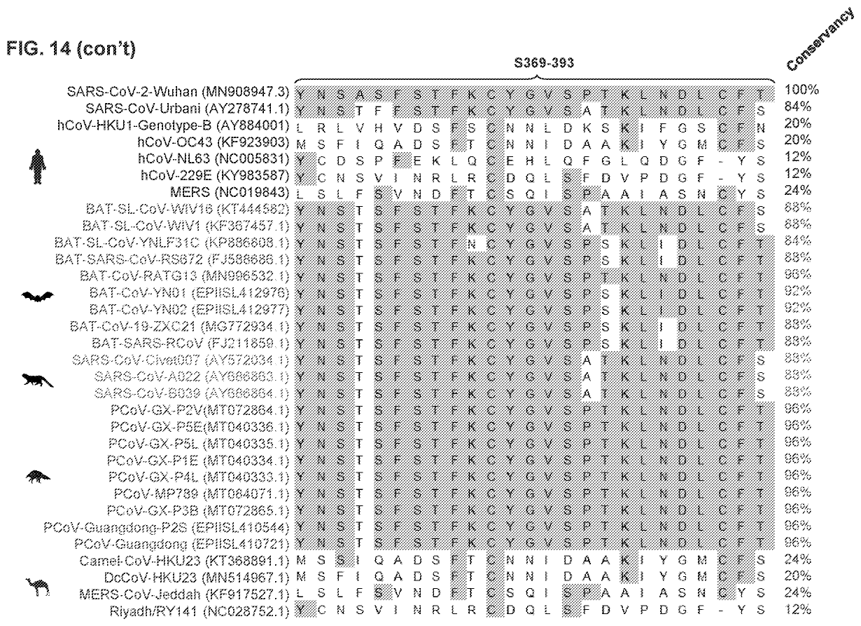

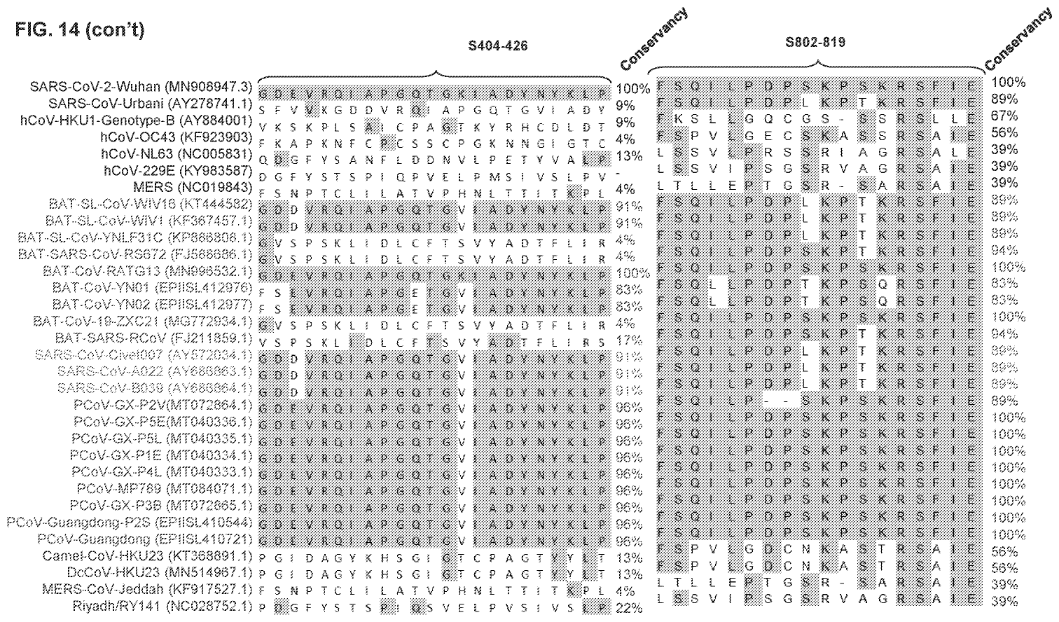

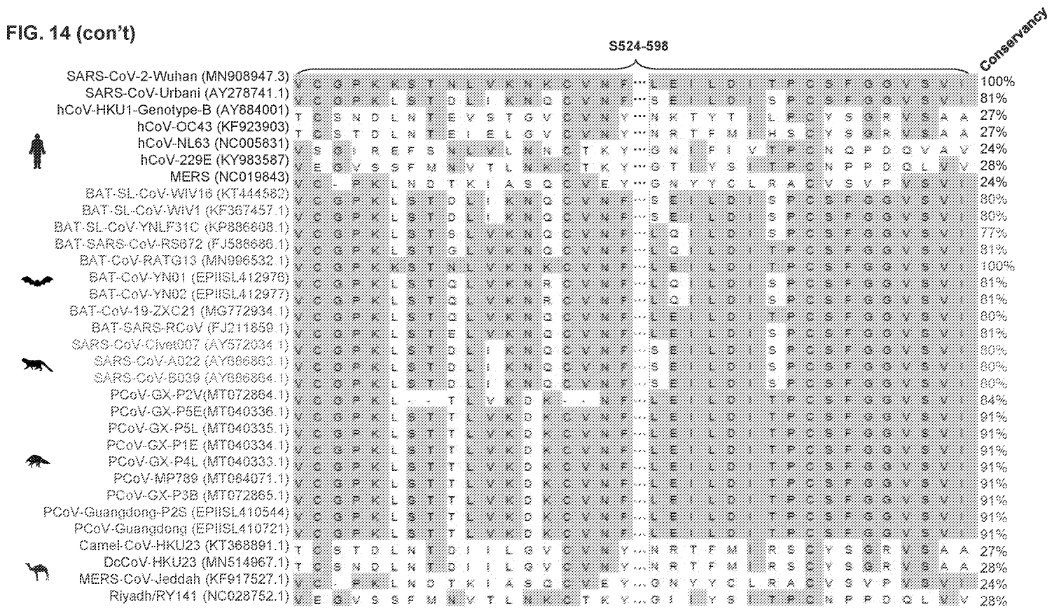

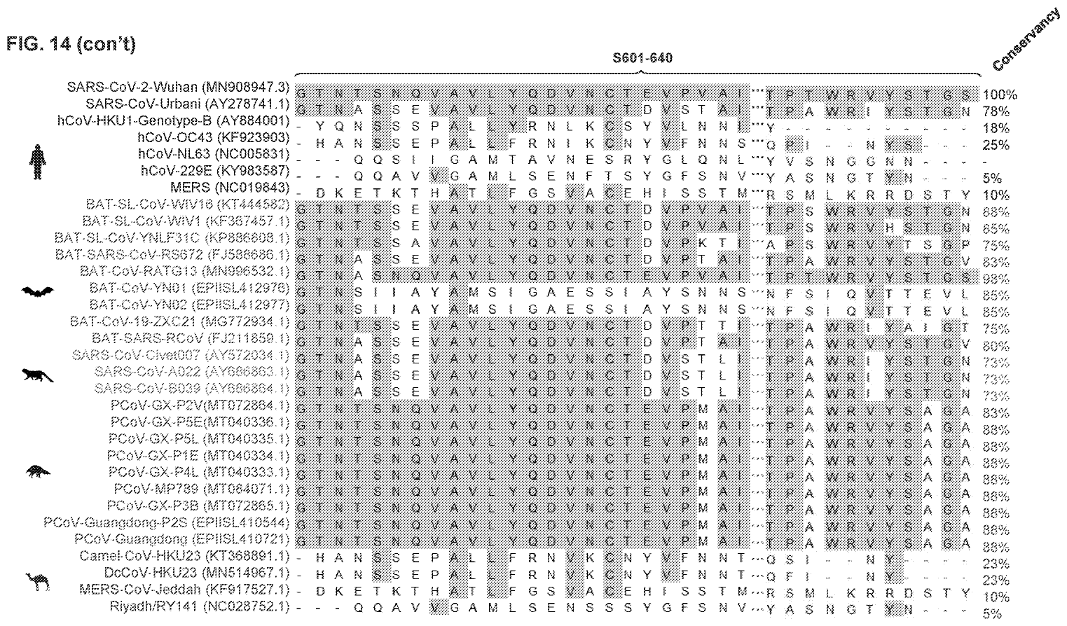

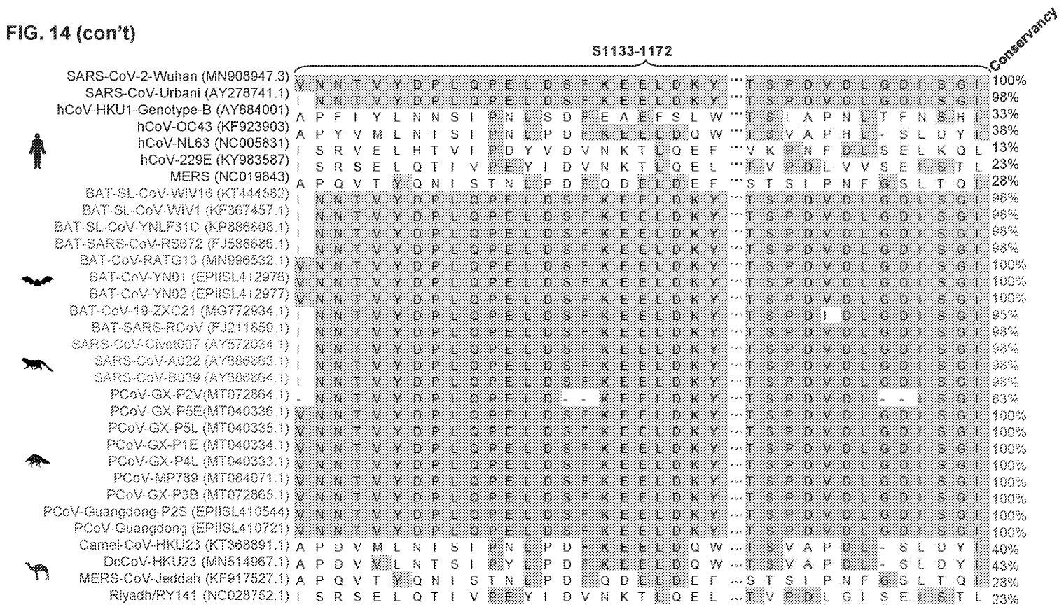

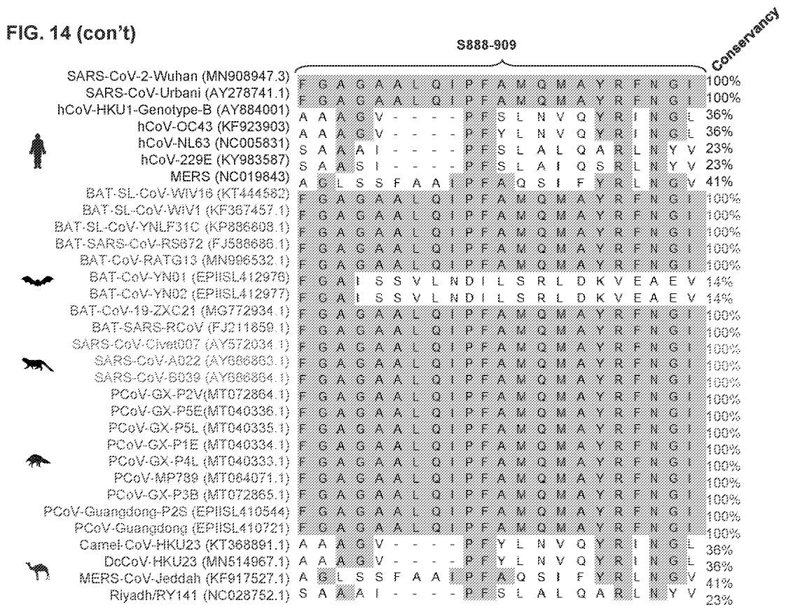

FIG. 14 shows the conservation of Spike-derived B cell epitopes among human, bat, civet cat, pangolin, and camel coronavirus strains. Multiple sequence alignment performed using ClustalW among 29 strains of SARS coronavirus (SARS-CoV) obtained from human, bat, civet, pangolin, and camel. This includes 7 human SARS/MERS-CoV strains (SARS-CoV-2-Wuhan (MN908947.3), SARS-HCoV-Urbani (AY278741.1), CoV-HKU1-Genotype-B (AY884001), CoV-OC43 (KF923903), CoV-NL63 (NC005831), CoV-229E (KY983587), MERS (NC019843)); 8 bat SARS-CoV strains (BAT-SL-CoV-WIV16 (KT444582), BAT-SL-CoV-WIV1 (KF367457.1), BAT-SL-CoV-YNLF31C (KP886808.1), BAT-SARS-CoV-RS672 (FJ588686.1), BAT-CoV-RATG13 (MN996532.1), BAT-CoV-YN01 (EPIISL412976), BAT-CoV-YNO2 (EPIISL412977). BAT-CoV-19-ZXC21 (MG772934.1); 3 Civet SARS-CoV strains (SARS-CoV-Civet007 (AY572034.1), SARS-CoV-A022 (AY686863.1), SARS-CoV-B039 (AY686864.1)); 9 pangolin SARS-CoV strains (PCoV-GX-P2V(MT072864.1), PCoV-GX-P5E(MT040336.1), PCoV-GX-P5L (MT040335.1), PCoV-GX-P1E (MT040334.1), PCoV-GX-P4L (MT040333.1), PCoV-MP789 (MT084071.1), PCoV-GX-P3B (MT072865.1), PCoV-Guangdong-P2S (EPIISL410544), PCoV-Guangdong (EPIISL410721)); 4 camel SARS-CoV strains (Camel-CoV-HKU23 (KT368891.1), DcCoV-HKU23 (MN514967.1), MERS-CoV-Jeddah (KF917527.1), Riyadh/RY141 (NC028752.1)) and 1 recombinant strain (FJ211859.1)). Regions highlighted with blue color represent the sequence homology. The B cell epitopes, which showed at least 50% conservancy among two or more strains of the SARS Coronavirus or possess receptor-binding domain (RBD) specific amino acids were selected as candidate epitopes.

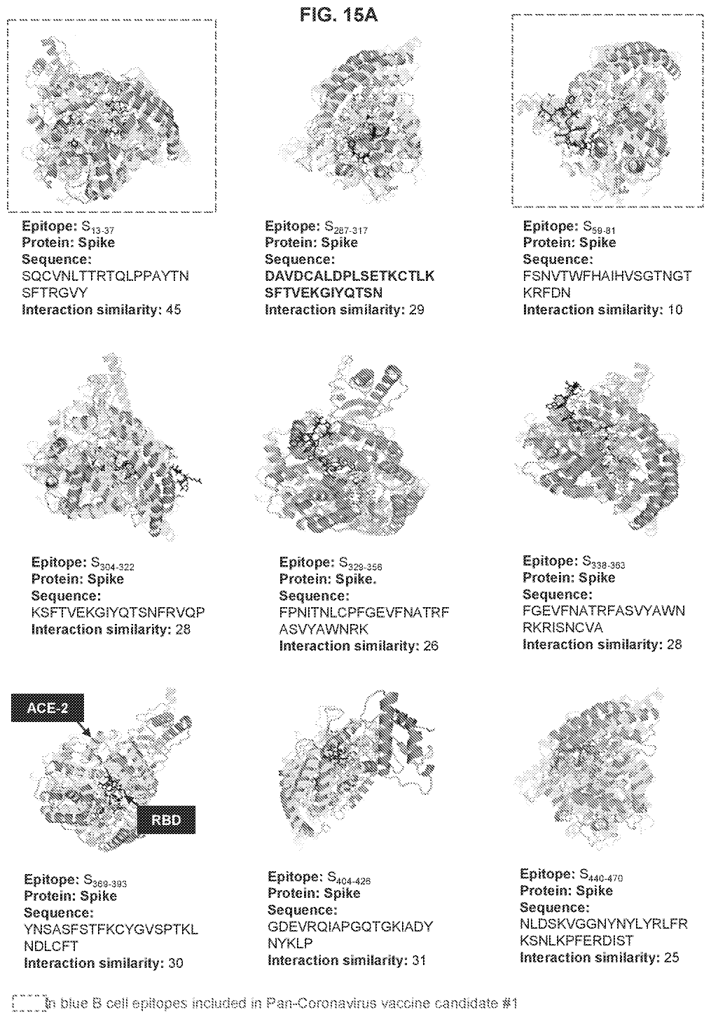

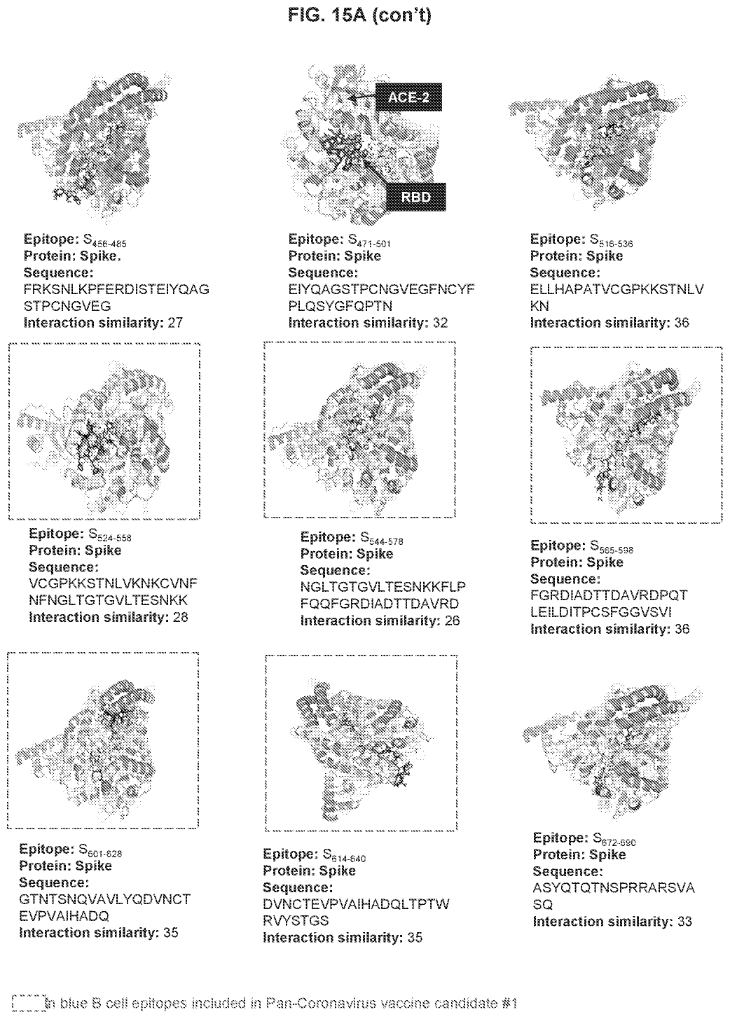

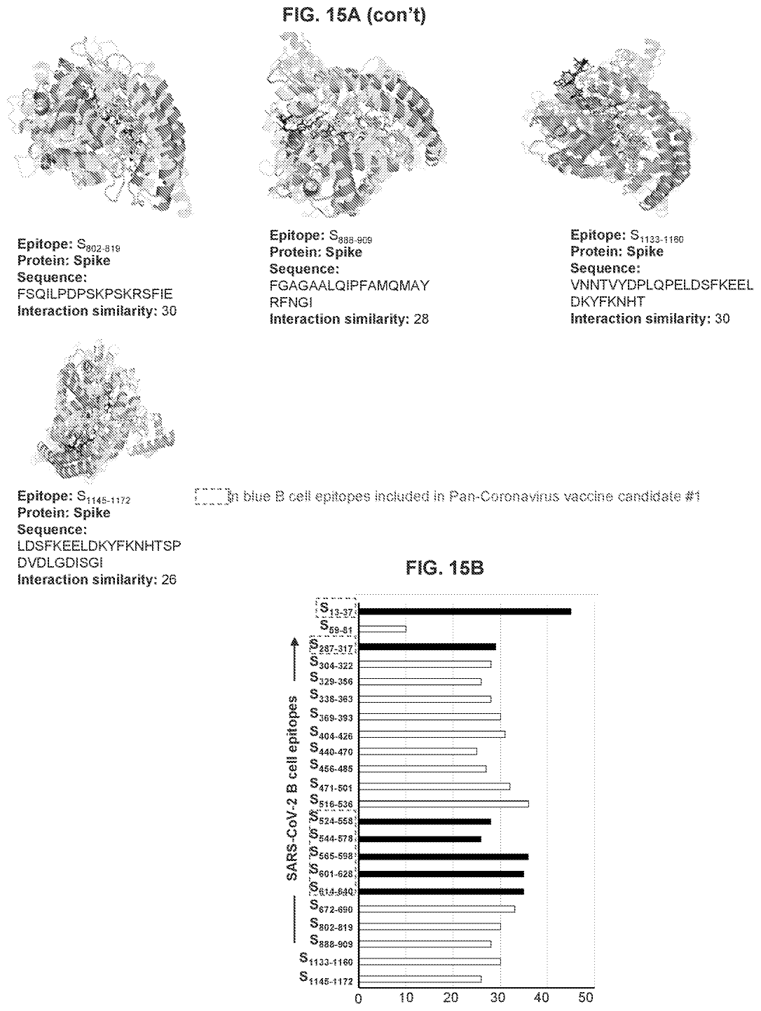

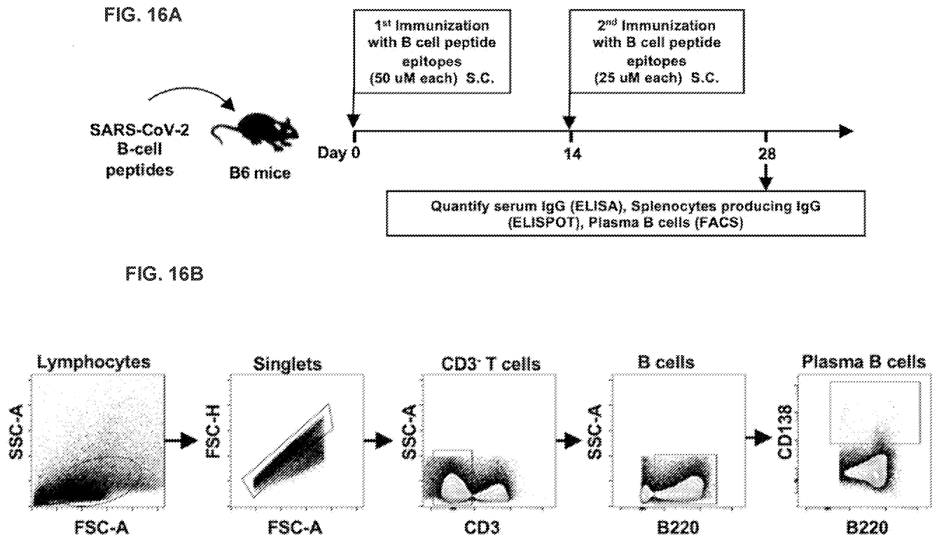

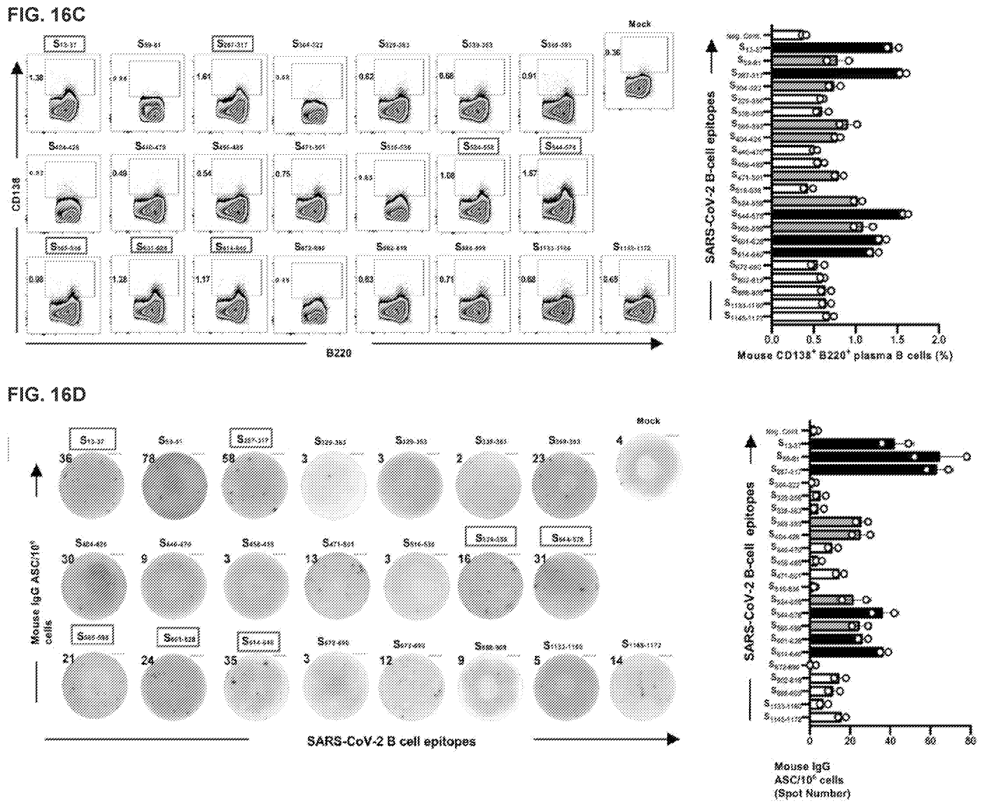

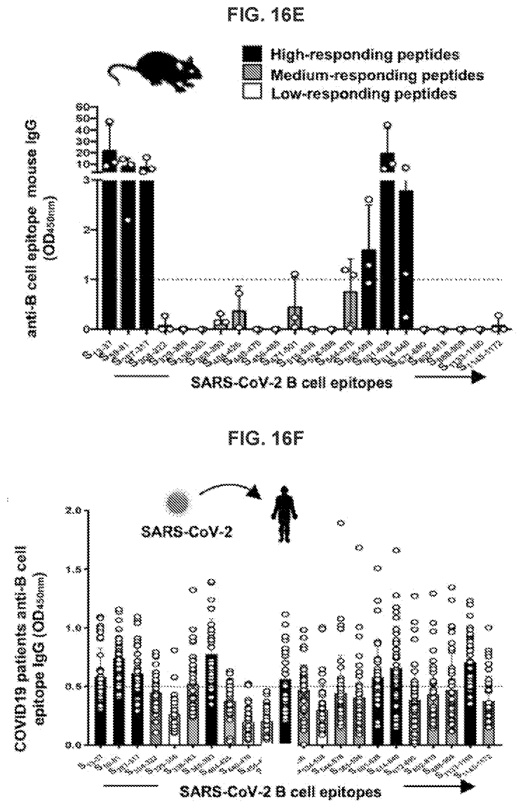

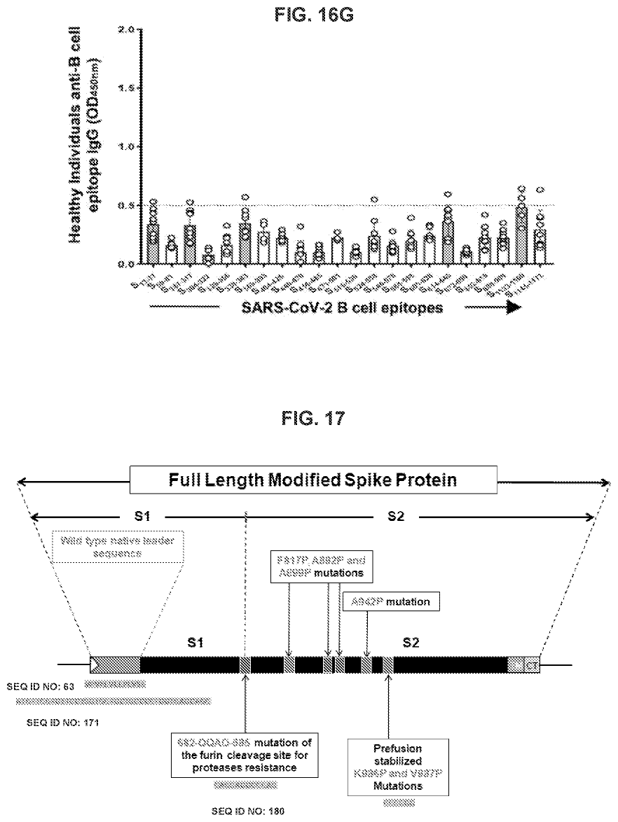

From the analysis, 22 B cell epitopes were selected as being highly mutated. FIG. 15A and FIG. 15B show the docking of the mutated epitopes to the ACE2 receptor as well as the interaction scores determined by protein-peptide molecular docking analysis. FIG. 16A, FIG. 16B, FIG. 16C, FIG. 16D, FIG. 16E, FIG. 16F, and FIG. 16G show immunogenicity of the identified SARS-CoV-2 B cell epitopes

The B cell target epitopes discussed above include S₂₈₇₋₃₁₇, S₅₂₄₋₅₉₈, S₈₀₁₋₆₄₀, S₈₀₂₋₈₁₉, S₈₈₈₋₉₀₉, S₃₆₉₋₃₉₃, S₄₄₀₋₅₀₁, S₁₁₃₃₋₁₁₇₂, S₃₂₉₋₃₆₃, S₅₉₋₈₁, and S₁₃₋₃₇. FIG. 9 shows the genome-wide location of the epitopes. Thus, in certain embodiments, the vaccine composition may comprise one or more B cell target epitopes selected from: S₂₈₇₋₃₁₇, S₅₂₄₋₅₉₈, S₆₀₁₋₆₄₀, S₈₀₂₋₈₁₉, S₈₈₈₋₉₀₉, S₃₆₉₋₃₉₃, S₄₄₀₋₅₀₁, S₁₁₃₃₋₁₁₇₂, S₃₂₉₋₃₆₃, S₅₉₋₈₉, and S₁₃₋₃₇. In some embodiments, the B cell epitope is whole spike protein. In some embodiments, the B cell epitope is a portion of the spike protein. Table 10 below describes the sequences for the aforementioned epitope regions.

TABLE 10

| B Cell | | SEQ ID |
|---|---|---|
| Epitope | Epitope Sequence | NO: |
| S₁₃₋₃₇ | SQCVNLTTRTQLPPAYTNSFTRGVY | 106 |
| S₅₉₋₈₁ | FSNVTWFHAIHVSGTNGTKRFDN | 107 |
| S₂₈₇₋₃₁₇ | DAVDCALDPLSETKCTLKSFTVEKGIYQTSN | 108 |
| S₆₀₁₋₆₄₀ | GTNTSNQVAVLYQDVNCTEVPVAIHADQLTPTWRVYSTGS | 109 |
| S₅₂₄₋₅₉₈ | VCGPKKSTNLVKNKCVNFNFNGLTGTGVLTESNKKFLPFQQFGRDI | 110 |
| | ADTTDAVRDPQTLEILDITPCSFGGVSVI | |
| S₄₄₀₋₅₀₁ | NLDSKVGGNYNYLYRLFRKSNLKPFERDISTEIYQAGSTPCNGVEG | 111 |
| | FNCYFPLQSYGFQPTE | |
| S₃₆₉₋₃₉₃ | YNSASFSTFKCYGVSPTKLNDLCFT | 112 |
| S₃₂₉₋₃₆₃ | FPNITNLCPFGEVFNATRFASVYAWNRKRISNCVA | 113 |
| S₁₁₃₃₋₁₁₇₂ | VNNTVYDPLQPELDSFKEELDKYFKNHTSPDVDLGDISGI | 114 |
| S₈₀₂₋₈₁₉ | FSQILPDPSKPSKRSFIE | 115 |
| S₈₈₈₋₉₀₉ | FGAGAALQIPFAMQMAYRFNGI | 116 |

The present invention is not limited to the aforementioned B cell epitopes. For example, the present invention also includes variants of the aforementioned B cell epitopes, for example sequences wherein the aforementioned B cell epitopes are truncated by one or more amino acids or extended by one or more amino acids (examples shown below in Table 11).

TABLE 11

| Origin of | | SEQ ID |
|---|---|---|
| Epitope | Sequence with AA Truncation | NO: |
| S₁₃₋₃₅ | SQCVNLTTRTQLPPAYTNSFTRG | 117 |
| S₅₉₋₇₉ | FSNVTWFHAIHVSGTNGTKRF | 118 |
| S₂₈₇₋₃₁₅ | DAVDCALDPLSETKCTLKSFTVEKGIYQT | 119 |
| S₆₀₁₋₆₃₈ | GTNTSNQVAVLYQDVNCTEVPVAIHADQLTPTWRVYST | 120 |
| S₅₂₄₋₅₉₆ | VCGPKKSTNLVKNKCVNFNFNGLTGTGVLTESNKKFLPFQQFGRDI | 121 |
| | ADTTDAVRDPQTLEILDITPCSFGGVS | |
| S₄₄₀₋₄₉₉ | NLDSKVGGNYNYLYRLFRKSNLKPFERDISTEIYQAGSTPCNGVEG | 122 |
| | FNCYFPLQSYGFQP | |
| S₃₆₉₋₃₉₁ | YNSASFSTFKCYGVSPTKLNDLC | 123 |
| S₃₂₉₋₃₆₁ | FPNITNLCPFGEVFNATRFASVYAWNRKRISNC | 124 |
| S₁₁₃₃₋₁₁₇₀ | VNNTVYDPLQPELDSFKEELDKYFKNHTSPDVDLGDIS | 12S |
| S₈₀₂₋₈₁₇ | FSQILPDPSKPSKRSF | 126 |
| S₈₈₈₋₉₀₇ | FGAGAALQIPFAMQMAYRFN | 127 |
| S₁₅₋₃₇ | CVNLTTRTQLPPAYTNSFTRGVY | 128 |
| S₆₁₋₈₁ | NVTWFHAIHVSGTNGTKRFDN | 129 |
| S₂₈₉₋₃₁₇ | VDCALDPLSETKCTLKSFTVEKGIYQTSN | 130 |
| S₆₀₃₋₆₄₀ | NTSNQVAVLYQDVNCTEVPVAIHADQLTPTWRVYSTGS | 131 |
| S₅₂₆₋₅₉₈ | GPKKSTNLVKNKCVNFNFNGLTGTGVLTESNKKFLPFQQFGRDIAD | 132 |
| | TTDAVRDPQTLEILDITPCSFGGVSVI | |
| S₄₄₂₋₅₀₁ | DSKVGGNYNYLYRLFRKSNLKPFERDISTEIYQAGSTPCNGVEGFN | 133 |
| | CYFPLQSYGFQPTE | |
| S₃₇₁₋₃₉₃ | SASFSTFKCYGVSPTKLNDLCFT | 134 |
| S₃₃₁₋₃₆₃ | NITNLCPFGEVFNATRFASVYAWNRKRISNCVA | 135 |
| S₁₁₃₅₋₁₁₇₂ | NTVYDPLQPELDSFKEELDKYFKNHTSPDVDLGDISGI | 136 |
| S₈₀₄₋₈₁₉ | QILPDPSKPSKRSFIE | 137 |
| S₈₉₀₋₉₀₉ | AGAALQIPFAMQMAYRFNGI | 138 |

As previously discussed, in some embodiments, the B cell epitope is in the form of whole spike protein. In some embodiments, the B cell epitope is in the form of a portion of spike protein. In some embodiments, the transmembrane anchor of the spike protein has an intact S1-S2 cleavage site. In some embodiments, the spike protein is in its stabilized conformation. In some embodiments, the spike protein is stabilized with proline substitutions at amino acid positions 988 and 987 at the top of the central helix in the S2 subunit. In some embodiments, the composition comprises a trimerized SARS-CoV-2 receptor-binding domain (RBD). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain Increases immunogenicity by multivalent display. FIG. 17 shows a non-limiting example of a spike protein comprising one or more mutations

In some embodiments, the spike protein comprises Tyr-489 and Asn-487 (e.g., Tyr-489 and Asn-487 help with interaction with Tyr 83 and Gln-24 on ACE-2). In some embodiments, the spike protein comprises Gln-493 (e.g., Gln-493 helps with interaction with Glu-35 and Lys-31 on ACE-2). In some embodiments, the spike protein comprises Tyr-505 (e.g., Tyr-505 helps with interaction with Glu-37 and Arg-393 on ACE-2). In some embodiments, the composition comprises a mutation 882-RRAR-885→682-QQAQ-685 in the S1-S2 cleavage site.

In some embodiments, the composition comprises at least one proline substitution. In some embodiments, the composition comprises at least two proline substitutions. For example, the proline substitution may be at position K988 and V987.

### Vaccine Candidates

As previously discussed, the present invention provides vaccine compositions comprising at least one B cell epitope and at least one CD4+ T cell epitope, at least one B cell epitope and at least one CD8+ T cell epitope, at least one CD4+ T cell epitope and at least one CD8+ T cell epitope, or at least one B cell epitope, at least one CD4+ T cell epitope, and at least one CD8+ T cell epitope.

In certain embodiments, at least one epitope is derived from a non-spike protein. In certain embodiments, the composition induces immunity to only the epitopes.

Table 12 and FIG. 18 show examples of vaccine compositions described herein. The present invention is not limited to the examples in Table 12

TABLE 12

| Vaccine | | SEQ ID |
|---|---|---|
| Candidate | Sequence: | NO: |
| 1 | CTCGACATTGATTATTGACTAGTTATTAATAGTAATCAATTACGGGGTCATT | 139 |
| promoter, | AGTTCATAGCCCATATATGGAGTTCCGCGTTACATAACTTACGGTAAATGG | |
| 5′UTR and | CCCGCCTGGCTGACCGCCCAACGACCCCCGCCCATTGACGTCAATAATGA | |
| leader | CGTATGTTCCCATAGTAACGCCAATAGGGACTTTCCATTGACGTCAATGGG | |
| sequence, | TGGAGTATTTACGGTAAACTGCCCACTTGGCAGTACATCAAGTGTATCATA | |
| Spike | TGCCAAGTACGCCCCCTATTGACGTCAATGACGGTAAATGGCCCGCCTGG | |
| glycoprotein | CATTATGCCCAGTACATGACCTTATGGGACTTTCCTACTTGGCAGTACATC | |
| (with 36 | TACGTATTAGTCATCGCTATTACCATGGTCGAGGTGAGCCCCACGTTCTGC | |
| mutations | TTCACTCTCCCCATCTCCCCCCCCTCCCCACCCCCAATTTTGTATTTATTTA | |
| and 6 | TTTTTTAATTATTTTGTGCAGCGATGGGGGCGGGGGGGGGGGGGGGGCG | |
| deletions, | CGCGCCAGGCGGGGCGGGGCGGGGCGAGGGGCGGGGCGGGGCGAGG | |
| stop | CGGAGAGGTGCGGCGGCAGCCAATCAGAGCGGCGCGCTCCGAAAGTTTC | |
| codon, | CTTTTATGGCGAGGCGGCGGCGGCGGCGGCCCTATAAAAAGCGAAGCGC | |
| 3′UTR. | GCGGCGGGCGGGAGTCGCTGCGCGCTGCCTTCGCCCCGTGCCCCGCTC | |
| polyA tail | CGCCGCCGCCTCGCGCCGCCCGCCCCGGCTCTGACTGACCGCGTTACTC | |
| | CCACAGGTGAGCGGGCGGGACGGCCCTTCTCCTCCGGGCTGTAATTAGC | |
| | GCTTGGTTTAATGACGGCTTGTTTCTTTTCTGTGGCTGCGTGAAAGCCTTG | |
| | AGGGGCTCCGGGAGGGCCCTTTGTGCGGGGGGAGCGGCTCGGGGGGTG | |
| | CGTGCGTGTGTGTGTGCGTGGGGAGCGCCGCGTGCGGCTCCGCGCTGC | |
| | CCGGCGGCTGTGAGCGCTGCGGGCGCGGCGCGGGGCTTTGTGCGCTCC | |
| | GCAGTGTGCGCGAGGGGAGCGCGGCCGGGGGCGGTGCCCCGCGGTGC | |
| | GGGGGGGGCTGCGAGGGGAACAAAGGCTGCGTGCGGGGTGTGTGCGTG | |
| | GGGGGGTGAGCAGGGGGTGTGGGCGCGTCGGTCGGGCTGCAACCCCCC | |
| | CTGCACCCCCCTCCCCGAGTTGCTGAGCACGGCCCGGCTTCGGGTGCGG | |
| | GGCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCCGG | |
| | GGAGGGCTCGGGGGAGGGGCGCGGCGGCCCCCGGAGCGCCGGCGGCT | |
| | GTCGAGGCGCGGCGAGCCGCAGCCATTGCCTTTTATGGTAATCGTGCGA | |
| | GAGGGCGCAGGGACTTCCTTTGTCCCAAATCTGTGCGGAGCCGAAATCTG | |
| | GGAGGCGCCGCCGCACCCCCTCTAGCGGGCGCGGGGCGAAGCGGTGCG | |
| | GCGCCGGCAGGAAGGAAATGGGCGGGGAGGGCCTTCGTGCGTCGCCGC | |
| | GCCGCCGTCCCCTTCTCCCTCTCCAGCCTCGGGGCTGTCCGCGGGGGGA | |
| | CGGCTGCCTTCGGGGGGGACGGGGCAGGGCGGGGTTCGGCTTCTGGCG | |
| | TGTGACCGGCGGCTCTAGAGCCTCTGCTAACCATGTTCATGCCTTCTTCTT | |
| | TTTCCTACAGCTCCTGGGCAACGTGCTGGTTATTGTGCTGTCTCATCATTT | |
| | TGGCAAAGAATTGGAGAATAAACTAGTATTCTTCTGGTCCCCACAGACTCA | |
| | GAGAGAACCCGCCACCATGTTCGTGTTCCTGGTGCTGCTGCCCCTGGTGA | |
| | GCAGCCAGTGCGTGAACTTCACCACCAGGACCCAGCTGCCCCCCGCCTA | |
| | CACCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCAAC | |
| | GTGACCTGGTTCCACGCCATCCACGTGAGCGGCACCAACGGCACCAAGA | |
| | GGTTCGACAACCCCGTGCTGCCCTTCAACGACGGCGTGTACTTCGCCAGC | |
| | ACCGAGAAGAGCAACATCATCAGGGGCTGGATCTTCGGCACCACCCTGG | |
| | ACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGT | |
| | ACTACCACAAGAACAACAAGAGCTGGATGGAGAGCGAGTTCAGGGTGTAC | |
| | AGCAGCGCCAACAACTGCACCTTCGAGTACGTGAGCCAGCCCTTCCTGAT | |
| | GGACCTGGAGGGCAAGCAGGGCAACTTCAAGAACCTGAGGGAGTTCGTG | |
| | TTCAAGAACATCGACGGCTACTTCAAGATCTACAGCAAGCACACCCCCATC | |
| | AACCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGGCC | |
| | CTGCACAGGAGCTACCTGACCCCCGGCGACAGCAGCAGCGGCTGGACCG | |
| | CCGGCGCCGCCGCCTACTACGTGGGCTACCTGCAGCCCAGGACCTTCCT | |
| | GCTGAAGTACAACGAGAACGGCACCATCACCGACGCCGTGGACTGCGCC | |
| | CTGGACCCCCTGAGCGAGACCAAGTGCACCCTGAAGAGCTTCACCGTGG | |
| | AGAAGGGCATCTACCAGACCAGCAACTTCAGGGTGCAGCCCACCGAGAG | |
| | CATCGTGAGGTTCCCCAACATCACCAACCTGTGCCCCTTCAGCGAGATCT | |
| | TCAACGCCACCAAGTTCAGCAGCGTGTACGCCTGGGACAGGAGGAAGAT | |
| | CAACAACTGCGTGGCCGACTACAGCTTCCTGTACAACAGCGCCAGCTTCA | |
| | GCACCTTCAAGTGCTACGGCGTGAGCCTGAACAAGCTGAACGACCTGTGC | |
| | TTCACCAACGTGTACGCCGACAGCTTCGTGATCAGGGGCGACCAGGTGAA | |
| | GCAGATCGCCCCCGGCCAGACCGGCAACATCGCCGACTACAACTACAAG | |
| | CTGCCCGACGACTTCACCGGCTGCGTGATCGCCTGGAACAGCAAGAAGC | |
| | TGGACAGCAAGGTGGTGGGCAACCACAAGTACAGGTTCAGGTTCTTCAGG | |
| | AAGAGCAACCTGAAGCCCTTCGAGAGGGACATCAGCACCGAGATCTACCA | |
| | GGTGGGCAACAAGCCCTGCAAGGGCGCCAAGGGCCTGAACTGCTACCTG | |
| | CCCCTGAAGAGCTACGGCTTCCAGCCCACCTACGGCGTGGGCTACCAGC | |
| | CCCACAGGGTGGTGGTGCTGAGCTTCGAGCTGCTGCACGCCAGCGCCAC | |
| | CGTGTGCGGCCCCAAGAAGAGCACCAACCTGGTGAAGAACAAGTGCGTG | |
| | AACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCC | |
| | CCTGCAGCTTCGGCGGCGTGAGCGTGATCACCCCCGGCACCAACACCAG | |
| | CAACCAGGTGGCCGTGCTGTACCAGGACGTGAACTGCACCGAGGTGCCC | |
| | GTGGCCATCCACGCCGACCAGCTGACCCCCACCTGGAGGGTGTACAGCA | |
| | CCGGCAGCAACGTGTTCCAGACCAGGGCCGGCTGCCTGATCGGCGCCGA | |
| | GCACGTGAACAACAGCTACGAGTGCGACATCCCCATCGGCGCCGGCATC | |
| | TGCGCCAGCTACCAGACCCAGACCAACAGCCCCAGGAGGGCCAGGAGCG | |
| | TGGCCAGCCAGAGCATCATCGCCTACACCATGAGCCTGGGCGCCGAGAA | |
| | CAGCGTGGCCTACAGCAACAACAGCATCGCCATCCCCACCAACTTCACCA | |
| | TCAGCGTGACCACCGAGATCCTGCCCGTGAGCATGACCAAGACCAGCGT | |
| | GGACTGCACCATGTACATCTGCGGCGACAGCACCGAGTGCAGCAACCTG | |
| | CTGCTGCAGTACGGCAGCTTCTGCACCCAGCTGAACAGGGCCCTGACCG | |
| | GCATCGCCGTGGAGCAGGACAAGAACACCCAGGAGGTGTTCGCCCAGGT | |
| | GAAGCAGATCTACAAGACCCCCCCCATCAAGTACTTCGGCGGCTTCAACT | |
| | TCAGCCAGATCCTGCCCGACCCCAGCAAGCCCAGCAAGAGGAGCTTCAT | |
| | CGAGGACCTGCTGTTCAACAAGGTGACCCTGGCCGACGCCGGCTTCATC | |
| | AAGCAGTACGGCGACTGCCTGGGCGACATCGCCGCCAGGGACCTGATCT | |
| | GCGCCCAGAAGTTCAACGGCCTGACCGTGCTGCCCCCCCTGCTGACCGA | |
| | CGAGATGATCGCCCAGTACACCAGCGCCCTGCTGGCCGGCACCATCACC | |
| | AGCGGCTGGACCTTCGGCGCCGGCGCCGCCCTGCAGATCCCCTTCGCCA | |
| | TGCAGATGGCCTACAGGTTCAACGGCATCGGCGTGACCCAGAACGTGCT | |
| | GTACGAGAACCAGAAGCTGATCGCCAACCAGTTCAACAGCGCCATCGGCA | |
| | AGATCCAGGACAGCCTGAGCAGCACCGCCAGCGCCCTGGGCAAGCTGCA | |
| | GGACGTGGTGAACCAGAACGCCCAGGCCCTGAACACCCTGGTGAAGCAG | |
| | CTGAGCAGCAACTTCGGCGCCATCAGCAGCGTGCTGAACGACATCCTGA | |
| | GCAGGCTGGACAAGGTGGAGGCCGAGGTGCAGATCGACAGGCTGATCAC | |
| | CGGCAGGCTGCAGAGCCTGCAGACCTACGTGACCCAGCAGCTGATCAGG | |
| | GCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCT | |
| | ACCACCTGATGAGCTTCCCCCAGAGCGCCCCCCACGGCGTGGTGTTCCT | |
| | GCACGTGACCTACGTGCCCGCCCAGGAGAAGAACTTCACCACCGCCCCC | |
| | GCCATCTGCCACGACGGCAAGGCCCACTTCCCCAGGGAGGGCGTGTTCG | |
| | TGAGCAACGGCACCCACTGGTTCGTGACCCAGAGGAACTTCTACGAGCCC | |
| | CAGATCATCACCACCGACAACACCTTCGTGAGCGGCAACTGCGACGTGGT | |
| | GATCGGCATCGTGAACAACACCGTGTACGACCCCCTGCAGCCCGAGCTG | |
| | GACAGCTTCAAGGAGGAGCTGGACAAGTACTTCAAGAACCACACCAGCCC | |
| | CGACGTGGACCTGGGCGACATCAGCGGCATCAACGCCAGCGTGGTGAAC | |
| | ATCCAGAAGGAGATCGACAGGCTGAACGAGGTGGCCAAGAACCTGAACG | |
| | AGAGCCTGATCGACCTGCAGGAGCTGGGCAAGTACGAGCAGTACATCAA | |
| | GTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCT | |
| | GAAGGGCTGCTGCAGCTGCGGCAGCTGCTGCAAGTTCGACGAGGACGAC | |
| | AGCGAGCCCGTGCTGAAGGGCGTGAAGCTGCACTACACC [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] | |
| 2 | CTCGACATTGATTATTGACTAGTTATTAATAGTAATCAATTACGGGGTCATT | 140 |
| promoter, | ATGTTCATAGCCCATATATGGAGTTCCGCGTTACATAACTTACGGTAAATGG | |
| 5’UTR and | CCCGCCTGGCTGACCGCCCAACGACCCCCGCCCATTGACGTCAATAATGA | |
| leader | CGTATGTTCCCATAGTAACGCCAATAGGGACTTTCCATTGACGTCAATGGG | |
| sequence, | TGGAGTATTTACGGTAAACTGCCCACTTGGCAGTACATCAAGTGTATCATA | |
| Spike | TGCCAAGTACGCCCCCTATTGACGTCAATGACGGTAAATGGCCCGCCTGG | |
| glycoprotein | CATTATGCCCAGTACATGACCTTATGGGACTTTCCTACTTGGCAGTACATC | |
| (with 36 | TACGTATTAGTCATCGCTATTACCATGGTCGAGGTGAGCCCCACGTTCTGC | |
| mutations | TTCACTCTCCCCATCTCCCCCCCCTCCCCACCCCCAATTTTGTATTTATTTA | |
| and 6 | TTTTTTAATTATTTTGTGCAGCGATGGGGGCGGGGGGGGGGGGGGGGCG | |
| deletions; | CGCGCCAGGCGGGGCGGGGCGGGGCGAGGGGCGGGGCGGGGCGAGG | |
| 6 | CGGAGAGGTGCGGCGGCAGCCAATCAGAGCGGCGCGCTCCGAAAGTTTC | |
| stabilizing | CTTTTATGGCGAGGCGGCGGCGGCGGCGGCCCTATAAAAAGCGAAGCGC | |
| mutations), | GCGGCGGGCGGGAGTCGCTGCGCGCTGCCTTCGCCCCGTGCCCCGCTC | |
| stop | CGCCGCCGCCTCGCGCCGCCCGCCCCGGCTCTGACTGACCGCGTTACTC | |
| codon, | CCACAGGTGAGCGGGCGGGACGGCCCTTCTCCTCCGGGCTGTAATTAGC | |
| 3′UTR. | GCTTGGTTTAATGACGGCTTGTTTCTTTTCTGTGGCTGCGTGAAAGCCTTG | |
| polyA tail | AGGGGCTCCGGGAGGGCCCTTTGTGCGGGGGGAGCGGCTCGGGGGGTG | |
| | CGTGCGTGTGTGTGTGCGTGGGGAGCGCCGCGTGCGGCTCCGCGCTGC | |
| | CCGGCGGCTGTGAGCGCTGCGGGCGCGGCGCGGGGCTTTGTGCGCTCC | |
| | GCAGTGTGCGCGAGGGGAGCGCGGCCGGGGGCGGTGCCCCGCGGTGC | |
| | GGGGGGGGCTGCGAGGGGAACAAAGGCTGCGTGCGGGGTGTGTGCGTG | |
| | GGGGGGTGAGCAGGGGGTGTGGGCGCGTCGGTCGGGCTGCAACCCCCC | |
| | CTGCACCCCCCTCCCCGAGTTGCTGAGCACGGCCCGGCTTCGGGTGCGG | |
| | GGCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCCGG | |
| | GGAGGGCTCGGGGGAGGGGCGCGGCGGCCCCCGGAGCGCCGGCGGCT | |
| | GTCGAGGCGCGGCGAGCCGCAGCCATTGCCTTTTATGGTAATCGTGCGA | |
| | GAGGGCGCAGGGACTTCCTTTGTCCCAAATCTGTGCGGAGCCGAAATCTG | |
| | GGAGGCGCCGCCGCACCCCCTCTAGCGGGCGCGGGGCGAAGCGGTGCG | |
| | GCGCCGGCAGGAAGGAAATGGGCGGGGAGGGCCTTCGTGCGTCGCCGC | |
| | GCCGCCGTCCCCTTCTCCCTCTCCAGCCTCGGGGCTGTCCGCGGGGGGA | |
| | CGGCTGCCTTCGGGGGGGACGGGGCAGGGCGGGGTTCGGCTTCTGGCG | |
| | TGTGACCGGCGGCTCTAGAGCCTCTGCTAACCATGTTCATGCCTTCTTCTT | |
| | TTTCCTACAGCTCCTGGGCAACGTGCTGGTTATTGTGCTGTCTCATCATTT | |
| | TGGCAAAGAATTGGAGAATAAACTAGTATTCTTCTGGTCCCCACAGACTCA | |
| | GAGAGAACCCGCCACCATGTTCGTGTTCCTGGTGCTGCTGCCCCTGGTGA | |
| | GCAGCCAGTGCGTGAACTTCACCACCAGGACCCAGCTGCCCCCCGCCTA | |
| | CACCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCAAC | |
| | GTGACCTGGTTCCACGCCATCCACGTGAGCGGCACCAACGGCACCAAGA | |
| | GGTTCGACAACCCCGTGCTGCCCTTCAACGACGGCGTGTACTTCGCCAGC | |
| | ACCGAGAAGAGCAACATCATCAGGGGCTGGATCTTCGGCACCACCCTGG | |
| | ACAGCAAGACCCAGAGCCTGCTGATCGTGAACAACGCCACCAACGTGGT | |
| | GATCAAGGTGTGCGAGTTCCAGTTCTGCAACGACCCCTTCCTGGGCGTGT | |
| | ACTACCACAAGAACAACAAGAGCTGGATGGAGAGCGAGTTCAGGGTGTAC | |
| | AGCAGCGCCAACAACTGCACCTTCGAGTACGTGAGCCAGCCCTTCCTGAT | |
| | GGACCTGGAGGGCAAGCAGGGCAACTTCAAGAACCTGAGGGAGTTCGTG | |
| | TTCAAGAACATCGACGGCTACTTCAAGATCTACAGCAAGCACACCCCCATC | |
| | AACCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGGCC | |
| | CTGCACAGGAGCTACCTGACCCCCGGCGACAGCAGCAGCGGCTGGACCG | |
| | CCGGCGCCGCCGCCTACTACGTGGGCTACCTGCAGCCCAGGACCTTCCT | |
| | GCTGAAGTACAACGAGAACGGCACCATCACCGACGCCGTGGACTGCGCC | |
| | CTGGACCCCCTGAGCGAGACCAAGTGCACCCTGAAGAGCTTCACCGTGG | |
| | AGAAGGGCATCTACCAGACCAGCAACTTCAGGGTGCAGCCCACCGAGAG | |
| | CATCGTGAGGTTCCCCAACATCACCAACCTGTGCCCCTTCAGCGAGATCT | |
| | TCAACGCCACCAAGTTCAGCAGCGTGTACGCCTGGGACAGGAGGAAGAT | |
| | CAACAACTGCGTGGCCGACTACAGCTTCCTGTACAACAGCGCCAGCTTCA | |
| | GCACCTTCAAGTGCTACGGCGTGAGCCTGAACAAGCTGAACGACCTGTGC | |
| | TTCACCAACGTGTACGCCGACAGCTTCGTGATCAGGGGCGACCAGGTGAA | |
| | GCAGATCGCCCCCGGCCAGACCGGCAACATCGCCGACTACAACTACAAG | |
| | CTGCCCGACGACTTCACCGGCTGCGTGATCGCCTGGAACAGCAAGAAGC | |
| | TGGACAGCAAGGTGGTGGGCAACCACAAGTACAGGTTCAGGTTCTTCAGG | |
| | AAGAGCAACCTGAAGCCCTTCGAGAGGGACATCAGCACCGAGATCTACCA | |
| | GGTGGGCAACAAGCCCTGCAAGGGCGCCAAGGGCCTGAACTGCTACCTG | |
| | CCCCTGAAGAGCTACGGCTTCCAGCCCACCTACGGCGTGGGCTACCAGC | |
| | CCCACAGGGTGGTGGTGCTGAGCTTCGAGCTGCTGCACGCCAGCGCCAC | |
| | CGTGTGCGGCCCCAAGAAGAGCACCAACCTGGTGAAGAACAAGTGCGTG | |
| | AACTTCAACTTCAACGGCCTGACCGGCACCGGCGTGCTGACCGAGAGCA | |
| | ACAAGAAGTTCCTGCCCTTCCAGCAGTTCGGCAGGGACATCGCCGACACC | |
| | ACCGACGCCGTGAGGGACCCCCAGACCCTGGAGATCCTGGACATCACCC | |
| | CCTGCAGCTTCGGCGGCGTGAGCGTGATCACCCCCGGCACCAACACCAG | |
| | CAACCAGGTGGCCGTGCTGTACCAGGACGTGAACTGCACCGAGGTGCCC | |
| | GTGGCCATCCACGCCGACCAGCTGACCCCCACCTGGAGGGTGTACAGCA | |
| | CCGGCAGCAACGTGTTCCAGACCAGGGCCGGCTGCCTGATCGGCGCCGA | |
| | GCACGTGAACAACAGCTACGAGTGCGACATCCCCATCGGCGCCGGCATC | |
| | TGCGCCAGCTACCAGACCCAGACCAACAGCCCCGGCAGCGCCAGCAGC | |
| | GTGGCCAGCCAGAGCATCATCGCCTACACCATGAGCCTGGGCGCCGAGA | |
| | ACAGCGTGGCCTACAGCAACAACAGCATCGCCATCCCCACCAACTTCACC | |
| | ATCAGCGTGACCACCGAGATCCTGCCCGTGAGCATGACCAAGACCAGCG | |
| | TGGAOTGCACCATGTACATCTGCGGCGACAGCACCGAGTGCAGCAACCT | |
| | GCTGCTGCAGTACGGCAGCTTCTGCACCCAGCTGAACAGGGCCCTGACC | |
| | GGCATCGCCGTGGAGCAGGACAAGAACACCCAGGAGGTGTTCGCCCAGG | |
| | TGAAGCAGATCTACAAGACCCCCCCCATCAAGTACTTCGGCGGCTTCAAC | |
| | TTCAGCCAGATCCTGCCCGACCCCAGCAAGCCCAGCAAGAGGAGCCCCA | |
| | TCGAGGACCTGCTGTTCAACAAGGTGACCCTGGCCGACGCCGGCTTCATC | |
| | AAGCAGTACGGCGACTGCCTGGGCGACATCGCCGCCAGGGACCTGATCT | |
| | GCGCCCAGAAGTTCAACGGCCTGACCGTGCTGCCCCCCCTGCTGACCGA | |
| | CGAGATGATCGCCCAGTACACCAGCGCCCTGCTGGCCGGCACCATCACC | |
| | AGCGGCTGGACCTTCGGCGCCGGCCCCGCCCTGCAGATCCCCTTCCCCA | |
| | TGCAGATGGCCTACAGGTTCAACGGCATCGGCGTGACCCAGAACGTGCT | |
| | GTACGAGAACCAGAAGCTGATCGCCAACCAGTTCAACAGCGCCATCGGCA | |
| | AGATCCAGGACAGCCTGAGCAGCACCCCCAGCGCCCTGGGCAAGCTGCA | |
| | GGACGTGGTGAACCAGAACGCCCAGGCCCTGAACACCCTGGTGAAGCAG | |
| | CTGAGCAGCAACTTCGGCGCCATCAGCAGCGTGCTGAACGACATCCTGA | |
| | GCAGGCTGGACCCCCCCGAGGCCGAGGTGCAGATCGACAGGCTGATCAC | |
| | CGGCAGGCTGCAGAGCCTGCAGACCTACGTGACCCAGCAGCTGATCAGG | |
| | GCCGCCGAGATCAGGGCCAGCGCCAACCTGGCCGCCACCAAGATGAGC | |
| | GAGTGCGTGCTGGGCCAGAGCAAGAGGGTGGACTTCTGCGGCAAGGGCT | |
| | ACCACCTGATGAGCTTCCCCCAGAGCGCCCCCCACGGCGTGGTGTTCCT | |
| | GCACGTGACCTACGTGCCCGCCCAGGAGAAGAACTTCACCACCGCCCCC | |
| | GCCATCTGCCACGACGGCAAGGCCCACTTCCCCAGGGAGGGCGTGTTCG | |
| | TGAGCAACGGCACCCACTGGTTCGTGACCCAGAGGAACTTCTACGAGCCC | |
| | CAGATCATCACCACCGACAACACCTTCGTGAGCGGCAACTGCGACGTGGT | |
| | GATCGGCATCGTGAACAACACCGTGTACGACCCCCTGCAGCCCGAGCTG | |
| | GACAGCTTCAAGGAGGAGCTGGACAAGTACTTCAAGAACCACACCAGCCC | |
| | CGACGTGGACCTGGGCGACATCAGCGGCATCAACGCCAGCGTGGTGAAC | |
| | ATCCAGAAGGAGATCGACAGGCTGAACGAGGTGGCCAAGAACCTGAACG | |
| | AGAGCCTGATCGACCTGCAGGAGCTGGGCAAGTACGAGCAGTACATCAA | |
| | GTGGCCCTGGTACATCTGGCTGGGCTTCATCGCCGGCCTGATCGCCATC | |
| | GTGATGGTGACCATCATGCTGTGCTGCATGACCAGCTGCTGCAGCTGCCT | |
| | GAAGGGCTGCTGCAGCTGCGGCAGCTGCTGCAAGTTCGACGAGGACGAC | |
| | AGCGAGCCCGTGCTGAAGGGCGTGAAGCTGCACTACACC [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] | |
| 3 | CTCGACATTGATTATTGACTAGTTATTAATAGTAATCAATTACGGGGTCATT | 141 |
| promoter, | AGTTCATAGCCCATATATGGAGTTCCGCGTTACATAACTTACGGTAAATGG | |
| 5’UTR and | CCCGCCTGGCTGACCGCCCAACGACCCCCGCCCATTGACGTCAATAATGA | |
| leader | CGTATGTTCCCATAGTAACGCCAATAGGGACTTTCCATTGACGTCAATGGG | |
| sequence, | TGGAGTATTTACGGTAAACTGCCCACTTGGCAGTACATCAAGTGTATCATA | |
| linker, CD | TGCCAAGTACGCCCCCTATTGACGTCAATGACGGTAAATGGCCCGCCTGG | |
| 8⁺ T cell | CATTATGCCCAGTACATGACCTTATGGGACTTTCCTACTTGGCAGTACATC | |
| epitopes, | TACGTATTAGTCATCGCTATTACCATGGTCGAGGTGAGCCCCACGTTCTGC | |
| CD4⁺ T | TTCACTCTCCCCATCTCCCCCCCCTCCCCACCCCCAATTTTGTATTTATTTA | |
| cell | TTTTTTAATTATTTTGTGCAGCGATGGGGGCGGGGGGGGGGGGGGGGCG | |
| epitopes, | CGCGCCAGGCGGGGCGGGGCGGGGCGAGGGGCGGGGCGGGGCGAGG | |
| B Cell | CGGAGAGGTGCGGCGGCAGCCAATCAGAGCGGCGCGCTCCGAAAGTTTc | |
| epitopes, | CTTTTATGGCGAGGCGGCGGCGGCGGCGGCCCTATAAAAAGCGAAGCGC | |
| stop | GCGGCGGGCGGGAGTCGCTGCGCGCTGCCTTCGCCCCGTGCCCCGCTC | |
| codon, | CGCCGCCGCCTCGCGCCGCCCGCCCCGGCTCTGACTGACCGCGTTACTC | |
| 3′UTR, | CCACAGGTGAGCGGGCGGGACGGCCCTTCTCCTCCGGGCTGTAATTAGC | |
| polyA tail | GCTTGGTTTAATGACGGCTTGTTTCTTTTCTGTGGCTGCGTGAAAGCCTTG | |
| | AGGGGCTCCGGGAGGGCCCTTTGTGCGGGGGGAGCGGCTCGGGGGGTG | |
| | CGTGCGTGTGTGTGTGCGTGGGGAGCGCCGCGTGCGGCTCCGCGCTGC | |
| | CCGGCGGCTGTGAGCGCTGCGGGCGCGGCGCGGGGCTTTGTGCGCTCC | |
| | GCAGTGTGCGCGAGGGGAGCGCGGCCGGGGGCGGTGCCCCGCGGTGC | |
| | GGGGGGGGCTGCGAGGGGAACAAAGGCTGCGTGCGGGGTGTGTGCGTG | |
| | GGGGGGTGAGCAGGGGGTGTGGGCGCGTCGGTCGGGCTGCAACCCCCC | |
| | CTGCACCCCCCTCCCCGAGTTGCTGAGCACGGCCCGGCTTCGGGTGCGG | |
| | GGCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCCGG | |
| | GGAGGGCTCGGGGGAGGGGCGCGGCGGCCCCCGGAGCGCCGGCGGCT | |
| | GTCGAGGCGCGGCGAGCCGCAGCCATTGCCTTTTATGGTAATCGTGCGA | |
| | GAGGGCGCAGGGACTTCCTTTGTCCCAAATCTGTGCGGAGCCGAAATCTG | |
| | GGAGGCGCCGCCGCACCCCCTCTAGCGGGCGCGGGGCGAAGCGGTGCG | |
| | GCGCCGGCAGGAAGGAAATGGGCGGGGAGGGCCTTCGTGCGTCGCCGC | |
| | GCCGCCGTCCCCTTCTCCCTCTCCAGCCTCGGGGCTGTCCGCGGGGGGA | |
| | CGGCTGCCTTCGGGGGGGACGGGGCAGGGCGGGGTTCGGCTTCTGGCG | |
| | TGTGACCGGCGGCTCTAGAGCCTCTGCTAACCATGTTCATGCCTTCTTCTT | |
| | TTTCCTACAGCTCCTGGGCAACGTGCTGGTTATTGTGCTGTCTCATCATTT | |
| | TGGCAAAGAATTGGAGAATAAACTAGTATTCTTCTGGTCCCCACAGACTCA | |
| | GAGAGAACCCGCCACCATGTTCGTGTTCCTGGTGCTGCTGCCCCTGGTGA | |
| | GCAGCCAGTGCGTGGAGGCCGCCGCCAAGAAGAGCTACGGCTTCCAGCC | |
| | CACCTACGCCGCCTACGTGGTGGGCAACCACAAGTACAGGTTCGCCGCC | |
| | TACTACCAGGTGGGCAACAAGCCCTGCAAGGCCGCCTACTGCGTGATCG | |
| | CCTGGAACAGCAAGAAGGCCGCCTACAAGGGCGCCAAGGGCCTGAACTG | |
| | CTACGCCGCCTACAGCCAGTGCGTGAACTTCACCACCAGGGCCGCCTAC | |
| | AACATCGCCGACTACAACTACAAGCTGGCCGCCTACTACCTGCCCCTGAA | |
| | GAGCTACGGCTTCGCCGCCTACAAGTGCTACGGCGTGAGCCTGAACAAG | |
| | GCCGCCTACTGCGTGATCGCCTGGAACAGCAAGAAGGCCGCCTACATCTA | |
| | CAAGACCCCCCCCATCAAGTACGCCGCCTACTGCGTGGCCGACTACAGCT | |
| | TCCTGTACGCCGCCTACAGCGTGTACGCCTGGGACAGGAGGAAGGCCGC | |
| | CTACAGGTTCTTCAGGAAGAGCAACCTGAAGGCCGCCTACGACATCAGCA | |
| | CCGAGATCTACCAGGTGGCCGCCTACTACCAGCCCCACAGGGTGGTGGT | |
| | GCTGGCCGCCTACGTGGTGGGCAACCACAAGTACAGGTTCGCCGCCTAC | |
| | TTCGTGATCAGGGGCGACCAGGTGAAGGCCGCCTACAACGCCACCAAGT | |
| | TCAGCAGCGTGTACGCCGCCTACTACCAGGTGGGCAACAAGCCCTGCAA | |
| | GGCCGCCTACAACGCCACCAAGTTCAGCAGCGTGTACGCCGCCTACTTC | |
| | GTGATCAGGGGCGACCAGGTGAAGGCCGCCTACAAGGGCGCCAAGGGC | |
| | CTGAACTGCTACGCCGCCTACAACCTGTGCCCCTTCAGCGAGATCTTCGC | |
| | CGCCTACGCCAGCGCCACCGTGGGAAGCGGAGCCACGAACTTCTCTCTG | |
| | TTAAAGCAAGCAGGAGATGTTGAAGAAAACCCCGGGCCTCAACTGCTACC | |
| | TGCCCCTGAAGAGCTACGGCTTCCAGCCCACCTACGGCCCCGGCCCCG | |
| | GCGGCAACCACAAGTACAGGTTCAGGTTCTTCAGGAAGAGCAACCTGG | |
| | GCCCCGGCCCCGGCCCCTTCGAGAGGGACATCAGCACCGAGATCTACC | |
| | AGGTGGGCAACGGCCCCGGCCCCGGCAAGAAGCTGGACAGCAAGGTG | |
| | GTGGGCAACCACAAGTACAGGTTCGGCCCCGGCCCCGGCAAGGGCCTG | |
| | AACTGCTACCTGCCCCTGAAGAGCTACGGCTTCCAGGGCCCCGGCCCC | |
| | GGCCTGGTGCTGCTGCCCCTGGTGAGCAGCCAGTGCGTGAACTTCACCG | |
| | GCCCCGGCCCCGGCAGGGGCGACCAGGTGAAGCAGATCGCCCCCGGC | |
| | CAGACCGGCAACGGCCCCGGCCCCGGCAGCGCCAGCTTCAGCACCTTC | |
| | AAGTGCTACGGCGTGAGCCTGAACGGCCCCGGCCCCGGCAAGCTGGAC | |
| | AGCAAGGTGGTGGGCAACCACAAGTACAGGTTCAGGGGCCCCGGCCCC | |
| | GGCTTCGCCCAGGTGAAGCAGATCTACAAGACCCCCCCCATCAAGTACG | |
| | GCCCCGGCCCCGGCGCCGACTACAGCTTCCTGTACAACAGCGCCAGCTT | |
| | CAGCACCTTCGGCCCCGGCCCCGGCGCCACCAAGTTCAGCAGCGTGTA | |
| | CGCCTGGGACAGGAGGAAGATCGGCCCCGGCCCCGGCCCCCACAGGG | |
| | TGGTGGTGCTGAGCTTCGAGCTGCTGCACGCCAGCGGCCCCGGCCCCG | |
| | GCTTCGAGAGGGACATCAGCACCGAGATCTACCAGGTGGGCAACAAGG | |
| | GCCCCGGCCCCGGCGCCAAGGGCCTGAACTGCTACCTGCCCCTGAAGA | |
| | GCTACGGCTTCGGCCCCGGCCCCGGCAGCATCGTGAGGTTCCCCAACAT | |
| | CACCAACCTGTGCCCCTTCAGCGGCCCCGGCCCCGGCAACAACTGCGT | |
| | GGCCGACTACAGCTTCCTGTACAACAGCGCCAGCGGCCCCGGCCCCGG | |
| | CAAGGGCGCCAAGGGCCTGAACTGCTACCTGCCCCTGAAGAGCTACGG | |
| | CCCCGGCCCCGGCCTGTGCCCCTTCAGCGAGATCTTCAACGCCACCAAG | |
| | TTCAGCAGCGGAAGCGGAGCCACGAACTTCTCTCTGTTAAAGCAAGCAGG | |
| | AGATGTTGAAGAAAACCCCGGGCCT [symbol] [symbol] [symbol] | |
| | [symbol] [symbol] [symbol] [symbol] [symbol] [symbol] | |
| | AAGAAG [symbol] [symbol] [symbol] [symbol] [symbol] | |
| | [symbol] [symbol] [symbol] [symbol] AAGAAG [symbol] | |
| | [symbol] [symbol] [symbol] [symbol] [symbol] [symbol] | |
| | [symbol] [symbol] [symbol] [symbol] [symbol] AAGAAG | |
| | [symbol] [symbol] [symbol] [symbol] [symbol] [symbol] | |
| | [symbol] AAGAAG [symbol] [symbol] [symbol] [symbol] | |
| | [symbol] [symbol] [symbol] [symbol] [symbol] | |
| | [symbol] [symbol] [symbol] [symbol] [symbol] | |
| | AGAAG [symbol] [symbol] AAGAAG [symbol] [symbol] | |
| | [symbol] AAGAAG [symbol] AAGAAG [symbol] [symbol] | |
| | [symbol] [symbol] [symbol] [symbol] [symbol] [symbol] | |
| | [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] [symbol] | |
| | [symbol] [symbol] [symbol] [symbol] [symbol] | |

### Molecular Adjuvants and T Cell Enhancements

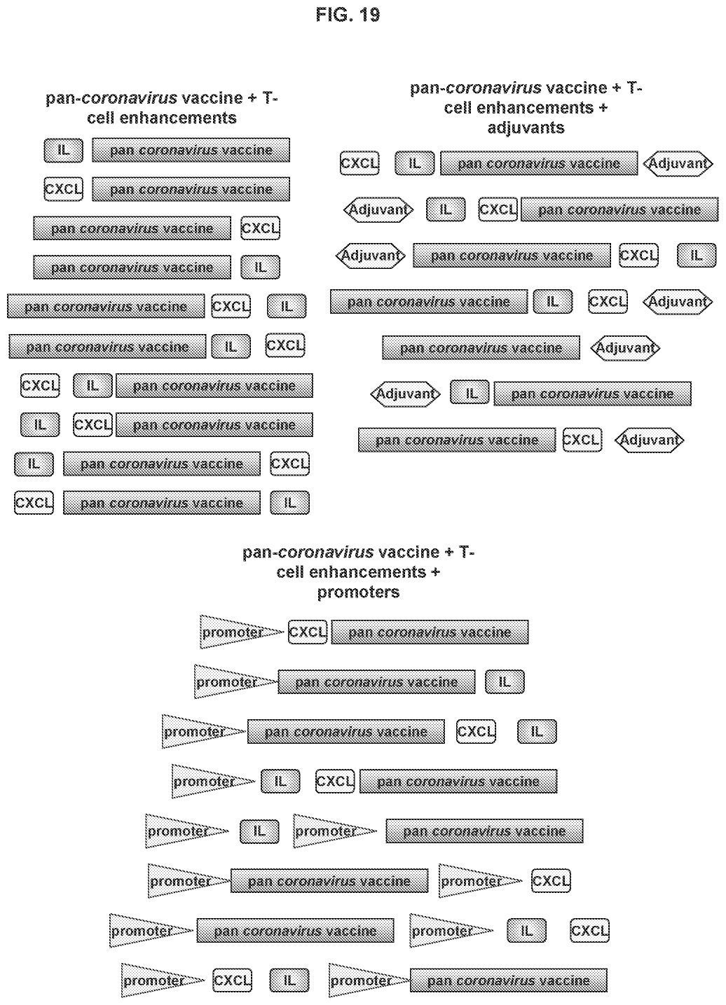

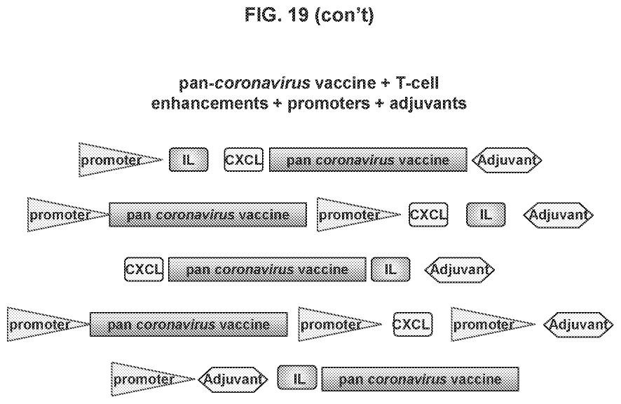

In certain embodiments, the vaccine composition comprises a molecular adjuvant and/or one or more T Cell enhancement compositions (FIG. 19). The adjuvant and/or enhancement compositions may help improve the immunogenicity and/or long-term memory of the vaccine composition. Non-limiting examples of molecular adjuvants include CpG, such as a CpG polymer, and flagellin.

In some embodiments, the vaccine composition comprises a T cell attracting chemokine. The T cell attracting chemokine helps pull the T cells from the circulation to the appropriate tissues, e.g., the lungs, heart, kidney, and brain. Non-limiting examples of T cell attracting chemokines Include CCL5, CXCL9, CXCL10, CXCL11, CCL25, CCL28, CXCL14, CXCL17, or a combination thereof.

In some embodiments, the vaccine composition comprises a composition that promotes T cell proliferation. Non-limiting examples of compositions that promote T cell proliferation include IL-7, IL-15, IL-2, or a combination thereof.

In some embodiments, the vaccine composition comprises a composition that promotes T cell homing in the lungs. Non-limiting examples of compositions that promote T cell homing include CCL25, CCL28, CXCL14, CXCL17 or a combination thereof.

Table 13 shows non-limiting examples of T-cell enhancements that may be used to create a vaccine composition described herein:

TABLE 13

| T-cell | | SEQ |
|---|---|---|
| enhancement | Sequence | ID NO: |
| CXCL11 | ATGAACAGGAAGGTGACCGCCATCGCCCTGGCCGCCATCATCTGGGCCA | 156 |
| | CCGCCGCCCAGGGCTTCCTGATGTTCAAGCAGGGCAGGTGCCTGTGCAT | |
| | CGGCCCCGGCATGAAGGCCGTGAAGATGGCCGAGATCGAGAAGGCCAG | |
| | CGTGATCTACCCCAGCAACGGCTGCGACAAGGTGGAGGTGATCGTGACC | |
| | ATGAAGGCCCACAAGAGGCAGAGGTGCCTGGACCCCAGGAGCAAGCAGG | |
| | CCAGGCTGATCATGCAGGCCATCGAGAAGAAGAACTTCCTGAGGAGGCA | |
| | GAACATGTGA | |
| CCL5 | ATGAAGGTCTCCGCGGCAGCCCTCGCTGTCATCCTCATTGCTACTGCCCT | 157 |
| | CTGCGCTCCTGCATCTGCCTCCCCATATTCCTCGGACACCACACCCTGCT | |
| | GCTTTGCCTACATTGCCCGCCCACTGCCCCGTGCCCACATCAAGGAGTAT | |
| | TTCTACACCAGTGGCAAGTGCTCCAACCCAGCAGTCGTCCACAGGTCAAG | |
| | GATGCCAAAGAGAGAGGGACAGCAAGTCTGGCAGGATTTCCTGTATGACT | |
| | CCCGGCTGAACAAGGGCAAGCTTTGTCACCCGAAAGAACCGCCAAGTGT | |
| | GTGCCAACCCAGAGAAGAAATGGGTTCGGGAGTACATCAACTCTTTGGAG | |
| | ATGAGCTAGGATGGAGAGTCCTTGAACCTGAACTTACACAAATTTGCCTGT | |
| | TTCTGCTTGCTCTTGTCCTAGCTTGGGAGGCTTCCCCTCACTATCCTACCC | |
| | CACCCGCTCCTTGA | |
| CXCL9 | ATGAAGAAAAGTGGTGTTCTTCCTCTTGGGCATCATCTTGCTGGTTCTG | 158 |
| | ATTGGAGTGCAAGGAACCCCAGTAGTGAGAAAGGGTCGCTGTTCCTGCAT | |
| | CAGCACCAACCAAGGGACTATCCACCTACAATCCTTGAAAGACCTTAAACA | |
| | ATTTGCCCCAAGCCCTTCCTGCGAGAAAATTGAAATCATTGCTACACTGAA | |
| | GAATGGAGTTCAAACATGTCTAAACCCAGATTCAGCAGATGTGAAGGAACT | |
| | GATTAAAAAGTGGGAGAAACAGGTCAGCCAAAAGAAAAAGCAAAAGAATG | |
| | GGAAAAAACATCAAAAAAAGAAAGTTCTGAAAGTTCGAAAATCTCAACGTT | |
| | CTCGTCAAAAGAAGACTACATAA | |
| CXCL10 | ATGAATCAAACTGCCATTCTGATTTGCTGCCTTATCTTTCTGACTCTAAGTG | 159 |
| | GCATTCAAGGAGTACCTCTCTCTAGAACTGTACGCTGTACCTGCATCAGCA | |
| | TTAGTAATCAACCTGTTAATCCAAGGTCTTTAGAAAAACTTGAAATTATTCC | |
| | TGCAAGCCAATTTTGTCCACGTGTTGAGATCATTGCTACAATGAAAAAGAA | |
| | GGGTGAGAAGAGATGTCTGAATCCAGAATCGAAGGCCATCAAGAATTTAC | |
| | TGAAAGCAGTTAGCAAGGAAAGGTCTAAAAGATCTCCTTAA | |
| CXCL14 | ATGAGGCTCCTGGCGGCCGCGCTGCTCCTGCTGCTGCTGGCGCTGTACA | 160 |
| | CCGCGCGTGTGGACGGGTCCAAATGCAAGTGCTCCCGGAAGGGACCCAA | |
| | GATCCGCTACAGCGACGTGAAGAAGCTGGAAATGAAGCCAAAGTACCCGC | |
| | ACTGCGAGGAGAAGATGGTTATCATCACCACCAAGAGCGTGTCCAGGTAC | |
| | CGAGGTCAGGAGCACTGCCTGCACCCCAAGCTGCAGAGCACCAAGCGCT | |
| | TCATCAAGTGGTACAACGCCTGGAACGAGAAGCGCAGGGTCTACGAAGAA | |
| | TAG | |
| CXCL17 | ATGAAAGTTCTAATCTCTTCCCTCCTCCTGTTGCTGCCACTAATGCTGATG | 161 |
| | TCCATGGTCTCTAGCAGCCTGAATCCAGGGGTCGCCAGAGGCCACAGGG | |
| | ACCGAGGCCAGGCTTCTAGGAGATGGCTCCAGGAAGGCGGCCAAGAATG | |
| | TGAGTGCAAAGATTGGTTCCTGAGAGCCCCGAGAAGAAAATTCATGACAG | |
| | TGTCTGGGCTGCCAAAGAAGCAGTGCCCCTGTGATCATTTCAAGGGCAAT | |
| | GTGAAGAAAACAAGACACCAAAGGCACCACAGAAAGCCAAACAAGCATTC | |
| | CAGAGCCTGCCAGCAATTTCTCAAACAATGTCAGCTAAGAAGCTTTGCTCT | |
| | GCCTTTGTAG | |
| CCL25 | ATGAACCTGTGGCTCCTGGCCTGCCTGGTGGCCGGCTTCCTGGGAGCCT | 162 |
| | GGGCCCCCGCTGTCCACACCCAAGGTGTCTTTGAGGACTGCTGCCTGGC | |
| | CTACCACTACCCCATTGGGTGGGCTGTGCTCCGGCGCGCCTGGACTTAC | |
| | CGGATCCAGGAGGTGAGCGGGAGCTGCAATCTGCCTGCTGCGATATTCTA | |
| | CCTCCCCAAGAGACACAGGAAGGTGTGTGGGAACCCCAAAAGCAGGGAG | |
| | GTGCAGAGAGCCATGAAGCTCCTGGATGCTCGAAATAAGGTTTTTGCAAA | |
| | GCTCCACCACAACACGCAGACCTTCCAAGCAGGCCCTCATGCTGTAAAGA | |
| | AGTTGAGTTCTGGAAACTCCAAGTTATCATCGTCCAAGTTTAGCAATCCCA | |
| | TCAGCAGCAGTAAGAGGAATGTCTCCCTCCTGATATCAGCTAATTCAGGAC | |
| | TGTGA | |
| CCL28 | ATGCAGCAGAGAGGACTCGCCATCGTGGCCTTGGCTGTCTGTGCGGCCC | 163 |
| | TACATGCCTCAGAAGCCATACTTCCCATTGCCTCCAGCTGTTGCACGGAG | |
| | GTTTCACATCATATTTCCAGAAGGCTCCTGGAAAGAGTGAATATGTGTCGC | |
| | ATCCAGAGAGCTGATGGGGATTGTGACTTGGCTGCTGTCATCCTTCATGT | |
| | CAAGCGCAGAAGAATCTGTGTCAGCCCGCACAACCATACTGTTAAGCAGT | |
| | GGATGAAAGTGCAAGCTGCCAAGAAAAATGGTAAAGGAAATGTTTGCCAC | |
| | AGGAAGAAACACCATGGCAAGAGGAACAGTAACAGGGCACATCAGGGGA | |
| | AACACGAAACATACGGCCATAAAACTCCTTATTAG | |
| IL-7 | ATGTTCCACGTGAGCTTCAGGTACATCTTCGGCATCCCCCCCCTGATCCT | 164 |
| | GGTGCTGCTGCCCGTGACCAGCAGCGAGTGCCACATCAAGGACAAGGAG | |
| | GGCAAGGCCTACGAGAGCGTGCTGATGATCAGCATCGACGAGCTGGACA | |
| | AGATGACCGGCACCGACAGCAACTGCCCCAACAACGAGCCCAACTTCTTC | |
| | AGGAAGCACGTGTGCGACGACACCAAGGAGGCCGCCTTCCTGAACAGGG | |
| | CCGCCAGGAAGCTGAAGCAGTTCCTGAAGATGAACATCAGCGAGGAGTTC | |
| | AACGTGCACCTGCTGACCGTGAGCCAGGGCACCCAGACCCTGGTGAACT | |
| | GCACCAGCAAGGAGGAGAAGAACGTGAAGGAGCAGAAGAAGAACGACGC | |
| | CTGCTTCCTGAAGAGGCTGCTGAGGGAGATCAAGACCTGCTGGAACAAGA | |
| | TCCTGAAGGGCAGCATCTGA | |
| IL-15 | ATGAGAATTTCGAAACCACATTTGAGAAGTATTTCCATCCAGTGCTACTTGT | 165 |
| | GTTTACTTCTAAACAGTCATTTTCTAACTGAAGCTGGCATTCATGTCTTCAT | |
| | TTTGGGCTGTTTCAGTGCAGGGCTTCCTAAAACAGAAGCCAACTGGGTGA | |
| | ATGTAATAAGTGATTTGAAAAAAATTGAAGATCTTATTCAATCTATGCATATT | |
| | GATGCTACTTTATATACGGAAAGTGATGTTCACCCCAGTTGCAAAGTAACA | |
| | GCAATGAAGTGCTTTCTCTTGGAGTTACAAGTTATTTCACTTGAGTCCGGA | |
| | GATGCAAGTATTCATGATACAGTAGAAAATCTGATCATCCTAGCAAACAAC | |
| | AGTTTGTCTTCTAATGGGAATGTAACAGAATCTGGATGCAAAGAATGTGAG | |
| | GAACTGGAGGAAAAAAATATTAAAGAATTTTTGCAGAGTTTTGTACATATTG | |
| | TCCAAATGTTCATCAACACTTCTTGA | |
| IL-2 | ATGTACAGGATGCAACTCCTGTCTTGCATTGCACTAAGTCTTGCACTTGTC | 166 |
| | ACAAACAGTGCACCTACTTCAAGTTCTACAAAGAAAACACAGCTACAACTG | |
| | GAGCATTTACTGCTGGATTTACAGATGATTTTGAATGGAATTAATAATTACA | |
| | AGAATCCCAAACTCACCAGGATGCTCACATTTAAGTTTTACATGCCCAAGA | |
| | AGGCCACAGAACTGAAACATCTTCAGTGTCTAGAAGAAGAACTCAAACCTC | |
| | TGGAGGAAGTGCTAAATTTAGCTCAAAGCAAAAACTTTCACTTAAGACCCA | |
| | GGGACTTAATCAGCAATATCAACGTAATAGTTCTGGAACTAAAGGGATCTG | |
| | AAACAACATTCATGTGTGAATATGCTGATGAGACAGCAACCATTGTAGAAT | |
| | TTCTGAACAGATGGATTACCTTTTGTCAAAGCATCATCTCAACACTGACTTG | |
| | A | |

In some embodiments, the T-cell enhancement compositions described herein (e.g. CXCL9, CXCL10, IL-7, IL-2) may be integrated Into a separate delivery system from the vaccine compositions. In other embodiments, the T-cell enhancement compositions described herein (e.g. CXCL9, CXCL10, IL-7, IL-2) may be integrated into the same delivery system as the vaccine compositions.

In certain embodiments, the composition comprises a tag. For example, in some embodiments, the composition comprises a His tag. The present invention is not limited to a His tag and Includes other tags such as those known to one of ordinary skill in the art, such as a fluorescent tag (e.g., GFP, YFP, etc.), etc.

### Antigen Delivery System

The present invention also features vaccine compositions in the form of an antigen delivery system. Any appropriate antigen delivery system may be considered for delivery of the antigens described herein. The present invention is not limited to the antigen delivery systems described herein.

In certain embodiments, the antigen delivery system is for targeted delivery of the vaccine composition, e.g., for targeting to the tissues of the body where the virus replicates.

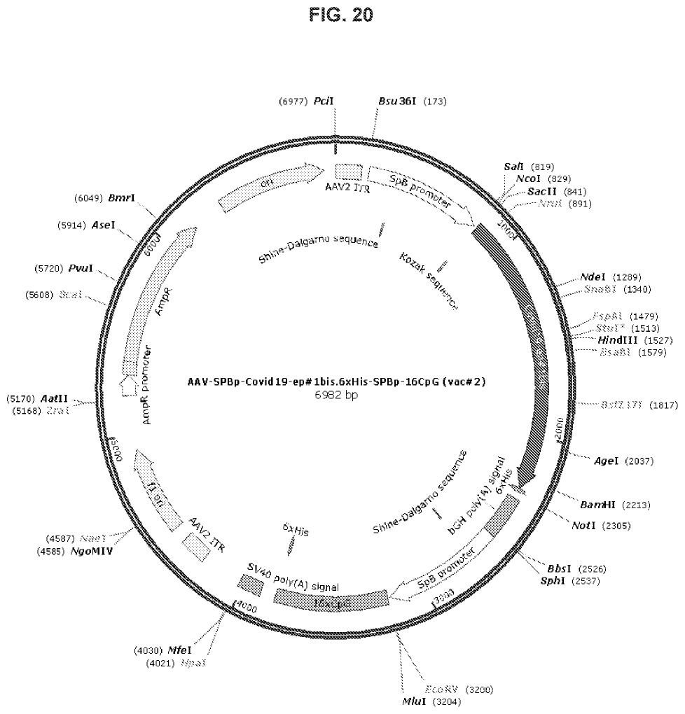

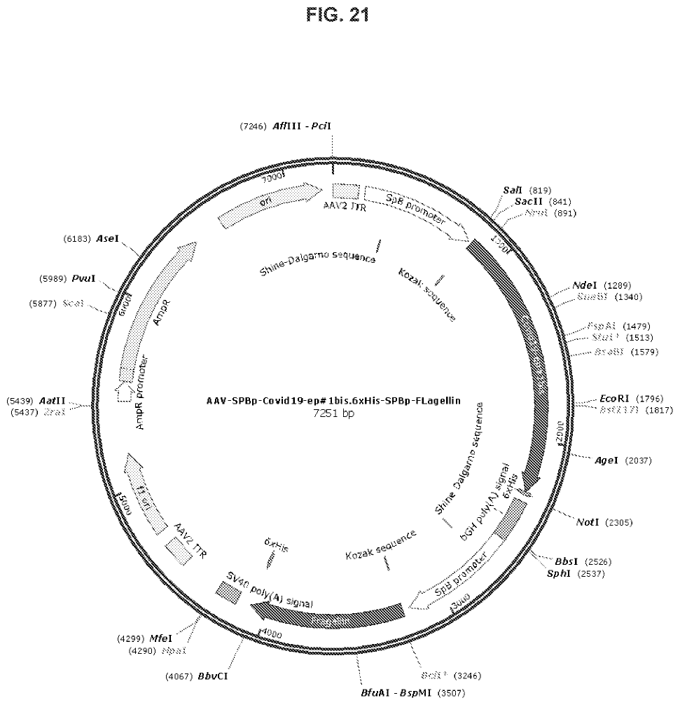

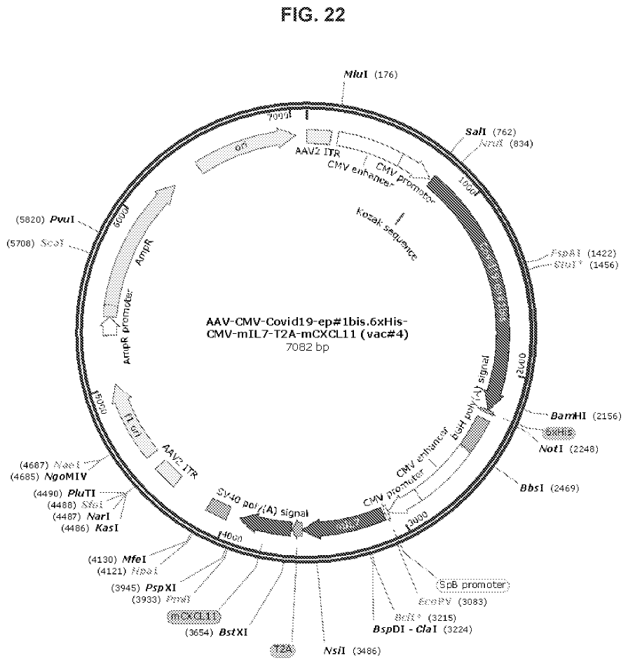

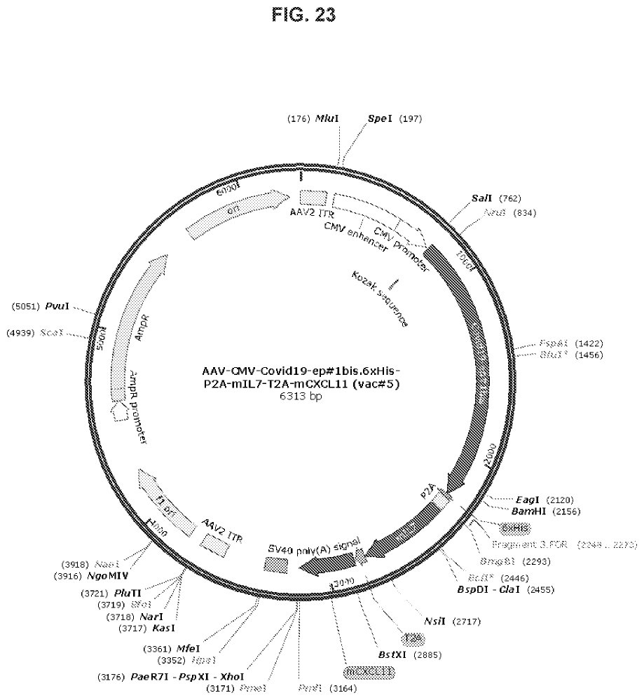

In certain embodiments, the antigen delivery system comprises an adeno-associated virus vector-based antigen delivery system, such as but not limited to the adeno-associated virus vector type 9 (AAV9 serotype), AAV type 8 (AAV8 serotype), etc. (see, for example, FIG. 20, FIG. 21, FIG. 22, and FIG. 23). In certain embodiments, the adeno-associated virus vectors used are tropic, e.g., tropic to lungs, brain, heart and kidney, e.g., the tissues of the body that express ACE2 receptors (FIG. 3A)). For example, AAV9 is known to be neurotropic, which would help the vaccine composition to be expressed in the brain.

The present invention is not limited to adeno-associated virus vector-based antigen delivery systems. Examples of other antigen delivery systems include: adenoviruses such as but not limited to Ad5, Ad26, Ad35, etc., as well as carriers such as lipid nanoparticles, polymers, peptides, etc. In other embodiments, the antigen delivery system comprises a vesicular stomatitis virus (VSV) vector.

In the antigen delivery system, the antigen or antigens (e.g., epitopes) are operatively linked to a promoter. In certain embodiments, the antigen or antigens (e.g., epitopes) are operatively linked to a generic promoter. For example, in certain embodiments, the antigen or antigens (e.g., epitopes) are operatively linked to a CMV promoter. In certain embodiments, the antigen or antigens (e.g., epitopes) are operatively linked to a CAG, EFIA, EFS, CBh, SFFV, MSCV, mPGK, hPGK, SV40, UBC, or other appropriate promoter.

In some embodiments, the antigen or antigens (e.g., epitopes) are operatively linked to a tissue-specific promoter (e.g., a lung-specific promoter). For example, the antigen or antigens (e.g., epitopes) are may be operatively linked to a SpB promoter or a CD144 promoter.

As discussed, in certain embodiments, the vaccine composition comprises a molecular adjuvant. In certain embodiments, the molecular adjuvant is operatively linked to a generic promoter, e.g., as described above. In certain embodiments, the molecular adjuvant is operatively linked to a tissue-specific promoter, e.g., a lung-specific promoter, e.g., SpB or CD144 (see FIG. 20, FIG. 21).

As discussed, in certain embodiments, the vaccine composition comprises a T cell attracting chemokine. In certain embodiments, the T cell attracting chemokine is operatively linked to a generic promoter, e.g., as described above. In certain embodiments, the T cell attracting chemokine is operatively linked to a tissue-specific promoter, e.g., a lung-specific promoter, e.g., SpB or CD144 (e.g., see FIG. 20).

As discussed, in certain embodiments, the vaccine composition comprises a composition for promoting T cell proliferation. In certain embodiments, the composition for promoting T cell proliferation is operatively linked to a generic promoter, e.g., as described above. In certain embodiments, the composition for promoting T cell proliferation is operatively linked to a tissue-specific promoter, e.g., a lung-specific promoter, e.g., SpB or CD144 (e.g., see FIG. 21).

Table 14 shows non-limiting examples of promoters that may be used to create a vaccine composition described herein.

TABLE 14

| | | SEQ |
|---|---|---|
| Promoter | Sequence | ID NO: |
| CAG | CTCGACATTGATTATTGACTAGTTATTAATAGTAATCAATTACGGGGTCATTAGTT | 167 |
| | CATAGCCCATATATGGAGTTCCGCGTTACATAACTTACGGTAAATGGCCCGCCTG | |
| | GCTGACCGCCCAACGACCCCCGCCCATTGACGTCAATAATGACGTATGTTCCCA | |
| | TAGTAACGCCAATAGGGACTTTCCATTGACGTCAATGGGTGGAGTATTTACGGTA | |
| | AACTGCCCACTTGGCAGTACATCAAGTGTATCATATGCCAAGTACGCCCCCTATT | |
| | GACGTCAATGACGGTAAATGGCCCGCCTGGCATTATGCCCAGTACATGACCTTA | |
| | TGGGACTTTCCTACTTGGCAGTACATCTACGTATTAGTCATCGCTATTACCATGG | |
| | TCGAGGTGAGCCCCACGTTCTGCTTCACTCTCCCCATCTCCCCCCCCTCCCCAC | |
| | CCCCAATTTTGTATTTATTTATTTTTTAATTATTTTGTGCAGCGATGGGGGCGGGG | |
| | GGGGGGGGGGGGCGCGCGCCAGGCGGGGCGGGGCGGGGCGAGGGGCGGG | |
| | GCGGGGCGAGGCGGAGAGGTGCGGCGGCAGCCAATCAGAGCGGCGCGCTCC | |
| | GAAAGTTTCCTTTTATGGCGAGGCGGCGGCGGCGGCGGCCCTATAAAAAGCGA | |
| | AGCGCGCGGCGGGCGGGAGTCGCTGCGCGCTGCCTTCGCCCCGTGCCCCGCT | |
| | CCGCCGCCGCCTCGCGCCGCCCGCCCCGGCTCTGACTGACCGCGTTACTCCCA | |
| | CAGGTGAGCGGGCGGGACGGCCCTTCTCCTCCGGGCTGTAATTAGCGCTTGGT | |
| | TTAATGACGGCTTGTTTCTTTTCTGTGGCTGCGTGAAAGCCTTGAGGGGCTCCG | |
| | GGAGGGCCCTTTGTGCGGGGGGAGCGGCTCGGGGGGTGCGTGCGTGTGTGTG | |
| | TGCGTGGGGAGCGCCGCGTGCGGCTCCGCGCTGCCCGGCGGCTGTGAGCGCT | |
| | GCGGGCGCGGCGCGGGGCTTTGTGCGCTCCGCAGTGTGCGCGAGGGGAGCG | |
| | CGGCCGGGGGCGGTGCCCCGCGGTGCGGGGGGGGCTGCGAGGGGAACAAAG | |
| | GCTGCGTGCGGGGTGTGTGCGTGGGGGGGTGAGCAGGGGGTGTGGGCGCGT | |
| | CGGTCGGGCTGCAACCCCCCCTGCACCCCCCTCCCCGAGTTGCTGAGCACGGC | |
| | CCGGCTTCGGGTGCGGGGCTCCGTACGGGGCGTGGCGCGGGGCTCGCCGTGC | |
| | CGGGCGGGGGGTGGCGGCAGGTGGGGGTGCCGGGCGGGGCGGGGCCGCCT | |
| | CGGGCCGGGGAGGGCTCGGGGGAGGGGCGCGGCGGCCCCCGGAGCGCCGG | |
| | CGGCTGTCGAGGCGCGGCGAGCCGCAGCCATTGCCTTTTATGGTAATCGTGCG | |
| | AGAGGGCGCAGGGACTTCCTTTGTCCCAAATCTGTGCGGAGCCGAAATCTGGG | |
| | AGGCGCCGCCGCACCCCCTCTAGCGGGCGCGGGGCGAAGCGGTGCGGCGCC | |
| | GGCAGGAAGGAAATGGGCGGGGAGGGCCTTCGTGCGTCGCCGCGCCGCCGTC | |
| | CCCTTCTCCCTCTCCAGCCTCGGGGCTGTCCGCGGGGGGACGGCTGCCTTCGG | |
| | GGGGGACGGGGCAGGGCGGGGTTCGGCTTCTGGCGTGTGACCGGCGGCTCTA | |
| | GAGCCTCTGCTAACCATGTTCATGCCTTCTTCTTTTTCCTACAGCTCCTGGGCAA | |
| | CGTGCTGGTTATTGTGCTGTCTCATCATTTTGGCAAAGAATTG | |
| CMV | TAGTTATTAATAGTAATCAATTACGGGGTCATTAGTTCATAGCCCATATATGGAGT | 168 |
| | TCCGCGTTACATAACTTACGGTAAATGGCCCGCCTGGCTGACCGCCCAACGACC | |
| | CCCGCCCATTGACGTCAATAATGACGTATGTTCCCATAGTAACGCCAATAGGGA | |
| | CTTTOCATTGACGTCAATGGGTGGAGTATTTACGGTAAACTGCCCACTTGGCAGT | |
| | ACATCAAGTGTATCATATGCCAAGTACGCCCCCTATTGACGTCAATGACGGTAAA | |
| | TGGCCCGCCTGGCATTATGCCCAGTACATGACCTTATGGGACTTTCCTACTTGG | |
| | CAGTACATCTACGTATTAGTCATCGCTATTACCATGGTGATGCGGTTTTGGCAGT | |
| | ACATCAATGGGCGTGGATAGCGGTTTGACTCACGGGGATTTCCAAGTCTCCACC | |
| | CCATTGACGTCAATGGGAGTTTGTTTTGGCACCAAAATCAACGGGACTTTCCAAA | |
| | ATGTCGTAACAACTCCGCCCCATTGACGCAAATGGGCGGTAGGCGTGTACGGTG | |
| | GGAGGTCTATATAAGCAGAGCTGGTTTAGTGAACCGTCAGATC | |
| SP-B | GTATAGGGCTGTCTGGGAGCCACTCCAGGGCCACAGAAATCTTGTCTCTGACTC | 169 |
| | AGGGTATTTTGTTTTCTGTTTTGTGTAAATGCTCTTCTGACTAATGCAAACCATGT | |
| | GTCCATAGAACCAGAAGATTTTTCCAGGGGAAAAGGTAAGGAGGTGGTGAGAGT | |
| | GTCCTGGGTCTGCCCTTCCAGGGCTTGCCCTGGGTTAAGAGCCAGGCAGGAAG | |
| | CTCTCAAGAGCATTGCTCAAGAGTAGAGGGGGCCTGGGAGGCCCAGGGAGGG | |
| | GATGGGAGGGGAACACCCAGGCTGCCCCCAACCAGATGCCCTCCACCCTCCTC | |
| | AACCTCCCTCCCACGGCCTGGAGAGGTGGGACCAGGTATGGAGGCTTGAGAGC | |
| | CCCTGGTTGGAGGAAGCCACAAGTCCAGGAACATGGGAGTCTGGGCAGGGGGC | |
| | AAAGGAGGCAGGAACAGGCCATCAGCCAGGACAGGTGGTAAGGCAGGCAGGA | |
| | GTGTTCCTGCTGGGAAAAGGTGGGATCAAGCACCTGGAGGGCTCTTCAGAGCA | |
| | AAGACAAACACTGAGGTCGCTGCCACTCCTACAGAGCCCCCACGCCCCGCCCA | |
| | GCTATAAGGGGCCATGCACCAAGCAGGGTACCCAGGCTGCAGAGGTGCC | |
| CD144 | CATCCATGCCCATGGCCTCAGATGCCAGCCATAAGCTGTTGGGTTCCAAACCTC | 170 |
| | GACTCCAGGCTGGACTCACCCCTGTCTCCCCCACCAGCCTGACACCTCCACCTG | |
| | GGTATCTAACGAGCATCTCAAACTCAACCTGCCTGAGACAGAGGAATCACTATCC | |
| | CCTCCTCCTCCAAAAATATCCTTCCATCACACTCCCCATCTTGTGCTCTGATTTAC | |
| | TAAACGGCCCTGGGCCCTCTCTTTCTCAGGGTCTCTGCTTGCCCAGCTATATAAT | |
| | AAAACAAGTTTGGGACTTCCCAACCATTCACCCATGGAAAAACAGAAGCAACTCT | |
| | TCAAAGGACAGATTCCCAGGATCTGCCCTGGGAGATTCCAAATCAGTTGATCTG | |
| | GGGTGAGCCCAGTCCTCTGTAGTTTTTAGAAGCTCCTCCTATGTCTCTCCTGGTC | |
| | AGCAGAATCTTGGCCCCTCCCTTCCCCCCAGCCTCTTGGTTCTTCTGGGCTCTG | |
| | ATCCAGCCTCAGCGTCACTGTCTTCCACGCCCCTCTTTGATTCTCGTTTATGTCA | |
| | AAAGCCTTGTGAGGATGAGGCTGTGATTATCCCCATTTTACAGATGAGGAAACTG | |
| | TGGCTCCAGGATGACACAACTGGCCAGAGGTCACATCAGAAGCAGAGCTGGGT | |
| | CACTTGACTCCACCCAATATCCCTAAATGCAAACATCCCCTACAGACCGAGGCTG | |
| | GCACCTTAGAGCTGGAGTCCATGCCCGCTCTGACCAGGAGAAGCCAACCTGGT | |
| | CCTCCAGAGCCAAGAGCTTCTGTCCCTTTCCCATCTCCTGAAGCCTCCCTGTCA | |
| | CCTTTAAAGTCCATTCCCACAAAGACATCATGGGATCACCACAGAAAATCAAGCT | |
| | CTGGGGCTAGGCTGACCCCAGCTAGATTTTTGGCTCTTTTATACCCCAGCTGGG | |
| | TGGACAAGCACCTTAAACCCGCTGAGCCTCAGCTTCCCGGGCTATAAAATGGGG | |
| | GTGATGACACCTGCCTGTAGCATTCCAAGGAGGGTTAAATGTGATGCTGCAGCC | |
| | AAGGGTCCCCACAGCCAGGCTCTTTGCAGGTGCTGGGTTCAGAGTCCCAGAGC | |
| | TGAGGCCGGGAGTAGGGGTTCAAGTGGGGTGCCCCAGGCAGGGTCCAGTGCC | |
| | AGCCCTCTGTGGAGACAGCCATCCGGGGCCGAGGCAGCCGCCCACCGCAGGG | |
| | CCTGCCTATCTGCAGCCAGCCCAGCCCTCACAAAGGAACAATAACAGGAAACCA | |
| | TCCCAGGGGGAAGTGGGCCAGGGCCAGCTGGAAAACCTGAAGGGGAGGCAGC | |
| | CAGGCCTCCCTCGCCAGCGGGGTGTGGCTCCCCTCCAAAGACGGTCGGCTGAC | |
| | AGGCTCCACAGAGCTCCACTCACGCTCAGCCCTGGACGGACAGGCAGTCCAAC | |
| | GGAACAGAAACATCCCTCAGCCCACAGGCACGGTGAGTGGGGGCTCCCACACT | |
| | CCCCTCCACCCCAAACCCGCCACCCTGCGCCCAAGATGGGAGGGTCCTCAGCT | |
| | TCCCCATCTGTAGAATGGGCATCGTCCCACTCCCATGACAGAGAGGCTCC | |
| wild type | ATGTTCGTGTTCCTGGTGCTGCTGCCCCTGGTGAGCAGC | 171 |
| native | | |
| leader | | |
| sequence | | |

In certain embodiments, the T cell attracting chemokine and the composition that promotes T cell proliferation are driven by the same promoter (e.g., the T cell attracting chemokine and the composition that promotes T cell proliferation are synthesized as a peptide). In certain embodiments, the T cell attracting chemokine and the composition that promotes T cell proliferation are driven by different promoters. In certain embodiments, the antigen, the T cell attracting chemokine, and the composition that promotes T cell proliferation are driven by the same promoter. In certain embodiments, the antigen or antigens, the T cell attracting chemokine, and the composition that promotes T cell proliferation are driven by the different promoters. In certain embodiments, the T cell attracting chemokine and the composition that promotes T cell proliferation are driven by the same promoter, and the antigen or antigens are driven by a different promoter.

In some embodiments, the antigen delivery system comprises one or more linkers between the T cell attracting chemokine and the composition that promotes T cell proliferation. In certain embodiments, linkers are used between one or more of the epitopes. The linkers may allow for cleavage of the separate molecules (e.g., chemokine). For example, in some embodiments, a linker is positioned between IL-7 (or IL-2) and CCL5, CXCL9, CXCL10, CXCL11, CCL25, CCL28, CXCL14, CXCL17, etc. In some embodiments, a linker is positioned between IL-15 and CCL5, CXCL9, CXCL10, CXCL11, CCL25, CCL28, CXCL14, CXCL17, etc. In some embodiments, a linker is positioned between the antigen and another composition, e.g., IL-15, IL-7, IL-2, CCL5, CXCL9, CXCL10, CXCL11, CCL25, CCL28, CXCL14, CXCL17, etc. A non-limiting example of a linker is T2A, E2A, P2A (see Table 15), or the like (e.g., see FIG. 22). The composition may feature a different linker between each open reading frame.

TABLE 15

| | | SEQ |
|---|---|---|
| | SEQUENCE | ID NO: |
| T2A Linker | GGAAGCGGAGAGGGCAGGGGAAGTCTTCTAACATGCGGGGACGTGG | 142 |
| | AGGAAAATCCCGGCCCC | |
| E2A Linker | GGAAGCGGACAGTGTACTAATTATGCTCTCTTGAAATTGGCTGGAGAT | 152 |
| | GTTGAGAGCAACCCAGGTCCC | |
| P2A Linker | GGAAGCGGAGCCACGAACTTCTCTCTGTTAAAGCAAGCAGGAGATGT | 180 |
| | TGAAGAAAACCCCGGGCCT | |
| 6-His Tag | CATCACCATCACCATCAC | 181 |

The present invention includes mRNA sequences encoding any of the vaccine compositions or portions thereof herein. The present invention also includes modified mRNA sequences encoding any of the vaccine compositions or portions thereof herein. The present invention also includes DNA sequence encoding any of the vaccine compositions or portions thereof herein.

In certain embodiments, nucleic acids of a vaccine composition herein are chemically modified. In some embodiments, the nucleic acids of a vaccine composition therein are unmodified. In some embodiments, all or a portion of the uracil in the open reading frame has a chemical modification. In some embodiments, a chemical modification is in the 5-position of the uracil. In some embodiments, a chemical modification is a N1-methyl pseudouridine. In some embodiments, all or a portion of the uracil in the open reading frame has a N1-methyl pseudouridine in the 5-position of the uracil.

In certain embodiments, an open reading frame of a vaccine composition herein encodes one antigen or epitopes. In some embodiments, an open reading frame of a vaccine composition herein encodes two or more antigens or epitopes. In some embodiments, an open reading frame of a vaccine composition herein encodes five or more antigens or epitopes. In some embodiments, an open reading frame of a vaccine composition herein encodes ten or more antigens or epitopes. In some embodiments, an open reading frame of a vaccine composition herein encodes 50 or more antigens or epitopes.

### Epitope Arrangements

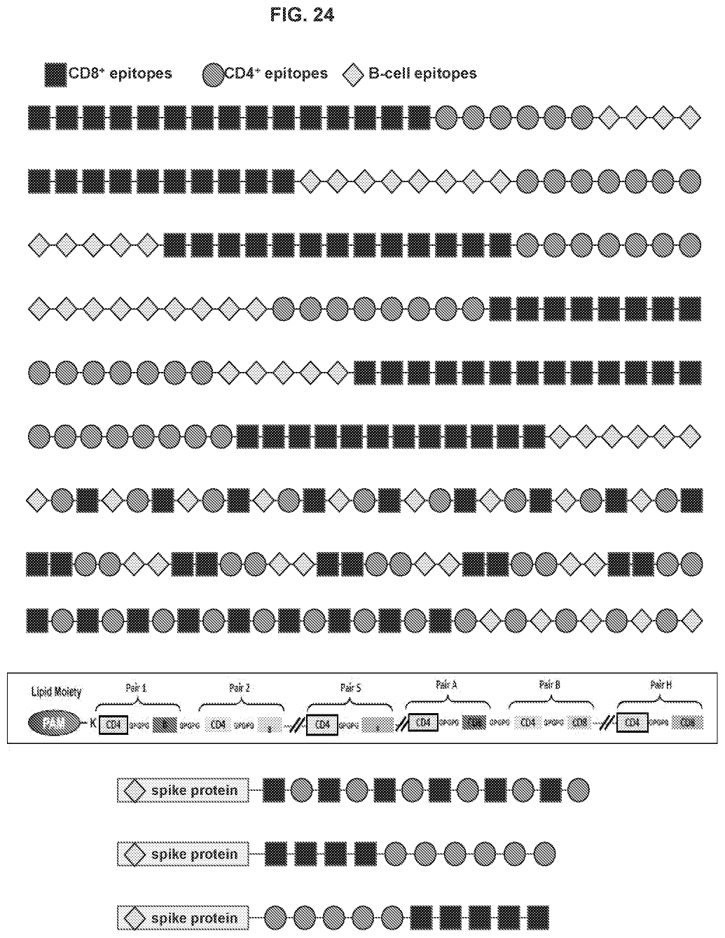

The target epitopes of the compositions described may be arranged in various configurations (see, for example, FIG. 24 and FIG. 19). In some embodiments, the target epitopes may be arranged such that one or more CD8+ T cell epitopes are followed by one or more CD4+ T cell epitopes followed by one or more B cell epitopes. In some embodiments, the target epitopes may be arranged such that one or more CD8+ T cell epitopes are followed by one or more B cell epitopes followed by one or more CD4+ T cell epitopes. In other embodiments, the target epitopes may be arranged such that one or more CD4+ T cell epitopes are followed by one or more CD8+ T cell epitopes followed by one or more B cell epitopes. In other embodiments, the target epitopes may be arranged such that one or more CD4+ T cell epitopes are followed by one or more B cell epitopes followed by one or more CD8+ T cell epitopes. In further embodiments, the target epitopes may be arranged such that one or more B cell epitopes are followed by one or more CD4+ T cell epitopes followed by one or more CD8+ T cell epitopes. In other embodiments, the target epitopes may be arranged such that one or more B cell epitopes are followed by one or more CD8+ T cell epitopes followed by one or more CD4+ T cell epitopes.

In some embodiments, the target epitopes may be arranged such that one or more pairs of CD4+-CD8+ T cell epitopes are followed by one or more pairs of CD4+ T cell-B cell epitopes. In other embodiments, the target epitopes may be arranged such that CD8+ T cell, CD4+ T cell, and B cell epitopes are repeated one or more times.

In other embodiments, the target epitopes may be arranged such that one or more CD4+ T cell epitopes are followed by one or more CD8+ T cell epitopes. In embodiments, the target epitopes may be arranged such that one or more CD8+ T cell epitopes are followed by one or more CD4+ T cell epitopes. In some embodiments, the target epitopes may be arranged such that one or more CD4+ T cell epitopes are followed by one or more B cell target epitopes. In some embodiments, the target epitopes may be arranged such that one or more CD8+ T cell epitopes are followed by one or more B cell target epitopes. In other embodiments, the target epitopes may be arranged such that one or more B cell epitopes are followed by one or more CD4+ T cell target epitopes. In some embodiments, the target epitopes may be arranged such that one or more B cell epitopes are followed by one or more CD8+ T cell target epitopes.

Likewise, the other components of the vaccine composition may be arranged in various configurations. For example, in certain embodiments, the T cell attracting chemokine is followed by the composition for promoting T cell proliferation. In certain embodiments, the composition for promoting T cell proliferation is followed by the T cell attracting chemokine.

### Methods

The present invention also features methods for designing and/or producing a pan-coronavirus composition. Briefly, the method may comprise determining target epitopes, selecting desired target epitopes (e.g., two or more, etc.), and synthesizing an antigen comprising the selected target epitopes. The method may comprise determining target epitopes, selecting desired target epitopes, and synthesizing a nucleotide composition (e.g., DNA, modified DNA, mRNA, modified mRNA, antigen delivery system, etc.) encoding the antigen comprising the selected target epitopes. In some embodiments, the method further comprises creating a vaccine composition comprising the antigen, nucleotide compositions, and/or antigen delivery system and a pharmaceutical carrier.

The methods herein may also include the steps of designing the antigen delivery system. For example, the methods may comprise inserting molecular adjuvants, chemokines, linkers, tags, etc. into the antigen delivery system. In some embodiments, one or more components is inserted into a different antigen delivery system from the antigen or antigens (e.g., the epitopes). For example, the present invention provides embodiments wherein the antigen or antigens (e.g., the epitopes) are within a first antigen delivery system and one or more additional components (e.g., chemokine, etc.) are within a second delivery system. In some embodiments, the antigen or antigens (e.g., the epitopes) and one or more additional components are within a first delivery system, and one or more additional components are within a second delivery system. In some embodiments, the antigen or antigens (e.g., the epitopes) and one or more additional components are within a first delivery system, and the antigen or antigens (e.g., the epitopes) and one or more additional components are within a second delivery system.

In some embodiments, the method comprises determining target epitopes from at least two of the following 1. coronavirus B-cell epitopes, 2. coronavirus CD4+ T cell epitopes, and/or 3. coronavirus CD8+ T cell epitopes. In some embodiments, each of the target epitopes are mutated epitopes, e.g., as described herein. For example, the target epitopes may be mutated among two or a combination of at least one SARS-CoV-2 human strains in current circulation, at least one coronavirus that has caused a previous human outbreak, at least one coronavirus isolated from bats, at least one coronavirus Isolated from pangolin, at least one coronavirus isolated from civet cats, at least one coronavirus strain isolated from mink, and at least one coronavirus strain isolated from camels or any other animal that is receptive to coronavirus. In some embodiments, the composition comprises at least two of the following: one or more coronavirus B-cell target epitopes, one or more coronavirus CD4⁺ T cell target epitopes, and/or one or more coronavirus CD8⁺ T cell target epitopes.

In certain embodiments, the method comprises selecting at least one epitope from at least two of: one or more mutated coronavirus B-cell epitopes; one or more mutated coronavirus CD4+ T cell epitopes; and one or more mutated coronavirus CD8+ T cell epitopes: and synthesizing an antigen comprising the selected epitopes. In certain embodiments, the method comprises selecting at least one epitope from at least two of: one or more mutated coronavirus B-cell epitopes; one or more mutated coronavirus CD4+ T cell epitopes; and one or more mutated coronavirus CD8+ T cell epitopes; and synthesizing an antigen delivery system that encodes an antigen comprising the selected epitopes.

In some embodiments, the method comprises determining one or more mutated large sequences that are derived from coronavirus sequences (e.g., SARS-CoV-2, variants, common cold coronaviruses, previously known coronavirus strains, animal coronaviruses, etc.). The method may comprise selecting at least one large mutated sequence and synthesizing an antigen comprising the selected large mutated sequence(s). The method may comprise synthesizing a nucleotide composition (e.g., DNA, modified DNA. mRNA, modified mRNA, antigen delivery system, etc.) encoding the antigen comprising the selected large mutated sequence(s). In some embodiments, the method further comprises creating a vaccine composition comprising the antigen, nucleotide compositions, and/or antigen delivery system and a pharmaceutical carrier. In some embodiments, the large sequences comprise one or more mutated epitopes described herein, e.g., one or more mutated B-cell target epitopes and/or one or more mutatedCD4+ T cell target epitopes and/or one or more mutatedCD8+ T cell target epitopes.

In some embodiments, each of the large sequences are mutated among two or a combination of: at least two SARS-CoV-2 human strains in current circulation, at least one coronavirus that has caused a previous human outbreak, at least one coronavirus isolated from bats, at least one coronavirus isolated from pangolin, at least one coronavirus Isolated from civet cats, at least one coronavirus strain isolated from mink, and at least one coronavirus strain isolated from camels or any other animal that is receptive to coronavirus.

As previously discussed, the compositions described herein, e.g., the epitopes, the vaccine compositions, the antigen delivery systems, the chemokines, the adjuvants, etc. may be used to prevent a coronavirus disease in a subject. In some embodiments, the compositions described herein, e.g., the antigen or antigens (e.g., epitopes), the vaccine compositions, the antigen delivery systems, the chemokines, the adjuvants, etc. may be used to prevent a coronavirus infection prophylactically in a subject. In some embodiments, the compositions described herein, e.g., the epitopes, the vaccine compositions, the antigen delivery systems, the chemokines, the adjuvants, etc. may elicit an immune response in a subject. In some embodiments, the compositions described herein, e.g., the epitopes, the vaccine compositions, the antigen delivery systems, the chemokines, the adjuvants, etc. may prolong an immune response induced by the multi-epitope pan-coronavirus vaccine composition and increases T-cell migration to the lungs.

Methods for preventing a coronavirus disease in a subject may comprise administering to the subject a therapeutically effective amount of a pan-coronavirus vaccine composition according to the present invention. In some embodiments, the composition elicits an immune response in the subject. In some embodiments, the composition induces memory B and T cells. In some embodiments, the composition induces resident memory T cells (Tᵣₘ). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response. e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for preventing a coronavirus infection prophylactically in a subject may comprise administering to the subject a prophylactically effective amount of a pan-coronavirus vaccine composition according to the present invention. In some embodiments, the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition Improves homing and retention of T cells, e.g., in the areas where the virus normally replicates such as lungs, brain, heart, and kidney.

Methods for eliciting an immune response in a subject may comprise administering to the subject a vaccine composition according to the present invention, wherein the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents Inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for prolonging an immune response induced by a vaccine composition of the present invention and increasing T cell migration to particular tissues (e.g., lung, brain, heart, kidney, etc.) may comprise co-expressing a T-cell attracting chemokine, a composition that promotes T cell proliferation, and a vaccine composition (e.g., antigen) according to the present invention.

Methods for prolonging the retention of memory T-cell into the lungs induced by a vaccine composition of the present invention and increasing virus-specific tissue resident memory T-cells (TRM cells) may comprise co-expressing a T-cell attracting chemokine, a composition that promotes T cell proliferation, and a vaccine composition (e.g., antigen) according to the present invention.

The vaccine composition may be administered through standard means, e.g., through an intravenous route (i.v.), an Intranasal route (i.n.), or a sublingual route (s.l.) route.

In certain embodiments, the method comprises administering to the subject a second (e.g., booster) dose. The second dose may comprise the same vaccine composition or a different vaccine composition. Additional doses of one or more vaccine compositions may be administered.

### Sequential Vaccine Delivery Methodology

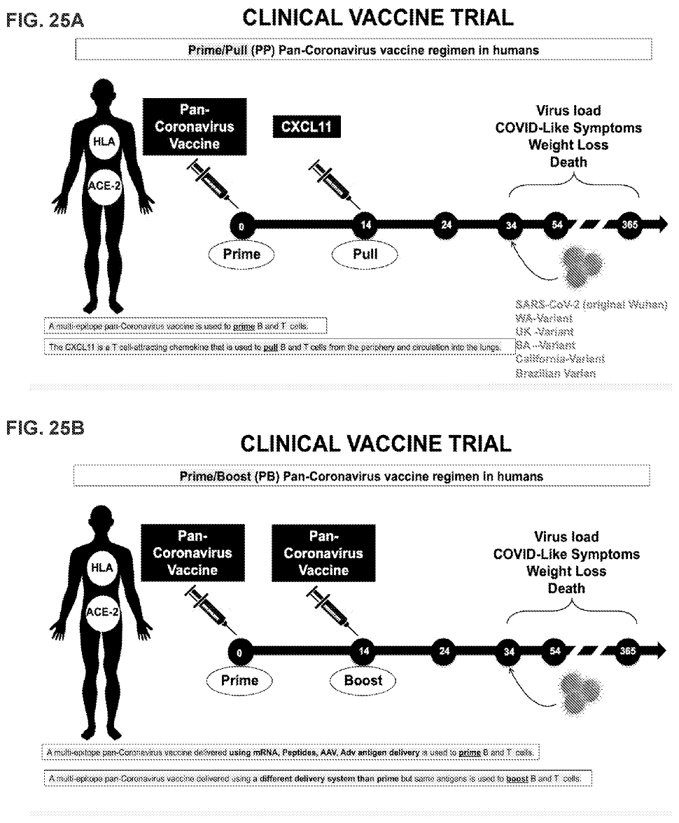

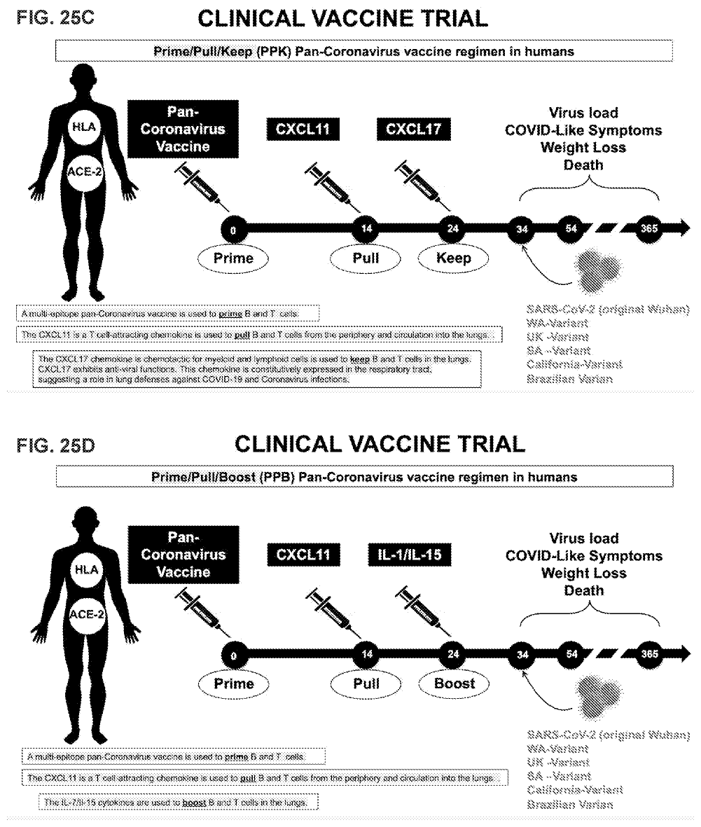

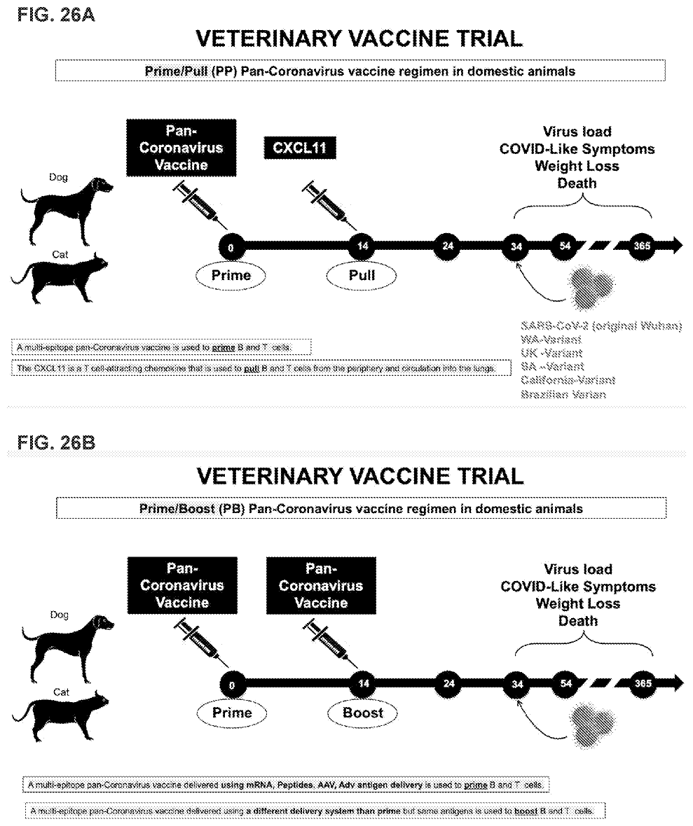

In some embodiments, the present invention features a method of delivering the vaccine to induce heterologous immunity in a subject (e.g., prime/boost, see FIG. 25B and FIG. 26B). In some embodiments, the method comprises administering a first composition, e.g., a first pan-coronavirus recombinant vaccine composition dose using a first delivery system and further administering a second composition, e.g., a second vaccine composition dose using a second delivery system. In other embodiments, the first delivery system and the second delivery system are different. In some embodiments, the second composition is administered 8 days after administration of the first composition. In some embodiments, the second composition is administered 9 days after administration of the first composition. In some embodiments, the second composition is administered 10 days after administration of the first composition. In some embodiments, the second composition is administered 11 days after administration of the first composition. In some embodiments, the second composition is administered 12 days after administration of the first composition. In some embodiments, the second composition is administered 13 days after administration of the first composition. In some embodiments, the second composition is administered 14 days after administration of the first composition. In some embodiments, the second composition is administered from 14 to 30 days after administration of the first composition. In some embodiments, the second composition is administered from 30 to 60 days after administration of the first composition.

In some embodiments, the first delivery system or the second delivery system comprises an mRNA, a modified mRNA or a peptide vector. In other embodiments, the peptide vector comprises adenovirus or an adeno-associated virus vector.

In some embodiments, the present invention features a method of delivering the vaccine to induce heterologous immunity in a subject (e.g., prime/pull, see FIG. 25A and FIG. 26A). In some embodiments, the method comprises administering a pan-coronavirus recombinant vaccine composition and further administering at least one T-cell attracting chemokine after administering the pan-coronavirus recombinant vaccine composition. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition.

The present invention also features a novel “prime, pull, and boost” strategy. In other embodiments, the present invention features a method to increase the size and maintenance of lung-resident B-cells, CD4+ T cells and CD8+ T cells to protect against SARS-CoV-2 (FIG. 25D and FIG. 26D). In some embodiments, the method comprises administering a pan-coronavirus recombinant vaccine composition and administering at least one T-cell attracting chemokine after administering the pan-coronavirus recombinant vaccine composition. In some embodiments, the method further comprises administering at least one cytokine after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the cytokine is administered 8 days after administering the T-cell attracting chemokine. In some embodiments, the cytokine is administered 9 days after administering the T-cell attracting chemokine. In some embodiments, the cytokine is administered 10 days after administering the T-cell attracting chemokine. In some embodiments, the cytokine is administered 11 days after administering the T-cell attracting chemokine. In some embodiments, the cytokine is administered 12 days after administering the T-cell attracting chemokine. In some embodiments, the cytokine is administered 13 days after administering the T-cell attracting chemokine. In some embodiments, the cytokine is administered 14 days after administering the T-cell attracting chemokine. In some embodiments, the cytokine is administered from 14 to 30 days after administering the T-cell attracting chemokine. In some embodiments, the cytokine is administered from 30 to 60 days after administering the T-cell attracting chemokine.

The present invention further features a novel “prime, pull, and keep” strategy (FIG. 25C and FIG. 26C). For example, the present invention features a method to increase the size and maintenance of lung-resident B-cells, CD4+ T cells and CD8+ T cells to protect against SARS-CoV-2. In some embodiments, the method comprises administering a pan-coronavirus recombinant vaccine composition and administering at least one T-cell attracting chemokine after administering the pan-coronavirus recombinant vaccine composition. In some embodiments, the method further comprises administering at least one mucosal chemokine after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the mucosal chemokine is administered 8 days after administering the T-cell attracting chemokine. In some embodiments, the mucosal chemokine is administered 9 days after administering the T-cell attracting chemokine. In some embodiments, the mucosal chemokine is administered 10 days after administering the T-cell attracting chemokine. In some embodiments, the mucosal chemokine is administered 11 days after administering the T-cell attracting chemokine. In some embodiments, the mucosal chemokine is administered 12 days after administering the T-cell attracting chemokine. In some embodiments, the mucosal chemokine is administered 13 days after administering the T-cell attracting chemokine. In some embodiments, the mucosal chemokine is administered 14 days after administering the T-cell attracting chemokine. In some embodiments, the mucosal chemokine is administered from 14 to 30 days after administering the T-cell attracting chemokine. In some embodiments, the mucosal chemokine is administered from 30 to 60 days after administering the T-cell attracting chemokine.

In some embodiments, the mucosal chemokines may comprise CCL25, CCL28, CXCL14, CXCL17, or a combination thereof. In some embodiments, the T-cell attracting chemokines may comprise CCL5, CXCL9, CXCL10, CXCL11, or a combination thereof. In some embodiments, the cytokines may comprise IL-15, IL-7, IL-2, or a combination thereof.

In some embodiments, the efficacy (or effectiveness) of a vaccine composition herein is greater than 60%. In some embodiments, the efficacy (or effectiveness) of a vaccine composition herein is greater than 70%. In some embodiments, the efficacy (or effectiveness) of a vaccine composition herein is greater than 80%. In some embodiments, the efficacy (or effectiveness) of a vaccine composition herein is greater than 90%. In some embodiments, the efficacy (or effectiveness) of a vaccine composition herein is greater than 95%.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU−ARV)/ARUx100; and Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100.

In some embodiments, the vaccine immunizes the subject against a coronavirus for up to 1 year. In some embodiments, the vaccine immunizes the subject against a coronavirus for up to 2 years. In some embodiments, the vaccine immunizes the subject against a coronavirus for more than 1 year, more than 2 years, more than 3 years, more than 4 years, or for 5-10 years.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a vaccine may be administered later in life, for example, at the age of about 6 months to about 5 years, or older.

In some embodiments, the subject is pregnant (e.g., in the first, second or third trimester) when administered a vaccine.

In some embodiments, the subject has a chronic pulmonary disease (e.g., chronic obstructive pulmonary disease (COPD) or asthma) or is at risk thereof. Two forms of COPD include chronic bronchitis, which involves a long-term cough with mucus, and emphysema, which involves damage to the lungs over time. Thus, a subject administered a vaccine may have chronic bronchitis or emphysema.

In some embodiments, the subject has been exposed to a coronavirus. In some embodiments, the subject is infected with a coronavirus. In some embodiments, the subject is at risk of infection by a coronavirus.

In some embodiments, the subject is immunocompromised (has an impaired immune system, e.g., has an immune disorder or autoimmune disorder).

### Pharmaceutical Carriers

In certain embodiments, the vaccine composition further comprises a pharmaceutical carrier. Pharmaceutical carriers are well known to one of ordinary skill in the art. For example, in certain embodiments, the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof. In some embodiments, the pharmaceutical carrier may comprise a lipid nanoparticle, an adenovirus vector, or an adeno-associated virus vector. In some embodiments, the vaccine composition is constructed using an adeno-associated virus vectors-based antigen delivery system.

Also provided herein is vaccine of any one of the foregoing paragraphs, formulated in a nanoparticle (e.g., a lipid nanoparticle). In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoy)oxy)heptadecanedioate (L319).

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-514. (canceled)

515. A coronavirus recombinant vaccine composition, the composition comprising at least two of:
a. one or more coronavirus B-cell target epitopes;
b. one or more coronavirus CD4⁺ T cell target epitopes;
c. one or more coronavirus CD8⁺ T cell target epitopes;
wherein at least one epitope has a mutation as compared to its corresponding epitope in SARS-CoV-2 isolate Wuhan-Hu-1, wherein the epitopes are derived from a human coronavirus, an animal coronavirus, or a combination thereof; wherein at least one epitope is derived from a non-spike protein.

516. The composition of claim 515, wherein the human coronavirus is SARS-CoV-2 original strain or a SARS-CoV-2 variant and wherein the animal coronavirus is a bat coronavirus, a pangolin coronavirus, a civet cat coronavirus, a mink coronavirus, a camel coronavirus, or a coronavirus from another animal susceptible to coronavirus infection.

517. The composition of claim 515, wherein non-spike proteins are encoded by ORF1ab, ORF3a, ORF6, ORF7a, ORF7b, ORF8, ORF10, or Envelope protein, Membrane protein, Nucleocapsid protein.

518. The composition of claim 515, wherein one or more of the at least two target epitopes is in the form of a large sequence, wherein the large sequence is a whole protein expressed by SARS-CoV-2 or a SARS-CoV-2 variant, or derive from a partial protein sequence expressed by SARS-CoV-2 or a SARS-CoV-2 variant, or a combination thereof.

519. The composition of claim 518, wherein the large sequence is selected from a group consisting of SEQ ID NO: 143-151.

520. The composition of claim 515, wherein target epitopes are derived from a SARS-CoV-2 protein selected from a group consisting of: proteins encoded by ORF1ab, ORF3a, ORF6, ORF7a, ORF7b, ORF8, ORF10, or an Envelope protein, a Membrane protein, a Nucleocapsid protein, and a Spike protein.

521. The composition of claim 515, wherein the mutated epitopes are derived from one or more of: one or more SARS-CoV-2 human strains or variants in current circulation; one or more coronaviruses that have caused a previous human outbreak; one or more coronaviruses isolated from animals selected from a group consisting of bats, pangolins, civet cats, minks, camels, and other animals receptive to coronaviruses; or one or more coronaviruses that cause the common cold, wherein the one or more SARS-CoV-2 human strains or variants in current circulation are selected from: strain B.1.177; strain B.1.160, strain B.1.1.7; strain B.1.351; strain P.1; strain B.1.427/B.1.429; strain B.1.258; strain B.1.221; strain B.1.367; strain B.1.1.277; strain B.1.1.302; strain B.1.525; strain B.1.526, strain S:677H, and strain S:677P, and wherein the one or more coronaviruses that cause the common cold are selected from: 229E alpha coronavirus, NL63 alpha coronavirus, OC43 beta coronavirus, and HKU1 beta coronavirus.

522. The composition of claim 515, wherein the one or more coronavirus CD8+ T cell target epitopes are selected from: SEQ ID NO: 2-29, SEQ ID NO: 30-57, SEQ ID NO: 153, or a combination thereof, wherein the one or more coronavirus CD4+ T cell target epitopes are selected from: SEQ ID NO: 58-73, SEQ ID NO: 74-105, SEQ ID NO: 154, or a combination thereof, and wherein one or more coronavirus B-cell target epitopes are selected from: SEQ ID NO: 106-116, SEQ ID NO: 117-138, SEQ ID NO: 155, SEQ ID NO: 172-178, or a combination thereof.

523. The composition of claim 515, wherein the mutated epitope is in a spike (S) protein, wherein the mutation is one or a combination of A22V, S477N, H69-, V70-, Y144-, N501Y, A570D, P681H, D80A, D215G, L241-, L242-, A243-, K417N, E484K, N501Y, A701V, L18F, K417T, E484K, N501Y, H655Y, S13I, W152C, L452R, S439K, S98F, D80Y, A626S, V1122L, A67V, H69-, V70-, Y144-, E484K, Q677H, F888L, L5F, T95I, D253G, E484K, A701V, Q677H, or Q677P.

524. The composition of claim 515, wherein the mutated epitope is in a nucleocapsid (N) protein, wherein the mutation is one or a combination of A220V, M234I, A376T, R203K, G204R, T205I, P80R, R203K, G204R, P199L, S186Y, D377Y, S2-, D3Y, A12G, P199L, M234I, P67S, P199L, D377Y, P67S, or P199L.

525. The composition of claim 515, wherein the mutated epitope is in an Envelope (E) protein, wherein the mutation is P71L.

526. The composition of claim 515, wherein the mutated epitope is in a protein encoded by ORF3a, wherein the mutation is one or a combination of Q38R, G172R, V202L, or P42L.

527. The composition of claim 515, wherein the mutated epitope is in a protein encoded by ORF7a, wherein the mutation is R80I.

528. The composition of claim 515, wherein the mutated epitope is in a protein encoded by ORF8, wherein the mutation is Q27*, T11I, or a combination thereof.

529. The composition of claim 515, wherein the mutated epitope is in a protein encoded by ORF10, wherein the mutation is V30L.

530. The composition of claim 515, wherein the mutated epitope is in a protein encoded by ORF1b protein, wherein the mutation is one or a combination of A176S, V767L, K1141R, E1184D, D1183Y, P255T, Q1011H, N1653D, R2613C, N1653D, or R2613C.

531. The composition of claim 515, wherein the mutated epitope is in a protein encoded by ORF1a protein, wherein the mutation is one or a combination of S3675-, G3676-, F3677-, S3675-, G3676-, F3677-, S3675-, G3676-, F3677-, 14205V, I2501T, T945I, T1567I, Q3346K, V3475F, M3862I, S3675-, G3676-, F3677-, S3675-, G3676-, F3677-, T265I, L3352F, T265I, or L3352F.

532. The composition of claim 515 further comprising a T cell attracting chemokine, wherein the T cell attracting chemokine is CCL5, CXCL9, CXCL10, CXCL11, or a combination thereof.

533. The composition of claim 515 further comprising a composition that promotes T cell proliferation and T-cell memory, wherein the composition that promotes T cell proliferation and memory is IL-7, IL-2, or IL-15.

534. The composition of claim 515, wherein the composition comprises one of SEQ ID NO: 139-141.
